(19) United States
(12) Patent Application Publication
(10) Pub. No.: US 2019/0071668 A1
(43) Pub. Date: Mar. 7, 2019

(54) COMPOSITIONS AND METHODS FOR IDENTIFICATION, ASSESSMENT, PREVENTION, AND TREATMENT OF CANCER USING SLNCR ISOFORMS

(71) Applicant: Dana-Farber Cancer Institute, Inc., Boston, MA (US)

(72) Inventors: Karyn Schmidt, Brighton, MA (US); Carl Novina, Newton, MA (US)

(21) Appl. No.: 15/741,317

(22) PCT Filed: Jul. 7, 2016
(86) PCT No.: PCT/US16/41343

§ 371 Date: Jan. 2, 2018

Related U.S. Application Data
(60) Provisional application No. 62/190,023, filed on Jul. 8, 2015.
(60) Provisional application No. 62/319,902, filed on Apr. 8, 2016.

(51) Int. Cl. C12N 15/113; C12N 15/11; C12N 15/62; C12Q 1/6886; A61P 35/02

(52) U.S. Cl. CPC C12N 15/113; C12N 15/111; C12N 15/62; C12Q 1/6886; A61P 35/02; C12N 2320/30; C12N 2310/113; C12N 2310/3231; C12N 2310/3233; C12N 2310/3525; C12Q 2600/136

(57) ABSTRACT

The present invention relates to compositions and methods for identifying, assessing, preventing, and treating cancer and modulating immune responses using SLNCR isoforms.

### CROSS-REFERENCE TO RELATED APPLICATIONS

This application claims the benefit of priority to U.S. Provisional Application No. 62/190,023, filed 8 Jul. 2015, and U.S. Provisional Application No. 62/319,902, filed 8 Apr. 2016, the entire contents of each of said applications are incorporated herein in their entirety by this reference.

### STATEMENT OF RIGHTS

This invention was made with government support under Grants R01 CA140986 and T32 AI007386 awarded by the National Institutes of Health. The U.S. government has certain rights in the invention.

### BACKGROUND OF THE INVENTION

Long noncoding RNAs (LncRNAs) play integral structural and functional roles in the cell, particularly by coordinating complex gene expression patterns in a highly regulated fashion. Dysregulated lncRNA expression has recently been linked to many complex human diseases, including various cancers (Li et al. (2013) Intl. J. Mol. Sci. 14:18790-18808). LncRNAs may act as either oncogenes or tumor suppressors and play an emerging role specifically in cancer metastasis (Serviss et al. (2014) Front. Genet. 5:234). The critical regulatory roles that lncRNAs play in the cell make them ideal candidates for novel therapies (Li and Chen (2013) Intl. J. Biochem. Cell Biol. 45:1895-1910). First, many lncRNAs regulate expression of multiple downstream genes such that targeting lncRNAs allows for modulation of entire gene expression patterns with a single target. Second, their highly-specific expression limits off-target effects in healthy tissues. Third, unlike proteins which need to be translated, modulation of lncRNAs confers immediate effects. Finally, there are many currently available methods for targeting lncRNAs, such as small interfering RNAs (siRNAs), small hairpin RNAs (shRNAs), locked nucleic acids (LNAs), morpholinos, 2′-methyoxyethyl oligos, microRNAs, bicyclic compounds, antisense oligos (ASO), and small molecule inhibitors (Li and Chen (2013) Intl. J. Biochem. Cell Biol. 45:1895-1910). Moreover, traditional therapies for treating important maladies have been ineffective or become ineffective over the course of treatment. For example, metastatic melanoma is a highly-lethal skin cancer with a five-year survival of 9-15% (Jerant et al. (2000) Amer. Fam. Phys. 62:357-368, 375-356, and 381-352). The critical stage of melanoma progression is the transition to invasive growth when surgical excision is no longer a viable treatment option. The advent of targeted drug therapies, such as RAF/MEK inhibitors, was initially promising. However, resistance to these therapies invariably occurs within a few months of treatment. Given the diagnostic, prognostic, and therapeutic benefits associated with identifying lncRNAs that are associated with and modulate the development and progression of maladies, such as cancer, there is an urgent need to identify specific lncRNAs that can be targeted.

### SUMMARY OF THE INVENTION

The present invention is based, at least in part, on the discovery that SLNCR, as well as several isoforms and biologically active fragments thereof (collectively referred to as SLNCR as described further herein) are lncRNAs useful as biomarkers for the identification, assessment, prevention, and/or treatment of cancers and other conditions in which aberrant transcription factor signaling is associated. SLNCR functions as a coordinator of transcription factors and associated co-activators and/or co-repressors, that modulate gene expression to regulate cohorts of genes involved in various functions, such as cellular invasion and inflammation. In addition to robust expression in melanomas, SLNCR is detectable in other important cancers, such as cervical, ovarian and uterine cancers, pancreatic cancer, and lower grade glioma and glioblastoma multiforme. Increased SLNCR expression also correlates with breast, bladder, thyroid and lung cancers. Overexpression of SLNCR increases invasiveness of cancer cells, such as melanoma cells, such that SLNCR expression levels correlate with cancer stage and severity and is useful as a prognostic marker for clinical outcome. Moreover, quantification of SLNCR expression can also be used to determining an appropriate course of treatment. For example, inhibition of SLNCR decreases invasiveness of cancer cells, which is the critical stage of development for many cancers, such as melanoma. In another example, SLNCR is a co-activator of nuclear receptors, such as the androgen receptor (AR), such that treatment of patients expressing high levels of SLNCR would benefit from the use of nuclear receptor inhibitors like AR inhibitors.

In one aspect, an isolated non-coding nucleic acid molecule selected from the group consisting of: a) an isolated nucleic acid molecule comprising a sequence having at least 80% identity to the nucleic acid sequence of SEQ ID NO: 1, or a fragment thereof, and does not comprise the sequence of SEQ ID NO: 16; b) an isolated nucleic acid molecule comprising a sequence having at least 80% identity to the nucleic acid sequence of SEQ ID NO: 1, or a fragment thereof, and comprises at most a sequence having 99% identity to the sequence of SEQ ID NO: 16; c) an isolated nucleic acid molecule comprising a sequence having at least 80% identity to the nucleic acid sequence of SEQ ID NO: 1 and having at least one of nucleotides G228, A231, T243, C244, T245, C246, C247, A248, T258, C259, T260, C261, C262, and T263, or a fragment thereof, wherein the isolated nucleic acid molecule does not comprise the sequence of SEQ ID NO: 16; d) an isolated nucleic acid molecule comprising a sequence having at least 80% identity to the nucleic acid sequence of SEQ ID NO: 1 and having at least one of nucleotides G228, A231, T243, C244, T245, C246, C247, A248, T258, C259, T260, C261, C262, and T263, or a fragment thereof, wherein the isolated nucleic acid molecule comprises at most a sequence having 99% identity to the sequence of SEQ ID NO: 16; e) an isolated nucleic acid molecule comprising a sequence having not more than 61 nucleotide substitutions, deletions, or insertions as compared with the nucleic acid sequence of SEQ ID NO: 1, or fragments thereof, and does not comprise the sequence of SEQ ID NO: 16; f) an isolated nucleic acid molecule comprising a sequence having not more than 61 nucleotide substitutions, deletions, or insertions as compared with the nucleic acid sequence of SEQ ID NO: 1, or fragments thereof, and comprises at most a sequence having 99% identity to the sequence of SEQ ID NO: 16; and g) an isolated nucleic acid molecule comprising a sequence having at least 80% identity to the nucleic acid sequence of SEQ ID NO: 22 or 41, or a fragment thereof, and does not comprise the sequence of SEQ ID NO: 16, is provided.

The compositions of the present invention are characterized by many embodiments and each such embodiment can be applied to any combination of embodiments described herein. For example, in one embodiment, the isolated non-coding nucleic acid molecule, or fragment thereof, is less than 2,257 nucleotides in length. In another embodiment, the isolated nucleic acid molecule, or fragment thereof, is 301 nucleotides in length or shorter, or is 111 nucleotides in length or shorter. In still another embodiment, the nucleic acid molecule, or fragment thereof, is at least 111 nucleotides in length and is less than 2,257 nucleotides in length. In yet another embodiment, the isolated nucleic acid molecule, or fragment thereof, comprises a domain selected from the group consisting of an SRA1 H2 helix domain, an SRA1 H3 helix domain, a Brn3a binding domain, an androgen receptor (AR) binding domain, a PXR binding domain, a PAX5 binding domain, an SRA1 H5 helix domain, an SRA1 H6 helix domain, a SLNCR autoregulation domain, a SLNCR cons 2 domain, a SLNCR2 isoform-specific domain, and a SLNCR3 isoform-specific domain. In another embodiment, the isolated nucleic acid molecule, or fragment thereof, comprises a Brn3a binding domain and an androgen receptor (AR) binding domain. In still another embodiment, the isolated nucleic acid molecule, or fragment thereof, comprises an SRA H6 helix domain. In yet another embodiment, the isolated non-coding nucleic acid molecule, or fragment thereof, has the ability to directly bind to at least one protein transcription factor selected from the group consisting of SRC-1/NCOA-1, PXR/NR1I2, PAX, EGR-1, AR, E2F-1, CAR/NR1I3, PBX1, ATF2, C/EBP, BRN-3/POU4F1, HNF4, NF-kB, AP2, OCT4/POU5F1, SP1, STAT5, p53, TFIID, SLIRP, STAT3, REST, REST4, and DAX1. In another embodiment, the isolated non-coding nucleic acid molecule, or fragment thereof, has the ability to bind to at least one protein transcription factor selected from the group consisting of SRC-1/NCOA-1, PXR/NR1I2, PAX5, EGR-1, AR, E2F-1, CAR/NR1I3, PBX1, ATF2, C/EBP, BRN-3/POU4F1, HNF4, NF-kB, AP2, OCT4/POU5F1, SP1, STAT5, p53, TFIID, SLIRP, STAT3, REST, REST4, and DAX1, wherein the nucleic acid molecule-protein transcription factor complex has the ability to translocate to the nucleus. In still another embodiment, the isolated non-coding nucleic acid molecule, or fragment thereof, has the ability to promote one or more biological activities selected from the group consisting of: 1) the expression or activity of MMP9; 2) downregulation of naturally-occurring SLNCR isoforms; 3) modulation of the expression of one or more genes listed in FIGS. 7, 14, 16, 17, 19, and 31; 4) the expression of PLA2G4C, CT45A6, EGR2, RP11-820L6.1, EGR1, ATF3, VCX3A, SPCS2, FABP5, MAGEA2B, RPL41P1, RPS17, HNRNPA1P10, TXNIP, RPL21P75, EIF3CL, RPL7, CT45A3, GTF2IP1, CDK7, HIST1H1C, CT45A1, BTG2, RPS27, RP11-3P17.3, FDCSP, CITED4, IL34, and PD-L1; 5) cellular proliferation; 6) cell death; 7) cellular migration; 8) genomic replication and/or instability; 9) angiogenesis induction; 10) cellular invasion; 11) cancer metastasis; 12) regulation of immune response and/or immune evasion; 13) modulation of one or more genes listed in Tables S5 and S6 affected by SLNCR overexpression; and 14) binding to one or more of transcriptin factors selected from the group consisting of SRC-1/NCOA-1, PXR/NR1I2, PAX, EGR-1, AR, E2F-1, CAR/NR1I3, PBX1, ATF2, C/EBP, BRN-3/POU4F1, HNF4, NF-kB, AP2, OCT4/POU5F1, SP1, STAT5, p53, TFIID, SLIRP, STAT3, REST, REST4, and DAX1. In yet another embodiment, the isolated non-coding nucleic acid molecule, or fragment thereof, comprises the sequence of SEQ ID NO: 1. In another embodiment, the isolated non-coding nucleic acid molecule, or fragment thereof, comprises the sequence of SEQ ID NO: 2. In still another embodiment, the isolated non-coding nucleic acid molecule, or fragment thereof, comprises the sequence of SEQ ID NO: 3. In yet another embodiment, the isolated non-coding nucleic acid molecule, or fragment thereof, consists essentially of the sequence of SEQ ID NO: 1. In another embodiment, the isolated non-coding nucleic acid molecule, or fragment thereof, consists essentially of the sequence of SEQ ID NO: 2. In still another embodiment, the isolated non-coding nucleic acid molecule, or fragment thereof, consists essentially of the sequence of SEQ ID NO: 3. In yet another embodiment, the sequence of the nucleic acid molecule, or fragment thereof, is not derived from a single contiguous locus of a genome. In another embodiment, the isolated non-coding nucleic acid molecule, or fragment thereof, is an RNA. In still another embodiment, the isolated non-coding nucleic acid molecule, or fragment thereof, is non-naturally occurring. In yet another embodiment, the isolated non-coding nucleic acid molecule, or fragment thereof, further comprises a heterologous nucleic acid sequence. In another embodiment, the isolated non-coding nucleic acid molecule, or fragment thereof, is operably linked to a nucleic acid expression promoter.

In another aspect, a pharmaceutical composition comprising an isolated non-coding nucleic acid molecule, or fragment thereof, of the present invention, and a pharmaceutically acceptable agent selected from the group consisting of excipients, diluents, and carriers, is provided. In one embodiment, the pharmaceutical composition comprises the isolated non-coding nucleic acid at a purity of at least 75%. In another embodiment, the pharmaceutical composition further comprises a nuclear receptor targeting drug. In still another embodiment, the nuclear receptor targeting drug is selected from the group consisting of luteinizing hormone-releasing hormone (LHRH) analogs, androgen receptor inhibitors, anti-androgens, hormone blocking drugs, nuclear receptor agonists, nuclear receptor antagonists, selective receptor modulators, selective androgen receptor modulators (SARMs), selective estrogen receptor modulators (SERMs), selective progesterone receptor modulators (SPRMs), selective glucocorticoid receptor agonists (SEGRAs), and selective glucocorticoid receptor modulators (SEGRMs). In yet another embodiment, the nuclear receptor target drug is selected from the group consisting of leuprolide (Lupron®, Eligard®), goserelin (Zoladex®), triptorelin (Trelstar®), histrelin (Vantas®), degarelix (Firmagon®), bicalutamide (Casode®), enzalutamide (Xtandi®), flutamide (Eulexin®), nilutamide (Nilandron®), ketoconazole (Nizoral®), abiraterone (Zytiga®), dexamethasone, megestrol acetate (Megace®), medroxyprogesterone acetate (MPA), ethisterone, norethindrone acetate, norethisterone, norethynodrel, ethynodiol diacetate, norethindrone, norgestimate, norgestrel, levonorgestrel, medroxyprogesterone acetate, desogestrel, etonogestrel, drospirenone, norelgestromin, desogestrel, etonogestrel, gestodene, dienogest, drospirenone, elcometrine, nomegestrol acetate, trimegestone, tanaproget, BMS948, mifepristone, 4-hydroxytamoxifen, CINPA1, Cyproterone acetate (Androcur®, Cyprostat®, Siterone®), chlormadinone acetate (Clordion®, Gestafortin®, Lormin®, Non-Ovlon®, Normenon®, Verton®), 17-hydroxyprogesterone (17-OHP), THC, clotrimazole, PK11195 [1-(2-chlorophenyl)-N-methyl-N-(1-methylpropyl)-3-isoquinolinecarboxamide], meclizine, androstanol, CITCO [6-(4-chlorophenyl)imidazo [2,1-b][1,3] thiazole-5-carbaldehyde O-(3,4-dichlorobenzyl) oxime], zearalenone (ZEN), T0901317, S07662, enobosarm, BMS-564,929, LGD-4033, AC-262,356, JNJ-28330835, LGD-2226, LGD-3303, S-40503, S-23, clomifene, femarelle, ormeloxifene, raloxifene, tamoxifen, toremifene, lasofoxifene, ospemifene, afimoxifene, arzoxifene, bazedoxifene, gulvestrant (Faslodex®, ICI-182780), CDB-4124, asoprisnil, proellex, mapracorat (BOL-303242-X, ZK 245186), fosdagrocorat (PF-04171327), ZK 216348, and 55D1E1.

In still another aspect, a vector comprising an isolated non-coding nucleic acid molecule, or fragment thereof, of the present invention is provided. In one embodiment, the vector is an expression vector.

In yet another aspect, a host cell transfected with a vector or an expression vector described herein is provided.

In another aspect, a method of producing a non-coding nucleic acid molecule comprising culturing a host cell described herein in an appropriate culture medium to, thereby, produce the non-coding nucleic acid molecule, is provided. In one embodiment, the host cell is a bacterial cell or a eukaryotic cell. In still another embodiment, the method further comprises the step of isolating the isolated non-coding nucleic acid molecule, or fragment thereof, of the present invention from the medium or host cell.

In still another aspect, a method of treating a subject afflicted with a cancer comprising administering to the subject anti-SLNCR therapy comprising an agent that inhibits the genomic copy number, amount, and/or activity of SLNCR, thereby treating the subject afflicted with the cancer.

The methods of the present invention are characterized by many embodiments and each such embodiment can be applied to any method described herein and in any combination. For example, in one embodiment, the agent is administered in a pharmaceutically acceptable formulation. In another embodiment, the agent directly binds SLNCR. In still another embodiment, SLNCR is human SLNCR. In yet another embodiment, the method further comprises administering one or more additional anti-cancer agents, optionally wherein the one or more additional anti-cancer agents comprises a nuclear receptor targeting drug.

In yet another aspect, a method of inhibiting hyperproliferative growth, migration, invasiveness, angiogenesis induction, metastasis, or immune evasion of a cancer cell, or modulating immune responses in a cancer or immune cell, the method comprising contacting the cancer cell or cells with anti-SLNCR therapy comprising an agent that inhibits the genomic copy number, amount, and/or activity of SLNCR, thereby inhibiting hyperproliferative growth, migration, invasiveness, angiogenesis induction, metastasis, or immune evasion of the cancer cell, or modulating immune responses in a cancer or immune cell, optionally wherein the immune response is upregulated, is provided. In one embodiment, the step of contacting occurs in vivo, ex vivo, or in vitro. In another embodiment, the agent is administered in a pharmaceutically acceptable formulation. In still another embodiment, the agent directly binds SLNCR. In yet another embodiment, SLNCR is human SLNCR. In another embodiment, the method further comprises administering one or more additional anti-cancer agents, optionally wherein the one or more additional anti-cancer agents comprises a nuclear receptor targeting drug.

In another aspect, a method of determining whether a subject is afflicted with an invasive or metastatic cancer or at risk for developing an invasive or metastatic cancer comprising: a) obtaining a biological sample from the subject; b) determining the presence, copy number, amount, and/or activity of at least one biomarker listed in Table 1A or 1B in a subject sample; c) determining the presence, copy number, amount, and/or activity of the at least one biomarker in a control; and d) comparing the presence, copy number, amount, and/or activity of said at least one biomarker detected in steps b) and c), wherein the presence or a significant increase in the copy number, amount, and/or activity of the at least one biomarker in the subject sample relative to the control indicates that the subject is afflicted with the invasive or metastatic cancer or at risk for developing the invasive or metastatic cancer, is provided. In one embodiment, the method further comprises recommending, prescribing, or administering an agent that inhibits the copy number, amount, and/or activity of SLNCR if the subject is afflicted with the invasive or metastatic cancer or at risk for developing the invasive or metastatic cancer. In another embodiment, the agent is administered in a pharmaceutically acceptable formulation, the agent directly binds SLNCR, or SLNCR is human SLNCR. In still another embodiment, the control sample is determined from a cancerous or non-cancerous sample from either the patient or a member of the same species to which the patient belongs, optionally wherein the cancerous or non-cancerous sample is obtained from the same tissue type as the biological sample. In yet another embodiment, the control sample comprises cells. In another embodiment, the method further comprises determining responsiveness to anti-immune checkpoint inhibitor therapy measured by at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

In still another aspect, a method of assessing the efficacy of an agent for treating a cancer in a subject comprising: a) detecting in a first subject sample and maintained in the presence of the agent the presence, copy number, amount and/or activity of at least one biomarker listed in Table 1A or 1B; b) detecting the presence, copy number, amount and/or activity of the at least one biomarker listed in Table 1A or 1B in a second subject sample and maintained in the absence of the test compound; and c) comparing the presence, copy number, amount and/or activity of the at least one biomarker listed in Table 1A or 1B from steps a) and b), wherein the absence or a significantly decreased copy number, amount, and/or activity of the at least one biomarker listed in Table 1A or 1B in the first subject sample relative to the second subject sample, indicates that the agent treats the cancer in the subject, is provided.

In yet another aspect, a method of monitoring the progression of a cancer in a subject comprising: a) detecting in a subject sample at a first point in time the presence, copy number, amount, and/or activity of at least one biomarker listed in Table 1A or 1B; b) repeating step a) during at least one subsequent point in time after administration of a therapeutic agent; and c) comparing the presence, copy number, amount, and/or activity detected in steps a) and b), wherein the presence or a significantly increased copy number, amount, and/or activity of the at least one biomarker listed in Table 1 in the first subject sample relative to at least one subsequent subject sample, indicates that the agent treats the cancer in the subject, is provided. In one embodiment, the subject has undergone treatment, completed treatment, and/or is in remission for the cancer in between the first point in time and the subsequent point in time. In another embodiment, the subject has undergone anti-SLNCR therapy in between the first point in time and the subsequent point in time. In still another embodiment, the first and/or at least one subsequent sample is selected from the group consisting of ex vivo and in vivo samples. In yet another embodiment, the first and/or at least one subsequent sample is obtained from an animal model of the cancer, a human model of the cancer, or a primary human cancer. In another embodiment, the first and/or at least one subsequent sample is a portion of a single sample or pooled samples obtained from the subject.

In another aspect, a cell-based method for identifying an agent that inhibits a cancer, wherein the method comprises: a) contacting a cancer cell expressing at least one biomarker listed in Table 1A or 1B with a test agent; and b) determining the effect of the test agent on the copy number, level of expression, and/or level of activity of the at least one biomarker in Table 1A or 1B to thereby identify an agent that inhibits the cancer, is provided. In one embodiment, the cells are isolated from an animal model of a cancer, a human model of the cancer, or a primary human cancer. In another embodiment, the step of contacting occurs in vivo, ex vivo, or in vitro.

As described above. the methods of the present invention are characterized by many embodiments and each such embodiment can be applied to any method described herein and in any combination. For example, in one embodiment, the method further comprises determining the ability of the test agent to bind to the at least one biomarker listed in Table 1A or 1B before or after determining the effect of the test agent on the copy number, level of expression, or level of activity of the at least one biomarker listed in Table 1A or 1B. In another embodiment, the sample comprises cells, cell lines, histological slides, paraffin embedded tissue, fresh frozen tissue, fresh tissue, biopsies, skin, blood, plasma, serum, buccal scrape, saliva, cerebrospinal fluid, urine, stool, mucus, or bone marrow, obtained from the subject. In still another embodiment, the presence or copy number is assessed by microarray, quantitative PCR (qPCR), high-throughput sequencing, comparative genomic hybridization (CGH), or fluorescent in situ hybridization (FISH). In yet another embodiment, the amount of the at least one biomarker listed in Table 1A or 1B is assessed by detecting the presence in the samples of a polynucleotide molecule encoding the biomarker or a portion of said polynucleotide molecule. In another embodiment, the polynucleotide molecule is a mRNA, cDNA, or functional variants or fragments thereof. In still another embodiment, the step of detecting further comprises amplifying the polynucleotide molecule. In yet another embodiment, the amount of the at least one biomarker is assessed by annealing a nucleic acid probe with the sample of the polynucleotide encoding the one or more biomarkers or a portion of said polynucleotide molecule under stringent hybridization conditions. In another embodiment, the amount of the at least one biomarker is assessed using a reagent which specifically binds with said biomarker. In still another embodiment, the reagent is selected from the group consisting of a natural protein binding partner, an aptamer, an antibody, an antibody derivative, and an antibody fragment. In yet another embodiment, the activity of the at least one biomarker is assessed by determining the magnitude of cellular proliferation, cell death, cellular migration, replication, induction of angiogenesis, cellular invasion/metastasis, immune response, or immune evasion. In another embodiment, the agent or anti-SLNCR therapy is selected from the group consisting of a small molecule, antisense nucleic acid, interfering RNA, shRNA, siRNA, aptamer, ribozyme, dominant-negative protein, blocking antibody, CRISPR, and combinations thereof. In still another embodiment, the agent or anti-SLNCR therapy is shRNA or siRNA, antisense oligos (ASO) including RNase-H dependent methods, bicyclic compounds, locked nucleic acids (LNAs), morpholinos, 2′-methyoxyethyl modified nuclei acids, microRNAs, and small molecule inhibitors. In yet another embodiment, the agent or anti-SLNCR therapy further comprises a nuclear receptor targeting drug. In another embodiment, the nuclear receptor targeting drug is selected from the group consisting of luteinizing hormone-releasing hormone (LHRH) analogs, androgen receptor inhibitors, anti-androgens, hormone blocking drugs, nuclear receptor agonists, nuclear receptor antagonists, selective receptor modulators, selective androgen receptor modulators (SARMs), selective estrogen receptor modulators (SERMs), selective progesterone receptor modulators (SPRMs), selective glucocorticoid receptor agonists (SEGRAs), and selective glucocorticoid receptor modulators (SEGRMs). In still another embodiment, the nuclear receptor target drug is selected from the group consisting of leuprolide (Lupron®, Eligard®), goserelin (Zoladex®), triptorelin (Trelstar®), histrelin (Vantas®), degarelix (Firmagon®), bicalutamide (Casode®), enzalutamide (Xtandi®), flutamide (Eulexin®), nilutamide (Nilandron®), ketoconazole (Nizoral®), abiraterone (Zytiga®), dexamethasone, megestrol acetate (Megace®), medroxyprogesterone acetate (MPA), ethisterone, norethindrone acetate, norethisterone, norethynodrel, ethynodiol diacetate, norethindrone, norgestimate, norgestrel, levonorgestrel, medroxyprogesterone acetate, desogestrel, etonogestrel, drospirenone, norelgestromin, desogestrel, etonogestrel, gestodene, dienogest, drospirenone, elcometrine, nomegestrol acetate, trimegestone, tanaproget, BMS948, mifepristone, 4-hydroxytamoxifen, CINPA1, Cyproterone acetate (Androcur®, Cyprostat®, Siterone®), chlormadinone acetate (Clordion®, Gestafortin®, Lormin®, Non-Ovlon®, Normenon®, Verton®), 17-hydroxyprogesterone (17-OHP), THC, clotrimazole, PK11195 [1-(2-chlorophenyl)-N-methyl-N-(1-methylpropyl)-3-isoquinolinecarboxamide], meclizine, androstanol, CITCO [6-(4-chlorophenyl)imidazo [2,1-b][1,3] thiazole-5-carbaldehyde O-(3,4-dichlorobenzyl) oxime], zearalenone (ZEN), T0901317, S07662, enobosarm, BMS-564,929, LGD-4033, AC-262,356, JNJ-28330835, LGD-2226, LGD-3303, S-40503, S-23, clomifene, femarelle, ormeloxifene, raloxifene, tamoxifen, toremifene, lasofoxifene, ospemifene, afimoxifene, arzoxifene, bazedoxifene, gulvestrant (Faslodex®, ICI-182780), CDB-4124, asoprisnil, proellex, mapracorat (BOL-303242-X, ZK 245186), fosdagrocorat (PF-04171327), ZK 216348, and 55D1E1. In yet another embodiment, the at least one biomarker is selected from the group consisting of 1, 2, 3, 4, 5, 6, 7, 8, 9, 10, or more biomarkers. In another embodiment, the at least one biomarker is human SLNCR selected from the group consisting of human SLNCR, human SLNCR2, or human SLNCR3. In still another embodiment, the cancer is selected from the group consisting of melanoma, lung adenocarcinoma, lung squamous cell carcinoma, cervical cancer, ovarian cancer, uterine cancer, pancreatic cancer, colorectal cancer, lower grade glioma, glioblastoma multiforme, breast cancer, endometrial cancer, prostate cancer, testicular cancer, thyroid cancer, osteosarcoma, esophageal cancer, liver cancer and bladder cancer. In yet another embodiment, the subject is a mammal (e.g., an animal model of cancer or a human).

In still another aspect, any method described herein can be adapted with respect to diagnosing, prognosing, preventing, screening, and/or treating conditions associated with modulated immune responses. Such immune responses can be in cancer cells, immune cells, or other cell types. Such immune responses can be upregulated or downregulated. For example, the diagnostic assays and screening assays can be used to identify SLNCR activity modulation effects on immune response modulation. Similarly, SLNCR activity modulation can be used to modulate immune responses (e.g., anti-SLNCR agents can be used to upregulate immune responses).

### BRIEF DESCRIPTION OF FIGURES

FIG. 1 includes 8 panels identified as panels A, B, C, D, E, F, G, H, and I, which shows SLNCR is associated with worse overall survival. Panel A shows relative expression of SLNCR across multiple melanocytes and melanomas, as measured by RT-qPCR, compared to A375 after normalization to GAPDH. Error bars represent standard deviations calculated from 3 reactions. Panel B shows schematic of SLNCR1's exon structure. The highly-conserved and SRA1-like sequences are highlighted. Panel C shows box plot of SLNCR expression from 150 TCGA melanomas categorized based on tumor thickness at diagnosis. Data are represented as mean±SEM. Significance was calculated using the Student's t-test: * p-value<0.05. Panel D shows Kaplan-Meier survival analysis of high or low SLNCR expressing TCGA melanomas, defined by the median SLNCR expression (RPKM=14.1). Panels E through F show that SLNCR is highly-conserved and expressed in multiple cancers. Panel E depicts MiTranscriptome expression data for SLNCR (linc00673) across all available cancer and normal tissue type cohorts. Panel F shows melanomas express three transcripts from the SLNCR chromosomal 17 locus. Integrated Genome Viewer plot (middle) displaying melanoma RNA-seq read intensities corresponding to the indicated patient-derived melanomas. The three SLNCR isoforms are depicted below. (Panels G, H, and I) Alignments were performed using Clustal Omega and viewed in JALVIEW. Panels G and H show alignment of SLNCR (nucleotides 462-572) with confirmed or predicted lncRNAs from the indicated species. Residues are shaded according to percent identity; dark blue >80%, blue >60%, light blue >40%. Panel I shows alignment of SLNCR (nucleotides 441-672) with SRA1. Purines are highlighted in pink, and pyrimidines are highlighted in teal. Asterisks denote identical nucleotides.

FIG. 2 includes 13 panels, identified as panels A, B, C, D, E, F, G, H, I, J, K, L, and M. Panels A and B show isoform and genomic location information for SLNCR. Panel A shows the detection of 3 isoforms of SLNCR in patient-derived melanomas. Following tumor excision, cells were separated by limiting dilution to plate 1-2 cells per well and propagated over 3-4 months. cDNA libraries were prepped using TruSeq® RNA Sample Prep kit v2 (Illumina) and sequenced on the HiSeq® 2500 (Illumina) The histogram represents the frequency of mapped RNA-seq reads as exported from the Integrated Genome Viewer (Broad Institute). Panel B shows a schematic illustration of the genomic location of SLNCR within human chromosome 17. Panels C-K show SLNCR's highly-conserved sequence increases melanoma invasion. Panels C and D show the schematic highlights SLNCR1-specific siRNAs targeting the exon 3-4 junction. Left: relative expression of SLNCR1 upon siRNA knockdown in the melanoma short-term culture WM1976. RT-qPCR data is represented as the fold change compared to scramble siRNA control, normalized to GAPDH. Error bars represent standard deviations calculated from 3 reactions. (E-H) Matrigel invasion assays of WM1976 (E and F) or WM1575 (G and H) cells transfected with the indicated siRNA. Invasion is calculated as the percent of invading cells compared to mobile cells as counted in 8 fields of view. Top panels show representative images of the indicated invading and mobile cells. Quantification from 3 independent replicates, represented as mean±SD, is shown at the bottom. (I-K) As in (E-H) but with A375 melanoma cells transfected with the indicated plasmids. The schematic (top) denotes the SLNCR1 sequences expressed from the indicated plasmids. The bottom left panel shows representative images, while quantification from 3 independent replicates is shown at the right. Significance was calculated using the Student's t-test: * p-value<0.05, ** p-value<0.005, *** p-value<0.0005, ns=not significant. Panels L and M show knockdown of SLNCR1 does not affect melanoma cell proliferation. (Panel L) WM1976 or (Panel M) WM1575 melanoma short term cultures were transfected with the indicated siRNAs at Day 0, and proliferation was measured using WST-1 reagent every day for 4 days. The proliferation assay was repeated three times, and one representative assay is shown. Error bars represent standard deviation from 3 replicates. The apparent decrease in cell proliferation observed with si-SLNCR1 (2) is likely due to toxicity of the siRNA.

FIG. 3 includes 14 panels, identified as panels A, B, C, D, E, F, G, H, I, J, K, L, M, and N. Panels A and B show that SLNCR expression is correlated with several cancers as compared to normal tissue and that SLNCR regulates certain cancers. Panel A shows cancers that are predicted to be regulated by SLNCR based on expression analyses. Panel B shows that stage II melanomas from females show significantly higher SLNCR expression than from stage II males and that SLNCR expression is highest in stage II melanomas overall. RNA-seq reads from 100 randomly sampled melanoma patients were obtained from The Cancer Genome Atlas (TCGA, available on the world wide web at cancergenome.nih gov). RPKM (reads per kilobase per million) represents the numbers of reads from each patient samples mapped to SLNCR. T-test statistics were performed using GraphPad Prism version 6.00 for Windows (GraphPad Software, La Jolla Calif. USA). Panels C to I show SLNCR1 transcriptionally upregulates MMP9 to increase melanoma invasion. Panel C shows heat map of differentially expressed genes significantly regulated by SLNCR1 and SLNCR1^{cons}, but not SLNCR1Δ^{cons}, in the melanoma cell line A375. The shading represents the log 2 fold change compared vector only control. Panel D shows relative MMP9 expression in A375 cells transfected with the indicated plasmids. RT-qPCR data is represented as the fold change compared to a vector control, normalized to GAPDH. Error bars represent standard deviations calculated from 3 reactions. (Panel E-G) MMP9 activity from supernatants of cells transfected with the indicated plasmids or siRNAs were quantified using gelatin zymography. Percent MMP9 activity is represented as fold change compared to the vector control, normalized to MMP2 activity. Error bars represent standard deviations from three independent replicates. Panel E shows percent MMP9 activity in supernatants of A375 cells transfected with the indicated plasmids. Panel F shows percent MMP9 activity of WM1976 supernatant upon knockdown of SLNCR1. Panel G shows percent MMP9 activity of WM1575 supernatant upon knockdown of SLNCR1. Panel H shows matrigel invasion assay of A375 melanoma cells transfected with the indicated plasmids and siRNAs, as in FIG. 2 (Panel E-Panel K). Panel I shows A375 cells, grown in steroid-deprived conditions, were transfected with a MMP9-firefly (FL) reporter plasmid, a CMV-RL (renilla luciferase) control, and the indicated SLNCR1 expression plasmids. Luciferase activity was measured 24 hours post-transfection. Relative FL activity was calculated as a fold-change compared to vector only control cells, after normalization to RL activity. Shown is one representative assay from at least three independent replicates. Error bars represent standard deviation from four reactions. Significance was calculated using the Student's t-test: * p-value<0.05, ** p-value<0.005, *** p-value<0.0005, ns=not significant. Panels J and N show SLNCR1 increases melanoma invasion through transcriptional upregulation of MMP9. Panel J shows heat map representing the log 2 fold change of transcripts from A375 cells expressing SLNCR1, SLNCR1^{cons} or SLNCR1^{Δcons}, as compared to a vector only control. Shown are transcripts significantly regulated by SLNCR1 (adjusted p-value<0.05, fold change>2). (B) Box plot of MMP9 expression from 150 TCGA, categorized by the tumor cell type submitted for sequencing. Primary=primary tumor, regional=regional metastasis, and distant=distant metastasis. Data are represented as mean±SEM. (L and M) As described in FIG. 3 (E-G). Panel L shows quantification of three independent zymograms of cell supernatants from A375 cells transfected with the indicated siRNAs. Panel M shows quantification of three independent zymograms of cell supernatants from A375 cells transfected with the indicated siRNAs. Panel N shows relative SLNCR1 expression in A375 cells transfected with the indicated plasmids and siRNAs. RT-qPCR data is represented as the fold change compared A375 transfected with a vector and scramble control, normalized to GAPDH. Error bars represent standard deviations calculated from 3 reactions. Significance was calculated using the Student's t-test: * p-value<0.05, ** p-value<0.005, *** p-value<0.0005, ns=not significant.

FIG. 4 includes 8 panels, identified as panels A, B, C, D, E, F, G, and H. Panels A to E depicts AR and Brn3a bind to SLNCR1's conserved sequence. Panel A shows schematic depicting the RATA approach for identification of TFs associated with SLNCR1. SLNCR1-MS2 RNP complexes were immunoprecipitated with α-FLAG antibody, eluted from beads using FLAG peptide, and the eluate was immediately subjected to the TF Activation Profiling Plate Array I (Signosis). TF-bound probes were isolated through column separation and analyzed through hybridization with a plate whose wells are pre-coated with complementary DNA. Panel B shows ectopically expressed FLAG-tagged MS2 was immunoprecipitated from A375 cells transfected with the indicated MS2-loop containing SLNCR1 construct, compared to control cells expressing untagged SLNCR1 constructs. Left panel: total protein input or bound proteins following IP with α-FLAG antibody was subjected to Western blot analysis. The blot was probed with α-FLAG and α-GAPDH antibodies. Middle and right: relative enrichment of the indicated transcripts as measured by RT-qPCR compared to RNA enriched from cells expressing SLNCR1 without MS2 stem loops. Bound FLAG-MS2 RNPs were eluted using FLAG peptide. Panel C shows fold enrichment of TF-specific probes with MS2-based purification of SLNCR1 or SLNCR1^{Δcons} from A375 cells. Probe enrichment is represents fold enrichment compared to an untagged RNA control IP, after normalization to the signal of GATA-specific probes. Shown is one representative assay of TFspecific probes showing significant (>7-fold) enrichment in at least two out of three replicates. Panel D shows immunoprecipitations from HEK293T transfected with GFP-tagged AR and the indicated SLNCR1 expressing plasmids using either α-AR antibody or an IgG nonspecific control. Top panel: western blot analysis of input (I), IgG bound (IgG) or α-AR bound (AR) proteins. Bottom panels: relative enrichment of the indicated transcripts from AR-IPs, compared to an IgG nonspecific control. HEK293T cells were transfected with GFP-tagged AR and either SLNCR1 (bottom left panel) or SLNCR1^{Δcons} (bottom middle panel) or SLNCR1^{Δ568-637} (bottom right panel) expression plasmids. Panel E shows immunoprecipitations from UV-crosslinked HEK293T transfected with Brn3a and the indicated SLNCR1 expression plasmid using either anti-Brn3a antibody or an IgG nonspecific control. Top panel: western blot of input (I), IgG bound (IgG) or α-Brn3a bound (Brn3a) proteins. Bottom panels: relative enrichment of the indicated transcripts from Brn3a-IPs. HEK293T cells were transfected with GFP-AR and either SLNCR1 (bottom left panel) or SLNCR1^{Δcons} (bottom right panel). To control for differences in the efficiency of proteinase K digestion, enrichment was calculated compared to input transcript levels after normalization to levels of the 18s RNA. All RT-qPCR are represented as mean±SD from three replicates. Panels F and G show MS2-based IP of the nuclear fraction of SLNCR1. Panel F shows fractionation of the melanoma short term culture WM1976 reveals that SLNCR1 is located in both the cytoplasm and nucleus. Left: western blot of cytoplasmic (C) and nuclear (N) fractions of WM1976. The blot was probed with α-Hsp90 and α-snRNP70 antibodies to confirm successful fractionation. Right: RT-qPCR of cytoplasmic and nuclear RNAs from the fractionation shown at left. Cytoplasmic enrichment of β-ACTIN mRNA and nuclear enrichment of the NEAT1 lncRNA confirms successful fractionation of RNA. Panel G shows FLAG-tagged MS2 was immunoprecipitated from A375 cells transfected with plasmids expressing FLAG-tagged MS2 and the indicated SLNCR1 construct, compared to control cells expressing SLNCR1 without encoded MS2 stem loop structures. Relative enrichment of the indicated transcripts as measured by RT-qPCR compared to RNA enriched from cells expressing SLNCR1 without MS2 stem loops. Total proteins and RNAs were released by incubating in Laemmli buffer at 95° C. for 5 minutes. Panel H shows that SLNCR localizes to both the cytoplasm and the nucleus. WM1976 cells were fractionated using the NEPER™ Nuclear and Cytoplasmic Extraction Kit (Thermo Scientific). One half of each fraction was used for RNA isolation using Trizol® (Life Technologies) and Qiagen RNeasy® Mini Kit. RNA was treated with DNase treated and cDNA was generated using SuperScript® III (Invitrogen). RT-qPCR analysis revealed the correct localization of β-Actin mRNA to the cytoplasm, and NEAT1 lncRNA to the nucleus, indicating successful fractionation.

FIG. 5 includes 14 panels, identified as panels A, B, C, D, E, F, G, H, I, J, K, L, M, and N. Panels A to F shows SLNCR1-mediated invasion requires both AR and Brn3a. Panels A and D show MMP9 activity of A375 cells transfected with the indicated plasmids and siRNAs, as in FIG. 3 (E-G). Panels B and E show relative luciferase activity of A375 cells transfected with an MMP9-RL reporter, as well as the indicated plasmids and siRNAs. Quantification was performed as in FIG. 3I. Panel C and F show matrigel invasion assay of A375 melanoma cells transfected with the indicated plasmids and siRNAs. The top panel shows representative images of the indicated invading or mobile cells, and the quantification from 3 independent replicates is shown at the bottom. Significance was calculated using the Student's t-test: * p-value<0.05, n.s.=not significant. Panels G to M show knockdown of AR or Brn3a does not affect SLNCR1 overexpression. Panel G shows relative AR expression in A375 cells transfected with the indicated siRNAs. Panel H shows Western blot of A375 cell lysates following transfection with the indicated siRNAs. Left panel: representative blot probed with α-AR and α-GAPDH antibodies. Right panel: quantification from three independent replicates, normalized to GAPDH. Panel I shows relative SLNCR expression in A375 cells co-transfected with the indicated plasmids and siRNAs. Panel J same as in Panel I, but for relative AR expression. Panel K shows relative Brn3a expression in A375 cells transfected with the indicated siRNAs. Panel L shows elative SLNCR expression in A375 cells co-transfected with the indicated plasmids and siRNAs. Panel M same as in Panel L, but for relative BRN3a expression. All RT-qPCR represent the fold change compared to scramble or vector/scramble controls after normalization to GAPDH. Error bars represent standard deviations calculated from 3 reactions. Significance was calculated using the Student's t-test: *** p-value<0.0005, **** p-value<0.0001. Panel N shows that SLNCR is highly conserved. Nucleotide BLAST (available on the world wide web at www.ncbi.nlm.nih.gov) was used to identify putative homologs to SLNCR. Alignment of the listed sequences was performed using Clustal Omega (EMBL-EBI) and viewed in Jalview. Only the most highly conserved 60 nucleotides are shown

FIG. 6 includes 4 panels, identified as panels A, B, C, and D, which show that SLNCR knockdown slows proliferation of melanoma cells. The melanoma short-term culture, WM1575, was transfected in duplicate (Lipofectamine® RNAiMAX; Life Technologies) with siRNAs targeting SLNCR (LINC00673 siRNAs SI05482540 and SI05482519; Qiagen). Panel A shows the results of proliferation measured using the WST-1 reagent (Roche) according to the manufacturer's instructions. Cells were incubated for 1 hour prior to measurement. Panel B show the results of RNA isolated 48 post-transfection, treated with DNase, and generation of cDNA using SuperScript® III (Invitrogen). RT-qPCR analysis revealed significant knockdown of SLNCR. Panel C is a replicate of Panel A. Panel D shows the results of proliferation from an additional melanoma short term culture WM1976.

FIG. 7 shows the results of transcripts differentially expressed upon SLNCR overexpression. The stable melanoma cell line, A375, was transfected in duplicate using Lipofectamine® 2000 (Life Technologies) with a plasmid expressing SLNCR. RNA was isolated 48 post-transfection, treated with DNase, and prepared for production of cDNA libraries using the TruSeq® RNA Sample Prep Kit v2 (Illumina). Sequencing was performed using a HiSeq® 2500 machine (Illumina). Cuffdiff (Trapnell et al. (2010) Nat. Biotech. 28:511-515) was used to identify genes significantly altered from cells transfected with a plasmid control.

FIG. 8 shows that SLNCR increases enzymatic activity of MMP9 through its highly conserved region. The term “cons” represents the ˜300 nucleotide SLNCR conserved region. A375 cells were transfected with vectors encoding the indicated SLNCR constructs 24 hours post-seeding using Lipofectamine® 2000 (Life Technologies). Media was replaced with serum-free media 24 hours post-transfection and was collected 48 hours post-transfection. Conditioned media was concentrated 5-fold using Amicon® Ultra Centrifugal Filters (Millipore) and run on a 10% gelatin zymography gel (BioRad). Gels were stained with Coomassie Blue after developing overnight at 37° C. and images were quantified using ImageJ software. MMP9 activity was normalized to MMP2 activity and shown as a fold change relative to vector control. Error bars represent standard deviations (SDs) from independent replicates.

FIG. 9 includes 2 panels, identified as panels A and B, which show that a highly conserved region of SLNCR is required for increasing the invasiveness of A375 melanoma cells. The term “cons” represents the ˜300 nucleotide SLNCR conserved region. Panel A shows the results of 2.5×10⁴ A375 cells transfected with the indicated plasmids using Lipofectamine® 2000 (Life Technolgoies) and plated in either BD Biocoat matrigel inserts (top panel) or uncoated control inserts (bottom panel), and incubated for 16 hours. Representative images are shown. Panel B shows quantification of the number of invaded or migrant cells counted on 20× magnification in 8 fields of view for 3 independent replicates. The bars represent the standard deviation, the * represents a P-value<0.05 versus control, the ** represents a P-value<0.005 versus control, and ns represents non-significance.

FIG. 10 shows that SLNCR-mediated invasion requires MMP9. A375 cells were transfected with the indicated plasmids and siRNAs and assay was completed as in FIG. 9. The *** presents a P-value<0.001 and ns represents non-significance.

FIG. 11 includes 4 panels, identified as panels A, B, C, and D, which show that knockdown of SLNCR decreases invasiveness of melanoma short-term cultures. Panel A shows the results of 10×10⁴ WM1976 cells transfected with the indicated siRNAs using Lipofectamine® RNAiMax (Life Technologies) and plated in either BD Biocoat matrigel inserts (top panel) or uncoated control inserts (bottom panel), and incubated for 22 hours. Representative images are shown. Panel B shows quantification of the number of invaded or migrant cells counted on 20× magnification in 8 fields of view for 3 independent replicates. The bars represent the standard deviation, the * represents a P-value<0.05 versus control, the ** represents a P-value<0.005 versus control, and ns represents non-significance. Panel C shows the same information as in Panel A, except that 7.5×10⁴ WM1575 cells were used per chamber. Panel D shows the same quantification as in Panel B for WM1575 invasion assays.

FIG. 12 includes 3 panels, identified as panels A, B, and C, which show that SLNCR binds to SRC-1, SRC-1 increases MMP9 activity, and TGF-β regulates SLNCR expression. Panel A shows the results of A375 cells co-transfected with a plasmid expressing NLS-FLAG-MS2 and a plasmid expressing MS2-tagged or untagged RNA. Following formaldehyde crosslinking, IP was performed using Sigma anti-FLAG antibody and protein G dynabeads. Panel B shows the RT-PCR quantification of SRC-1 knockdown upon expression of anti-SRC-1 siRNAs. In addition, Panel B shows the results of triplicate experiments in which serum-free media from A375 cells transfected with the indicated siRNA were separated on a 10% gelatin gel. Following incubation overnight at 37° C., bands corresponding to MMP9 gelatinase activity were imaged from 3 replicates and quantified using ImageJ software. Panel C shows the results of WM1975 cells pre-starved fro 6 hours in serum-free media (SFM). At time (t)=0, media was replaced with SFM containing DMSO, TGF-β, or TGF-β with the TGF-β receptor I inhibitor, SB-431542 (from left to right, respectively, in the bar graph for each time shown). RT-PCR data is shown indicating the fold change of SLNCR compared to t=0 and normalized to GAPDH expression. Error bars represent the standard deviation calculated from three reactions.

FIG. 13 includes 3 panels, identified as panels A, B, and C, which show that SLNCR is a nuclear receptor coactivator. The indicated vectors were co-transfected in A375 cells with a MMTV-luciferase reporter (pGL4.36; Promega). Panel A shows the results of dexamethasone (Dex) addition and Panel B shows the results of dihydrotestosterone (DHT) addition relative to a vehicle control, each of which were added 24 hours post-transfection. The portion of the SLNCR sequence mutated in SLNCR mut is shown at the bottom of Panel A with numbers indicating the position within SLNCR sequence. The bold capitalized bases were mutated through site directed mutagenesis. Panel C shows that SLNCR requires DAX1 (NR0B1, NM_000475, NP_000466.2, ENSG00000169297) for full functionality as a nuclear receptor coactivator, as knockdown of DAX1 abolishes the ability of SLNCR to increase dexamethasone induction of the MMTV-luciferase reporter construct. This data confirm that DAX1 and SLNCR functionally interact, and possible physically interact as well. The indicated vectors (empty or SLNCR1-expressing vector) were co-transfected in Hela cells with a MMTV-luciferase reporter (pGL4.36; Promega), along with the indicated siRNAs against DAX1 (Qiagen SI03066014 and SI00010220) using Lipofectamine 2000. The graph shows the results of dexamethasone (Dex) addition relative to a vehicle control, each of which were added 24 hours post-transfection.

FIG. 14 lists transcripts differentially expressed upon SLNCR knockdown. The melanoma short-term culture, WM1976, was transfected in duplicate using Lipofectamine® RNAiMAX (Life Technologies) with siRNAs targeting SLNCR (Qiagen LINC00673 siRNAs SI05482540 and SI05482519). RNA was isolated 48 hours post-transfection, treated with DNase, and prepared for cDNA library generation using TruSeq® RNA Sample Prep Kit v2 (Illumina) Sequencing was performed on the HiSeq 2500 (Illumina) Cuffdiff (Trapnell et al. (2010) Nat. Biotech. 28:511-515) was used to identify genes significantly altered from cells transfected with a plasmid control.

FIG. 15 includes 2 panels, identified as panels A and B, which show that SLNCR regulates genes involved in skin and skeletal muscle development, catabolic and metabolic processes, RNA pol II transcription, hormone and defense responses, cell proliferation, apoptosis and chemotaxis. Gene Ontology (GO) Enrichment analysis of genes regulated by SLNCR were performed using MetaCore™ (Thomson Reuters). Panel A shows GO terms enriched among genes differentially expressed upon SLNCR overexpression shown in FIG. 7. Panel B shows GO terms enriched among genes differentially expressed upon SLNCR knockdown shown in FIG. 14.

FIG. 16 shows that SLNCR2 regulates genes involved in immune and stress responses and kidney development. Transcripts regulated by SLNCR2, categorized into Gene Ontology (GO) Enrichment terms, are shown. Transcripts highlighted in bold are also regulated by SLNCR2.

FIG. 17 shows that SLNCR3 regulates genes involved in immune and stress responses. Transcripts regulated by SLNCR3, categorized into Gene Ontology (GO) Enrichment terms, are shown. Transcripts highlighted in bold are also regulated by SLNCR2.

FIG. 18 includes 2 panels, identified as panels A and B, which show that SLNCR2 and SLNCR3 regulate genes involved in immune and stress responses. The stable melanoma cell line, A375, was transfected in duplicate with a plasmid expressing SLNCR2 or SLNCR3 using Lipofectamine® 2000 (Life Technologies). RNA was isolated 48 post-transfection, treated with DNase, and prepared for cDNA library generation using the TruSeq® RNA Sample Prep Kit v2 (Illumina). Sequencing was performed on a HiSeq® 2500 machine (Illumina). Cuffdiff (Trapnell et al. (2010) Nat. Biotech. 28:511-515) was used to identify genes significantly altered from cells transfected with a plasmid control. Gene Ontology (GO) Enrichment analysis of genes regulated by SLNCR2 and SLNCR3 were performed using MetaCore™ (Thomson Reuters). Panel A shows GO terms enriched among genes differentially expressed upon SLNCR2 overexpression in A375 melanoma cells. Panel B shows GO terms enriched among genes differentially expressed upon SLNCR3 overexpression in A375 melanoma cells.

FIG. 19 shows transcripts differentially expressed upon SLNCR2 or SLNCR3 overexpression. with a plasmid expressing SLNCR2 or SLNCR3 using Lipofectamine® 2000 (Life Technologies). RNA was isolated 48 post-transfection, treated with DNase, and prepared for cDNA library generation using the TruSeq® RNA Sample Prep Kit v2 (Illumina) Sequencing was performed on a HiSeq® 2500 machine (Illumina). Cuffdiff (Trapnell et al. (2010) Nat. Biotech. 28:511-515) was used to identify genes significantly altered from cells transfected with a plasmid control. Listed are differentially expressed genes not already contained within FIGS. 16 and 17 with the Log 2 fold change (compared to a vector control) for both experimental conditions.

FIG. 20 includes 3 panels, identified as panels A, B, and C, which show that SLNCR, SLNCR2 and SLNCR3 transcriptional networks are associated with multiple diseases, including cancers and autoimmune disease. Disease enrichment analysis of genes regulated by SLNCR, SLNCR2 and SLNCR3 were performed using MetaCore™ (Thomson Reuters). Diseases correlated with genes differentially expressed upon SLNCR (Panel A), SLNCR2 (Panel B), or SLNCR3 (Panel C) overexpression in A375 cells are shown.

FIG. 21 show that SLNCR, SLNCR2 and SLNCR3 transcriptional networks map to multiple transcription factors. Network analysis was performed using MetaCore™ (Thomson Reuters). Differentially expressed transcripts used as input lists were identified via RNA-seq analysis of cells over expressing SLNCR, SLNCR2 or SLNCR3, or after siRNA mediated knockdown of total SLNCR, as described above. An “X” indicates that the listed transcription factor is implicated in mediating downstream transcriptional changes as observed through RNA-Seq analysis. Transcription factor networks implicated in more than one experimental condition are shown.

FIG. 22 includes 2 panels, identified as panels A and B, show the results of A375 cells co-transfected with a plasmid expressing NLS-FLAG-MS2 and a plasmid expressing MS2 tagged or untagged RNA. Following cell lysis and protein isolation, immunoprecipitation (IP) was performed using Sigma anti-FLAG antibody and protein G dynabeads. RNA-protein complexes were eluated from beads using FLAG peptide. Eluate from SLNCR tagged or untagged control IPs were incubated with a Signosis biotinylated DNA probe mixture (Signosis, Inc.), and subjected to Transcription Factor Activiation Array analysis according to the manufacturers instruction's. Bars represent fold enrichment of each indicated TF compared to the untagged SLNCR control and normalized to GATA measurements. Panel A shows that SLNCR forms a complex with multiple transcription factors. Panel B shows that BRN-3, C/EBP, ATF2, PBX1, E2F-1, AR, and EGR-1 associate with the SLNCR highly conserved region (SLNCR cons), while CAR, PAX5, and PXR associate with other regions of SLNCR.

FIG. 23 includes 3 panels, identified as panels A, B, and C, which shows sequence requirements for AR binding to SLNCR and for Brn3a/Pou4F1 binding to SLNCR. Panel A shows the alignment of conserved SLNCR sequence (top) with PCGEM1 lncRNA sequence required for AR binding (bottom) (Yang et al. (2013) Nature 500:598-602) is shown. The sequence alignment was performed using MultiAlin (Corpet (1988) Nucl. Acids Res. 16:10881-10890). The shaded sequence indicates the approximate minimal SLNCR sequence predicted to be required for AR binding. Panel B shows the predicted DNA consensus sequence for Brn3 (top) (Gruber et al. (1997) Mol. Cell. Biol. 17:2391-2400). The lowercase “gc” sequence in the predicted DNA consensus sequence denotes a slight preference for those nucleotides. The bottom sequence shows the minimally conserved SLNCR sequence in which the shaded section, especially in uppercase letters, represents the sequence predicted to bind to Brn3 based on similarity to the DNA sequence specificity. Panel C shows a schematic diagram illustrating how SLNCR directly interacts with AR and Brn3a. The Brn3 and AR predicted binding sequences are shown in shaded text. According to the MS2 pulldown/TF array assays results described herein, SLNCR also associates with C/EBP, E2F1, and ATF2. It is believed that these associations are indirect with respect to SLNCR and are mediated through interactions with AR. Previously, AR has been shown to bind to (A.) Brn3a (Berwick et al. (2010) J. Biol. Chem. 285:15286-15295), (B.) C/EBP (Zhang et al. (2010) Oncogene 29:723-738), (C.) E2F-1 (Altintas et al. (2012) Mol. Endocrinol. 26:1531-1541), and (D.) ATF2 (Jorgensen and Nilson (2001) Mol. Endocrinol. 15:1496-1504). EGR-1 is known to bind to the cJUN TF complex containing ATF2 (Verger et al. (2001) J. Biol. Chem. 276:17181-17189).

FIG. 24 shows that SLNCR is orthologous to helix 5 (H5) and helix 6 (H6) of SRA1. SRA1 encodes a long non-coding RNA that is known to bind and coordinate multiple TFs and associates coactivators and/or corepressors (Colley And Leedman (2011) Biochimie 93:1966-1972). The structure of SRA1 has been solved (Novikova et al. (2012) Nucl. Acids Res. 40:5034-5051) and SLNCR contains a sequence with substantially sequence homology to helix 5 and helix 6 of SRA1. Bases conserved in SLNCR are shown in circles, while co-variant basepairs (i.e., G-C to A-T, etc.) are shown in boxes.

FIG. 25 shows sequence requirements for PXR and/or CAR binding to SLNCR. CAR and PXR are related transcription factors that regulate many overlapping targets, suggesting that their DNA binding preferences are similar. Indeed, both TFs heterodimerize with RXR and show a DNA sequence preference for the motif, AGTTCA (Vyhlidal et al. (2004) J. Biol. Chem. 279:46779-46786; Frank et al. (2003) J. Biol. Chem. 278:43299-43310). Given that both TFs bind to similar DNA sequences and that SLNCR is a co-activator for at least one dexamethasone-inducible TF, it is believed that SLNCR binds directly to PXR, a known target of dexamethasone, and that the SLNCR SRA helix 6-like sequence mediates this activity. The capitalized nucleotides represent bases mutated in the SLNCR SRA helix 6-like sequence such that they are required for this interaction.

FIG. 26 includes 2 panels, identified as panels A and B, which show sequence requirements for PAX5 binding to SLNCR. Panel A shows a sequence alignment of SLNCR and Epstein-Barr Virus (EBV) EBER2 performed using MultiAlin (Corpet (1988) Nucl. Acids Res. 16:10881-10890). EBER2 is a long non-coding RNA produced from the viral genome that is known to bind to the host TF PAX5 (Lee et al. (2015) Cell 160:607-618). Alignment of SLNCR and EBER2 reveals areas of sequence similarity, including within a region predicted to indirectly bind to PAX5 (nucleotides ˜20-40). Panel B shows the SLNCR sequence predicted to be associated with PAX5 binding based on similarity to the EBV EBER2 sequence (Panel A). The interaction between EBER2 and PAX5 is likely indirect and mediated through an unknown mediator (Lee et al. (2015) Cell 160:607-618). The SLNCR-PAX5 interaction may be direct or indirect.

FIG. 27 includes 3 panels, identified as panels A, B, and C, which show sequence requirements for SLNCR autoregulation. Panels A and B show that SLNCR autoregulates expression of various SLNCR isoforms. WM1976 cells were transfected with the indicated siRNAs using Lipofectamine® RNAiMax (Life technologies). RNA was isolated using Trizol® (Life Technologies) and the Qiagen RNeasy® Mini Kit, treated with DNase, and prepared for cDNA generation using SuperScript® III (Invitrogen). qRT-PCR data are represented as the fold change compared scrambled siRNA transfected cells as normalized to GAPDH. The error bars represent standard deviations calculated from 3 reactions. SLNCR-specific siRNA sequences are as follows: si-SLNCR (1): AAGAGGATGGGAAGGACTGAT and si-SLNCR (2): CTGATGGGAAGGACTGATCCA (Panel A). SLNCR2/3 specific siRNA sequences are as follows: si-SLNCR2/3 (1): GGGCTGCTTAGTGAAATACAA and si-SLNCR2/3 (2): CTCCGTCGAATCTGCAGTGAA (Panel B). Panel C shows the SLNCR sequence that mediates autoregulation of SLNCR isoforms. SLNCR, SLNCR2 and SLNCR3 contain an Alu element in their final exon and Alu elements have been shown to mediate inter-RNA interactions resulting in the degradation of one of the Alu containing RNAs (Gong and Maquat (2011) Nature 470:284-288).

FIG. 28 includes 2 panels, identified as panels A and B, which ARE and Brn3a binding sites are required for SLNCR1-mediated induction of the MMP9 promoter. Panel A shows schematic of the 2 kb MMP9 promoter cloned upstream of the firefly luciferase reporter. The black box denotes a predicted Brn3a binding site. The wild-type and mutated sequences are shown below. The black box denotes a functional ARE, with wild-type and mutated sequences below. The grey circles denote additional predicted AREs. Panel B shows mutation of either the Brn3a binding site (MMP9p-FL BBS mut) or the ARE (MMP9p-FL ARE mut) prevents SLNCR1-mediated upregulation of the MMP9 promoter. Assay was completed as in FIG. 3I. Error bars represent standard deviation from four reactions, ** p-value<0.005, n.s.=not significant.

FIG. 29 includes 3 panels, identified as panels A, B, and C. Panel A shows a model for SLNCR1 function in melanoma invasion. Upon expression of SLNCR1 in melanomas, AR and Brn3a bind to conserved, adjacent regions of the lncRNA. The SLNCR1/AR/Brn3a ternary complex has high affinity for adjacent Brn3a binding site and ARE located upstream of the MMP9 transcriptional start site and cooperatively binds to the promoter. Binding of AR and Brn3a increases MMP9 expression and activity, subsequently increasing invasion of melanoma cells. Panel B shows expression of SLNCR1 does not affect localization of AR. Western blot of cytoplasmic (C) and nuclear (N) fractions of A375 cells transfected with an empty or SLNCR1-expressing vector. The blot was probed with α-Hsp90 and α-snRNP70 antibodies to confirm successful fractionation, and localization of AR was determined using an α-AR antibody. Panel C shows AR associated lncRNAs display a region of high similarity, related to FIG. 7. Alignment of ARbound regions of SLNCR1, HOTAIR, PCGEM1 and a similar region of SRA1. Alignment was performed as in FIGS. 1G to 1I. Numbers in parentheses indicate the lncRNA nucleotides shown in the alignment.

FIG. 30 includes 4 panels, identified as panels A, B, C, and D. Panel A shows the single stranded RNA oligos that were tested, with the short name of the oligos in the first column and the sequences tested in the second column. The third column (AR binding) summarizes results from the REMSAs, with ‘+’ indicating binding to AR, and ‘−’ indicating no binding. Binding events appear as a defined upward shift or smear in the RNA band. The fourth column (Secondary structure) indicates if a strong hairpin structure is predicted to form in the given sequence, as predicted by RNAStructure (Reuter et al. (2010) BMC Bioinformatics 11:129). For REMSAs shown in panels B and C, 20 μl reactions were assembled containing 200 nM purified AR protein (Abcam ab82609), 0.5 nM of the indicated biotinylated RNA probes, 2 μg of non-specific tRNA in 1×REMSA binding buffer from the LightShift Chemiluminescent RNA kit (Thermo Scientific). For competition experiments shown in panel C, unlabeled RNA oligo was added prior to addition of AR protein at a final concentration of 10 μM. The REMSA in panel D were performed with the indicated final concentration of AR protein, with biotinylated AR min 2 as the probe. Reactions were incubated for 30 minutes at room temperature before resolving on a 5% TBE (Bio-Rad, 4565013). The gel was transferred to Amersham Hybond-N+ membrane (GE Lifesciences) for 30 minutes at 400 mA in 0.5×TBE, and blot was crosslinked and probed following manufacturer's instructions (LightShift Chemiluminescent RNA kit, Thermo Scientific).

FIG. 31 includes 8 panels, identified as panels A, B, C, D, E, F, G, and H. The melanoma short-term culture WM1575 expresses high constitutive levels of PD-L1 (Panels A and B), and knockdown of SLNCR increases expression of PD-L1, while knockdown of AR decreases PD-L1 expression. The melanoma cells lines SK-MEL-28, A375 and RPMI-7951 express low levels of PD-L1; however, addition of interferon gamma (INF-γ) induced expression of PD-L1 (Panels E-H). While knockdown of SLNCR or AR has little effect on PD-L1 levels in uninduced SK-MEL-28 or A375 cells (data not shown), knockdown of SLNCR increases INF-γ induced PD-L1 expression (Panels C-F). Interestingly, knockdown of SLNCR attenuates INF-γ induction in RPMI-7951, possibly a consequence of longer knockdown before induction or cell type specific differences (Panels G and H). Knockdown of AR attenuates INF-γ induction of PD-L1 in SK-MEL-28, A375 and RPMI-7951, confirming that AR is critical for expression of PD-L1 (Panels C-H).

FIG. 32 includes two panels, identified as panels A and B. SLNCR is expressed more highly in basal (Panel A) and ER-negative (Panel B) breast cancers, showing that SLNCR is a biomarker for breast cancer subtypes. Expression of the lncRNA is on a Log 2 scale. For subtype classification, the p-value is 2.630867e-82. For ER status, the p-value is 1.151872e-53. The data were generated from the TANRIC database using SLNCR genomic coordinates (17:70399463-70400957;70424377-70424439;70427269-70427365;70588342-70588943) as the input (Li et al. (2015) Cancer research 75:3728-3737). Expression of the lncRNA is on a Log 2 scale.

FIG. 33 includes 3 panels, identified as panels A, B, and C. Kaplan-Meier survival curves are provided indicating that high SLNCR expression is associated with worse overall survival in overall breast cancers (Panel A, univariate Cox proportional hazards model p-value=0.56 and log-rank test p-value=0.55), ER-positive breast cancers (Panel B, cox p-value=0.2 and log-rank p-value=0.45), and LumA breast cancers (Panel C, cox p-value=0.67 and log-rank p-value=0.44). Survival time is shown in days. The data were generated from the TANRIC database using SLNCR genomic coordinates (17:70399463-70400957;70424377-70424439;70427269-70427365;70588342-70588943) as the input (Li et al. (2015) Cancer research 75:3728-3737).

FIG. 34 shows that SLNCR is significantly upregulated in Stages II-IV bladder urothelial cancer compared to Stage I (p-value=0.15), indicating that SLNCR is a biomarker for progression of bladder urothelial cancer. The data was generated from the TANRIC database using SLNCR genomic coordinates (17:70399463-70400957;70424377-70424439;70427269-70427365;70588342-70588943) as the input (Li et al. (2015) Cancer research 75:3728-3737). Expression of the lncRNA is on a Log 2 scale.

FIG. 35 indicates that high SLNCR expression is associated with better overall survival of glioblastoma multiforme (cox p-value=0.32 and log-rank p-value=0.16). The data was generated from the TANRIC database using SLNCR genomic coordinates (17:70399463-70400957;70424377-70424439;70427269-70427365;70588342-70588943) as the input (Li et al. (2015) Cancer research 75:3728-3737).

FIG. 36 includes 2 panels, identified as panels A and B. Panel A shows that SLNCR expression is significantly associated with histological tumor grade (p-value=8.687363e-7). Panel B shows that SLNCR expression is associated with worse overall survival in kidney renal clear cell carcinoma (cox p-value=0.0001 and log-rank p-value=0.0002), indicating that SLNCR is a diagnostic and prognostic biomarker of kidney renal clear cell carcinoma. The data was generated from the TANRIC database using SLNCR genomic coordinates (17:70399463-70400957;70424377-70424439;70427269-70427365;70588342-70588943) as the input (Li et al. (2015) Cancer research 75:3728-3737). Expression of the lncRNA is on a Log 2 scale.

FIG. 37 includes 2 panels, identified as panels A and B. Panel A shows that SLNCR expression is significantly correlated (p-value=0.007) with stage of lung squamous cell carcinoma. Higher SLNCR expression is associated with better overall survival in Stage III lung squamous cell carcinoma (Panel B, cox p-value=0.039 and log-rank p-value=0.076), indicating that SLNCR is a diagnostic and prognostic biomarker of lung squamous cell carcinoma. The data was generated from the TANRIC database using SLNCR genomic coordinates (17:70399463-70400957;70424377-70424439;70427269-70427365;70588342-70588943) as the input (Li et al. (2015) Cancer research 75:3728-3737). Expression of the lncRNA is on a Log 2 scale.

FIG. 38 includes 2 panels, identified as panels A and B. Panel A shows that SLNCR expression is significantly correlated (p-value=0.04) with stage of ovarian serous cystadenocarcinoma. Higher SLNCR expression is associated with better overall survival in ovarian serous cystadenocarcinoma patients (Panel B, cox p-value=0.01 and log-rank p-value=0.04), indicating that SLNCR is a diagnostic and prognostic biomarker of ovarian serous cystadenocarcinoma. The data was generated from the TANRIC database using SLNCR genomic coordinates (17:70399463-70400957;70424377-70424439;70427269-70427365;70588342-70588943) as the input (Li et al. (2015) Cancer research 75:3728-3737). Expression of the lncRNA is on a Log 2 scale.

FIG. 39 includes 2 panels, identified as panels A and B. Panel A shows that SLNCR expression is significantly correlated (p-value=7.190241e-11) with molecular subtype of lung adenocarcinoma. Higher SLNCR expression is associated with better overall survival in stomach adenocarcinoma (Panel B, cox p-value=0.05 and log-rank p-value=0.095), indicating that SLNCR is a diagnostic and prognostic biomarker of stomach adenocarcinoma. The data was generated from the TANRIC database using SLNCR genomic coordinates (17:70399463-70400957;70424377-70424439;70427269-70427365;70588342-70588943) as the input (Li et al. (2015) Cancer research 75:3728-3737). Expression of the lncRNA is on a Log 2 scale.

FIG. 40 includes 2 panels, identified as panels A and B. Knockdown of AR reduces proliferation of melanoma cells. The decrease in proliferation observed with knockdown of AR is similar to the effects upon knockdown of SLNCR (see FIG. 6 above), suggesting that SLNCR increases proliferation of melanoma cells through regulation of AR activity. The melanoma short term cultures WM1575 (Panel A) and WM1976 (Panel B) were transfected in duplicate (Lipofectamine® RNAiMAX; Life Technologies) with siRNAs targeting AR (siRNAs SI02757265 and SI04434178; Qiagen). The graphs represent the rate of proliferation measured using the WST-1 reagent (Roche) according to the manufacturer's instructions. Cells were incubated for 1 hour prior to measurement.

FIG. 41 includes 2 panels, identified as panels A and B. Knockdown of SLNCR (all isoforms) reduces proliferation of breast cancer cells. Specifically, these cells are believed to be triple negative breast cancer cells, although it does not rule out that SLNCR regulates proliferation of other breast cancer subtypes as well. This data confirms that SLNCR regulates cancer phenotypes in multiple human cancers. The breast cancer cell lines HCC70 (Panel A) and SUM149 (Panel B) were transfected in duplicate (Lipofectamine® RNAiMAX; Life Technologies) with siRNAs targeting SLNCR (LINC00673 siRNAs SI05482540 and SI05482519; Qiagen). The graphs represent the rate of proliferation measured using the WST-1 reagent (Roche) according to the manufacturer's instructions. Cells were incubated for 1 hour prior to measurement.

FIG. 42 shows that SLNCR likely binds to and regulates the activity of the transcription factor STAT3 (Signal transducer and activator of transcription 3, NP_003141.2, NP_644805.1, NP_998827.1). This interaction is functionally supported by the immune genes that are regulated by various SLNCR isoforms (FIG. 16-FIG. 19), as well network analysis of RNA-seq hits indicating an enrichment of JAK-STAT regulated transcripts (FIG. 21). A region of SLNCR1 (nucleotides 971-1013, and 1053-1133, also present in SLNCR2 and SLNCR3) show high similarity to lncRNA-DC, a lncRNA that has been shown to bind to and regulate the activity of STAT3 (Wang et al. (2014 Science 344:310-313). Based on the alignments we predict that STAT3 binds to RNAs containing repeats of GGAG, GGGA, and GAGG. The alignment of the indicated nucleotides of lnc-DC and SLNCR1 was performed in Clustal Omega and viewed in JALVIEW (as in FIG. 1G).

FIG. 43 includes 2 panels, identified as panels A and B. SLNCR contains a second region of very high conservation located downstream of SLNCR cons. This high degree of conservation strongly suggests that this region is required for SLNCR function, especially the short motif GTGG G/C T/C G. The GTGGGTG motif is a known binder of the hnRNP F and H families, highly suggesting that SLNCR interacts with members of this protein family, possible to regulate splicing of the lncRNA. Panel A shows an alignment of SLNCR1 nucleotides 647-756, while Panel B continues the alignment from SLNCR1 nucleotides 757-807. The alignment of SLNCR homologs was performed in Clustal Omega and viewed in JALVIEW (as in FIG. 1G).

FIG. 44 shows sequences surrounding the peak summits for AR ChIP were analyzed for conserved motifs using MEME (http://meme-suite.org/tools/meme). Sixty basepair sequences of the top 1000 peaks were used as input. This sequence motif matches the binding motif of the RE1-silencing transcription factor, REST (or NRSF). Because overexpression of SLNCR affects ARs binding to these sites (summarized in AR ChIP data), this suggests that SLNCR either forms a DNA-RNA triplex structure with DNA sites containing this motif, thereby recruiting AR to these regions of the chromatin, or SLNCR directly interacts with AR and NRSF.

FIG. 45 highlights significant similarities between the str7 (structure 7) of SRA1 and the alternative exon sequence of SLNCR3. The structure of SRA1 was solved by Novikova, Hennelly, and Sanbonmatsu, N A R, 2012 (Novikova et al. (2012) Nucleic acids research 40: 5034-5051). Str7 of SRA1 is known to bind to the transcriptional co-repressor SLIRP (Hatchell et al. (2006) Molecular cell 22:657-668), and therefore we believe that SLNCR3, specifically the region shown below, also binds to SLIRP. The alignment of SRA1 and SLNCR3 was performed in Clustal Omega, and viewed in VARNA (http://varna.lri.fr/). Bases highlighted in RED are conserved between SRA1, while base pairs highlighted in GREEN boxes denote covariant base pairs (i.e., G-C to C-G, or G-C to U-A, etc). The sequence ucagguucaaguugccagccagacucugggcuuccaggaggagugggcuguggauggccugg underneath is the SLNCR3 sequence reflected in the figure.

### DETAILED DESCRIPTION OF THE PRESENT INVENTION

The present invention is based, at least in part, on the discovery of SLNCR lncRNAs, as well as isoforms and fragments thereof, that are useful in diagnosing, prognosing, assessing, preventing, and treating various indications, including cancer.

### I. Definitions

The articles “a” and “an” are used herein to refer to one or to more than one (i.e., to at least one) of the grammatical object of the article. By way of example, “an element” means one element or more than one element.

The term “altered amount” of a marker or “altered level” of a marker refers to increased or decreased copy number of the marker and/or increased or decreased expression level of a particular marker gene or genes in a cancer sample, as compared to the expression level or copy number of the marker in a control sample. The term “altered amount” of a marker also includes an increased or decreased protein level of a marker in a sample, e.g., a cancer sample, as compared to the protein level of the marker in a normal, control sample. The “amount” of a marker, e.g., expression or copy number of a marker, or protein level of a marker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker. In some embodiments, the amount of the marker in the subject can be considered “significantly” higher or lower than the normal amount if the amount is 10%, 15%, 20%, 25%, 30%, 35%, 40%, 45%, 50% or more, higher or lower, respectively, than the normal amount of the marker.

The term “altered level of expression” of a marker refers to an expression level or copy number of a marker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subject not having the associated disease) and preferably, the average expression level or copy number of the marker in several control samples.

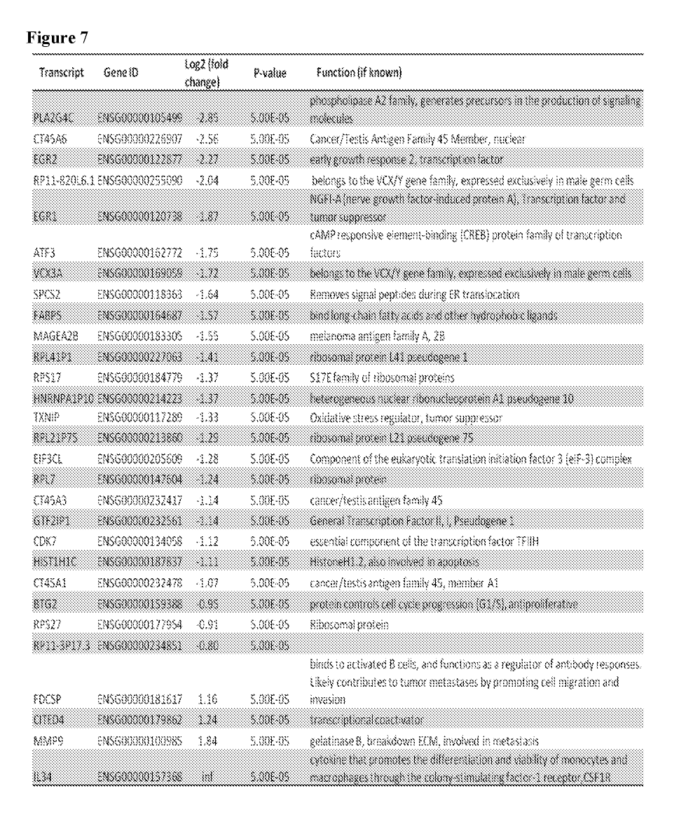

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors. For example, the term “SLNCR activity” includes, but is not limited to, SLNCR-mediated 1) expression or activity of MMP9; 2) downregulation of naturally-occurring SLNCR isoforms; 3) modulation of the expression of one or more genes listed in FIGS. 7, 14, 16, 17, 19, and 31; 4) the expression of PLA2G4C, CT45A6, EGR2, RP11-820L6.1, EGR1, ATF3, VCX3A, SPCS2, FABP5, MAGEA2B, RPL41P1, RPS17, HNRNPA1P10, TXNIP, RPL21P75, EIF3CL, RPL7, CT45A3, GTF2IP1, CDK7, HIST1H1C, CT45A1, BTG2, RPS27, RP11-3P17.3, FDCSP, CITED4, IL34, and PD-L1; 5) cellular proliferation; 6) cell death; 7) cellular migration; 8) genomic replication; 9) angiogenesis induction; 10) cellular invasion; 11) cancer metastasis; 12) binding to one or more protein transcription factors (TF) selected from the group consisting of SRC-1/NCOA-1 (e.g., REFSEQ: NP_671766.1, NP_671756.1, and NP_003734.3); PXR/NR1I2 (e.g., REFSEQ: NP_003880.3, NP_071285.1, and NP_148934.1); PAX5 (e.g., REFSEQ: NP_001267476.1, NP_001267477.1, NP_001267480.1, NP_001267482.1, NP_001267483.1, NP_001267484.1, NP_001267485.1, NP_001267479.1, NP_001267478.1, NP_057953.1, and NP_001267481.1); EGR-1 (e.g., REFSEQ: NP_001955.1); AR (e.g., REFSEQ: NP_000035); E2F-1 (e.g., REFSEQ: NP_005216.1); CAR/NR1I3 (e.g., REFSEQ: NP_001070950.1, NP_001070949.1, NP_001070949.1, NP_001070947.1, NP_001070945.1, NP_001070946.1, NP_001070944.1, NP_001070942.1, NP_001070941.1, NP_001070940.1, and NP_001070939.1); PBX1 (e.g., REFSEQ: NP_001191892.1, NP_001191890.1, and NP_002576.1); ATF2 (e.g., REFSEQ: NP_001243021.1, NP_001243019.1, NP_001871.2, NP_001243023.1, NP_001243022.1, and NP_001243020.1); C/EBP (e.g., REFSEQ: NP_001272758, NP_001272807, NP_001239225, NP_005186, NP_005751, and NP_001796); BRN-3/POU4F1 (e.g., REFSEQ: NP_006228.3); HNF4 (e.g., REFSEQ: NP_000448 and NP_004124); NF-kB (e.g., REFSEQ: NP_001158884, NP_001138610, NP_001070962, NP_006500, and NP_001278675); AP2 (e.g., REFSEQ: NP_001027451, NP_003212.2, NP_001025177.1, NP_001273.1, NP_003213.1, NP_758438.2, and NP_848643.2); OCT4/POU5F1 (e.g., REFSEQ: NP_001272916.1, NP_001272915.1, NP_001167002.1, NP_976034.4, NP_002692.2), SP1 (REFSEQ: NP_001238754.1, NP_003100.1, and NP_612482.2); STAT5 (e.g., REFSEQ: NP_001275649.1, NP_001275648.1, NP_001275647.1, NP_003143.2, and NP_036580.2); p53 (e.g., REFSEQ: NP_001119584.1, NP_000537.3, NP_001263626.1, NP_001263690.1, NP_001263689.1, NP_001119590.1, NP_001119587.1, NP_001119586.1, NP_001119585.1, NP_001263628.1, NP_001263627.1, NP_001263625.1, NP_001263624.1, NP_001119589.1, and NP_001119588.1); TFIID (e.g., REFSEQ: NP_001165556.1, NP_001273003.1, NP_003175.1, NP_114129.1, NP_003176.1, NP_001280654.1, NP_008882.1, NP_001177344.1, NP_005633.1, NP_612639.1, NP_001015892.1, NP_057059.1, NP_006275.1, NP_001257417.1, NP_001128690.1, NP_005636.1, and NP_003478.1); SLIRP (e.g., REFSEQ: NP_112487.1, NP_001254792.1, NP_001254793.1); STAT5 (e.g., REFSEQ: NP_003141.2, NP_644805.1, NP_998827.1); REST (e.g., REFSEQ: NP_005603.3, NP_001180437.1, including isoforms of REST, such as REST4 (e.g., REFSEQ: AEJ31941.1 and UniProt: L0B3Z2, A0A087X1C2, L0B1S6, and A0A087X1C2)); and DAX1 (e.g., REFSEQ: NP_000466.2), optionally wherein the SLNCR-TF complex can translocate to the nucleus; 13) regulation of immune response and/or immune evasion; and 14) modulation of one or more genes listed in Tables S5 and S6 affected by SLNCR overexpression.

The term “altered structure” of a marker refers to the presence of mutations or allelic variants within the marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

The term “altered cellular localization” of a marker refers to the mislocalization of the marker within a cell relative to the normal localization within the cell e.g., within a healthy and/or wild-type cell. An indication of normal localization of the marker can be determined through an analysis of cellular localization motifs known in the field that are harbored by marker polypeptides. For example, SLNCR is a nuclear transcription factor coordinator and naturally functions to present combinations of nuclear transcription factors within the nucleus such that function is abrogated if nuclear import and/or export is inhibited.

Unless otherwise specified herein, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “antisense” nucleic acid polypeptide comprises a nucleotide sequence which is complementary to a “sense” nucleic acid encoding a protein, e.g., complementary to the coding strand of a double-stranded cDNA polypeptide, complementary to an mRNA sequence or complementary to the coding strand of a gene. Accordingly, an antisense nucleic acid polypeptide can hydrogen bond to a sense nucleic acid polypeptide.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, vomit). In a preferred embodiment, body fluids are restricted to blood-related fluids, including whole blood, serum, plasma, and the like.

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer is generally associated with uncontrolled cell growth, invasion of such cells to adjacent tissues, and the spread of such cells to other organs of the body by vascular and lymphatic menas. Cancer invasion occurs when cancer cells intrude on and cross the normal boundaries of adjacent tissue, which can be measured by assaying cancer cell migration, enzymatic destruction of basement membranes by cancer cells, and the like. In some embodiments, a particular stage of cancer is relevant and such stages can include the time period before and/or after angiogenesis, cellular invasion, and/or metastasis. Cancer cells are often in the form of a solid tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present invention is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present invention is used in the treatment, diagnosis, and/or prognosis of melanoma and its subtypes.

The term “classifying” includes “to associate” or “to categorize” a sample with a disease state. In certain instances, “classifying” is based on statistical evidence, empirical evidence, or both. In certain embodiments, the methods and systems of classifying use of a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment. It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal Immune cells that have received a cell-receptor mediated signal, e.g., via an activating receptor are referred to herein as “activated immune cells.”

The term “diagnosing cancer” includes the use of the methods, systems, and code of the present invention to determine the presence or absence of a cancer or subtype thereof in an individual. The term also includes methods, systems, and code for assessing the level of disease activity in an individual. Diagnosis can be performed directly by the agent providing therapeutic treatment. Alternatively, a person providing therapeutic agent can request the a diagnostic assay be performed. The diagnostician and/or the therapeutic interventionist can interpret the diagnostic assay results to determine a therapeutic strategy. Similarly, such alternative processes can apply to other assays, such as prognostic assays.

As used herein, the term “diagnostic marker” includes markers described herein which are useful in the diagnosis of cancer, e.g., over- or under-activity, emergence, expression, growth, remission, recurrence or resistance of tumors before, during or after therapy. The predictive functions of the marker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression (e.g., by ISH, Northern Blot, or qPCR), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human cancers types or cancer samples; (2) its presence or absence in a biological sample, e.g., a sample containing tissue, whole blood, serum, plasma, buccal scrape, saliva, cerebrospinal fluid, urine, stool, or bone marrow, from a subject, e.g., a human, afflicted with cancer; (3) its presence or absence in clinical subset of subjects with cancer (e.g., those responding to a particular therapy or those developing resistance). Diagnostic markers also include “surrogate markers,” e.g., markers which are indirect markers of cancer progression. Such diagnostic markers may be useful to identify populations of subjects amenable to treatment with modulators of SLNCR levels, either alone or in combination with modulators of nuclear transcription factors and/or receptors, to thereby treat such stratified patient populations.

A molecule is “fixed” or “affixed” to a substrate if it is covalently or non-covalently associated with the substrate such the substrate can be rinsed with a fluid (e.g., standard saline citrate, pH 7.4) without a substantial fraction of the molecule dissociating from the substrate.

The term “gene expression data” or “gene expression level” as used herein refers to information regarding the relative or absolute level of expression of a gene or set of genes in a cell or group of cells. The level of expression of a gene may be determined based on the level of RNA, such as mRNA, encoded by the gene. Alternatively, the level of expression may be determined based on the level of a polypeptide or fragment thereof encoded by the gene. Gene expression data may be acquired for an individual cell, or for a group of cells such as a tumor or biopsy sample. Gene expression data and gene expression levels can be stored on computer readable media, e.g., the computer readable medium used in conjunction with a microarray or chip reading device. Such gene expression data can be manipulated to generate gene expression signatures.

The term “gene expression signature” or “signature” as used herein refers to a group of coordinately expressed genes. The genes making up this signature may be expressed in a specific cell lineage, stage of differentiation, or during a particular biological response. The genes can reflect biological aspects of the tumors in which they are expressed, such as the cell of origin of the cancer, the nature of the non-malignant cells in the biopsy, and the oncogenic mechanisms responsible for the cancer.

The term “homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “host cell” is intended to refer to a cell into which a nucleic acid of the present invention, such as a recombinant expression vector of the present invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody,” as used herein, is intended to include antibodies made by a non-human cell having variable and constant regions which have been altered to more closely resemble antibodies that would be made by a human cell, for example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs.

The term “humanized antibody”, as used herein, also includes antibodies in which CDR sequences derived from the germline of another mammalian species, such as a mouse, have been grafted onto human framework sequences.

As used herein, the term “immune cell” refers to cells that play a role in the immune response Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein. Numerous anti-cancer agents in the immunotherapeutic agent class are well known in the art and include, without limitation, antibodies that block or inhibit the function of PD-1, PD-L1, PD-L2, CTLA4, and the like.

As used herein, the term “inhibit” includes the decrease, limitation, or blockage, of, for example a particular action, function, or interaction. For example, cancer is “inhibited” if at least one symptom of the cancer, such as hyperproliferative growth, is alleviated, terminated, slowed, or prevented. As used herein, cancer is also “inhibited” if recurrence or metastasis of the cancer is reduced, slowed, delayed, or prevented.

As used herein, the term “inhibitory signal” refers to a signal transmitted via an inhibitory receptor (e.g., CTLA-4 or PD-1) for a polypeptide on an immune cell. Such a signal antagonizes a signal via an activating receptor (e.g., via a TCR, CD3, BCR, or Fc polypeptide) and can result in, e.g., inhibition of second messenger generation; an inhibition of proliferation; an inhibition of effector function in the immune cell, e.g., reduced phagocytosis, reduced antibody production, reduced cellular cytotoxicity, the failure of the immune cell to produce mediators, (such as cytokines (e.g., IL-2) and/or mediators of allergic responses); or the development of anergy.

As used herein, the term “interaction,” when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules. Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

An “isolated antibody,” as used herein, is intended to refer to an antibody that is substantially free of other antibodies having different antigenic specificities. Moreover, an isolated antibody may be substantially free of other cellular material and/or chemicals.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations, in which compositions of the present invention are separated from cellular components of the cells from which they are isolated or recombinantly produced. In one embodiment, the language “substantially free of cellular material” includes preparations of having less than about 30%, 20%, 10%, or 5% (by dry weight) of cellular material. When an antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g., a package or container) comprising at least one reagent, e.g., a probe, for specifically detecting or modulating the expression of a marker of the present invention. The kit may be promoted, distributed, or sold as a unit for performing the methods of the present invention.

A “marker” or “biomarker” includes a nucleic acid or polypeptide whose altered level of expression in a tissue or cell from its expression level in a control (e.g., normal or healthy tissue or cell) is associated with a disease state, such as a cancer or subtype thereof (e.g., melanoma). A “marker nucleic acid” is a nucleic acid (e.g., mRNA, cDNA, mature miRNA, pre-miRNA, pri-miRNA, miRNA*, piwiRNA, anti-miRNA, or a miRNA binding site, or a variant thereof and other classes of small RNAs known to a skilled artisan) encoded by or corresponding to a marker of the present invention. Such marker nucleic acids include DNA (e.g., cDNA) comprising the entire or a partial sequence of any of the nucleic acid sequences set forth in Table 1, Figures, and the Examples, or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” includes a protein encoded by or corresponding to a marker of the present invention. A marker protein comprises the entire or a partial sequence of any of the sequences set forth in Table 1, the Figures, and the Examples. The terms “protein” and “polypeptide” are used interchangeably. In some embodiments, specific combinations of biomarkers are preferred. For example, a combination or subgroup of one or more of the biomarkers selected from the group shown in Table 1.

The term “melanoma” generally refers to cancers derived from melanocytes. Although melanocytes are predominantly located in skin, they are also found in other parts of the body, including the eye and bowel. Although cutaneous melanoma is most common, melanoma can originate from any melanocyte in the body. Though melanoma is less than five percent of the skin cancers, it is the seventh most common malignancy in the U.S. and is responsible for most of the skin cancer related deaths. The incidence has increased dramatically in the last several decades due to altered sun exposure habits of the population. Several hereditary risk factors are also known. Other important risk factors are the number of pigment nevi, the number dysplastic nevi, and skin type. An increased risk is coupled to many nevi, both benign and dysplastic, and fair skin. Familial history of malignant melanomas is a risk factor, and approximately 8-12% of malignant melanoma cases are familial Additional details are well known, such as described in US Pat. Pubis. 2012-0269764 and 2013-0237445.

Malignant melanomas are clinically recognized based on the ABCD(E) system, where A stands for asymmetry, B for border irregularity, C for color variation, D for diameter >5 mm, and E for evolving. Further, an excision biopsy can be performed in order to corroborate a diagnosis using microscopic evaluation. Infiltrative malignant melanoma is traditionally divided into four principal histopathological subgroups: superficial spreading melanoma (SSM), nodular malignant melanoma (NMM), lentigo maligna melanoma (LMM), and acral lentiginous melanoma (ALM). Other rare types also exists, such as desmoplastic malignant melanoma. A substantial subset of malignant melanomas appear to arise from melanocytic nevi and features of dysplastic nevi are often found in the vicinity of infiltrative melanomas. Melanoma is thought to arise through stages of progression from normal melanocytes or nevus cells through a dysplastic nevus stage and further to an in situ stage before becoming invasive. Some of the subtypes evolve through different phases of tumor progression, which are called radial growth phase (RGP) and vertical growth phase (VGP).

In a preferred embodiment, a melanoma subtype is melanoma resistant to treatment with inhibitors of BRAF and/or MEK. For example, the methods described herein are useful for diagnosing and/or prognosing melanoma subtypes that are resistant to treatment with inhibitors of BRAF and/or MEK. Inhibitors of BRAF and/or MEK, especially of mutant versions implicated in cancer (e.g., BRAF^{V600E}) are well-known in the art.

BRAF is a member of the Raf kinase family of serine/threonine-specific protein kinases. This protein plays a role in regulating the MAP kinase/ERKs signaling pathway, which affects cell division, differentiation, and secretion. BRAF transduces cellular regulatory signals from Ras to MEK in vivo. BRAF is also referred to as v-raf murine sarcoma viral oncogene homolog B1. BRAF mutants are a mutated form of BRAF that has increased basal kinase activity relative to the basal kinase activity of wild type BRAF is also an activated form of BRAF. More than 30 mutations of the BRAF gene that are associated with human cancers have been identified. The frequency of BRAF mutations in melanomas and nevi are 80%. In 90% of the cases, a Glu for Val substitution at position 600 (referred to as V600E) in the activation segment has been found in human cancers.

This mutation is observed in papillary thyroid cancer, colorectal cancer and melanoma. Other mutations which have been found are R462I, I463S, G464E, G464V, G466A, G466E, G466V, G469A, G469E, N581S, E585K, D594V, F595L, G596R, L597V, T599I, V600D, V600K, V600R, K601E or A728V. Most of these mutations are clustered to two regions: the glycine-rich P loop of the N lobe and the activation segment and flanking regions. A mutated form of BRAF that induces focus formation more efficiently than wild type BRAF is also an activated form of BRAF. As used herein, the term “inhibitor of BRAF” refers to a compound or agent, such as a small molecule, that inhibits, decreases, lowers, or reduces the activity of BRAF or a mutant version thereof. Examples of inhibitors of BRAF include, but are not limited to, vemurafenib (PLX-4032; also known as RG7204, R05185426, and vemurafenib, C23H18ClF2N3O3S), PLX 4720 (C17H14C1F2N303S), sorafenib (C21H16ClF3N4O3), GSK2118436, and the like. These and other inhibitors of BRAF, as well as non-limited examples of their methods of manufacture, are described in, for example, PCT Publication Nos. WO 2007/002325, WO 2007/002433, WO 2009/047505, WO 03/086467; WO 2009/143024, WO 2010/104945, WO 2010/104973, WO 2010/111527 and WO 2009/152087; U.S. Pat. Nos. 6,187,799 and 7,329,670; and U.S. Patent Application Publication Nos. 2005/0176740 and 2009/0286783, each of which is herein incorporated by reference in its entirety).

MEK1 is a known as dual specificity mitogen-activated protein kinase kinase 1, which is an enzyme that in human is encoded by the MAP2K1 gene. Mutations of MEK1 involved in cancer are known and include, for example, mutation selected from 59delK and P387S or Q56P or C121S or P124L or F129L, and a MAP2K1 gene having a 175-177 AAG deletion or C1159T. As used herein, the term “inhibitor of MEK” refers to a compound or agent, such as a small molecule, that inhibits, decreases, lowers, or reduces the activity of MEK or a mutant version thereof. Examples of inhibitors of MEK include, but are not limited to, AZD6244 (6-(4-Bromo-2-chloro-phenylamino)-7-fluoro-3-methyl-3H-benzoimida-zole-5-carboxylic acid (2-hydroxy-ethoxy)-amide; selumetinib; Structure IV), and U0126 (1,4-diamino-2,3-dicyano-1,4-bis [2-aminophenylthio]butadiene; ARRY-142886; Structure V). Further non-limiting examples of MEK inhibitors include PD0325901, AZD2171, GDC-0973/XL-518, PD98059, PD184352, GSK1120212, RDEA436, RDEA119/BAY869766, AS703026, BIX 02188, BIX 02189, CI-1040 (PD184352), PD0325901, and PD98059. These and other inhibitors of MEK, as well as non-limiting examples of their methods of manufacture, are described in, for example, U.S. Pat. Nos. 5,525,625; 6,251,943; 7,820,664; 6,809,106; 7,759,518; 7,485,643; 7,576,072; 7,923,456; 7,732,616; 7,271,178; 7,429,667; 6,649,640; 6,495,582; 7,001,905; US Patent Publication No. US2010/0331334, US2009/0143389, US2008/0280957, US2007/0049591, US2011/0118298, International Patent Application Publication No. WO98/43960, WO99/01421, WO99/01426, WO00/41505, WO00/42002, WO00/42003, WO00/41994, WO00/42022, WO00/42029, WO00/68201, WO01/68619, WO02/06213 and WO03/077914, each of which is herein incorporated by reference in their entirety.

Malignant melanomas are staged according to the American Joint Committee on Cancer (AJCC) TNM-classification system, where Clark level is considered in T-classification. The T stage describes the local extent of the primary tumor, i.e., how far the tumor has invaded and imposed growth into surrounding tissues, whereas the N stage and M stage describe how the tumor has developed metastases, with the N stage describing spread of tumor to lymph nodes and the M stage describing growth of tumor in other distant organs. Early stages include: T0-1, N0, M0, representing localized tumors with negative lymph nodes. More advanced stages include: T2-4, N0, M0, localized tumors with more widespread growth and T1-4, N1-3, M0, tumors that have metastasized to lymph nodes and T1-4, N1-3, M1, tumors with a metastasis detected in a distant organ.

Stages I and II represent no metastatic disease and for stage I (T1a/b-2a,N0,M0) prognosis is very good. The 5-year survival for stage I disease is 90-95%, for stage II (T2b-4-b,N0,M0) the corresponding survival rate ranges from 80 to 45%. Stages III (T1a-4-b,N1a-3,M0) and IV (T(aII),N(aII),M1a-c) represent spread disease, and for these stages 5-year survival rates range from 70 to 24%, and from 19 to 7%, respectively. “Clark's level” is a measure of the layers of skin involved in a melanoma and is a melanoma prognostic factor. For example, level I involves the epidermis. Level II involves the epidermis and upper dermis. Level III involves the epidermis, upper dermis, and lower dermis. Level IV involves the epidermis, upper dermis, lower dermis, and subcutis. When the primary tumor has a thickness of >1 mm, ulceration, or Clark level IV-V, sentinel node biopsy (SNB) is typically performed. SNB is performed by identifying the first draining lymph node/s (i.e., the SN) from the tumour. This is normally done by injection of radiolabelled colloid particles in the area around the tumour, followed by injection of Vital Blue dye. Rather than dissection of all regional lymph nodes, which was the earlier standard procedure, only the sentinel nodes are generally removed and carefully examined Following complete lymph node dissection is only performed in confirmed positive cases.

In addition to staging and diagnosis, factors like T-stage, Clark level, SNB status, Breslow's depth, ulceration, and the like can be used as endpoints and/or surrogates for analyses according to the present invention. For example, patients who are diagnosed at an advanced stage with metastases generally have a poor prognosis. For patients diagnosed with a localized disease, the thickness of the tumor measured in mm (Breslow) and ulceration can be endpoints for prognosis. Breslow's depth is determined by using an ocular micrometer at a right angle to the skin. The depth from the granular layer of the epidermis to the deepest point of invasion to which tumor cells have invaded the skin is directly measured. Clark level is important for thin lesions (<1 mm). Other prognostic factors include age, anatomic site of the primary tumor and gender. The sentinel node (SN) status can also be a prognostic factor, especially since the 5-year survival of SN-negative patients has been shown to be as high as 90%. Similarly, overall survival (OS) can be used as a standard primary endpoint. OS takes in to account time to death, irrespective of cause, e.g. if the death is due to cancer or not. Loss to follow-up is censored and regional recurrence, distant metastases, second primary malignant melanomas and second other primary cancers are ignored. Other surrogate endpoints for survival can be used, as described further herein, such as disease-free survival (DFS), which includes time to any event related to the same cancer, i.e. all cancer recurrences and deaths from the same cancer are events.

In addition to endpoints, certain diagnostic and prognostic markers can be analyzed in conjunction with the methods described herein. For example, lactate dehydrogenase (LDH) can be measured as a marker for disease progression. Patients with distant metastases and elevated LDH levels belong to stage IV M1c. Another serum biomarker of interest is S100B. High S100B levels are associated with disease progression, and a decrease in the S100B level is an indicator of treatment response. Melanoma-inhibiting activity (MIA) is yet another serum biomarker that has been evaluated regarding its prognostic value. Studies have shown that elevated MIA levels are rare in stage I and II disease, whereas in stage III or IV, elevation in MIA levels can be seen in 60-100% of cases. Additional useful biomarkers include RGS1 (associated with reduced relapse-free survival (RFS)), osteopontin (associated with both reduced RFS and disease-specific survival (DSS), and predictive of SLN metastases), HERS (associated with reduced survival), and NCOA3 (associated with poor RFS and DSS, and predictive of SLN metastases). In addition, HMB-45, Ki-67 (MIB1), MITF and MART-1/Melan-A or combinations of any described marker may be used for staining (Ivan & Prieto, 2010, Future Oncol. 6(7), 1163-1175; Linos et al., 2011, Biomarkers Med. 5(3) 333-360). In a literature review Rothberg et al. report that melanoma cell adhesion molecule (MCAM)/MUC18, matrix metalloproteinase-2, Ki-67, proliferating cell nuclear antigen (PCNA) and p16/INK4A are predictive of either all-cause mortality or melanoma specific mortality (Rothberg et al., 2009 J. Nat. Canc. Inst. 101(7) 452-474).

Currently, the typical primary treatment of malignant melanoma is radical surgery. Even though survival rates are high after excision of the primary tumour, melanomas tend to metastasize relatively early, and for patients with metastatic melanoma the prognosis is poor, with a 5-year survival rate of less than 10%. Radical removal of distant metastases with surgery can be an option and systemic chemotherapy can be applied, but response rates are normally low (in most cases less than 20%), and most treatment regiments fail to prolong overall survival. The first FDA-approved chemotherapeutic agent for treatment of metastatic melanoma was dacarbazine (DTIC), which can give response rates of approximately 20%, but where less than 5% may be complete responses. Temozolamid is an analog of DTIC that has the advantage of oral administration, and which have been shown to give a similar response as DTIC. Other chemotherapeutic agents, for example different nitrosureas, cisplatin, carboplatin, and vinca alkaloids, have been used, but without any increase in response rates. Since chemotherapy is an inefficient treatment method, immunotherapy agents have also been proposed. Most studied are interferon-alpha and interleukin-2. As single agents they have not been shown to give a better response than conventional treatment, but in combination with chemotherapeutic agents higher response rates have been reported. For patients with resected stage IIB or III melanoma, some studies have shown that adjuvant interferon alfa has led to longer disease free survival. For first- or second-line stage III and IV melanoma systemic treatments include: carboplatin, cisplatin, dacarbazine, interferon alfa, high-dose interleukin-2, paclitaxel, temozolomide, vinblastine or combinations thereof (NCCN Guidelines, ME-D, MS-9-13). Recently, the FDA approved Zelboraf™ (vemurafenib, also known as INN, PLX4032, RG7204 or R05185426) for unresectable or metastatic melanoma with the BRAF V600E mutation (Bollag et al. (2010) Nature 467:596-599 and Chapman et al. (2011) New Eng. J. Med. 364:2507-2516). Another recently approved drug for unresectable or metastatic melanoma is Yervoy®(ipilimumab) an antibody which binds to cytotoxic T-lymphocyte-associated antigen 4 (CTLA-4) (Hodi et al. (2010) New Eng. J. Med. 363:711-723). Others recently reported that patients with KIT receptor activating mutations or over-expression responded to Gleevac® (imatinib mesylate) (Carvajal et al. (2011) JAMA 305:2327-2334). In addition, radiation treatment may be given as an adjuvant after removal of lymphatic metastases, but malignant melanomas are relatively radioresistant. Radiation treatment might also be used as palliative treatment. Melanoma oncologists have also noted that BRAF mutations are common in both primary and metastatic melanomas and that these mutations are reported to be present in 50-70% of all melanomas. This has led to an interest in B-raf inhibitors, such as sorafenib, as therapeutic agents.

The term “modulate” includes up-regulation and down-regulation, e.g., enhancing or inhibiting a response.

The “normal” or “control” level of expression of a marker is the level of expression of the marker in cells of a subject, e.g., a human patient, not afflicted with a cancer. An “over-expression” or “significantly higher level of expression” of a marker refers to an expression level in a test sample that is greater than the standard error of the assay employed to assess expression, and is preferably at least 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the marker in a control sample (e.g., sample from a healthy subject not having the marker associated disease) and preferably, the average expression level of the marker in several control samples. A “significantly lower level of expression” of a marker refers to an expression level in a test sample that is at least 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the marker in a control sample (e.g., sample from a healthy subject not having the marker associated disease) and preferably, the average expression level of the marker in several control samples.

The term “nuclear receptor target drugs” refers to a agents that inhibit the expression and/or activity of nuclear receptors involved in regulating gene expression of gene sets. Nuclear receptors target drugs are well known in the art and include, without limitation, luteinizing hormone-releasing hormone (LHRH) analogs, androgen receptor inhibitors, anti-androgens, hormone blocking drugs, nuclear receptor agonists, nuclear receptor antagonists, selective receptor modulators, selective androgen receptor modulators (SARMs), selective estrogen receptor modulators (SERMs), selective progesterone receptor modulators (SPRMs), selective glucocorticoid receptor agonists (SEGRAs), and selective glucocorticoid receptor modulators (SEGRMs). Specific agents are also well known in the art and include, without limitation, leuprolide (Lupron®, Eligard®), goserelin (Zoladex®), triptorelin (Trelstar®), histrelin (Vantas®), degarelix (Firmagon®), bicalutamide (Casode®), enzalutamide (Xtandi®), flutamide (Eulexin®), nilutamide (Nilandron®), ketoconazole (Nizoral®), abiraterone (Zytiga®), dexamethasone, megestrol acetate (Megace®), medroxyprogesterone acetate (MPA), ethisterone, norethindrone acetate, norethisterone, norethynodrel, ethynodiol diacetate, norethindrone, norgestimate, norgestrel, levonorgestrel, medroxyprogesterone acetate, desogestrel, etonogestrel, drospirenone, norelgestromin, desogestrel, etonogestrel, gestodene, dienogest, drospirenone, elcometrine, nomegestrol acetate, trimegestone, tanaproget, BMS948, mifepristone, 4-hydroxytamoxifen, CINPA1, Cyproterone acetate (Androcur®, Cyprostat®, Siterone®), chlormadinone acetate (Clordion®, Gestafortin®, Lormin®, Non-Ovlon®, Normenon®, Verton®), 17-hydroxyprogesterone (17-OHP), THC, clotrimazole, PK11195 [1-(2-chlorophenyl)-N-methyl-N-(1-methylpropyl)-3-isoquinolinecarboxamide], meclizine, androstanol, CITCO [6-(4-chlorophenyl)imidazo [2,1-b][1,3] thiazole-5-carbaldehyde 0-(3,4-dichlorobenzyl) oxime], zearalenone (ZEN), T0901317, S07662, enobosarm, BMS-564,929, LGD-4033, AC-262,356, JNJ-28330835, LGD-2226, LGD-3303, S-40503, S-23, clomifene, femarelle, ormeloxifene, raloxifene, tamoxifen, toremifene, lasofoxifene, ospemifene, afimoxifene, arzoxifene, bazedoxifene, gulvestrant (Faslodex®, ICI-182780), CDB-4124, asoprisnil, proellex, mapracorat (BOL-303242-X, ZK 245186), fosdagrocorat (PF-04171327), ZK 216348, and 55D1E1. Additional exemplary agents include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of nuclear receptor targets, or fragments thereof.

The term “pre-malignant lesions” as described herein refers to a lesion that, while not cancerous, has potential for becoming cancerous. It also includes the term “pre-malignant disorders” or “potentially malignant disorders.” In particular this refers to a benign, morphologically and/or histologically altered tissue that has a greater than normal risk of malignant transformation, and a disease or a patient's habit that does not necessarily alter the clinical appearance of local tissue but is associated with a greater than normal risk of precancerous lesion or cancer development in that tissue (leukoplakia, erythroplakia, erytroleukoplakia lichen planus (lichenoid reaction) and any lesion or an area which histological examination showed atypia of cells or dysplasia.

The term “predictive” includes the use of a biomarker nucleic acid, protein, and/or metabolite status, e.g., over- or under-activity, emergence, expression, growth, remission, recurrence or resistance of tumors before, during or after therapy, for determining an outcome, such as the likelihood of response of a cancer to anti-SLNCR treatment, either alone or in combination with a nuclear receptor target drug or other anti-cancer agent. Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC) and/or biomarker metabolite, or increased or decreased activity (determined by, for example, modulation of the kynurenine pathway), e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular anti-cancer therapy or those developing resistance thereto).

The term “pre-determined” biomarker amount and/or activity measurement(s) may be a biomarker amount and/or activity measurement(s) used to, by way of example only, evaluate a subject that may be selected for a particular treatment, evaluate a response to a treatment such as an anti-immune checkpoint inhibitor therapy, and/or evaluate the disease state. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of a housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of melanoma, development of one or more clinical factors, development of intestinal cancer, or recovery from the disease. In some embodiments, the term “good prognosis” indicates that the expected or likely outcome after treatment of melanoma is good. The term “poor prognosis” indicates that the expected or likely outcome after treatment of melanoma is not good.

The term “response to cancer therapy” or “outcome of cancer therapy” relates to any response of the hyperproliferative disorder (e.g., cancer) to a cancer therapy, preferably to a change in tumor mass and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Response may also be assessed by caliper measurement or pathological examination of the tumor after biopsy or surgical resection for solid cancers. Responses may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to copy number, level of expression, level of activity, etc. of one or more biomarkers listed in Table 1, the Figures, and the Examples, or the Examples that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom the measurement values are known. In certain embodiments, the same doses of cancer therapeutic agents are administered to each subject. In related embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker threshold values that correlate to outcome of a cancer therapy can be determined using methods such as those described in the Examples section. Outcomes can also be measured in terms of a “hazard ratio” (the ratio of death rates for one patient group to another; provides likelihood of death at a certain time point), “overall survival” (OS), and/or “progression free survival.” In certain embodiments, the prognosis comprises likelihood of overall survival rate at 1 year, 2 years, 3 years, 4 years, or any other suitable time point. The significance associated with the prognosis of poor outcome in all aspects of the present invention is measured by techniques known in the art. For example, significance may be measured with calculation of odds ratio. In a further embodiment, the significance is measured by a percentage. In one embodiment, a significant risk of poor outcome is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. Thus, the present invention further provides methods for making a treatment decision for a cancer patient, comprising carrying out the methods for prognosing a cancer patient according to the different aspects and embodiments of the present invention, and then weighing the results in light of other known clinical and pathological risk factors, in determining a course of treatment for the cancer patient. For example, a cancer patient that is shown by the methods of the present invention to have an increased risk of poor outcome by combination chemotherapy treatment can be treated with more aggressive therapies, including but not limited to radiation therapy, peripheral blood stem cell transplant, bone marrow transplant, or novel or experimental therapies under clinical investigation.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal who is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., chemotherapeutic or radiation therapy. In some embodiments, normal cells are not affected to an extent that causes the normal cells to be unduly injured by the cancer therapy (e.g., chemotherapy or radiation therapy). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “SLNCR,” unless otherwise specified, refers to the known SLNCR (SLNCR1) lncRNA, as well as its isoforms, such as SLNCR2 (SLNCR4A) and SLNCR3 (SLNCR4B), and biologically active fragments thereof. SLNCR1, also known as LINC00673 in the art, is a 2,257 nucleotide sequence associated with Ref Seq Gene ID NR_036488.1 and Entrez Gene ID 100499467. It is located immediately downstream of another lncRNA known as LINC00511 on human chromosome 17. SLNCR2 (SLNCR4A) and SLNCR3 (SLNCR4B) are isoforms described herein. SLNCR and its isoforms act as a scaffold to bring together one or more transcription factors and associated co-activators and/or co-repressors for translocation to the nucleus and activation and/or repression of gene expression. In cancer cells, such as melanoma cells, SLNCR and its isoforms mediate one or more of the following functions: 1) expression or activity of MMP9; 2) downregulation of naturally-occurring SLNCR isoforms; 3) modulation of the expression of one or more genes listed in FIGS. 7, 14, 16, 17, 19, and 31; 4) the expression of PLA2G4C, CT45A6, EGR2, RP11-820L6.1, EGR1, ATF3, VCX3A, SPCS2, FABP5, MAGEA2B, RPL41P1, RPS17, HNRNPA1P10, TXNIP, RPL21P75, EIF3CL, RPL7, CT45A3, GTF2IP1, CDK7, HIST1H1C, CT45A1, BTG2, RPS27, RP11-3P17.3, FDCSP, CITED4, IL34, and PD-L1; 5) cellular proliferation; 6) cell death; 7) cellular migration; 8) genomic replication; 9) angiogenesis induction; 10) cellular invasion; 11) cancer metastasis; 12) binding to one or more protein transcription factors (TF) selected from the group consisting of SRC-1/NCOA-1 (e.g., REFSEQ: NP_671766.1, NP_671756.1, and NP_003734.3); PXR/NR1I2 (e.g., REFSEQ: NP_003880.3, NP_071285.1, and NP_148934.1); PAX5 (e.g., REFSEQ: NP_001267476.1, NP_001267477.1, NP_001267480.1, NP_001267482.1, NP_001267483.1, NP_001267484.1, NP_001267485.1, NP_001267479.1, NP_001267478.1, NP_057953.1, and NP_001267481.1); EGR-1 (e.g., REFSEQ: NP_001955.1); AR (e.g., REFSEQ: NP_000035); E2F-1 (e.g., REFSEQ: NP_005216.1); CAR/NR1I3 (e.g., REFSEQ: NP_001070950.1, NP_001070949.1, NP_001070949.1, NP_001070947.1, NP_001070945.1, NP_001070946.1, NP_001070944.1, NP_001070942.1, NP_001070941.1, NP_001070940.1, and NP_001070939.1); PBX1 (e.g., REFSEQ: NP_001191892.1, NP_001191890.1, and NP_002576.1); ATF2 (e.g., REFSEQ: NP_001243021.1, NP_001243019.1, NP_001871.2, NP_001243023.1, NP_001243022.1, and NP_001243020.1); C/EBP (e.g., REFSEQ: NP_001272758, NP_001272807, NP_001239225, NP_005186, NP_005751, and NP_001796); BRN-3/POU4F1 (e.g., REFSEQ: NP_006228.3); HNF4 (e.g., REFSEQ: NP_000448 and NP_004124); NF-kB (e.g., REFSEQ: NP_001158884, NP_001138610, NP_001070962, NP_006500, and NP_001278675); AP2 (e.g., REFSEQ: NP_001027451, NP_003212.2, NP_001025177.1, NP_001273.1, NP_003213.1, NP_758438.2, and NP_848643.2); OCT4/POU5F1 (e.g., REFSEQ: NP_001272916.1, NP_001272915.1, NP_001167002.1, NP_976034.4, NP_002692.2), SP1 (REFSEQ: NP_001238754.1, NP_003100.1, and NP_612482.2); STAT5 (e.g., REFSEQ: NP_001275649.1, NP_001275648.1, NP_001275647.1, NP_003143.2, and NP_036580.2); p53 (e.g., REFSEQ: NP_001119584.1, NP_000537.3, NP_001263626.1, NP_001263690.1, NP_001263689.1, NP_001119590.1, NP_001119587.1, NP_001119586.1, NP_001119585.1, NP_001263628.1, NP_001263627.1, NP_001263625.1, NP_001263624.1, NP_001119589.1, and NP_001119588.1); TFIID (e.g., REFSEQ: NP_001165556.1, NP_001273003.1, NP_003175.1, NP_114129.1, NP_003176.1, NP_001280654.1, NP_008882.1, NP_001177344.1, NP_005633.1, NP_612639.1, NP_001015892.1, NP_057059.1, NP_006275.1, NP_001257417.1, NP_001128690.1, NP_005636.1, and NP_003478.1); SLIRP (e.g., REFSEQ: NP_112487.1, NP_001254792.1, NP_001254793.1); STAT5 (e.g., REFSEQ: NP_003141.2, NP_644805.1, NP_998827.1); REST (e.g., REFSEQ: NP_005603.3, NP_001180437.1, including isoforms of REST, such as REST4 (e.g., REFSEQ: AEJ31941.1 and UniProt: L0B3Z2, A0A087X1C2, L0B1S6, and A0A087X1C2)); and DAX1 (e.g., REFSEQ: NP_000466.2), optionally wherein the SLNCR-TF complex can translocate to the nucleus; 13) regulation of immune response and/or immune evasion; and 14) modulation of one or more genes listed in Tables S5 and S6 affected by SLNCR overexpression. It has been determined herein that certain SLNCR structural features common or different among the SLNCR isoforms are related to SLNCR function. For example, a highly conserved approximately 301 nucleotide sequence common to the SLNCR 1-3 isoforms (referred to as “SLNCR cons” herein) is sufficient for cancer cell invasion. Similarly, a portion or all of an approximately 111 nucleotide sequence common to the SLNCR 1-3 isoforms (referred to as “SLNCR delta cons” herein) is required for cancer cell invasion. Table 1, the Figures, and the Examples, below provide representative SLNCR sequences, as well as annotations of structural domains and biologically active fragments associated with SLNCR function. For example, Table 1A provides SLNCR sequences encompassed within the scope of compositions-of-matter and methods of the present invention and Table 1B provides representative known SLNCR sequences useful according to the methods of the present invention.

The term “synergistic effect” refers to the combined effect of two or more anticancer agents or chemotherapy drugs can be greater than the sum of the separate effects of the anticancer agents or chemotherapy drugs alone.

The term “subject” refers to any healthy animal, mammal or human, or any animal, mammal or human afflicted with a condition of interest (e.g., cancer). The term “subject” is interchangeable with “patient.” In some embodiments, a subject does not have any cancer other than melanoma. In other embodiments, the subject has melanoma but does not have one or more other cancers of interest. For example, in some embodiments, a subject does not have renal cell carcinoma, head or neck cancer, and/or lung cancer.

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

The term “substantially pure cell population” refers to a population of cells having a specified cell marker characteristic and differentiation potential that is at least about 50%, preferably at least about 75-80%, more preferably at least about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

As used herein, the term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

A “transcribed polynucleotide” or “nucleotide transcript” is a polynucleotide (e.g., an mRNA, hnRNA, cDNA, mature miRNA, pre-miRNA, pri-miRNA, miRNA*, piwiRNA, anti-miRNA, or a miRNA binding site, or a variant thereof or an analog of such RNA or cDNA) which is complementary to or homologous with all or a portion of a mature mRNA made by transcription of a marker of the present invention and normal post-transcriptional processing (e.g., splicing), if any, of the RNA transcript, and reverse transcription of the RNA transcript. In some embodiments, transcribed polynucleotides are “long non-coding RNAs” or “lcRNAs” that are defined as transcribed polynucleotides that do not naturally encode a translated protein. lcRNAs are generally sequences longer than about 100 nucleotides and can be as long as up to tens of kilobases, although the length definition is a matter of convenience for distinguishing traditionally small nucleic acids like microRNAs, siRNAs, and piwi-associated RNAs. lcRNAs may be located separate from protein coding genes (long intergenic ncRNAs or lincRNAs), or reside near or within protein coding genes (Guttman et al. (2009) Nature 458:223-227; Katayama et al. (2005) Science 309:1564-1566; Kim et al. (2010) Nature 465:182-187; De Santa et al. (2010) PLoS Biol. 8:e1000384).

As used herein, the term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors.” In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

An “underexpression” or “significantly lower level of expression or copy number” of a marker refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with cancer) and preferably, the average expression level or copy number of the marker in several control samples.

As used herein, the term “unresponsiveness” includes refractivity of immune cells to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the present invention (or any portion thereof) can be used to derive the fusion protein or polypeptide amino acid sequence, using the genetic code to translate the DNA or RNA into an amino acid sequence. Likewise, for a fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Table 1, the Figures, and the Examples) are well known in the art and readily available on publicly available databases, such as the National Center for Biotechnology Information (NCBI). For example, exemplary nucleic acid and amino acid sequences derived from publicly available sequence databases are provided below.

TABLE 1A
SLNCR Isoforms and Biologically Active Fragments Thereof

SEQ ID NO: 1 Human SLNCR Cons cDNA Sequence
GAAGGCGGCCGCCTGAGGACCCCCGCCCGCGACCTCCGCGAGTCTGGAGCGCAGAGGACAG
GGTCTGGCTGCTCTTTGGCCTTGGATGGAAAGTGGGGAATTGGGTGGGGGGCTGCGGACCC
CTTAACGTGGATTACTTGGTGTGTATCAGCTGGGCTCAGAAGACCCACGACCTCTTCTCCA
TCCGTGGATTGATTTGTTCTGCTTAACAGCTGGGTCGCCAAGCTGGAGGTATTTTTCCCTC
TCCACCCTGGTCTTCTCCTGTAACGTGTGGCCGCCTTTTCCAGCACGGCCTCCTGCC
SEQ ID NO: 2 Human SLNCR2 (SLNCR4a) cDNA Sequence

| | |
|---|---|
| 1 | agggcgcgca ggcggcgcgg gtgcgcggtg cggcgctggt atccagagga cgcggtcacc |
| 61 | gcctctggca tttgtcgttc tgcgcttctc cgcaaggacc ctctgttagg caggcgccca |
| 121 | ccgtaagcct cccgggcctt gtgaacctgc aaacccaagt ctgagagacg atccgccttc |
| 181 | agcgctttcc agcttggcag agaggctttc ccggcgggga tctttggttg gcgctggcga |
| 241 | tgcgcgggga agaaaggcga ggagcggcgt ccaggctggg tgatgtccca gcacgagtag |
| 301 | gcgggatgcg ctcgcttggt cctccgggcg cccggtccct gcccgcgtcg cgcgcccacc |
| 361 | cctggggacg agaaggcggc cgcctgagga cccccgcccg cgacctccgc gagtctggag |
| 421 | cgcagaggac agggtctggc tgctctttgg ccttggatgg aaagtgggga attgggtggg |
| 481 | gggctgcgga ccccttaacg tggattactt ggtgtgtatc agctgggctc agaagaccca |
| 541 | cgacctcttc tccatccgtg gattgatttg ttctgcttaa cagctgggtc gccaagctgg |
| 601 | aggtattttt ccctctccac cctggtcttc tcctgtaacg tgtggccgcc ttttccagca |
| 661 | cggcctcctg ccttcctggt gcactttttg gagaacgtgg tggaatcaga ggtttctggc |
| 721 | tgactcggtg ggtgctttga accaggaaag gacaagaaag aggtgccacc tcctgagtcc |
| 781 | tggttcctgc ggctccgtcg aatctgcagt gaaggccttt gggaagcgtg ggttatgtgg |
| 841 | gtccaggggt cccatcctcc cgtggaggcc cttgacctga ggatcactaa ctcacacccc |
| 901 | accgacttcc ggcccccaac aagggcagtg ttcctcttgc tccattctgc tctgaagtcc |
| 961 | cccaaaccgc ttcctggggc tgcttagtga aatacaaggc tcatctctga ggacctctac |
| 1021 | agggctggat gggaaggact gatccacatt cccaccagga agtttagcag aacccccgcg |
| 1081 | tgccacctgg accccttgga aggacctggc tcaggctgga ccacctcttg agaggcagga |
| 1141 | gctctggatt tgatcaagaa ttctttgctg agcatggtgc ctcatgccta taatcccaac |
| 1201 | actttgggag gccagtgtgg gaggatctct tgagcccagg agttcaagac tagcctgggc |
| 1261 | aacacagaga gaccccatct ctaaaataat aataataata aaataaaaaa ttagcagggc |
| 1321 | atggtggcat gtgcctgtag tcccagctac ccaggaggct gaggcaagag gatggctgga |
| 1381 | gcctgggatg ttgaggctgc aatgaactgt gattacccca ctgcactcca gcctgggcaa |
| 1441 | aagagcgaga gaccctgtct caaataataa taataataat aatcttattt tggagaataa |
| 1501 | agagacctct ggatttgagg tgccatttgg gtagaaagaa aagacgttta caccgagaaa |
| 1561 | tagtctgtgt tgccctgaag gagcagaggg atgcatcgct ggaggtgacc tacagttgaa |
| 1621 | gaagactcat tatgacagac cttgtccttc ttccttgtgg aaagtgtttc ctctgctgct |
| 1681 | actgctcatg agactcttcc ccctccctgt cccagggaac caaagggctt tctaccacac |
| 1741 | cctttcttgc cccccgcctc ccatgtctgc tgtgcctttg tactcagcaa ttcttgtttg |
| 1801 | ctccattatc ttccagccgg atacagagtg aatagttaac cacacttagg tcaaatagga |
| 1861 | tctaaatttt tgttcctgct ccgtgtaaag aggccagtgt ttgtgtgttg caagcagcct |
| 1921 | tggaatagta actcttctca tttgtttggg atctggccac caagttccag aatgatacac |
| 1981 | ggatcagtgc agaagttcat caggctctcg gaccttaggg ctgttggaga aggcttcagc |
| 2041 | agcagaactg atggtgaagg ctcgtgttct ccatcctcaa ctttctttgc ttcgatcata |
| 2101 | cacaagaata catttggaag ggcaaaaaat gaacactgtc gttcattgca gccgtgtttt |
| 2161 | gtgacacaga tgcacagtct gctgtgaaga ccttctctca agtggcattt gggagtccat |
| 2221 | gccagatcat ggtgcttcat gagagactga cagctatcag gggttgtggc acttagtgag |
| 2281 | gactctcctc ccccagtgtg tgctgatgac acatacacac ctgacaatag cttgagtctt |
| 2341 | ctctgttcct tttactctgt agccaacata cacatgattt aaaacccttt ctaaatatct |
| 2401 | atcatggttc atccttgtcc aaatgcagag tcagagctat ttgtacttca ttattatttc |
| 2461 | caaggcgaat agttggcttt ctttttgcaa aaataattaa agtttttgta tgttgcagtt |
| 2521 | gc |

The underlined SLNCR2 sequence is believed to represent a domain that mediates
SLNCR2 isoform-specific functions
SEQ ID NO: 3 Human SLNCR3 (SLNCR4b) cDNA Sequence

| | |
|---|---|
| 1 | agggcgcgca ggcggcgcgg gtgcgcggtg cggcgctggt atccagagga cgcggtcacc |
| 61 | gcctctggca tttgtcgttc tgcgcttctc cgcaaggacc ctctgttagg caggcgccca |
| 121 | ccgtaagcct cccgggcctt gtgaacctgc aaacccaagt ctgagagacg atccgccttc |
| 181 | agcgctttcc agcttggcag agaggctttc ccggcgggga tctttggttg gcgctggcga |
| 241 | tgcgcgggga agaaaggcga ggagcggcgt ccaggctggg tgatgtccca gcacgagtag |
| 301 | gcgggatgcg ctcgcttggt cctccgggcg cccggtccct gcccgcgtcg cgcgcccacc |
| 361 | cctggggacg agaaggcggc cgcctgagga cccccgcccg cgacctccgc gagtctggag |
| 421 | cgcagaggac agggtctggc tgctctttgg ccttggatgg aaagtgggga attgggtggg |
| 481 | gggctgcgga ccccttaacg tggattactt ggtgtgtatc agctgggctc agaagaccca |
| 541 | cgacctcttc tccatccgtg gattgatttg ttctgcttaa cagctgggtc gccaagctgg |
| 601 | aggtattttt ccctctccac cctggtcttc tcctgtaacg tgtggccgcc ttttccagca |
| 661 | cggcctcctg ccttcctggt gcactttttg gagaacgtgg tggaatcaga ggtttctggc |
| 721 | tgactcggtg ggtgctttga accaggaaag gacaagaaag aggaagatca ggttcaagtt |
| 781 | gccagccaga ctctgggctt ccaggaggag tgggctgtgg atggcctggc ctcatttgca |
| 841 | tgtccctctc ctcccggccc tgcaggtgcc acctcctgag tcctggttcc tgcggctccg |
| 901 | tcgaatctgc agtgaaggcc tttgggaagc gtgggttatg tgggtccagg ggtcccatcc |
| 961 | tcccgtggag gcccttgacc tgaggatcac taactcacac cccaccgact tccggccccc |
| 1021 | aacaagggca gtgttcctct tgctccattc tgctctgaag tcccccaaac cgcttcctgg |
| 1081 | ggctgcttag tgaaatacaa ggctcatctc tgaggacctc tacagggctg gatgggaagg |
| 1141 | actgatccac attcccacca ggaagtttag cagaaccccc gcgtgccacc tggacccctt |
| 1201 | ggaaggacct ggctcaggct ggaccacctc ttgagaggca ggagctctgg atttgatcaa |
| 1261 | gaattctttg ctgagcatgg tgcctcatgc ctataatccc aacactttgg gaggccagtg |
| 1321 | tgggaggatc tcttgagccc aggagttcaa gactagcctg ggcaacacag agagacccca |
| 1381 | tctctaaaat aataataata ataaaataaa aaattagcag ggcatggtgg catgtgcctg |
| 1441 | tagtcccagc tacccaggag gctgaggcaa gaggatggct ggagcctggg atgttgaggc |
| 1501 | tgcaatgaac tgtgattacc ccactgcact ccagcctggg caaaagagcg agagaccctg |
| 1561 | tctcaaataa taataataat aataatctta ttttggagaa taaagagacc tctggatttg |
| 1621 | aggtgccatt tgggtagaaa gaaaagacgt ttacaccgag aaatagtctg tgttgccctg |
| 1681 | aaggagcaga gggatgcatc gctggaggtg acctacagtt gaagaagact cattatgaca |
| 1741 | gaccttgtcc ttcttccttg tggaaagtgt ttcctctgct gctactgctc atgagactct |
| 1801 | tccccctccc tgtcccaggg aaccaaaggg ctttctacca caccctttct tgccccccgc |
| 1861 | ctcccatgtc tgctgtgcct ttgtactcag caattcttgt ttgctccatt atcttccagc |
| 1921 | cggatacaga gtgaatagtt aaccacactt aggtcaaata ggatctaaat ttttgttcct |
| 1981 | gctccgtgta aagaggccag tgtttgtgtg ttgcaagcag ccttggaata gtaactcttc |
| 2041 | tcatttgttt gggatctggc caccaagttc cagaatgata cacggatcag tgcagaagtt |
| 2101 | catcaggctc tcggacctta gggctgttgg agaaggcttc agcagcagaa ctgatggtga |
| 2161 | aggctcgtgt tctccatcct caactttctt tgcttcgatc atacacaaga atacatttgg |
| 2221 | aagggcaaaa aatgaacact gtcgttcatt gcagccgtgt tttgtgacac agatgcacag |
| 2281 | tctgctgtga agaccttctc tcaagtggca tttgggagtc catgccagat catggtgctt |
| 2341 | catgagagac tgacagctat caggggttgt ggcacttagt gaggactctc ctcccccagt |
| 2401 | gtgtgctgat gacacataca cacctgacaa tagcttgagt cttctctgtt ccttttactc |
| 2461 | tgtagccaac atacacatga tttaaaaccc tttctaaata tctatcatgg ttcatccttg |
| 2521 | tccaaatgca gagtcagagc tatttgtact tcattattat ttccaaggcg aatagttggc |
| 2581 | tttctttttg caaaaataat taaagttttt gtatgttgca gttgc |

The underlined SLNCR3 sequence is believed to represent a domain that mediates
SLNCR3 isoform-specific functions, either alone or in combination with the SLNCR2
isoform-specific sequence
SEQ ID NO: 4 Human SLNCR Delta Cons Sequence
AAGTGGGGAATTGGGTGGGGGGCTGCGGACCCCTTAACGTGGATTACTTGGTGTGTATCAG
CTGGGCTCAGAAGACCCACGACCTCTTCTCCATCCGTGGATTGATTTGTT
SEQ ID NO: 5 Human SLNCR SRA1 H2 Helix Domain Sequence
GGACAGGGTCTGG
SEQ ID NO: 6 Human SLNCR SRA1 H3 Helix Domain Sequence
GGGCTGCGGACCC
SEQ ID NO: 7 Human SLNCR Brn3a Binding Domain Sequence
GGATTACTT
SEQ ID NO: 8 Human SLNCR AR RNA Binding Domain Consensus Sequence
TCTCC(A/T)
SEQ ID NO: 9 Human SLNCR PXR Binding Domain Sequence
GGAG
SEQ ID NO: 10 Human SLNCR SRA1 H5 Helix Domain Sequence
TTCTGCTTAACAGCTGGGTCGCCAA
SEQ ID NO: 11 Human SLNCR SRA1 H6 Helix Domain Sequence
GCTGGAGGTATTTTTCCCTCTCCACCCTGGTCTTCTCCTGTA
SEQ ID NO: 12 Human SLNCR Autoregulation Domain Sequence

| | |
|---|---|
| 1 | GCTGAGCATG GTGCCTCATG CCTATAATCC CAACACTTTG GGAGGCCAGT GTGGGAGGAT |
| 61 | CTCTTGAGCC CAGGAGTTCA AGACTAGCCT GGGCAACACA GAGAGACCCC ATCTCTAAAA |
| 121 | TAATAATAAT AATAAAATAA AAAATTAGCA GGGCATGGTG GCATGTGCCT GTAGTCCCAG |
| 181 | CTACCCAGGA GGCTGAGGCA AGAGGATGGC TGGAGCCTGG GATGTTGAGG CTGCAATGAA |
| 241 | CTGTGATTAC CCCACTGCAC TCCAGCCTGG GCAAAAGAGC GAGAGACCCT GTCTCAAATA |
| 301 | ATAATAATAA TAATAA |

SEQ ID NO: 13 Human SLNCR2 Isoform-Specific Domain Sequence
tgccacc tcctgagtcc
tggttcctgc ggctccgtcg aatctgcagt gaaggccttt gggaagcgtg ggttatgtgg
gtccaggggt cccatcctcc cgtggaggcc cttgacctga ggatcactaa ctcacacccc
accgacttcc ggcccccaac aagggcagtg ttcctcttgc tccattctgc tctgaagtcc
cccaaaccgc ttcctggggc tgcttagtga aatacaaggc tcatctctga ggacctctac
agggctggat
SEQ ID NO: 14 Human SLNCR3 Isoform-Specific Domain Sequence
aggaagatca ggttcaagtt
gccagccaga ctctgggctt ccaggaggag tgggctgtgg atggcctggc ctcatttgca
tgtccctctc ctcccggccc tgcagg
SEQ ID NO: 15 Human SLNCR Cons 2 Domain Sequence
tggtggaatcagaggtttctggctgactcggtgggtgctttgaaccaggaaaggacaagaaagaggatgggaa
The SEQ ID NO: 15 sequence is believed to replace and/or supplement some SLNCR
functions in addition to the human SLNCR cons cDNA sequence of SEQ ID NO: 1

In some embodiments, the following nucleotide positions and/or residues of SEQ ID NO: 1 are preferably conserved for function and remaining positions can be modified: 92, 94, 95, 96, 97, 99, 100, 101, 103, 104, 105, 106, 110, 111, 113, 114, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 178, 179, 180, 181, 182, 183, 184, 185, 186, 187, 188, 189, 190, 191, 192, 193, 194, 195, 196, 200, 238, 239, 240, 241, 242, 243, 244, 245, 246, 247, 248, 258, 259, 260, 261, 262, 263, 264, 265, and/or 266, or any combination thereof, inclusive (e.g., 92, 179, and 195)

In some embodiments, the following base pairs of nucleotide positions and/or residues of SEQ ID NO: 1 are preferably conserved for SLNCR secondary structure: Positions believed to be involved in secondary structure: 20, 21, 22, 23, 24, 25, 26, 27, 28, 29, 102, 103, 104, 105, 106, 107, 108, 109, 110, 111, 202, 203, 204, 205, 206, 207, 215, 216, 217, 218, 219, 220, 238, 239, 240, 241, 242, 243, 244, 245, 246, 247, 248, 258, 259, 260, 261, 262, 263, 264, 265, and/or 266, or any combination thereof, inclusive (e.g., 25 and 218)

In some embodiments, specific base pairing believed to be involved in secondary structure: 20-111, 21-110, 22-109, 23-108, 24-107, 25-106, 26-105, 27-104, 28-103, 29-102. A second stem structure forms with the indicated base pairs: 202-220, 203-219, 204-218, 205-217, 206-216, 207-215. Any combination of base pair is provided, inclusive (e.g., 20-111 and 23-108; or 23-108, 26-105, and 204-218)

In some embodiments, specific nucleotides positions and/or residues of SEQ ID NO: 1 believed to be involved in AR binding: 238, 239, 240, 241, 242, 243, 244, 245, 246, 247, 248, 258, 259, 260, 261, 262, 263, 264, 265, and/or 266, or any combination thereof, where nucleotides 238-247 and 264-266 can be modified from C to T's or C's to T's, but not C/T to A/G. Thus, in some embodiments, the region of SEQ ID NO:1 required for AR binding is about nucleotides 243-248 and about 258-263 along with a requirement for the presence of a polypyrimidine-rich sequence surrounding these nucleotides such that 1, 2, 3, 4, 5, 6, 7, 8, 9, 10, 11, 12, or 13 nucleotides of nucleotides 238-247 and 264-266 can be modified from C to T's or C's to T's, but not C/T to A/G. Specific consensus sequence of SEQ ID NO: 1 believed to be involved SLNCR function: 243-248, 258-263, and/or 238-265. Nucleotides 249 and 251 are not believed to be critical.

Included in Table 1A are variations of 1, 2, 3, 4, 5, 6, 7, 8, 9, 10, 11, 12, 13, 14, 15, 16, 17, 18, 19, 20, or more nucleotides on the 5′ end, on the 3′ end, or on both the 5′ and 3′ ends, of the domain sequences as long as the sequence variations maintain the recited function and/or homology

Included in Table 1A are nucleic acid molecules comprising, consisting essentially of, or consisting of:

1) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid sequence of SEQ ID NO: 1-3, or a biologically active fragment thereof;

2) a nucleic acid sequence having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3500, 4000, 4500, 5000, 5500, 6000, 6500, 7000, 7500, 8000, 8500, 9000, 9500, 10000, or more nucleotides, or any range in between, inclusive such as between 110 and 300 nucleotides;

3) a biologically active fragment of a nucleic acid sequence of SEQ ID NO: 1-3 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or more nucleotides, or any range in between, inclusive such as between 110 and 300 nucleotides;

4) a biologically active fragment of a nucleic acid sequence of SEQ ID NO: 1-3 having 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or fewer nucleotides, or any range in between, inclusive such as between 110 and 300 nucleotides;

5) one or more domains selected from the group consisting of an SRA1 H2 helix domain, an SRA1 H3 helix domain, a Brn3a binding domain, an androgen receptor (AR) binding domain, a PXR binding domain, a PAX5 binding domain, an SRA1 H5 helix domain, an SRA1 H6 helix domain, a SLNCR autoregulation domain, and a SLNCR Cons 2 domain, in any combination, inclusive such as an SRA1 H5 and H6 helix domains;

6) the ability to bind to at least one protein transcription factor selected from the group consisting of SRC-1/NCOA-1, PXR/NR1I2, PAX, EGR-1, AR, E2F-1, CAR/NR1I3, PBX1, ATF2, C/EBP, BRN-3/POU4F1, HNF4, NF-kB, AP2, OCT4/POU5F1, SP1, STAT5, p53, TFIID, SLIRP, STAT3, REST, REST4, and DAX1, optionally wherein the nucleic acid molecule-protein transcription factor complex has the ability to translocate to the nucleus;

7) one or more biological activities selected from the group consisting of 1) the expression or activity of MMP9; 2) downregulation of naturally-occurring SLNCR isoforms; 3) modulation of the expression of one or more genes listed in FIGS. 7, 14, 16, 17, 19, and 31; 4) the expression of PLA2G4C, CT45A6, EGR2, RP11-820L6.1, EGR1, ATF3, VCX3A, SPCS2, FABP5, MAGEA2B, RPL41P1, RPS17, HNRNPA1P10, TXNIP, RPL21P75, EIF3CL, RPL7, CT45A3, GTF2IP1, CDK7, HIST1H1C, CT45A1, BTG2, RPS27, RP11-3P17.3, FDCSP, CITED4, IL34, and PD-L1; 5) cellular proliferation; 6) cell death; 7) cellular migration; 8) genomic replication; 9) angiogenesis induction; 10) cellular invasion; 11) cancer metastasis; 12) regulation of immune response and/or immune evasion; 13) modulation of one or more genes listed in Tables S5 and S6 affected by SLNCR overexpression; and 14) binding to one or more of transcription factors selected from the group consisting of SRC-1/NCOA-1, PXR/NR1I2, PAX, EGR-1, AR, E2F-1, CAR/NR1I3, PBX1, ATF2, C/EBP, BRN-3/POU4F1, HNF4, NF-kB, AP2, OCT4/POU5F1, SP1, STAT5, p53, TFIID, SLIRP, STAT3, REST, REST4, and DAX1; and

8) any combination of 1) through 7), modifiable positions, conserved nucleotide pairs, and 5′ and/or 3′ variants, thereof of Table 1A, inclusive.

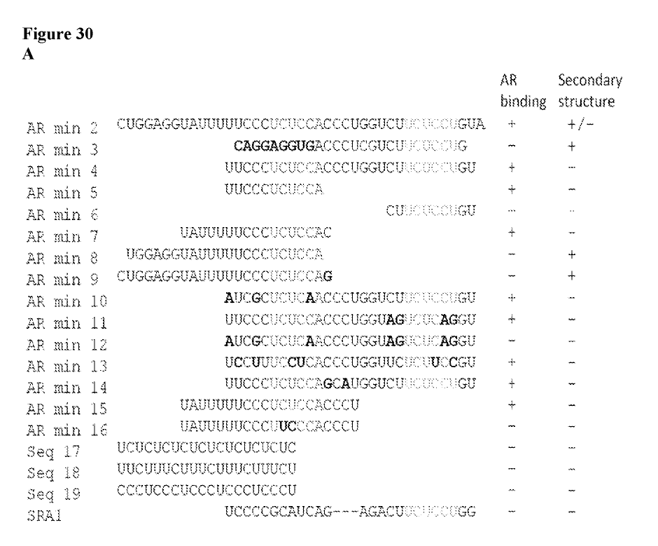

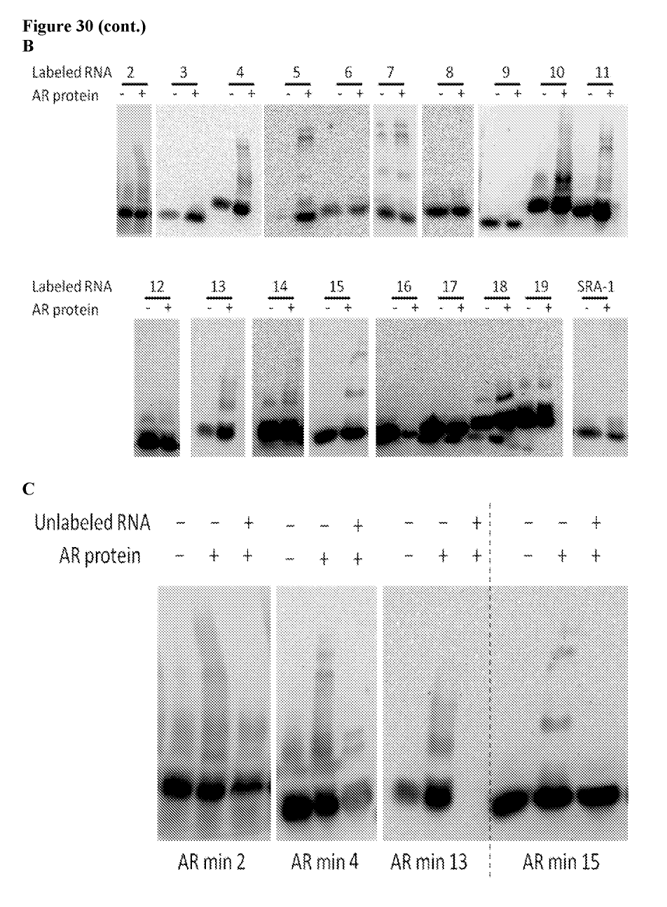

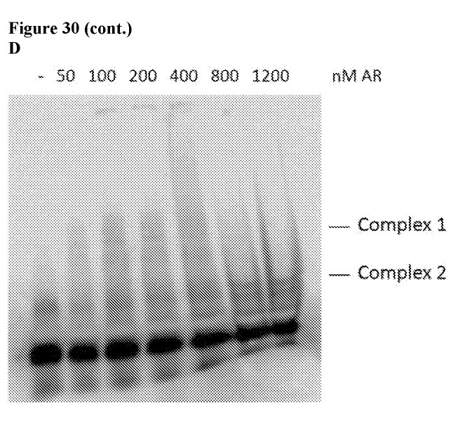

Any variation described in Table 1A can also apply to any other SLNCR sequence and/or SEQ ID NO described herein that is not already known, and such sequences and/or SEQ ID NOs, as well as any variations thereof, are included in Table 1A. For example, experiments shown in Example 17 and FIG. 30 describe SEQ ID NOs: 22 and 41 (e.g., UUCCCUCUCCACCCUGGUCUUCUCCUGU (expressed as RNA) and TTCCCTCTCCACCCTGGTCTTCTCCTGT (expressed as DNA), respectively) as a smaller sequence within SEQ ID NO: 1 that is believed to be required for AR binding and is believed to be important for SLNCR function. Without being bound by theory, it is believed that high conservation of this smaller region (e.g., at least 80%, 81%, 82%, 83%, 84%, 85%, 86%, 87%, 88%, 89%, 90%, 91%, 92%, 93%, 94%, 95%, 96%, 97%, 98%, 99%, or greater), as opposed to high conservation with the entirety of SEQ ID NO 1 is required for the majority of SLNCR function. This sequence contains both UCUCC(A/U) motifs, the minimal AR consensus binding motif as described in SEQ ID NO: 8. More specifically, the second motif (UCUCCU) is the only unstructured motif (see, for example, the downstream motif in FIG. 30), which is believed to be required for initial AR binding. After initial binding, RNA secondary structure likely relaxes, allowing for AR binding to the upstream UCUCCA motif, which is normally structured in the context of the overall SLNCR sequence. SEQ ID NO: 20 includes the nucleotides which form a stable stem-loop secondary structure with the upstream UCUCCA motif. Thus, in some embodiments, a SLNCR sequence of the present invention only contains a single UCUCC(A/U) minimal consensus AR binding motif of SLNCR. In other embodiments, both UCUCC(A/U) motifs are present within a SLNCR sequence of the present invention.

TABLE 1B
Known SLNCR Sequence

SEQ ID NO: 16 Human SLNCR (SLNCR1; LNC00673) cDNA Sequence

| | |
|---|---|
| 1 | agggcgcgca ggcggcgcgg gtgcgcggtg cggcgctggt atccagagga cgcggtcacc |
| 61 | gcctctggca tttgtcgttc tgcgcttctc cgcaaggacc ctctgttagg caggcgccca |
| 121 | ccgtaagcct cccgggcctt gtgaacctgc aaacccaagt ctgagagacg atccgccttc |
| 181 | agcgctttcc agcttggcag agaggctttc ccggcgggga tctttggttg gcgctggcga |
| 241 | tgcgcgggga agaaaggcga ggagcggcgt ccaggctggg tgatgtccca gcacgagtag |
| 301 | gcgggatgcg ctcgcttggt cctccgggcg cccggtccct gcccgcgtcg cgcgcccacc |
| 361 | cctggggacg agaaggcggc cgcctgagga cccccgcccg cgacctccgc gagtctggag |
| 421 | cgcagaggac agggtctggc tgctctttgg ccttggatgg aaagtgggga attgggtggg |
| 481 | gggctgcgga ccccttaacg tggattactt ggtgtgtatc agctgggctc agaagaccca |
| 541 | cgacctcttc tccatccgtg gattgatttg ttctgcttaa cagctgggtc gccaagctgg |
| 601 | aggtattttt ccctctccac cctggtcttc tcctgtaacg tgtggccgcc ttttccagca |
| 661 | cggcctcctg ccttcctggt gcactttttg gagaacgtgg tggaatcaga ggtttctggc |
| 721 | tgactcggtg ggtgctttga accaggaaag gacaagaaag aggatgggaa ggactgatcc |
| 781 | acattcccac caggaagttt agcagaaccc ccgcgtgcca cctggacccc ttggaaggac |
| 841 | ctggctcagg ctggaccacc tcttgagagg caggagctct ggatttgatc aagaattctt |
| 901 | tgctgagcat ggtgcctcat gcctataatc ccaacacttt gggaggccag tgtgggagga |
| 961 | tctcttgagc ccaggagttc aagactagcc tgggcaacac agagagaccc catctctaaa |
| 1021 | ataataataa taataaaata aaaaattagc agggcatggt ggcatgtgcc tgtagtccca |
| 1081 | gctacccagg aggctgaggc aagaggatgg ctggagcctg ggatgttgag gctgcaatga |
| 1141 | actgtgatta ccccactgca ctccagcctg ggcaaaagag cgagagaccc tgtctcaaat |
| 1201 | aataataata ataataatct tattttggag aataaagaga cctctggatt tgaggtgcca |
| 1261 | tttgggtaga aagaaaagac gtttacaccg agaaatagtc tgtgttgccc tgaaggagca |
| 1321 | gagggatgca tcgctggagg tgacctacag ttgaagaaga ctcattatga cagaccttgt |
| 1381 | ccttcttcct tgtggaaagt gtttcctctg ctgctactgc tcatgagact cttccccctc |
| 1441 | cctgtcccag ggaaccaaag ggctttctac cacacccttt cttgcccccc gcctcccatg |
| 1501 | tctgctgtgc ctttgtactc agcaattctt gtttgctcca ttatcttcca gccggataca |
| 1561 | gagtgaatag ttaaccacac ttaggtcaaa taggatctaa atttttgttc ctgctccgtg |
| 1621 | taaagaggcc agtgtttgtg tgttgcaagc agccttggaa tagtaactct tctcatttgt |
| 1681 | ttgggatctg gccaccaagt tccagaatga tacacggatc agtgcagaag ttcatcaggc |
| 1741 | tctcggacct tagggctgtt ggagaaggct tcagcagcag aactgatggt gaaggctcgt |
| 1801 | gttctccatc ctcaactttc tttgcttcga tcatacacaa gaatacattt ggaagggcaa |
| 1861 | aaaatgaaca ctgtcgttca ttgcagccgt gttttgtgac acagatgcac agtctgctgt |
| 1921 | gaagaccttc tctcaagtgg catttgggag tccatgccag atcatggtgc ttcatgagag |
| 1981 | actgacagct atcaggggtt gtggcactta gtgaggactc tcctccccca gtgtgtgctg |
| 2041 | atgacacata cacacctgac aatagcttga gtcttctctg ttccttttac tctgtagcca |
| 2101 | acatacacat gatttaaaac cctttctaaa tatctatcat ggttcatcct tgtccaaatg |
| 2161 | cagagtcaga gctatttgta cttcattatt atttccaagg cgaatagttg gctttctttt |
| 2221 | tgcaaaaata attaaagttt ttgtatgttg cagttgcaaa aaaaaaaaaa aaaaa |

### II. SLNCR Isoform Nucleic Acids, Biomarker Polypeptides, and Antibodies, Related Agents, and Compositions

Novel agents and compositions of the present invention are provided herein. Such agents and compositions can also be used for the diagnosis, prognosis, prevention, and treatment of cancers, such as melanoma, as well as conditions in which SLNCR is associated or aberrantly expressed. For example, such agents and compositions can detect and/or modulate, e.g., down-regulate, expression and/or activity of gene products or fragments thereof encoded by biomarkers of the present invention, including the biomarkers listed in Table 1, the Figures, and the Examples. Exemplary agents include antibodies, small molecules, peptides, peptidomimetics, natural ligands, and derivatives of natural ligands, that can either bind and/or activate or inhibit nucleic acid biomarkers of the present invention and/or protein biomarkers, such as nuclear receptors or transcription factors of the present invention, including the biomarkers listed in Table 1, the Figures, and the Examples, or biologically active fragments thereof; RNA interference, antisense, nucleic acid aptamers, etc. that can downregulate the expression and/or activity of the biomarkers of the present invention, including the biomarkers listed in Table 1, the Figures, and the Examples, or biologically active fragments thereof.

a. Isolated Nucleic Acids

In one embodiment, isolated nucleic acid molecules that specifically hybridize with or encode one or more biomarkers listed in Table 1, the Figures, and the Examples, or biologically active portions thereof, are presented. The nucleic acid molecules can be all of the nucleic acid molecules shown in Table 1, the Figures, and the Examples, or any subset thereof (e.g., the combination of SLNCR2, SLNCR3, SLNCR cons, SLNCR del cons, and the like). As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecules corresponding to the one or more biomarkers listed in Table 1, the Figures, and the Examples, can contain less than about 5 kb, 4kb, 3kb, 2kb, 1 kb, 0.5 kb or 0.1 kb of nucleotide sequences which naturally flank the nucleic acid molecule in genomic DNA of the cell from which the nucleic acid is derived (i.e., melanoma cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having the nucleotide sequence of one or more biomarkers listed in Table 1, the Figures, and the Examples or a nucleotide sequence which is at least about 50%, preferably at least about 60%, more preferably at least about 70%, yet more preferably at least about 80%, still more preferably at least about 90%, and most preferably at least about 95% or more (e.g., about 98%) homologous to the nucleotide sequence of one or more biomarkers listed in Table 1, the Figures, and the Examples or a portion thereof (i.e., 100, 200, 300, 400, 450, 500, or more nucleotides), can be isolated using standard molecular biology techniques and the sequence information provided herein. For example, a human cDNA can be isolated from a human cell line (from Stratagene, La Jolla, Calif., or Clontech, Palo Alto, Calif.) using all or portion of the nucleic acid molecule, or fragment thereof, as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of the nucleotide sequence of one or more biomarkers listed in Table 1, the Figures, and the Examples or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence, or fragment thereof, can be isolated by the polymerase chain reaction using oligonucleotide primers designed based upon the sequence of the one or more biomarkers listed in Table 1, the Figures, and the Examples, or a biologically active fragment thereof, or the homologous nucleotide sequence. For example, mRNA can be isolated from melanoma cells (i.e., by the guanidinium-thiocyanate extraction procedure of Chirgwin et al. (1979) Biochemistry 18: 5294-5299) and cDNA can be prepared using reverse transcriptase (i.e., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed according to well-known methods in the art. A nucleic acid of the present invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to the nucleotide sequence of one or more biomarkers listed in Table 1, the Figures, and the Examples can be prepared by standard synthetic techniques, i.e., using an automated DNA synthesizer.

Probes based on the nucleotide sequences of one or more biomarkers listed in Table 1, the Figures, and the Examples, can be used to detect transcripts or genomic sequences encoding the same or homologous sequences. In preferred embodiments, the probe further comprises a label group attached thereto, i.e., the label group can be a radioisotope, a fluorescent compound, an enzyme, or an enzyme co-factor. Such probes can be used as a part of a diagnostic test kit for identifying cells or tissue which express one or more biomarkers listed in Table 1, the Figures, and the Examples, such as by measuring a level of nucleic acid in a sample of cells from a subject, i.e., detecting mRNA levels of one or more biomarkers listed in Table 1, the Figures, and the Examples.

Nucleic acid molecules encoding lncRNAs corresponding to one or more biomarkers listed in Table 1, the Figures, and the Examples, from different species are also contemplated. For example, rat or monkey cDNA can be identified based on the nucleotide sequence of a human and/or mouse sequence and such sequences are well known in the art. In one embodiment, the nucleic acid molecule(s) of the present invention encodes an lncRNA or portion thereof which includes a nucleic acid sequence sufficiently similar to the nucleic acid sequence of one or more biomarkers listed in Table 1, the Figures, and the Examples, such that the lncRNA or portion thereof has SLNCR activity as described herein.

Such homologous nucleic acids and encoded polypeptides can be readily produced by the ordinarily skilled artisan based on the sequence information provided in Table 1, the Figures, and the Examples.

As used herein, the language “sufficiently homologous” refers to nucleic acids or portions thereof which have nucleic acid sequences which include a minimum number of identical or equivalent (e.g., a cognate pair of nucleotides for maintaining nucleic acid secondary structure) to a nucleic acid sequence of the biomarker, or fragment thereof, such that the nucleic acid thereof modulates (e.g., inhibits) one or more of the following biological activities: a) binding to the biomarker; b) modulating the copy number of the biomarker; c) modulating the expression level of the biomarker; and d) modulating the activity level of the biomarker.

Portions of nucleic acid molecules of the one or more biomarkers listed in Table 1, the Figures, and the Examples, are preferably biologically active portions of the protein. As used herein, the term “biologically active portion” of one or more biomarkers listed in Table 1, the Figures, and the Examples, is intended to include a portion, e.g., a domain/motif, that has one or more of the biological activities of the full-length protein. For example, the SLNCR autoregulation domain fragment is believed to confer the ability of one SLNCR isoform to regulate the expression of other SLNCR isoforms.

The invention further encompasses nucleic acid molecules that differ from the nucleotide sequence of the one or more biomarkers listed in Table 1, the Figures, and the Examples, or fragment thereof due to degeneracy of the genetic code and thus encode the same protein as that encoded by the nucleotide sequence, or fragment thereof. In another embodiment, an isolated nucleic acid molecule of the present invention has a nucleotide sequence having a nucleic acid sequence of one or more biomarkers listed in Table 1, the Figures, and the Examples, or fragment thereof, or having a nucleic acid sequence which is at least about 70%, 75%, 80%, 85%, 90%, 91%, 92%, 93%, 94%, 95%, 96%, 97%, 98%, 99% or more homologous to the amino acid sequence of the one or more biomarkers listed in Table 1, the Figures, and the Examples, or fragment thereof. In another embodiment, a nucleic acid encoding a polypeptide consists of nucleic acid sequence encoding a portion of a full-length fragment of interest that is at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3500, 4000, 4500, 5000, 5500, 6000, 6500, 7000, 7500, 8000, 8500, 9000, 9500, 10000, or more nucleotides, or any range in between, inclusive such as between 110 and 300 nucleotides; or more nucleotides, or any range in between, inclusive such as between 110 and 300 nucleotides; or 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or fewer nucleotides, or any range in between, inclusive such as between 110 and 300 nucleotides.

It will be appreciated by those skilled in the art that DNA sequence polymorphisms that lead to changes in the amino acid sequences of the one or more biomarkers listed in Table 1, the Figures, and the Examples, may exist within a population (e.g., a mammalian and/or human population). Such genetic polymorphisms may exist among individuals within a population due to natural allelic variation. As used herein, the terms “gene” and “recombinant gene” refer to nucleic acid molecules comprising an open reading frame encoding one or more biomarkers listed in Table 1, the Figures, and the Examples, preferably a mammalian, e.g., human, lncRNA. Such natural allelic variations can typically result in 1-5% variance in the nucleotide sequence of the one or more biomarkers listed in Table 1, the Figures, and the Examples. Any and all such nucleotide variations and resulting amino acid polymorphisms in the one or more biomarkers listed in Table 1, the Figures, and the Examples, that are the result of natural allelic variation and that do not alter the functional activity of the one or more biomarkers listed in Table 1, the Figures, and the Examples, are intended to be within the scope of the present invention. Moreover, nucleic acid molecules encoding one or more biomarkers listed in Table 1, the Figures, and the Examples, from other species.

In addition to naturally-occurring allelic variants of the one or more biomarkers listed in Table 1, the Figures, and the Examples, sequence that may exist in the population, the skilled artisan will further appreciate that changes can be introduced by mutation into the nucleotide sequence, or fragment thereof, thereby leading to changes in the amino acid sequence of the encoded one or more biomarkers listed in Table 1, the Figures, and the Examples, without altering the functional ability of the one or more biomarkers listed in Table 1, the Figures, and the Examples. For example, nucleotide substitutions leading to substitutions at “non-essential” nucleotide positions can be made in the sequence, or fragment thereof. A “non-essential” amino acid position is a position that can be altered from the wild-type sequence of the one or more biomarkers listed in Table 1, the Figures, and the Examples, without substnatiallyaltering the activity of the one or more biomarkers listed in Table 1, the Figures, and the Examples, whereas an “essential” amino acid residue is required for the activity of the one or more biomarkers listed in Table 1, the Figures, and the Examples. Other positions, however, (e.g., those that are not conserved or only semi-conserved between mouse and human) may not be essential for activity and thus are likely to be amenable to alteration without altering the activity of the one or more biomarkers listed in Table 1, the Figures, and the Examples.

The term “sequence identity or homology” refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In a preferred embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a protein homologous to one or more biomarkers listed in Table 1, the Figures, and the Examples, or fragment thereof, can be created by introducing one or more nucleotide substitutions, additions or deletions into the nucleotide sequence, or fragment thereof, or a homologous nucleotide sequence such that one or more amino acid substitutions, additions or deletions are introduced into the encoded protein. Mutations can be introduced by standard techniques, such as site-directed mutagenesis and PCR-mediated mutagenesis.

The levels of one or more biomarkers listed in Table 1, the Figures, and the Examples, levels may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In preferred embodiments, the levels of one or more biomarkers listed in Table 1, the Figures, and the Examples, levels are ascertained by measuring gene transcript (e.g., mRNA), by a measure of the quantity of translated protein, or by a measure of gene product activity. Expression levels can be monitored in a variety of ways, including by detecting lncRNA levels or activity, any of which can be measured using standard techniques. Detection can involve quantification of the level of gene expression (e.g., genomic DNA, cDNA, transcribed RNA, lncRNA activity), or, alternatively, can be a qualitative assessment of the level of gene expression, in particular in comparison with a control level. The type of level being detected will be clear from the context.

In a particular embodiment, the RNA expression level can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated RNA can be used in hybridization or amplification assays that include, but are not limited to, Southern or Northern analyses, polymerase chain reaction analyses and probe arrays. One preferred diagnostic method for the detection of RNA levels involves contacting the isolated RNA with a nucleic acid molecule (probe) that can hybridize to the RNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an RNA or genomic DNA encoding one or more biomarkers listed in Table 1, the Figures, and the Examples. Other suitable probes for use in the diagnostic assays of the present invention are described herein. Hybridization of an RNA with the probe indicates that one or more biomarkers listed in Table 1, the Figures, and the Examples, is being expressed.

In one format, the RNA is immobilized on a solid surface and contacted with a probe, for example by running the isolated RNA on an agarose gel and transferring the RNA from the gel to a membrane, such as nitrocellulose. In an alternative format, the probe(s) are immobilized on a solid surface and the RNA is contacted with the probe(s), for example, in a gene chip array, e.g., an Affymetrix™ gene chip array. A skilled artisan can readily adapt known RNA detection methods for use in detecting the level of the One or more biomarkers listed in Table 1, the Figures, and the Examples, RNA expression levels.

An alternative method for determining RNA expression level in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, RNA does not need to be isolated from the cells prior to detection. In such methods, a cell or tissue sample is prepared/processed using known histological methods. The sample is then immobilized on a support, typically a glass slide, and then contacted with a probe that can hybridize to the one or more biomarkers listed in Table 1, the Figures, and the Examples.

As an alternative to making determinations based on the absolute expression level, determinations may be based on the normalized expression level of one or more biomarkers listed in Table 1, the Figures, and the Examples. Expression levels are normalized by correcting the absolute expression level by comparing its expression to the expression of a non-biomarker gene, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of a protein corresponding to one or more biomarkers listed in Table 1, the Figures, and the Examples, can also be detected and/or quantified by detecting or quantifying the activity, such as effects on associate polypeptides like transcription factors or nuclear receptors. The associated polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express the biomarker of interest.

In some embodiments, vectors and/or host cells are further provided. Ine aspect of the present invention pertains to the use of vectors, preferably expression vectors, containing a nucleic acid encoding a biomarker listed in Table 1, the Figures, and the Examples, or a portion or ortholog thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. In one embodiment, adenoviral vectors comprising a biomarker nucleic acid molecule are used.

The recombinant expression vectors of the present invention comprise a nucleic acid of the present invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the present invention can be introduced into host cells to thereby produce proteins or peptides, including fusion proteins or peptides, encoded by nucleic acids as described herein.

The recombinant expression vectors of the present invention can be designed for expression of the desired biomarker in prokaryotic or eukaryotic cells. For example, a biomarker can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase. Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Examples of suitable yeast expression vectors include pYepSecl (Baldari, et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Examples of suitable baculovirus expression vectors useful for insect cell hosts include the pAc series (Smith et al. (1983) Mol. Cell Biol. 3:2156-2165) and the pVL series (Lucklow and Summers (1989) Virology 170:31-39). Examples of suitable mammalian expression vectors include pCDM8 (Seed, B. (1987) Nature 329:840) and pMT2PC (Kaufman et al. (1987) EMBO J. 6:187-195).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters such as in melanoma cancer cells are well-known in the art (see, for example, Pleshkan et al. (2011) Acta Nat. 3:13-21).

The present invention further provides a recombinant expression vector comprising a nucleic acid molecule of the present invention cloned into the expression vector in an antisense orientation. That is, the DNA molecule is operatively linked to a regulatory sequence in a manner which allows for expression (by transcription of the DNA molecule) of an RNA molecule which is antisense to a biomarker mRNA described herein. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, biomarker protein can be expressed in bacterial cells such as E. coli, insect cells, yeast or mammalian cells (such as Fao hepatoma cells, primary hepatocytes, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A cell culture includes host cells, media and other byproducts. Suitable media for cell culture are well known in the art. A biomarker polypeptide or fragment thereof, may be secreted and isolated from a mixture of cells and medium containing the polypeptide. Alternatively, a biomarker polypeptide or fragment thereof, may be retained cytoplasmically and the cells harvested, lysed and the protein or protein complex isolated. A biomarker polypeptide or fragment thereof, may be isolated from cell culture medium, host cells, or both using techniques known in the art for purifying proteins, including ion-exchange chromatography, gel filtration chromatography, ultrafiltration, electrophoresis, and inmmunoaffinity purification with antibodies specific for particular epitopes of a biomarker or a fragment thereof. In other embodiments, heterologous tags can be used for purification purposes (e.g., epitope tags and FC fusion tags), according to standards methods known in the art.

Thus, a nucleotide sequence encoding all or a selected portion of a biomarker polypeptide may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant biomarker polypeptides, or fragments thereof, by microbial means or tissue-culture technology in accord with the subject invention.

A host cell of the present invention, such as a prokaryotic or eukaryotic host cell in culture, can be used to produce (i.e., express) biomarker protein. Accordingly, the invention further provides methods for producing biomarker protein using the host cells of the present invention. In one embodiment, the method comprises culturing the host cell of invention (into which a recombinant expression vector encoding a biomarker has been introduced) in a suitable medium until biomarker protein is produced. In another embodiment, the method further comprises isolating the biomarker protein from the medium or the host cell.

The host cells of the present invention can also be used to produce nonhuman transgenic animals. The nonhuman transgenic animals can be used in screening assays designed to identify agents or compounds, e.g., drugs, pharmaceuticals, etc., which are capable of ameliorating detrimental symptoms of selected disorders such as glucose homeostasis disorders, weight disorders or disorders associated with insufficient insulin activity. For example, in one embodiment, a host cell of the present invention is a fertilized oocyte or an embryonic stem cell into which biomarker encoding sequences, or fragments thereof, have been introduced. Such host cells can then be used to create non-human transgenic animals in which exogenous biomarker sequences have been introduced into their genome or homologous recombinant animals in which endogenous biomarker sequences have been altered. Such animals are useful for studying the function and/or activity of biomarker, or fragments thereof, and for identifying and/or evaluating modulators of biomarker activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous biomarker gene has been altered by homologous recombination between the endogenous gene and an exogenous DNA molecule introduced into a cell of the animal, e.g., an embryonic cell of the animal, prior to development of the animal.

A transgenic animal of the present invention can be created by introducing nucleic acids encoding a biomarker, or a fragment thereof, into the male pronuclei of a fertilized oocyte, e.g., by microinjection, retroviral infection, and allowing the oocyte to develop in a pseudopregnant female foster animal. Human biomarker cDNA sequence can be introduced as a transgene into the genome of a nonhuman animal. Alternatively, a nonhuman homologue of the human biomarker gene can be used as a transgene. Intronic sequences and polyadenylation signals can also be included in the transgene to increase the efficiency of expression of the transgene. A tissue-specific regulatory sequence(s) can be operably linked to the biomarker transgene to direct expression of biomarker protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the biomarker transgene in its genome and/or expression of biomarker mRNA in tissues or cells of the animals. A transgenic founder animal can then be used to breed additional animals carrying the transgene. Moreover, transgenic animals carrying a transgene encoding a biomarker can further be bred to other transgenic animals carrying other transgenes.

To create a homologous recombinant animal, a vector is prepared which contains at least a portion of biomarker gene into which a deletion, addition or substitution has been introduced to thereby alter, e.g., functionally disrupt, the biomarker gene. The biomarker gene can be a human gene, but more preferably, is a nonhuman homologue of a human biomarker gene. For example, a mouse biomarker gene can be used to construct a homologous recombination vector suitable for altering an endogenous biomarker gene, respectively, in the mouse genome. In a preferred embodiment, the vector is designed such that, upon homologous recombination, the endogenous biomarker gene is functionally disrupted (i.e., no longer encodes a functional protein; also referred to as a “knock out” vector). Alternatively, the vector can be designed such that, upon homologous recombination, the endogenous biomarker gene is mutated or otherwise altered but still encodes functional protein (e.g., the upstream regulatory region can be altered to thereby alter the expression of the endogenous biomarker protein). In the homologous recombination vector, the altered portion of the biomarker gene is flanked at its 5′ and 3′ ends by additional nucleic acid of the biomarker gene to allow for homologous recombination to occur between the exogenous biomarker gene carried by the vector and an endogenous biomarker gene in an embryonic stem cell. The additional flanking biomarker nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced biomarker gene has homologously recombined with the endogenous biomarker gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic nonhuman animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

Nucleic acid molecules of the present invention can also be engineered as fusion constructs using recombinant DNA techniques. A “chimeric lncRNA” or “fusion lncRNA” comprises a biomarker polypeptide described herein operatively linked to a non-biomarker nucleic acid sequence. A “biomarker lncRNA” refers to a nucleic acid sequence having an amino acid sequence corresponding to a biomarker listed in Table 2, the Figures, or the Examples, or a fragment or ortholog thereof, whereas a “non-biomarker lncRNA” refers to a nucleic acid sequence not substantially homologous to the biomarker lncRNA, respectively, e.g., a nucleic acid sequence that is different from the biomarker nucleic acid sequence and that is derived from the same or a different organism. Within the fusion construct, the term “operatively linked” is intended to indicate that the biomarker nucleic acid sequence and the non-biomarker nucleic acid sequence are fused in a rame to each other. The non-biomarker polypeptide can be fused to the 5′ end, the 3′ end, or in between the 5′ and 3′ ends of the biomarker nucleic acid sequence. The fusion protein can function as a nucleic acid (e.g., a MS2 loop structure) or encode a protein for translation, such as using an internal ribosome entry sequence (IRES). For example, in one embodiment the fusion protein is a biomarker-GST and/or biomarker-Fc fusion protein. Such fusion proteins can facilitate the purification, expression, and/or bioavailability of recombinant biomarker constructs. In certain host cells (e.g., mammalian host cells), expression and/or secretion of the biomarker fusion construct can be increased through use of a heterologous signal sequence.

Preferably, a biomarker chimeric or fusion constructs of the present invention is produced by standard recombinant DNA techniques. For example, DNA fragments coding for the different sequences are ligated together in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A biomarker-encoding nucleic acid can be cloned into such an expression vector such that the fusion moiety is linked in-frame to the biomarker protein.

b. Antibodies and Other Useful Agents

As stated above, the present invention provides compositions related to producing, detecting, characterizing, or modulating the level or activity of biomarker lncRNAs, or fragments or orthologs thereof, such as nucleic acids, vectors, host cells, and the like. Such compositions may serve as compounds that modulate the expression and/or activity of one or more biomarkers listed in Table 1, the Figures, and the Examples. For example, anti-SLNCR antibodies that may bind specifically to SLNCR can be used for diagnostic, prognostic, and/or therapeutic (e.g., intrabodies) purposes. In one embodiment, the anti-SLNCR antibodies are specific for one or more of the SLNCR isoforms listed in Table 1, the Figures, and the Examples. In another embodiment, the anti-SLNCR antibodies are specific for a ribonucleoprotein complex mediated by one or more SLNCR isoforms (e.g., SLNCR2 complexed with AR and/or Brn3a).

An isolated polypeptide or a fragment thereof (or a nucleic acid encoding such a polypeptide) corresponding to one or more biomarkers of the present invention, including the biomarkers listed in Table 1, the Figures, and the Examples, or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i e, an antigenic peptide that spans a region of the polypeptide molecule that is not conserved across species is used as immunogen; such non conserved residues can be determined using an alignment such as that provided herein).

For example, a polypeptide immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well-known protocols used for fusing lymphocytes and immortalized cell lines can be applied for the purpose of generating a monoclonal antibody against one or more biomarkers of the present invention, including the biomarkers listed in Table 1, the Figures, and the Examples, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the present invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and

screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant polypeptide antibodies, such as chimeric and humanized monoclonal antibodies, comprising both human and non-human portions, which can be made using standard recombinant DNA techniques, are within the scope of the present invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. The use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

Additionally, fully human antibodies could be made against biomarkers of the present invention, including the biomarkers listed in Table 1, the Figures, and the Examples, or fragments thereof. Fully human antibodies can be made in mice that are transgenic for human immunoglobulin genes, e.g., according to Hogan, et al., “Manipulating the Mouse Embryo: A Laboratory Manuel,” Cold Spring Harbor Laboratory. Briefly, transgenic mice are immunized with purified immunogen. Spleen cells are harvested and fused to myeloma cells to produce hybridomas. Hybridomas are selected based on their ability to produce antibodies which bind to the immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers of the present invention, including one or more biomarkers listed in Table 1, the Figures, and the Examples, or a fragment thereof. In one embodiment, the bispecific antibody could specifically bind to both a polypeptide or a fragment thereof and its natural binding partner(s) or a fragment(s) thereof.

In another aspect of the present invention, nucleic acid mimetics can be used to antagonize or promote the activity of one or more biomarkers of the present invention, including one or more biomarkers listed in Table 1, the Figures, and the Examples, or a fragment(s) thereof. In one embodiment, variants of one or more biomarkers listed in Table 1, the Figures, and the Examples, which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477. In addition, libraries of fragments of a polypeptide coding sequence can be used to generate a variegated population of polypeptide fragments for screening and subsequent selection of variants of a given polypeptide. For example, dominant negative transcription factors can be identified that functionally sequester SLNCR lncRNAs to thereby prevent them from activating nuclear transcription. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with 51 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers of the present invention, including one or more biomarkers listed in Table 1, the Figures, and the Examples, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the present invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere, J. (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH₂S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivitization (e.g., labeling) of peptidomimetics should not substantially interfere with the desired biological or pharmacological activity of the peptidomimetic.

Also encompassed by the present invention are small molecules which can modulate (either enhance or inhibit) interactions, e.g., between biomarkers listed in Table 1, the Figures, and the Examples, and their natural binding partners, or inhibit activity. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g., multiple compounds in each testing sample) or as individual compounds.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, piwiRNA, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can enhance or upregulate one or more biological activities associated with the corresponding wild-type, naturally occurring, or synthetic small nucleic acids. In another embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers of the present invention, including one or more biomarkers listed in Table 1, the Figures, and the Examples, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g., cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences of the present invention may be associated with a second RNA sequence that may be located on the same RNA molecule or

on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin. In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, piwiRNA, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Table 1, the Figures, and the Examples,). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner, R. (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21,20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al. (1988) BioTechniques 6:958-976) or intercalating agents. (See, e.g., Zon (1988), Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

It is to be understood that additional well known nucleic acid architecture or chemistry can be applied. Different modifications can be placed at different positions to prevent the oligonucleotide from activating RNase H and/or being capable of recruiting the RNAi machinery. In another embodiment, they may be placed such as to allow RNase H activation and/or recruitment of the RNAi machinery. The modifications can be non-natural bases, e.g. universal bases. It may be modifications on the backbone sugar or phosphate, e.g., 2′-O-modifications including LNA or phosphorothioate linkages. As used herein, it makes no difference whether the modifications are present on the nucleotide before incorporation into the oligonucleotide or whether the oligonucleotide is modified after synthesis.

Preferred modifications are those that increase the affinity of the oligonucleotide for complementary sequences, i.e. increases the tm (melting temperature) of the oligonucleotide base paired to a complementary sequence. Such modifications include 2′-O-flouro, 2′-O-methyl, 2′-O-methoxyethyl. The use of LNA (locked nucleic acid) units, phosphoramidate, PNA (peptide nucleic acid) units or INA (intercalating nucleic acid) units is preferred. For shorter oligonucleotides, it is preferred that a higher percentage of affinity increasing modifications are present. If the oligonucleotide is less than 12 or 10 units long, it may be composed entirely of LNA units. A wide range of other non-natural units may also be build into the oligonucleotide, e.g., morpholino, 2′-deoxy-2′-fluoro-arabinonucleic acid (FANA) and arabinonucleic acid (ANA). In a preferred embodiment, the fraction of units modified at either the base or sugar relatively to the units not modified at either the base or sugar is selected from the group consisting of less than less than 99%, 95%, less than 90%, less than 85% or less than 75%, less than 70%, less than 65%, less than 60%, less than 50%, less than 45%, less than 40%, less than 35%, less than 30%, less than 25%, less than 20%, less than 15%, less than 10%, and less than 5%, less than 1%, more than 99%, more than 95%, more than 90%, more than 85% or more than 75%, more than 70%, more than 65%, more than 60%, more than 50%, more than 45%, more than 40%, more than 35%, more than 30%, more than 25%, more than 20%, more than 15%, more than 10%, and more than 5% and more than 1%.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g., mRNAs) sequences mediate degradation or in which sequences incompletely complementary to cellular nucleic acids (e.g., mRNAs) mediate translational repression when expressed within cells. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA

(dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™ In another embodiment, deletion, modification, editing of the gene (e.g., to modify SLNCR genomic sequence or mutate the AR binding site to abolish activity) can be processed by genome editing, optionally wherein the genome editing is expressed constitutively or inducibly (e.g., such as genome editing selected from the group consisting of CRISPR-Cas RNA-guided engineered nucleases (RGENs), zinc finger nucleases (ZFNs), transcription activator-like effectors (TALEs), homing meganucleases, and homologous recombination). In one embodiment, knock-out or clustered regularly interspaced short palindromic repeats (CRISPR) technology is used to effect the desired genome editing. In such embodiments, a CRISPR guide RNA and/or a Cas enzyme, such as a Cas9 enzyme, may be expressed. For example, a vector containing only the guide RNA can be administered to an animal or cells transgenic for the Cas9 enzyme. Such genome editing methods and systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47).

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods and compositions presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, piwiRNA, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. Based on the genetic pathway analyses described herein, it is believed that such combinations of agents is especially effective in diagnosing, prognosing, preventing, and treating melanoma. Thus, “single active agents” described herein can be combined with other pharmacologically active compounds (“second active agents”) known in the art according to the methods and compositions provided herein. It is believed that certain combinations work synergistically in the treatment of particular types of melanoma. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful in this invention include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alpha-n1, interferon alpha-n3, interferon beta-Ia, and interferon gamma-Ib; GM-CF and GM-CSF; and EPO.

Particular proteins that can be used in the methods and compositions provided herein include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.). Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

When antibodies are used, the therapy is called immunotherapy. Antibodies that can be used in combination with the methods described herein include monoclonal and polyclonal antibodies. Examples of antibodies include, but are not limited to, ipilimumab (Yervoy®), trastuzumab (Herceptin®), rituximab (Rituxan®), bevacizumab (Avastin®), pertuzumab (Omnitarg®), tositumomab (Bexxar®), edrecolomab (Panorex®), and G250. Compounds of the present invention can also be combined with, or used in combination with, anti-TNF-α antibodies. Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits provided herein. See, e.g., Emens, L. A., et al., Curr. Opinion Mol. Ther. 3(1):77-84 (2001).

Second active agents that are small molecules can also be used to in combination as provided herein. Examples of small molecule second active agents include, but are not limited to, anti-cancer agents, antibiotics, immunosuppressive agents, and steroids.

In some embodiments, well known “combination chemotherapy” regimens can be used. In one embodiment, the combination chemotherapy comprises a combination of two or more of cyclophosphamide, hydroxydaunorubicin (also known as doxorubicin or adriamycin), oncovorin (vincristine), and prednisone. In another preferred embodiment, the combination chemotherapy comprises a combination of cyclophsophamide, oncovorin, prednisone, and one or more chemotherapeutics selected from the group consisting of anthracycline, hydroxydaunorubicin, epirubicin, and motixantrone.

Examples of other anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cyclosporin A; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; rasfarnesyl protein transferase inhibitors; ras inhibitors; ras-GAP inhibitor; retelliptine demethylated; rhenium Re 186 etidronate; rhizoxin; ribozymes; RH retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer. Specific second active agents include, but are not limited to, chlorambucil, fludarabine, dexamethasone (Decadron®), hydrocortisone, methylprednisolone, cilostamide, doxorubicin (Doxil®), forskolin, rituximab, cyclosporin A, cisplatin, vincristine, PDE7 inhibitors such as BRL-50481 and IR-202, dual PDE4/7 inhibitors such as IR-284, cilostazol, meribendan, milrinone, vesnarionone, enoximone and pimobendan, Syk inhibitors such as fostamatinib disodium (R406/R788), R343, R-112 and Excellair® (ZaBeCor Pharmaceuticals, Bala Cynwyd, Pa.).

Moreover, anti-SLNCR agents in combination with nuclear receptor inhibitors are described herein.

### IV. Methods of Selecting Agents and Compositions

Another aspect of the present invention relates to methods of selecting agents (e.g., antibodies, fusion constructs, peptides, small molecules, and small nucleic acids) which bind to, upregulate, downregulate, or modulate one or more biomarkers of the present invention listed in Table 1, the Figures, and the Examples, and/or a cancer (e.g., melanoma). Such methods can use screening assays, including cell-based and non-cell based assays.

In one embodiment, the invention relates to assays for screening candidate or test compounds which bind to or modulate the expression or activity level of, one or more biomarkers of the present invention, including one or more biomarkers listed in Table 1, the Figures, and the Examples, or a fragment or ortholog thereof. Such compounds include, without limitation, antibodies, proteins, fusion proteins, nucleic acid molecules, and small molecules.

In one embodiment, an assay is a cell-based assay, comprising contacting a cell expressing one or more biomarkers of the present invention, including one or more biomarkers listed in Table 1, the Figures, and the Examples, or a fragment thereof, with a test compound and determining the ability of the test compound to modulate (e.g., stimulate or inhibit) the level of interaction between the biomarker and its natural binding partners as measured by direct binding or by measuring a parameter of cancer.

For example, in a direct binding assay, the biomarker polypeptide, a binding partner polypeptide of the biomarker, or a fragment(s) thereof, can be coupled with a radioisotope or enzymatic label such that binding of the biomarker polypeptide or a fragment thereof to its natural binding partner(s) or a fragment(s) thereof can be determined by detecting the labeled molecule in a complex. For example, the biomarker polypeptide, a binding partner polypeptide of the biomarker, or a fragment(s) thereof, can be labeled with ¹²⁵I, ³⁵S, ¹⁴C, or ³H, either directly or indirectly, and the radioisotope detected by direct counting of radioemmission or by scintillation counting. Alternatively, the polypeptides of interest a can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to modulate the interactions between one or more biomarkers of the present invention, including one or more biomarkers listed in Table 1, the Figures, and the Examples, or a fragment thereof, and its natural binding partner(s) or a fragment(s) thereof, without the labeling of any of the interactants (e.g., using a microphysiometer as described in McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In a preferred embodiment, determining the ability of the blocking agents (e.g., antibodies, fusion proteins, peptides, nucleic acid molecules, or small molecules) to antagonize the interaction between a given set of nucleic acid molecules and/or polypeptides can be accomplished by determining the activity of one or more members of the set of interacting molecules. For example, the activity of one or more biomarkers of the present invention, including one or more biomarkers listed in Table 1, the Figures, and the Examples, or a fragment thereof, can be determined by detecting induction of cytokine or chemokine response, detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the biomarker or a fragment thereof (e.g., modulations of biological pathways identified herein, such as modulated proliferation, apoptosis, cell cycle, and/or ligand-receptor binding activity). Determining the ability of the blocking agent to bind to or interact with said polypeptide can be accomplished by measuring the ability of an agent to modulate immune responses, for example, by detecting changes in type and amount of cytokine secretion, changes in apoptosis or proliferation, changes in gene expression or activity associated with cellular identity, or by interfering with the ability of said polypeptide to bind to antibodies that recognize a portion thereof.

In yet another embodiment, an assay of the present invention is a cell-free assay in which one or more biomarkers of the present invention, including one or more biomarkers listed in Table 1, the Figures, and the Examples, or a fragment thereof, e.g., a biologically active fragment thereof, is contacted with a test compound, and the ability of the test compound to bind to the polypeptide, or biologically active portion thereof, is determined. Binding of the test compound to the biomarker or a fragment thereof, can be determined either directly or indirectly as described above. Determining the ability of the biomarker or a fragment thereof to bind to its natural binding partner(s) or a fragment(s) thereof can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological polypeptides. One or more biomarkers polypeptide or a fragment thereof can be immobilized on a BIAcore chip and multiple agents, e.g., blocking antibodies, fusion proteins, peptides, or small molecules, can be tested for binding to the immobilized biomarker polypeptide or fragment thereof. An example of using the BIA technology is described by Fitz et al. (1997) Oncogene 15:613.

The cell-free assays of the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In one or more embodiments of the above described assay methods, it may be desirable to immobilize either the biomarker nucleic acid and/or polypeptide, the natural binding partner(s) of the biomarker, or fragments thereof, to facilitate separation of complexed from uncomplexed forms of the reactants, as well as to accommodate automation of the assay. Binding of a test compound in the assay can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-base fusion proteins, can be adsorbed onto glutathione Sepharose® beads (Sigma Chemical, St. Louis, Mo.) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

In an alternative embodiment, determining the ability of the test compound to modulate the activity of one or more biomarkers of the present invention, including one or more biomarkers listed in Table 1, the Figures, and the Examples, or a fragment thereof, or of natural binding partner(s) thereof can be accomplished by determining the ability of the test compound to modulate the expression or activity of a gene, e.g., nucleic acid, or gene product, e.g., polypeptide, that functions downstream of the interaction. For example, cellular migration or invasion can be determined by monitoring cellular movement, matrigel assays, induction of invasion-related gene expression, and the like, as described further herein.

In another embodiment, modulators of one or more biomarkers of the present invention, including one or more biomarkers listed in Table 1, the Figures, and the Examples, or a fragment thereof, are identified in a method wherein a cell is contacted with a candidate compound and the expression or activity level of the biomarker is determined. The level of expression of biomarker RNA or polypeptide or fragments thereof in the presence of the candidate compound is compared to the level of expression of biomarker RNA or polypeptide or fragments thereof in the absence of the candidate compound. The candidate compound can then be identified as a modulator of biomarker expression based on this comparison. For example, when expression of biomarker RNA or polypeptide or fragments thereof is greater (statistically significantly greater) in the presence of the candidate compound than in its absence, the candidate compound is identified as a stimulator of biomarker expression. Alternatively, when expression of biomarker RNA or polypeptide or fragments thereof is reduced (statistically significantly less) in the presence of the candidate compound than in its absence, the candidate compound is identified as an inhibitor of biomarker expression. The expression level of biomarker RNA or polypeptide or fragments thereof in the cells can be determined by methods described herein for detecting biomarker mRNA or polypeptide or fragments thereof.

In yet another aspect of the present invention, a biomarker of the present invention, including one or more biomarkers listed in Table 1, the Figures, and the Examples, or a fragment thereof, can be used as “bait” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other nucleic acids and/or polypeptides which bind to or interact with the biomarker or fragments thereof and are involved in activity of the biomarkers. Such biomarker-binding proteins are also likely to be involved in the propagation of signals by the biomarker polypeptides or biomarker natural binding partner(s) as, for example, downstream elements of one or more biomarkers-mediated signaling pathway.

The two-hybrid system is based on the modular nature of most transcription factors, which consist of separable DNA-binding and activation domains. Briefly, the assay utilizes two different DNA constructs. In one construct, the gene that codes for one or more biomarkers polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. If the “bait” and the “prey” polypeptides are able to interact, in vivo, forming one or more biomarkers-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with one or more biomarkers polypeptide of the present invention, including one or more biomarkers listed in Table 1, the Figures, and the Examples, or a fragment thereof.

In another aspect, the invention pertains to a combination of two or more of the assays described herein. For example, a modulating agent can be identified using a cell-based or a cell-free assay, and the ability of the agent to modulate the activity of one or more biomarkers polypeptide or a fragment thereof can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### V. Uses and Methods of the Present Invention

The biomarkers of the present invention described herein, including the biomarkers listed in Table 1, the Figures, and the Examples, or fragments thereof, can be used in one or more of the following methods: a) screening assays; b) predictive medicine (e.g., diagnostic assays, prognostic assays, and monitoring of clinical trials); and c) methods of treatment (e.g., therapeutic and prophylactic, e.g., by up- or down-modulating the copy number, level of expression, and/or level of activity of the one or more biomarkers).

The biomarkers described herein or agents that modulate the expression and/or activity of such biomarkers can be used, for example, to (a) express one or more biomarkers of the present invention, including one or more biomarkers listed in Table 1, the Figures, and the Examples, or a fragment thereof (e.g., via a recombinant expression vector in a host cell in gene therapy applications or synthetic nucleic acid molecule), (b) detect biomarker RNA or a fragment thereof (e.g., in a biological sample) or a genetic alteration in one or more biomarkers gene, and/or (c) modulate biomarker activity, as described further below. The biomarkers or modulatory agents thereof can be used to treat conditions or disorders characterized by insufficient or excessive production of one or more biomarkers polypeptide or fragment thereof or production of biomarker polypeptide inhibitors. In addition, the biomarker polypeptides or fragments thereof can be used to screen for naturally occurring biomarker binding partner(s), to screen for drugs or compounds which modulate biomarker activity, as well as to treat conditions or disorders characterized by insufficient or excessive production of biomarker polypeptide or a fragment thereof or production of biomarker polypeptide forms which have decreased, aberrant or unwanted activity compared to biomarker wild-type polypeptides or fragments thereof (e.g., melanoma).

A. Screening Assays

In one aspect, the present invention relates to a method for preventing in a subject, a disease or condition associated with an unwanted, more than desirable, or less than desirable, expression and/or activity of one or more biomarkers described herein. Subjects at risk for a disease that would benefit from treatment with the claimed agents or methods can be identified, for example, by any one or combination of diagnostic or prognostic assays known in the art and described herein (see, for example, agents and assays described in IV. Methods of Selecting Agents and Compositions).

B. Predictive Medicine

The present invention also pertains to the field of predictive medicine in which diagnostic assays, prognostic assays, and monitoring of clinical trials are used for prognostic (predictive) purposes to thereby treat an individual prophylactically.

Accordingly, one aspect of the present invention relates to diagnostic assays for determining the expression and/or activity level of biomarkers of the present invention, including biomarkers listed in Table 1, the Figures, and the Examples, or fragments thereof, in the context of a biological sample (e.g., blood, serum, cells, or tissue) to thereby determine whether an individual is afflicted with a disease or disorder, or is at risk of developing a disorder, associated with aberrant or unwanted biomarker expression or activity. The present invention also provides for prognostic (or predictive) assays for determining whether an individual is at risk of developing a disorder associated with biomarker polypeptide, nucleic acid expression or activity. For example, mutations in one or more biomarkers gene can be assayed in a biological sample.

Such assays can be used for prognostic or predictive purpose to thereby prophylactically treat an individual prior to the onset of a disorder characterized by or associated with biomarker polypeptide, nucleic acid expression or activity. For example, SLNCR expression and activity is associated with cancer invasion such that overexpression of SLNCR predicts cancer progression and differential therapy, such as anti-SLNCR therapy either alone or in combination with additional agents, including nuclear receptor inhibitors.

Another aspect of the present invention pertains to monitoring the influence of agents (e.g., drugs, compounds, and small nucleic acid-based molecules) on the expression or activity of biomarkers of the present invention, including biomarkers listed in Table 1, the Figures, and the Examples, or fragments thereof, in clinical trials. These and other agents are described in further detail in the following sections.

1. Diagnostic Assays

The present invention provides, in part, methods, systems, and code for accurately classifying whether a biological sample is associated with a melanoma or a clinical subtype thereof. In some embodiments, the present invention is useful for classifying a sample (e.g., from a subject) as a cancer sample using a statistical algorithm and/or empirical data (e.g., the presence or level of one or biomarkers described herein).

An exemplary method for detecting the level of expression or activity of one or more biomarkers of the present invention, including one or more biomarkers listed in Table 1, the Figures, and the Examples, or fragments thereof, and thus useful for classifying whether a sample is associated with melanoma or a clinical subtype thereof, involves obtaining a biological sample from a test subject and contacting the biological sample with a compound or an agent capable of detecting the biomarker (e.g., polypeptide or nucleic acid that encodes the biomarker or fragments thereof) such that the level of expression or activity of the biomarker is detected in the biological sample. In some embodiments, the presence or level of at least one, two, three, four, five, six, seven, eight, nine, ten, fifty, hundred, or more biomarkers of the present invention are determined in the individual's sample. In certain instances, the statistical algorithm is a single learning statistical classifier system. Exemplary statistical analyses are presented in the Examples and can be used in certain embodiments. In other embodiments, a single learning statistical classifier system can be used to classify a sample as a cancer sample, a cancer subtype sample, or a non-cancer sample based upon a prediction or probability value and the presence or level of one or more biomarkers described herein. The use of a single learning statistical classifier system typically classifies the sample as a cancer sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the cancer classification results to a clinician, e.g., an oncologist or hematologist.

In another embodiment, the method of the present invention further provides a diagnosis in the form of a probability that the individual has a cancer, such as melanoma, or a clinical subtype thereof. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having cancer or a clinical subtype thereof. In yet another embodiment, the method of the present invention further provides a prognosis of cancer in the individual. For example, the prognosis can be surgery, development of melanoma or a clinical subtype thereof, development of one or more symptoms, development of malignant cancer, or recovery from the disease. In some instances, the method of classifying a sample as a cancer sample is further based on the symptoms (e.g., clinical factors) of the individual from which the sample is obtained. The symptoms or group of symptoms can be, for example, those associated with the IPI. In some embodiments, the diagnosis of an individual as having melanoma or a clinical subtype thereof is followed by administering to the individual a therapeutically effective amount of a drug useful for treating one or more symptoms associated with melanoma or a clinical subtype thereof.

In some embodiments, an agent for detecting biomarker RNA, genomic DNA, or fragments thereof is a labeled nucleic acid probe capable of hybridizing to biomarker RNA, genomic DNA., or fragments thereof. The nucleic acid probe can be, for example, full-length biomarker nucleic acid, or a portion thereof, such as an oligonucleotide of at least 15, 30, 50, 100, 250 or 500 nucleotides in length and sufficient to specifically hybridize under stringent conditions well known to a skilled artisan to biomarker mRNA or genomic DNA. Other suitable probes for use in the diagnostic assays of the present invention are described herein. In some embodiments, the nucleic acid probe is designed to detect transcript variants (i.e., different splice forms) of a gene.

A preferred agent for detecting SLNCR bioimarkers in complex with biomarker proteins is an antibody capable of binding to the biomarker, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, and biological fluids isolated from a subject, as well as tissues, cells, and fluids present within a subject. That is, the detection method of the present invention can be used to detect biomarker mRNA, polypeptide, genomic DNA, or fragments thereof, in a biological sample in vitro as well as in vivo. For example, in vitro techniques for detection of biomarker mRNA or a fragment thereof include Northern hybridizations and in situ hybridizations. In vitro techniques for detection of biomarker polypeptide include enzyme linked immunosorbent assays (ELISAs), Western blots, immunoprecipitations and immunofluorescence. In vitro techniques for detection of biomarker genomic DNA or a fragment thereof include Southern hybridizations. Furthermore, in vivo techniques for detection of one or more biomarkers polypeptide or a fragment thereof include introducing into a subject a labeled anti-biomarker antibody. For example, the antibody can be labeled with a radioactive marker whose presence and location in a subject can be detected by standard imaging techniques.

In one embodiment, the biological sample contains polypeptide molecules from the test subject. Alternatively, the biological sample can contain RNA molecules from the test subject or genomic DNA molecules from the test subject. A preferred biological sample is a hematological tissue (e.g., a sample comprising blood, plasma, B cell, bone marrow, etc.) sample isolated by conventional means from a subject.

In another embodiment, the methods further involve obtaining a control biological sample from a control subject, contacting the control sample with a compound or agent capable of detecting polypeptide, RNA, cDNA, small RNAs, mature miRNA, pre-miRNA, pri-miRNA, miRNA*, piwiRNA, anti-miRNA, or a miRNA binding site, or a variant thereof, genomic DNA, or fragments thereof of one or more biomarkers listed in Table 1, the Figures, and the Examples, such that the presence of biomarker polypeptide, RNA, genomic DNA, or fragments thereof, is detected in the biological sample, and comparing the presence of biomarker polypeptide, RNA, cDNA, small RNAs, mature miRNA, pre-miRNA, pri-miRNA, miRNA*, piwiRNA, anti-miRNA, or a miRNA binding site, or a variant thereof, genomic DNA, or fragments thereof in the control sample with the presence of biomarker polypeptide, RNA, cDNA, small RNAs, mature miRNA, pre-miRNA, pri-miRNA, miRNA*, piwiRNA, piwiRNA, anti-miRNA, or a miRNA binding site, or a variant thereof, genomic DNA, or fragments thereof in the test sample.

The invention also encompasses kits for detecting the presence of a polypeptide, RNA, cDNA, small RNAs, mature miRNA, pre-miRNA, pri-miRNA, miRNA*, piwiRNA, anti-miRNA, or a miRNA binding site, or a variant thereof, genomic DNA, or fragments thereof, of one or more biomarkers listed in Table 1, the Figures, and the Examples, in a biological sample. For example, the kit can comprise a labeled compound or agent capable of detecting one or more biomarkers polypeptide, RNA, cDNA, small RNAs, mature miRNA, pre-miRNA, pri-miRNA, miRNA*, piwiRNA, anti-miRNA, or a miRNA binding site, or a variant thereof, genomic DNA, or fragments thereof, in a biological sample; means for determining the amount of the biomarker polypeptide, RNA, cDNA, small RNAs, mature miRNA, pre-miRNA, pri-miRNA, miRNA*, piwiRNA, anti-miRNA, or a miRNA binding site, or a variant thereof, genomic DNA, or fragments thereof,f in the sample; and means for comparing the amount of the biomarker polypeptide, RNA, cDNA, small RNAs, mature miRNA, pre-miRNA, pri-miRNA, miRNA*, piwiRNA, anti-miRNA, or a miRNA binding site, or a variant thereof, genomic DNA, or fragments thereof, in the sample with a standard. The compound or agent can be packaged in a suitable container. The kit can further comprise instructions for using the kit to detect the biomarker polypeptide, RNA, cDNA, small RNAs, mature miRNA, pre-miRNA, pri-miRNA, miRNA*, piwiRNA, anti-miRNA, or a miRNA binding site, or a variant thereof, genomic DNA, or fragments thereof.

In some embodiments, therapies tailored to treat stratified patient populations based on the described diagnostic assays are further administered, such as melanoma standards of treatment, immune therapy, and combinations thereof described herein.

2. Prognostic Assays

The diagnostic methods described herein can furthermore be utilized to identify subjects having or at risk of developing a disease or disorder associated with aberrant expression or activity of one or more biomarkers of the present invention, including one or more biomarkers listed in Table 1, the Figures, and the Examples, or a fragment thereof. As used herein, the term “aberrant” includes biomarker expression or activity levels which deviates from the normal expression or activity in a control.

The assays described herein, such as the preceding diagnostic assays or the following assays, can be used to identify a subject having or at risk of developing a disorder associated with a misregulation of biomarker activity or expression, such as in a cancer like melanoma. Alternatively, the prognostic assays can be used to identify a subject having or at risk for developing a disorder associated with a misregulation of biomarker activity or expression. Thus, the present invention provides a method for identifying and/or classifying a disease associated with aberrant expression or activity of one or more biomarkers of the present invention, including one or more biomarkers listed in Table 1, the Figures, and the Examples, or a fragment thereof. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with aberrant biomarker expression or activity. For example, such methods can be used to determine whether a subject can be effectively treated with an agent for a melanoma. Thus, the present invention provides methods for determining whether a subject can be effectively treated with an agent for a disease associated with aberrant biomarker expression or activity in which a test sample is obtained and biomarker polypeptide or nucleic acid expression or activity is detected (e.g., wherein a significant increase or decrease in biomarker polypeptide or nucleic acid expression or activity relative to a control is diagnostic for a subject that can be administered the agent to treat a disorder associated with aberrant biomarker expression or activity). In some embodiments, significant increase or decrease in biomarker expression or activity comprises at least 1.1, 1.2, 1.3, 1.4, 1.5, 1.6, 1.7, 1.8, 1.9, 2.0, 2 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher or lower, respectively, than the expression activity or level of the marker in a control sample.

The methods of the present invention can also be used to detect genetic alterations in one or more biomarkers of the present invention, including one or more biomarkers listed in Table 1, the Figures, and the Examples, or a fragment thereof, thereby determining if a subject with the altered biomarker is at risk for melanoma characterized by aberrant biomarker activity or expression levels. In preferred embodiments, the methods include detecting, in a sample of cells from the subject, the presence or absence of a genetic alteration characterized by at least one alteration affecting the integrity of a gene encoding one or more biomarkers, or the mis-expression of the biomarker (e.g., mutations and/or splice variants). For example, such genetic alterations can be detected by ascertaining the existence of at least one of 1) a deletion of one or more nucleotides from one or more biomarkers gene, 2) an addition of one or more nucleotides to one or more biomarkers gene, 3) a substitution of one or more nucleotides of one or more biomarkers gene, 4) a chromosomal rearrangement of one or more biomarkers gene, 5) an alteration in the level of a messenger RNA transcript of one or more biomarkers gene, 6) aberrant modification of one or more biomarkers gene, such as of the methylation pattern of the genomic DNA, 7) the presence of a non-wild type splicing pattern of an RNA transcript of one or more biomarkers gene, 8) a non-wild type level of one or more biomarkers polypeptide, 9) allelic loss of one or more biomarkers gene, and 10) inappropriate post-translational modification of one or more biomarkers polypeptide. As described herein, there are a large number of assays known in the art which can be used for detecting alterations in one or more biomarkers gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in one or more biomarkers gene (see Abravaya et al. (1995) Nucleic Acids Res. 23:675-682). This method can include the steps of collecting a sample of cells from a subject, isolating nucleic acid (e.g., genomic DNA, mRNA, cDNA, small RNA, mature miRNA, pre-miRNA, pri-miRNA, miRNA*, piwiRNA, anti-miRNA, or a miRNA binding site, or a variant thereof) from the cells of the sample, contacting the nucleic acid sample with one or more primers which specifically hybridize to one or more biomarkers gene of the present invention, including the biomarker genes listed in Table 1, the Figures, and the Examples, or fragments thereof, under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more biomarkers gene of the present invention, including one or more biomarkers listed in Table 1, the Figures, and the Examples, or a fragment thereof, from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in one or more biomarkers gene of the present invention, including a gene listed in Table 1, the Figures, and the Examples, or a fragment thereof, can be identified by hybridizing a sample and control nucleic acids, e.g., DNA, RNA, mRNA, small RNA, cDNA, mature miRNA, pre-miRNA, pri-miRNA, miRNA*, piwiRNA, anti-miRNA, or a miRNA binding site, or a variant thereof, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in one or more biomarkers can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence one or more biomarkers gene of the present invention, including a gene listed in Table 1, the Figures, and the Examples, or a fragment thereof, and detect mutations by comparing the sequence of the sample biomarker gene with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in one or more biomarkers gene of the present invention, including a gene listed in Table 1, the Figures, and the Examples, or fragments thereof, include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker genes of the present invention, including genes listed in Table 1, the Figures, and the Examples, or fragments thereof, obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in biomarker genes of the present invention, including genes listed in Table 1, the Figures, and the Examples, or fragments thereof. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA. In some embodiments, the hybridization reactions can occur using biochips, microarrays, etc., or other array technology that are well known in the art.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving one or more biomarkers of the present invention, including one or more biomarkers listed in Table 1, the Figures, and the Examples, or fragments thereof.

3. Monitoring of Effects During Clinical Trials Monitoring the influence of agents (e.g., drugs) on the expression or activity of one or more biomarkers of the present invention, including one or more biomarkers listed in Table 1, the Figures, and the Examples, or a fragment thereof (e.g., the modulation of a cancer state) can be applied not only in basic drug screening, but also in clinical trials. For example, the effectiveness of an agent determined by a screening assay as described herein to increase expression and/or activity of one or more biomarkers of the present invention, including one or more biomarkers listed in Table 1, the Figures, and the Examples, or a fragment thereof, can be monitored in clinical trials of subjects exhibiting decreased expression and/or activity of one or more biomarkers of the present invention, including one or more biomarkers of the present invention, including one or more biomarkers listed in Table 1, the Figures, and the Examples, or a fragment thereof, relative to a control reference. Alternatively, the effectiveness of an agent determined by a screening assay to decrease expression and/or activity of one or more biomarkers of the present invention, including one or more biomarkers listed in Table 1, the Figures, and the Examples, or a fragment thereof, can be monitored in clinical trials of subjects exhibiting decreased expression and/or activity of the biomarker of the present invention, including one or more biomarkers listed in Table 1, the Figures, and the Examples, or a fragment thereof relative to a control reference. In such clinical trials, the expression and/or activity of the biomarker can be used as a “read out” or marker of the phenotype of a particular cell.

In some embodiments, the present invention provides a method for monitoring the effectiveness of treatment of a subject with an agent (e.g., an agonist, antagonist, peptidomimetic, polypeptide, peptide, nucleic acid, small molecule, or other drug candidate identified by the screening assays described herein) including the steps of (i) obtaining a pre-administration sample from a subject prior to administration of the agent; (ii) detecting the level of expression and/or activity of one or more biomarkers of the present invention, including one or more biomarkers listed in Table 1, the Figures, and the Examples, or fragments thereof in the preadministration sample; (iii) obtaining one or more post-administration samples from the subject; (iv) detecting the level of expression or activity of the biomarker in the post-administration samples; (v) comparing the level of expression or activity of the biomarker or fragments thereof in the pre-administration sample with the that of the biomarker in the post administration sample or samples; and (vi) altering the administration of the agent to the subject accordingly. For example, increased administration of the agent may be desirable to increase the expression or activity of one or more biomarkers to higher levels than detected (e.g., to increase the effectiveness of the agent.) Alternatively, decreased administration of the agent may be desirable to decrease expression or activity of the biomarker to lower levels than detected (e.g., to decrease the effectiveness of the agent). According to such an embodiment, biomarker expression or activity may be used as an indicator of the effectiveness of an agent, even in the absence of an observable phenotypic response.

C. Methods of Treatment

The present invention provides for both prophylactic and therapeutic methods of treating a subject at risk of (or susceptible to) a disorder characterized by insufficient or excessive production of biomarkers of the present invention, including biomarkers listed in Table 1, the Figures, and the Examples, or fragments thereof, which have aberrant expression or activity compared to a control. Moreover, agents of the present invention described herein can be used to detect and isolate the biomarkers or fragments thereof, regulate the bioavailability of the biomarkers or fragments thereof, and modulate biomarker expression levels or activity.

1. Prophylactic Methods

In one aspect, the present invention provides a method for preventing in a subject, a disease or condition associated with an aberrant expression or activity of one or more biomarkers of the present invention, including one or more biomarkers listed in Table 1, the Figures, and the Examples, or a fragment thereof, by administering to the subject an agent which modulates biomarker expression or at least one activity of the biomarker. Subjects at risk for a disease or disorder which is caused or contributed to by aberrant biomarker expression or activity can be identified by, for example, any or a combination of diagnostic or prognostic assays as described herein. Administration of a prophylactic agent can occur prior to the manifestation of symptoms characteristic of the biomarker expression or activity aberrancy, such that a disease or disorder is prevented or, alternatively, delayed in its progression.

2. Therapeutic Methods Another aspect of the present invention pertains to methods of modulating the expression or activity or interaction with natural binding partner(s) of one or more biomarkers of the present invention, including one or more biomarkers listed in Table 1, the Figures, and the Examples, or fragments thereof, for therapeutic purposes. The biomarkers of the present invention have been demonstrated to correlate with cancer, such as melanoma. Accordingly, the activity and/or expression of the biomarker, as well as the interaction between one or more biomarkers or a fragment thereof and its natural binding partner(s) or a fragment(s) thereof can be modulated in order to modulate the immune response.

Modulatory methods of the present invention involve contacting a cell with one or more biomarkers of the present invention, including one or more biomarkers of the present invention, including one or more biomarkers listed in Table 1, the Figures, and the Examples, or a fragment thereof or agent that modulates one or more of the activities of biomarker activity associated with the cell. An agent that modulates biomarker activity can be an agent as described herein, such as a nucleic acid or a polypeptide, a naturally-occurring binding partner of the biomarker, an antibody against the biomarker, a combination of antibodies against the biomarker and antibodies against other immune related targets, one or more biomarkers agonist or antagonist, a peptidomimetic of one or more biomarkers agonist or antagonist, one or more biomarkers peptidomimetic, other small molecule, or small RNA directed against or a mimic of one or more biomarkers nucleic acid gene expression product.

An agent that modulates the expression of one or more biomarkers of the present invention, including one or more biomarkers of the present invention, including one or more biomarkers listed in Table 1, the Figures, and the Examples, or a fragment thereof is a nucleic acid molecule described herein, e.g., an antisense nucleic acid molecule, RNAi molecule, shRNA, mature miRNA, pre-miRNA, pri-miRNA, miRNA*, piwiRNA, anti-miRNA, or a miRNA binding site, or a variant thereof, or other small RNA molecule, triplex oligonucleotide, ribozyme, or recombinant vector for expression of one or more biomarkers polypeptide. For example, an oligonucleotide complementary to the area around one or more biomarkers polypeptide translation initiation site can be synthesized. One or more antisense oligonucleotides can be added to cell media, typically at 200 μg/ml, or administered to a patient to prevent the synthesis of one or more biomarkers polypeptide. The antisense oligonucleotide is taken up by cells and hybridizes to one or more biomarkers mRNA to prevent translation. Alternatively, an oligonucleotide which binds double-stranded DNA to form a triplex construct to prevent DNA unwinding and transcription can be used. As a result of either, synthesis of biomarker polypeptide is blocked. When biomarker expression is modulated, preferably, such modulation occurs by a means other than by knocking out the biomarker gene.

Agents which modulate expression, by virtue of the fact that they control the amount of biomarker in a cell, also modulate the total amount of biomarker activity in a cell.

In one embodiment, the agent stimulates one or more activities of one or more biomarkers of the present invention, including one or more biomarkers listed in Table 1, the Figures, and the Examples, or a fragment thereof. Examples of such stimulatory agents include active biomarker polypeptides or a fragment thereof, such as SLNCR binding partners, and/or a nucleic acid molecule encoding the biomarker or a fragment thereof that has been introduced into the cell (e.g., cDNA, mRNA, shRNAs, siRNAs, small RNAs, mature miRNA, pre-miRNA, pri-miRNA, miRNA*, piwiRNA, anti-miRNA, or a miRNA binding site, or a variant thereof, or other functionally equivalent molecule known to a skilled artisan). In another embodiment, the agent inhibits one or more biomarker activities. In one embodiment, the agent inhibits or enhances the interaction of the biomarker with its natural binding partner(s). Examples of such inhibitory agents include antisense nucleic acid molecules, anti-biomarker antibodies, biomarker inhibitors, and compounds identified in the screening assays described herein.

These modulatory methods can be performed in vitro (e.g., by contacting the cell with the agent) or, alternatively, by contacting an agent with cells in vivo (e.g., by administering the agent to a subject). As such, the present invention provides methods of treating an individual afflicted with a condition or disorder that would benefit from up- or down-modulation of one or more biomarkers of the present invention listed in Table 1, the Figures, and the Examples, or a fragment thereof, e.g., a disorder characterized by unwanted, insufficient, or aberrant expression or activity of the biomarker or fragments thereof. In one embodiment, the method involves administering an agent (e.g., an agent identified by a screening assay described herein), or combination of agents that modulates (e.g., upregulates or downregulates) biomarker expression or activity. In another embodiment, the method involves administering one or more biomarkers polypeptide or

nucleic acid molecule as therapy to compensate for reduced, aberrant, or unwanted biomarker expression or activity.

Stimulation of biomarker activity is desirable in situations in which the biomarker is abnormally downregulated and/or in which increased biomarker activity is likely to have a beneficial effect. Likewise, inhibition of biomarker activity is desirable in situations in which biomarker is abnormally upregulated and/or in which decreased biomarker activity is likely to have a beneficial effect.

In addition, these modulatory agents can also be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

### IV. Pharmaceutical Compositions

In another aspect, the present invention provides pharmaceutically acceptable compositions which comprise a therapeutically-effective amount of an agent that modulates (e.g., increases or decreases) SLNCR levels and/or activity, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) SLNCR levels and/or activity, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1% to about 99% of active ingredient, preferably from about 5% to about 70%, most preferably from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., increases or decreases) SLNCR levels and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., increases or decreases) SLNCR levels and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., increases or decreases) SLNCR levels and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., increases or decreases) SLNCR levels and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### V. Administration of Agents

The diagnostic, prognostic, prevention, and/or treatment modulating agents of the present invention are administered to subjects in a biologically compatible form suitable for pharmaceutical administration in vivo, to either enhance or suppress immune cell mediated immune responses. By “biologically compatible form suitable for administration in vivo” is meant a form of the protein to be administered in which any toxic effects are outweighed by the therapeutic effects of the protein. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of a blocking antibody may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The agents of the present invention described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The agent may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent of the present invention (e.g., an antibody, peptide, fusion protein or small molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present invention are dictated by, and directly dependent on, (a) the unique characteristics of the active compound and the particular therapeutic effect to be achieved, and (b) the limitations inherent in the art of compounding such an active compound for the treatment of sensitivity in individuals.

In one embodiment, an agent of the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays. In addition, an antibody of the present invention can also be administered in combination therapy with, e.g., chemotherapeutic agents, hormones, antiangiogens, radiolabelled, compounds, or with surgery, cryotherapy, and/or radiotherapy. An antibody of the present invention can also be administered in conjunction with other forms of conventional therapy, either consecutively with, pre- or post-conventional therapy. For example, the antibody can be administered with a therapeutically effective dose of chemotherapeutic agent. In another embodiment, the antibody can be administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular immune disorder, e.g., melanoma, being treated, the extent of the disease and other factors familiar to the physician of skill in the art and can be determined by the physician.

In addition, the agents of the present invention described herein can be administered using nanoparticle-based composition and delivery methods well known to the skilled artisan. For example, nanoparticle-based delivery for improved nucleic acid (e.g., small RNAs) therapeutics are well known in the art (Expert Opinion on Biological Therapy 7:1811-1822).

### EXEMPLIFICATION

This invention is further illustrated by the following examples, which should not be construed as limiting.

### Example 1: Materials and Methods for Examples 2-9

For cellular fractionation, cells were grown to ˜80% confluency in 10 cm tissue culture treated dishes and fractionated using Thermo Scientific™ NEPER™ Nuclear and Cytoplasmic Extraction Kit, according to manufacturer's instructions. Nuclear and cytoplasmic fractions were split for protein and RNA analysis.

For RNA-seq and qRT-PCR experiments, RNA was isolated using Trizol® (Life Technologies) and Qiagen RNeasy® Mini Kit and treated with DNase. cDNA was generated using SuperScript III (Invitrogen) reverse transcriptase. The indicated transcripts were quantified using Platinum® SYBR® Green qPCR SuperMix-UDG mix on a CFX384 Touch™ Real-Time PCR Detection System. Error bars represent standard deviations calculated from 3 reactions. For RNA-seq analyses, cDNA libraries were prepped using TruSeq® RNA Sample Prep kit v2 (Illumina) and sequenced on the HiSeq® 2500 (Illumina) at the BROAD institute. Cuffdiff (Trapnell et al. (2010) Nat. Biotech. 28:511-515) was used to identify differentially expressed genes.

Gelatinolytic activity in the culture media of cells transfected with the indicated plasmids or siRNAs was examined by gelatin zymography. A375 cells were seeded at a density of 25×10⁴ cells per well in 6-well dishes. Transfections were carried out using Lipofectamine® 2000 (Life Technologies) using 2,500 ng of the indicated plasmid along with the indicated siRNAs at a final concentration of 7.5 nM (for MMP-9 siRNAs) or 10 nM (for AR siRNAs). Cells were washed with PBS and transitioned to 900 μl of serum free media 24 hours post-transfection. Supernatant was removed 24 hours later and non-adherent cells were pelleted by centrifugation at 300×g for 5 minutes at 4° C. The remaining supernatant was then concentrated 5-fold using Millipore Amicon® Ultra 10 kDa cutoff centrifugal devices. Samples were incubated at room temperature for 10 minutes in SDS sample buffer without a reducing agent, and then electrophoresed on 10% Criterion™ Zymogram Gel (Bio-Rad). After electrophoresis, gels were washed briefly in dH₂O, followed by 40 minute washes (2 times) in 1× renaturation buffer (Bio-Rad), incubated for 18 hours at 37° C. in 1× development buffer (Bio-Rad), and stained with 0.1% Coomassie Brilliant Blue R250. Ratios of MMP-9 compared to MMP-2 were quantified by ImageJ software densitometric analysis of the 92-kDa and 72-kDa proteolytic bands, which correspond to MMP-9 and MMP-2, respectively.

For proliferation assays, the melanoma short-term culture WM1575 was seeded at a density of 0.75×10⁴ cells/well in a 96-well plate. Lipofectamine® RNAiMAX (Life Technologies) was used to transfect the indicated siRNAs at a final concentration of 10 nM. The rate of proliferation was measured using WST-1 reagent (Roche) according to the manufacturer's instructions. Cells were incubated for 1 hour prior to measurement.

For invasion assays, 25×10⁴ cells (either A375 or the melanoma short term cultures WM1976 or WM1575) were seeded in 6 well plates. Twenty-four hours later, either 2,500 ng of the indicated plasmid was transfected using Lipofectamine® 2000 (Life Technolgoies) or the indicated siRNAs at 10 nM final concentration were transfected using Lipofectamine® RNAiMax (Life Technologies). For invasions assays using A375 cells, 2.5×10⁴ cells in serum-free media were plated in either BD BioCoat™ matrigel inserts or uncoated control inserts (Corning), placed into DMEM with 30% FBS (fetal bovine serum), and incubated for 16 hours. For the melanoma short term cultures, 10×10⁴ or 7.5×10⁴ cells, for WM1976 or WM1575, respectively, in serum-free media were seeded in the chambers, placed into DMEM with 30% FBS (fetal bovine serum), and incubated for 22 hours. Cells that did not migrate or invade were removed using a cotton tipped swap, chambers were rinsed twice with PBS, and stained using Fisher HealthCare™ PROTOCOL™ Hema 3™ Fixative and Solutions. The number of invaded or migrant cells were imaged on 20× magnification in 8 fields of view for 3 independent replicates.

To generate a plasmid-encoded nuclear MS2 protein, the simian virus nuclear localization signal (SV40-NLS) was successfully cloned upstream of the MS2 ORF using BamHI sites in a FLAG tagged hMS2 expressing vector (kindly gifted by Dr. Lynne Maquat, University of Rochester Medical Center). Nuclear localization was confirmed via fractionation and Western blotting. For RNA pulldowns, A375 cells were grown to ˜80% confluency in 10 cm dishes, transfected with 10 μg of the plasmid encoding nuclear MS2 and 8 μg of the indicated MS2 stem-loop tagged SLNCR construct using Lipofectamine® 2000 (Life Technologies), and harvested 2 days post-transfection. RNA pull-downs were completed following a slightly modified protocol from Gong and Maquat (Gong and Maquat (2015) Meth. Mol. Biol. 1206:81-86). If indicated, cells were crosslinked using 1% formaldehyde in 1×PBS for 10 minutes at room temperature and quenched by addition of glycine to a final concentration of 0.25 M. Cells were scraped into 0.5 ml lysis buffer (20 mM Tris pH 7.4, 10 mM NaCl, 2 mM EDTA, 0.5% Triton X-100, 40 U/ml RNaseOUT, 1 mM PMSF and 1× Roche protease inhibitor EDTA free), incubated for 10 minutes at 4° C., supplementated with 150 mM NaCl, and incubated an additional 5 minutes on ice. Crosslinked cells were sonicated with 6 rounds of 30 second pulsed sonication (2 seconds on and 2 second off) at an output of 4 and duty cycle of 30% using a Branson Sonifer® 2500. All samples were centrigued at 18,000×g, for 10 minutes at 4° C. to clarify lysates. Extracts were rotated for 2 hours at 4° C. with Sigma monoclonal ANTI-FLAG® M2 antibody (5 μg antibody for 4 μg of protein), added to 25 μl of Protein G Dynabeads® (Life Technologies), and rotated for an additional 1 hour at 4° C. Supernatants were removed and beads were washed 5 times in 0.5 mL wash buffer (50 mM Tris pH 7.4, 500 mM NaCal, 0.05% Triton X-100). For samples immediately subjected to western blot analysis, beads were resuspended in 25 μl 2× Laemilli sample buffer and incubated at 5° C. for 5 minutes, 95° C. for 1 hour and 5° C. for 5 minutes. For pull-down extracts subjected to transcription factor (TF) array analysis, 25 μl of wash buffer containing flag peptide at final concentration of 0.1 mg/ml was added and beads were rotated for 30 minutes at 4° C. In order to identify bound transcription factors, 12 μl of eluate was incubated with biotinylated DNA probe mixture from the Signosis® TF Activation Profiling Plate Array I and subjected to downstream analysis according to manufacturer's instructions. The signal corresponding to each TF was normalized to that of GATA, and represented as a fold enrichment compared to a cells transfected with a plasmid encoding SLNCR without the MS2 stem loop tag.

For dexamethosone and DHT stimulation assays, A375 cells were plated at a density of 1×10⁴ cells per well and transfected with 50 ng of pGL.36 (Promega) and 50 ng of the indicated plasmid. Dexamethasone (Sigma) or DHT (Sigma) was added at the indicated conentration. Luciferase activity was measured using Dual-Glo@ Luciferase Assay System (Promega).

All T-test statistics were performed using GraphPad Prism version 6.00 for Windows (GraphPad Software, La Jolla Calif. USA). All image quantifications were performed using ImageJ software.

### Example 2: The lncRNA, SLNCR, is Dysregulated in Cancer, Including in Melanoma

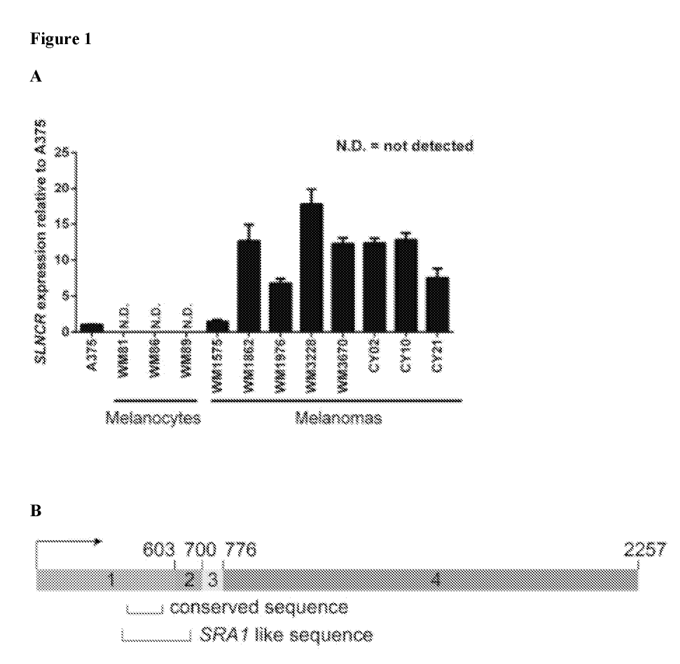

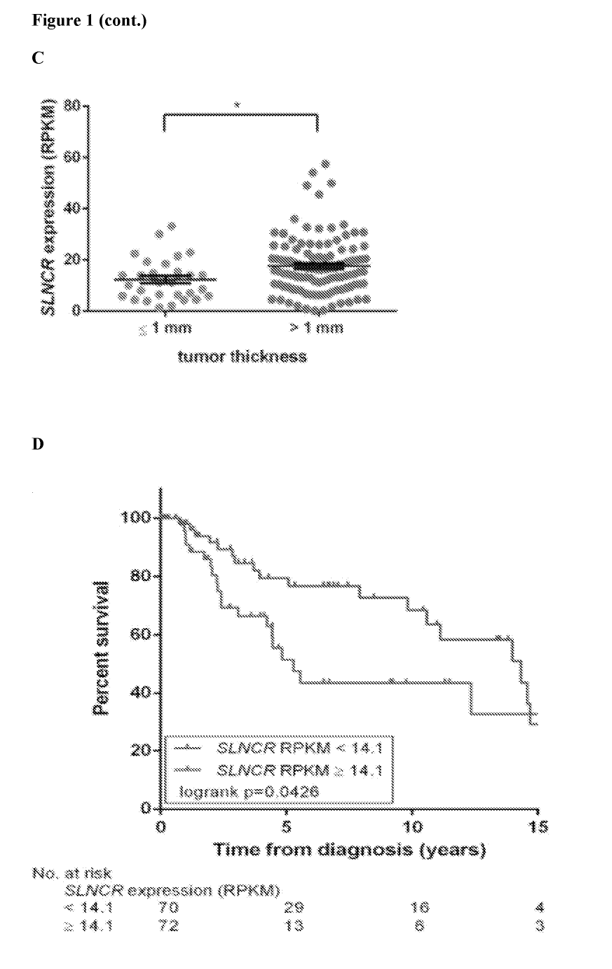

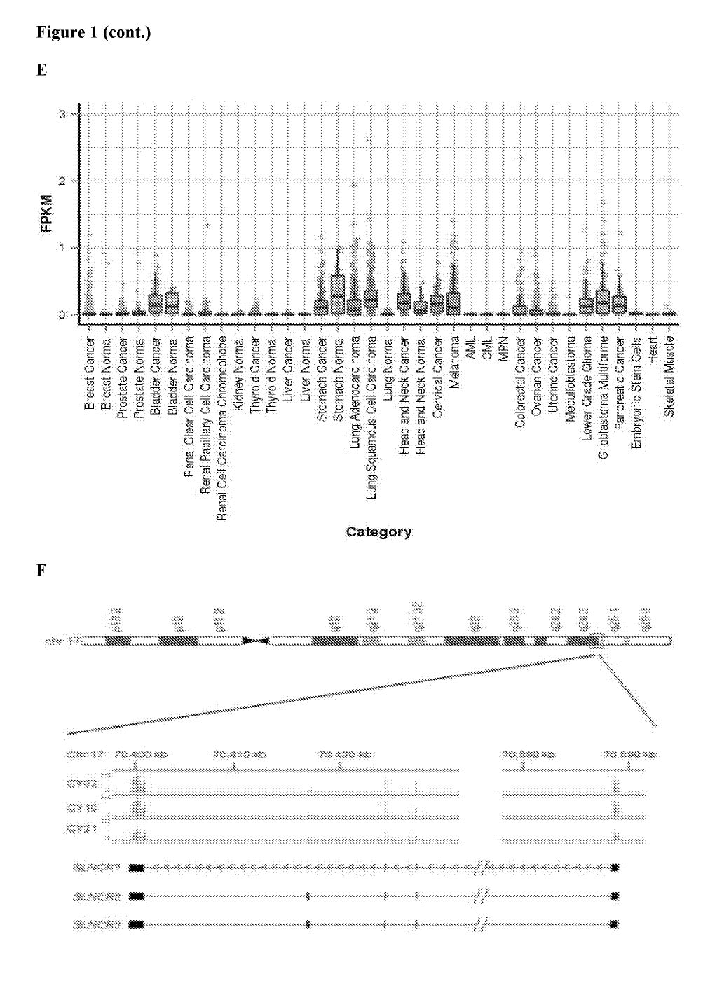

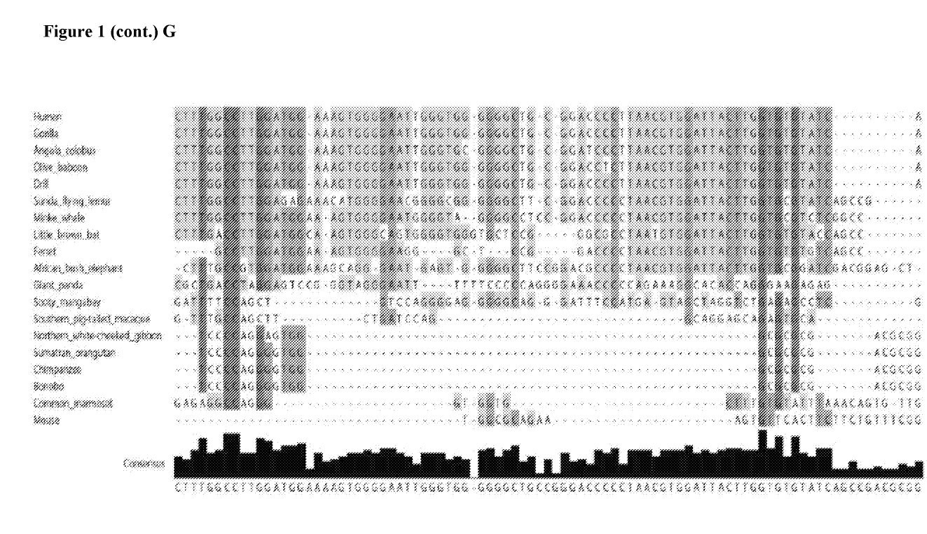

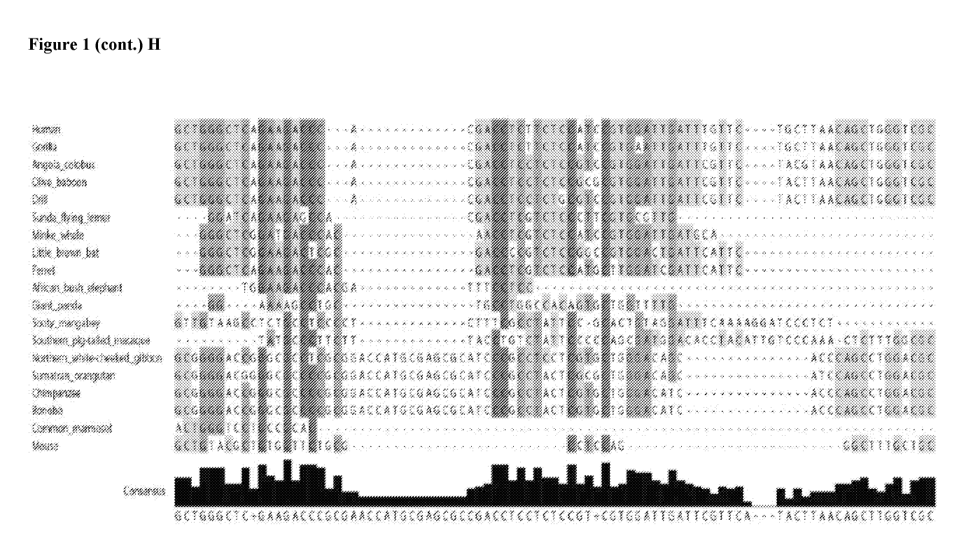

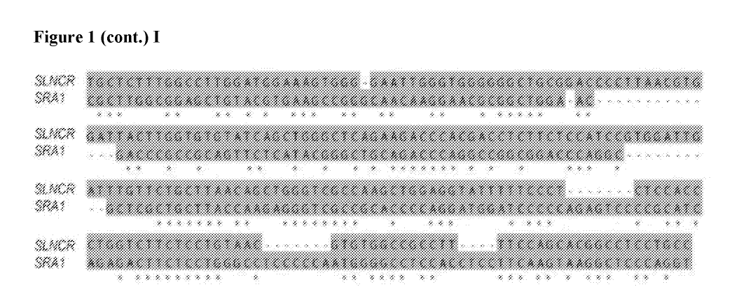

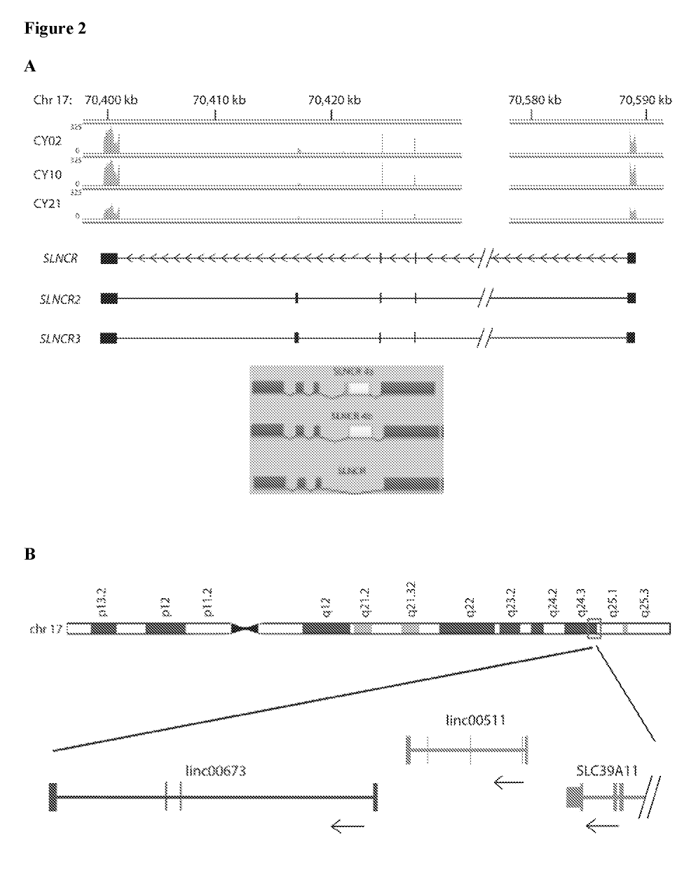

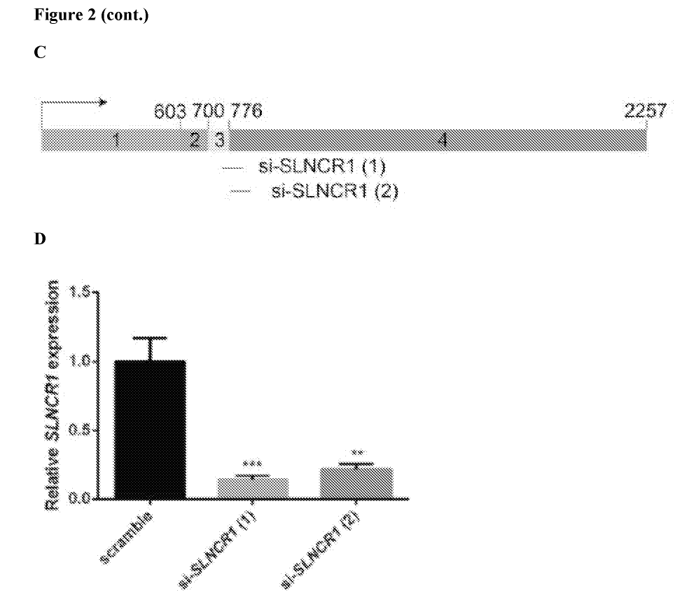

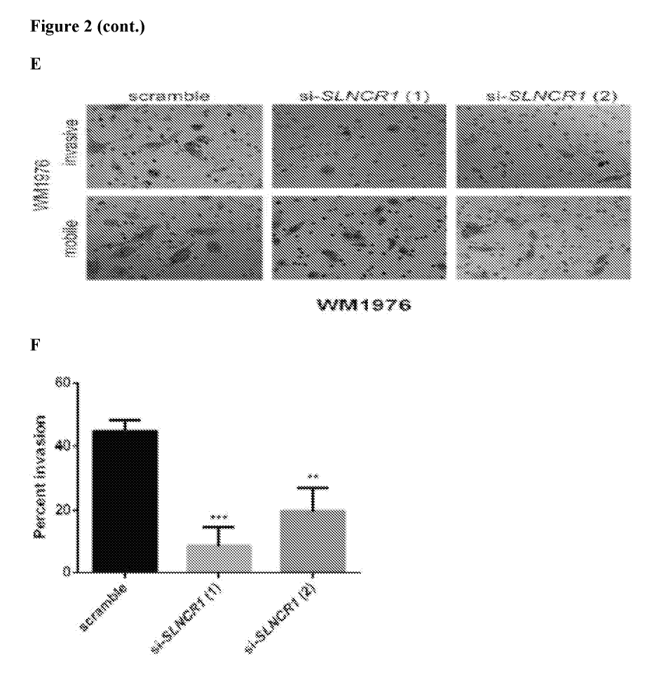

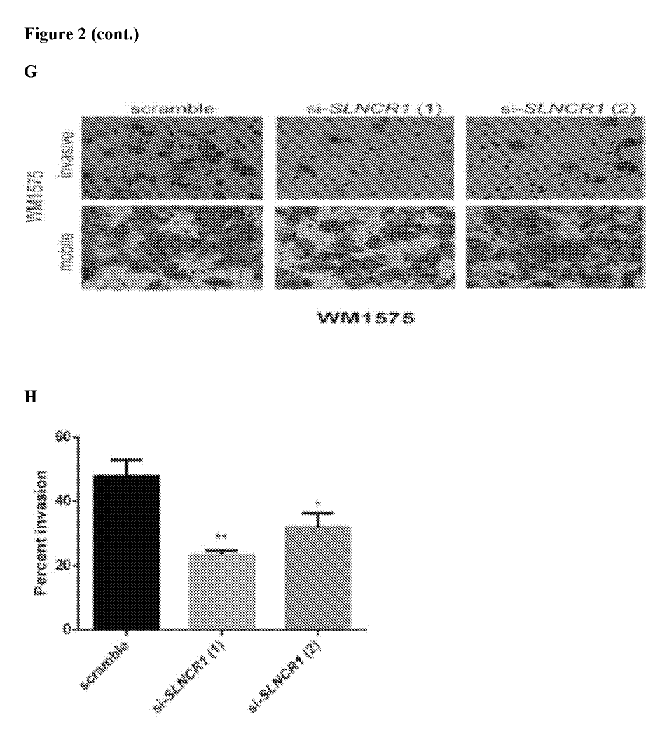

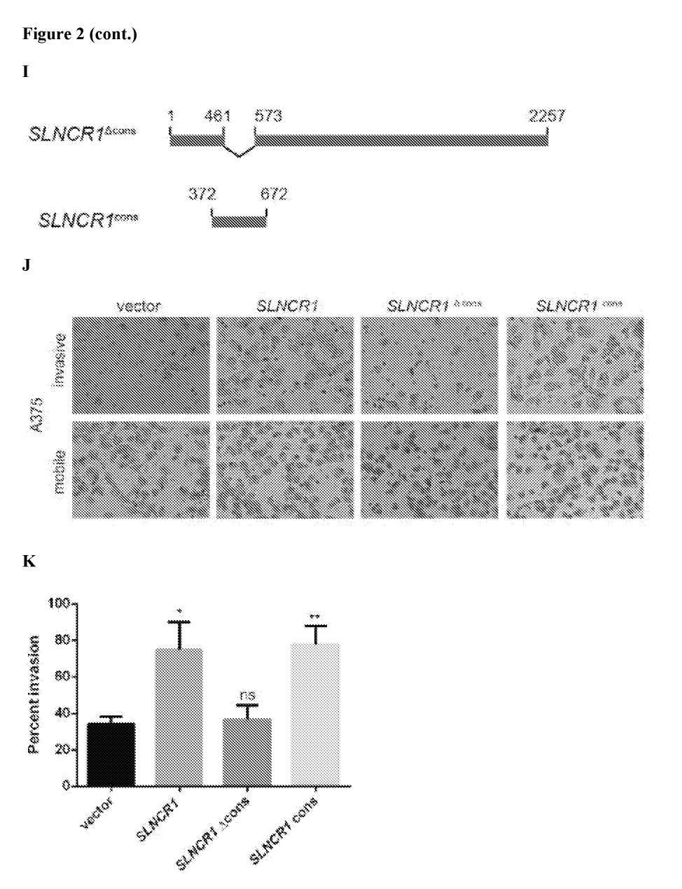

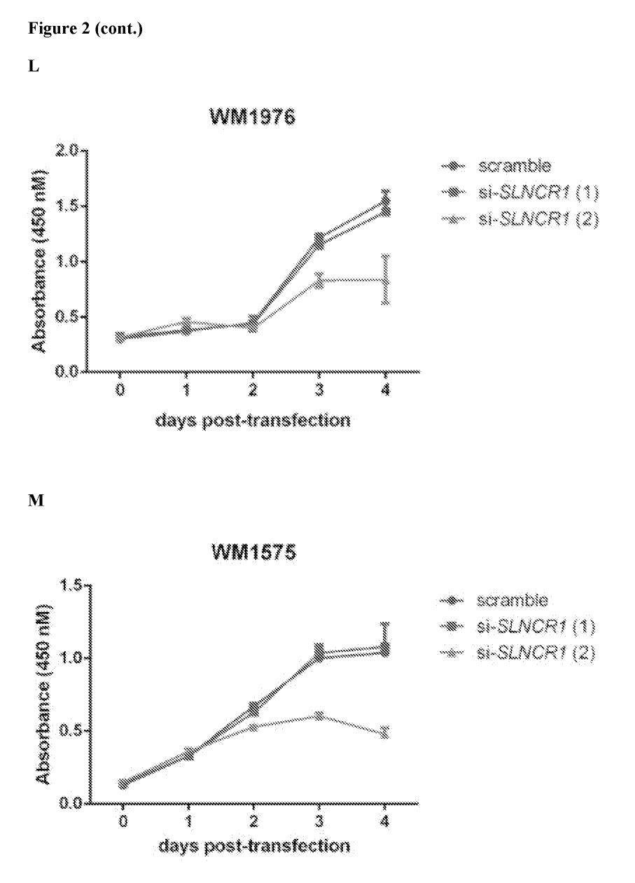

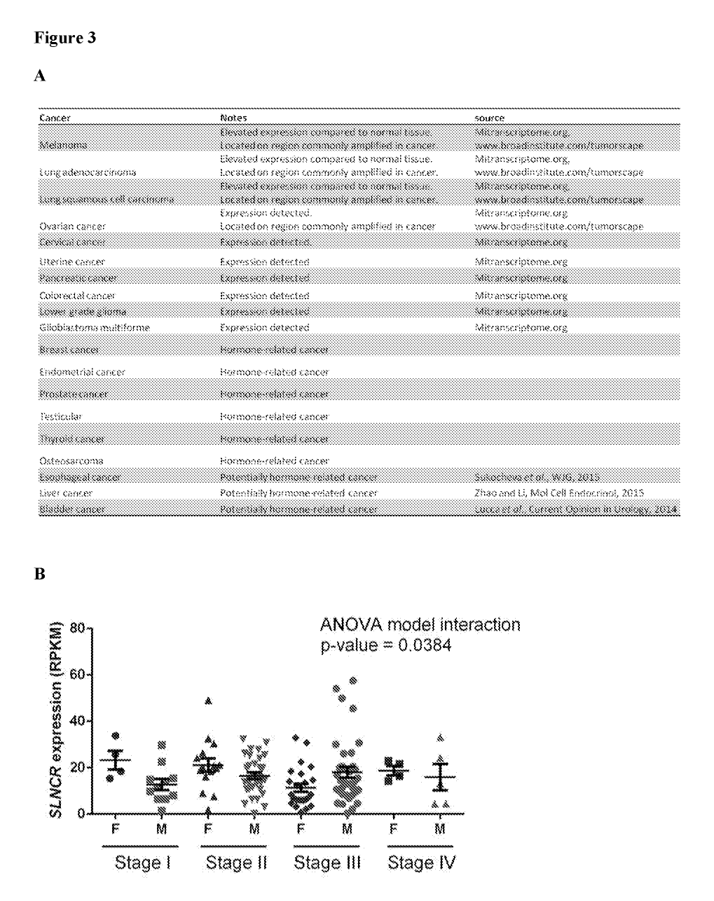

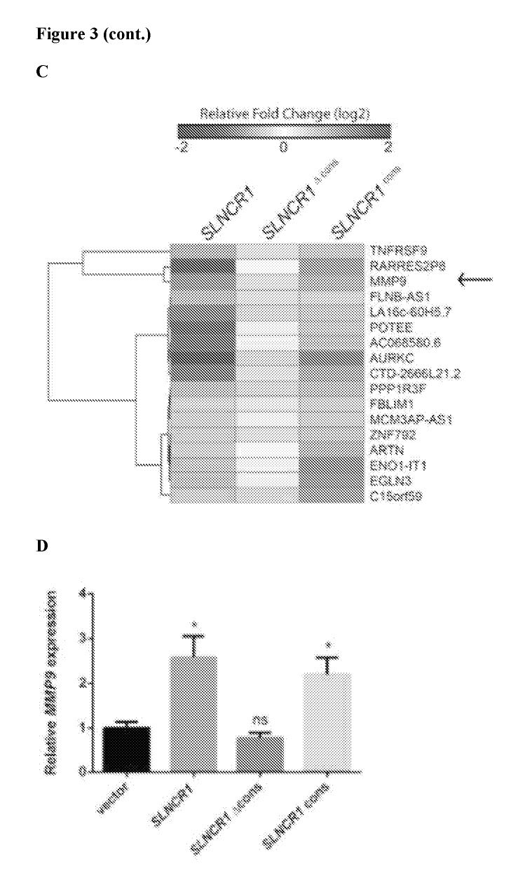

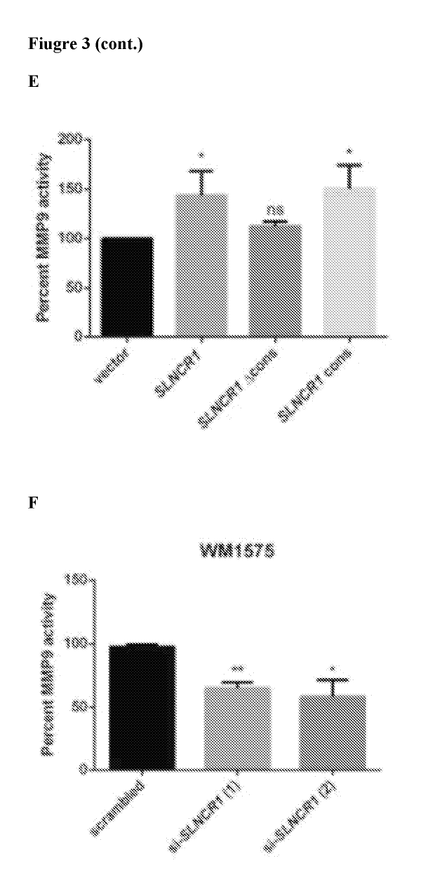

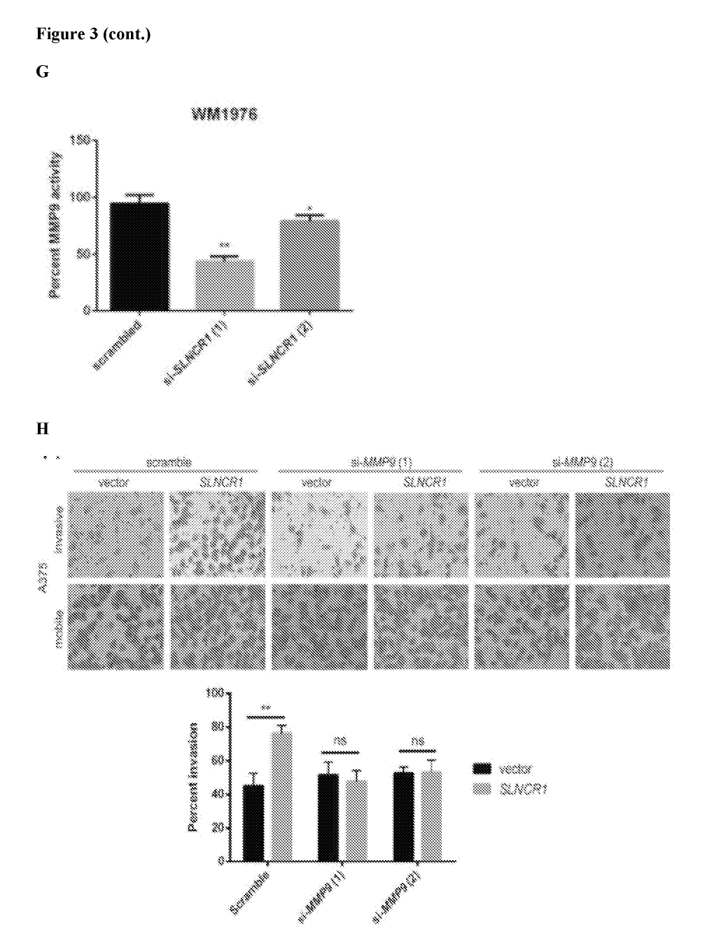

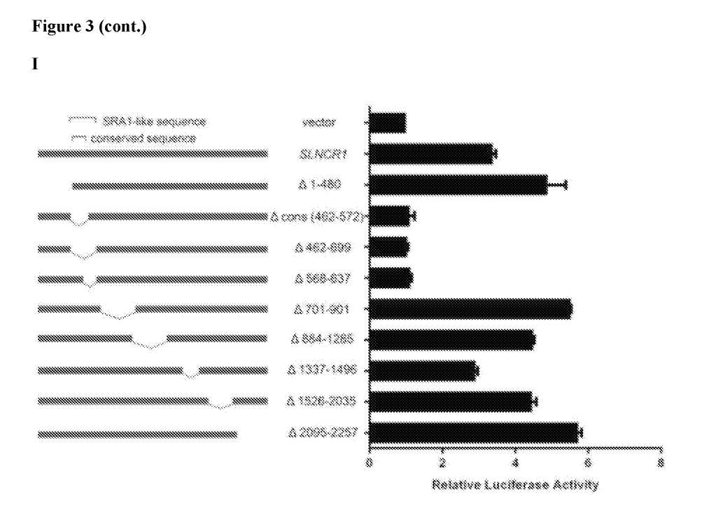

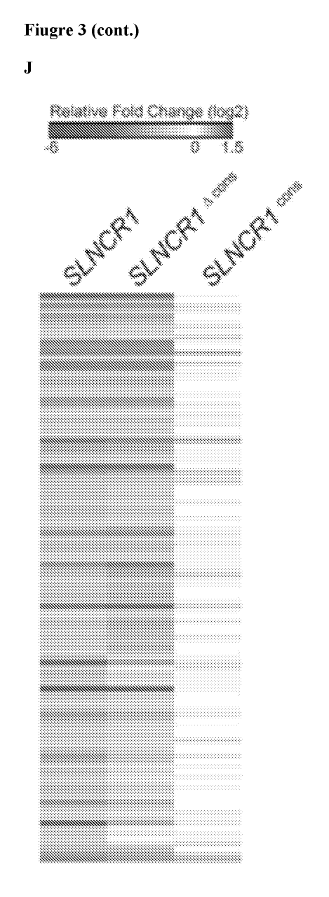

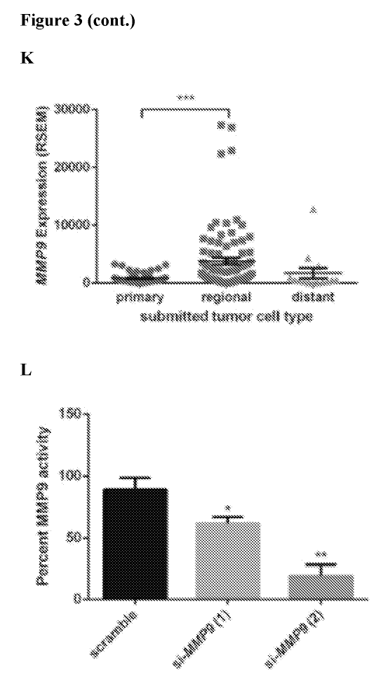

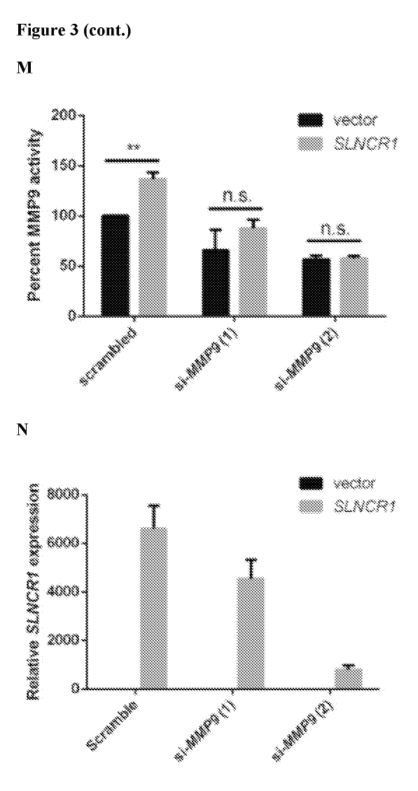

In order to identify candidate lncRNAs involved in melanomagenesis, RNA sequencing (RNA-seq) was used to profile lncRNAs in three patient-derived melanomas. Linc00673, known hereinafter as SLNCR (SRA-like non-coding RNA), was identified as being highly expressed in the patient-derived melanomas, as well as in four additional melanoma short-term cultures (FIG. 1). Three different isoforms of SLNCR were detected using RNA sequencing of patient-derived melanomas (FIG. 2A). The most prevalent form of the lncRNA, referred to as SLNCR or SLNCR1 in the examples, is 2,257 nucleotides long and is composed of 4 exons spanning human chr17:70399463-70588943 as annotated according to the Human Genome Assembly GRCH37/hg19. SLNCR2 (also referred to as SLNCR4a) and SLNCR3 (also referred to as SLNCR4b) contain an additional alternative short or long exon, respectively, located between exon 3 and 4. The SLNCR locus is located within a chromosomal region that is commonly amplified in melanomas, lung and ovarian cancers (see the broadinstitute.com/tumorscape website, at least at Chr17:41471733-78605474) (FIG. 2B). Furthermore, extensive RNA-seq analysis demonstrated that various isoforms of SLNCR are expressed in melanomas, as well as cervical cancer, ovarian cancer, uterinecancer, colorectalcancer, pancreatic cancer, lower grade glioma. and glioblastoma multiforme, and that increased expression correlates with lung adenocarcinoma and lung squamour cell carcinoma (Iyer et al. (2015) Nat. Genet. 47:199-208) (FIG. 3).

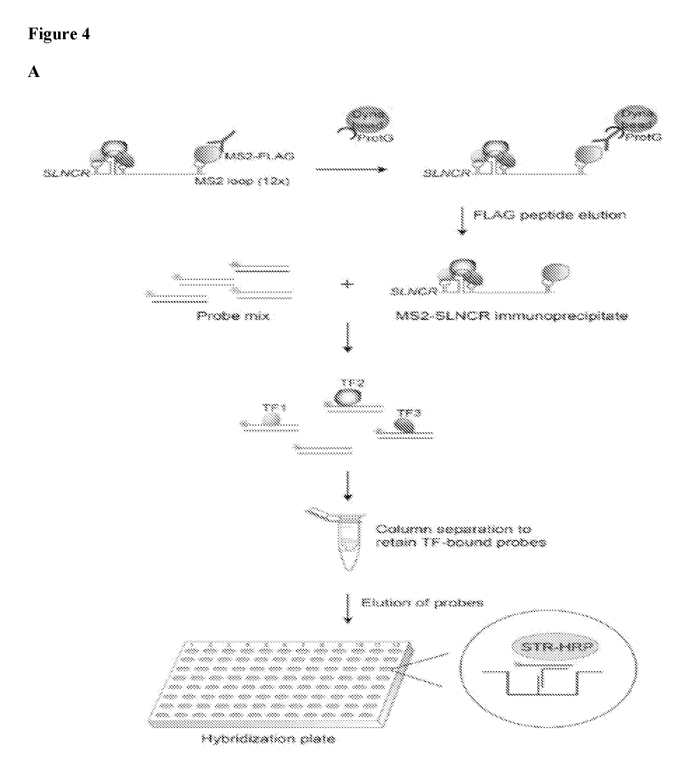

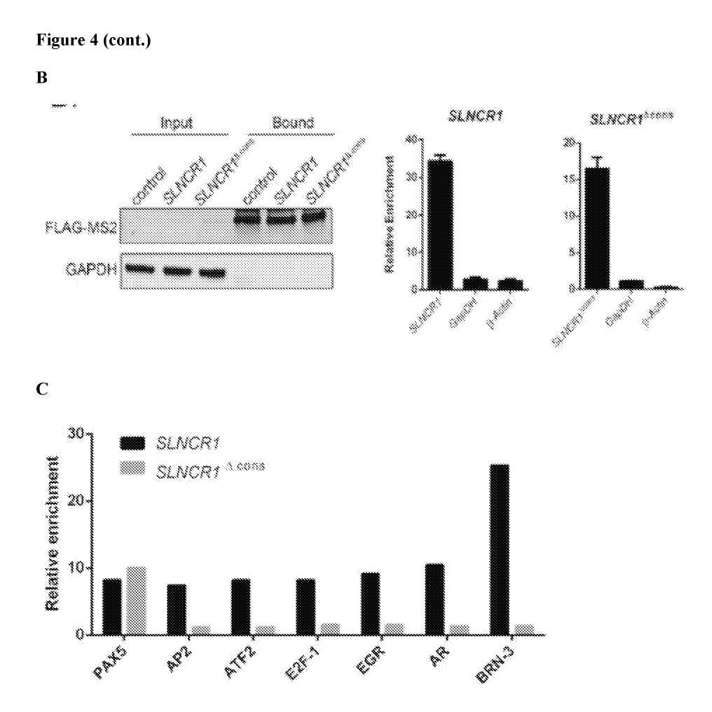

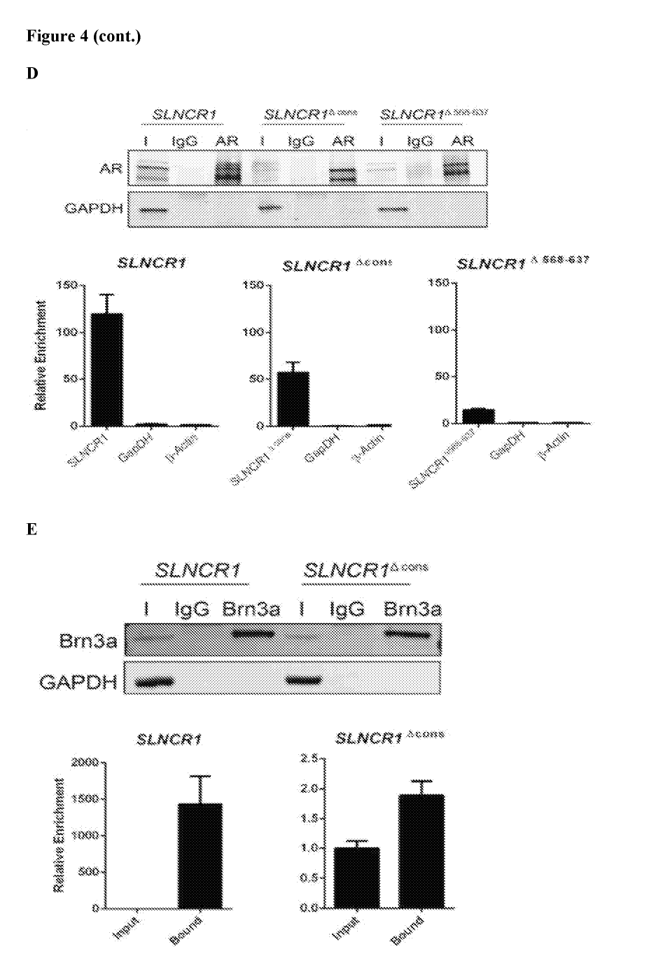

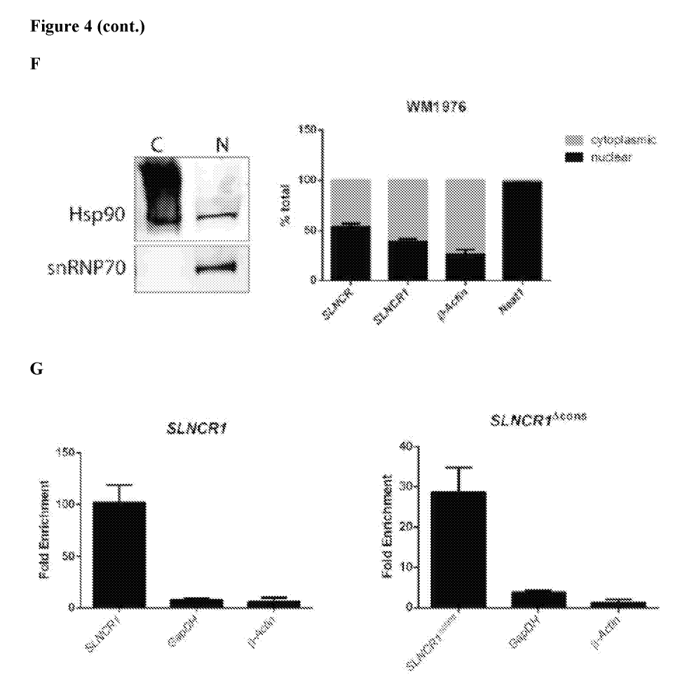

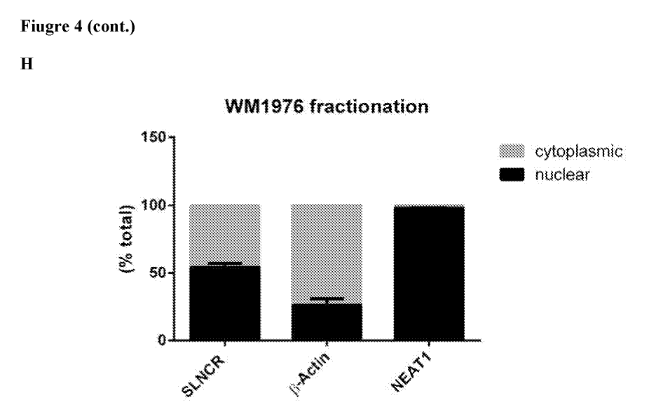

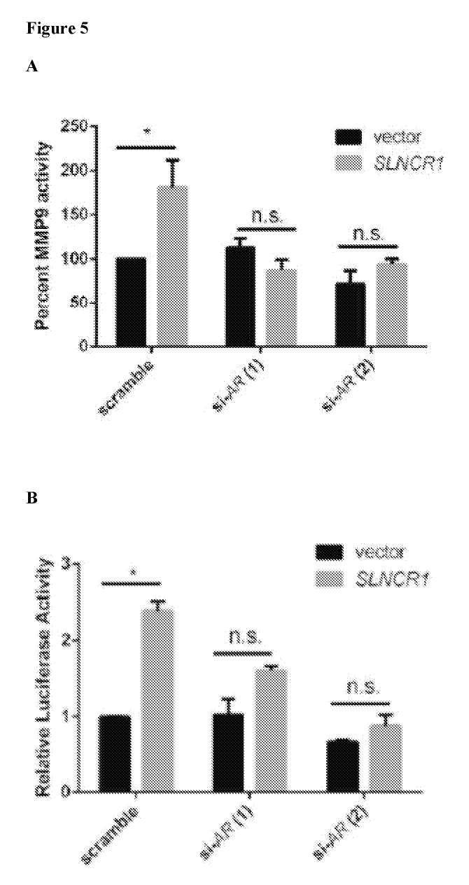

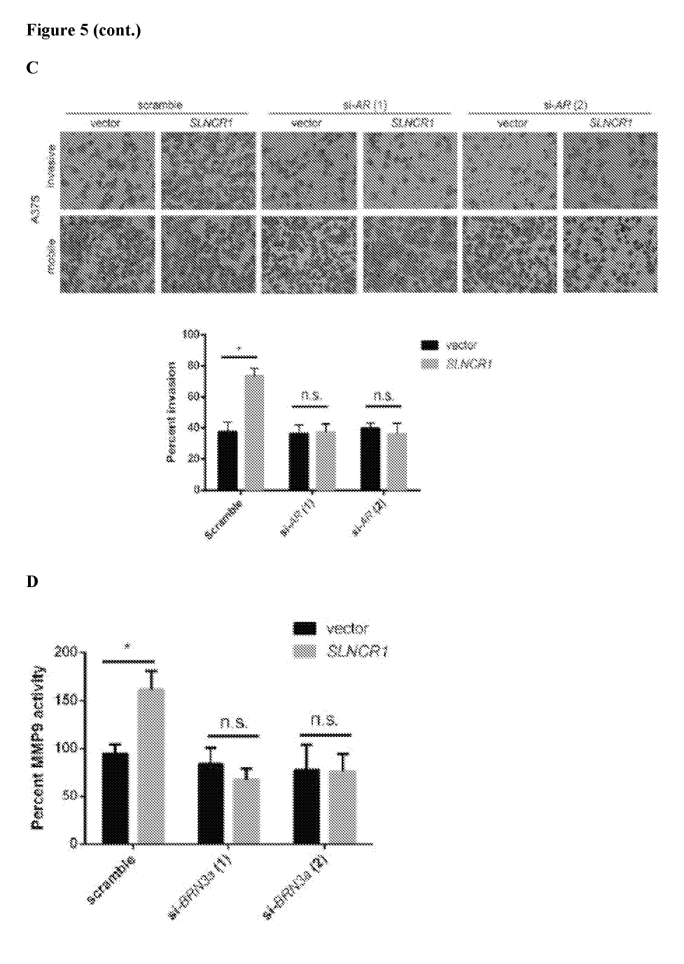

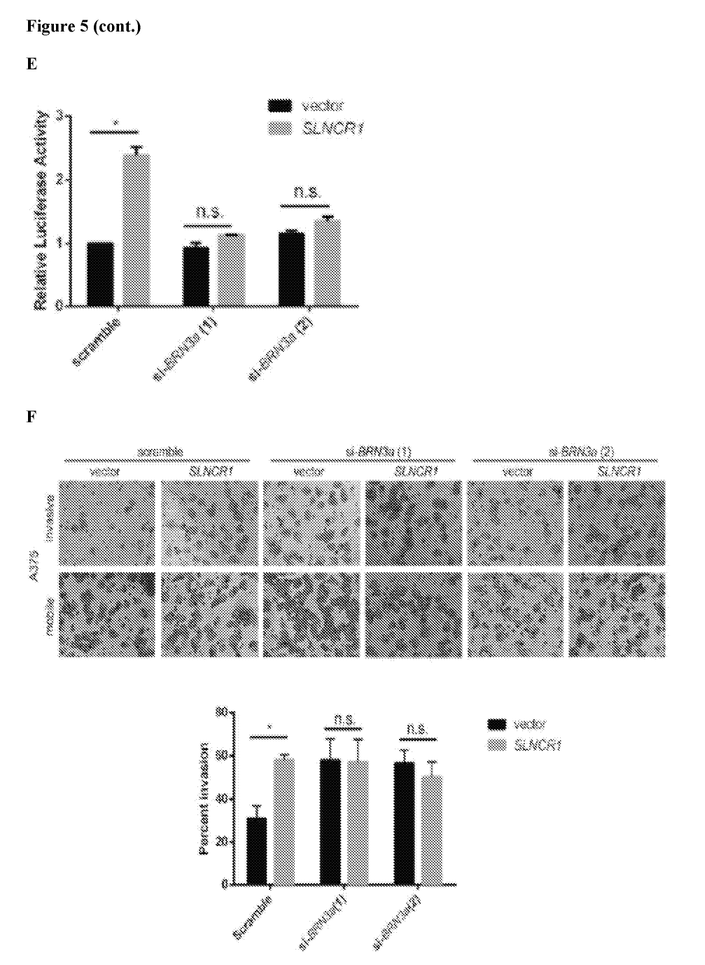

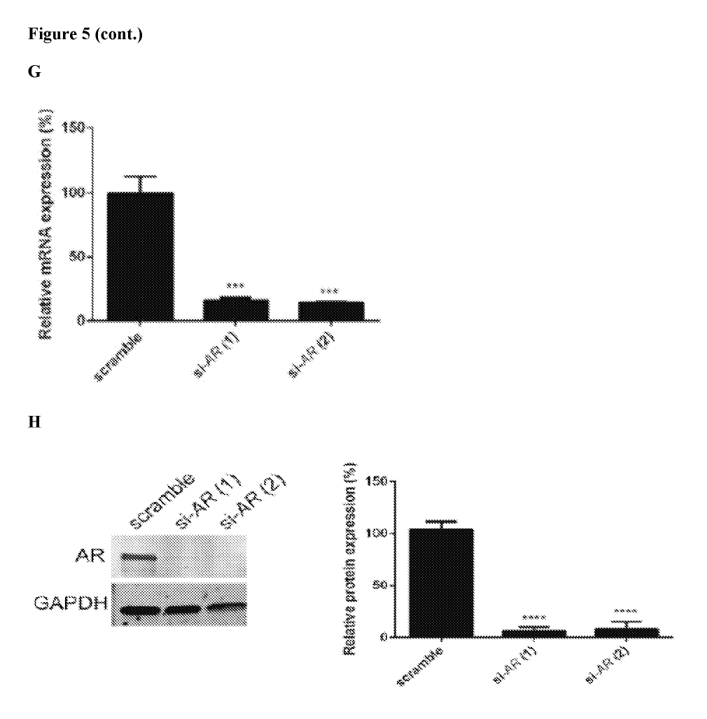

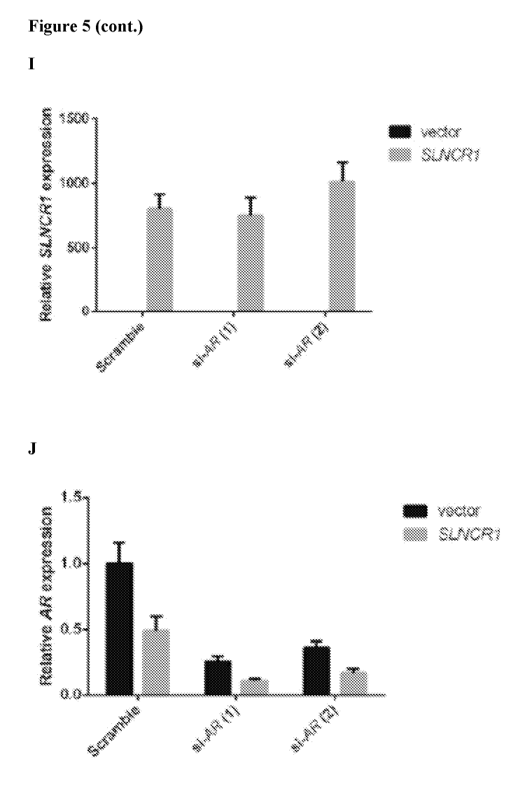

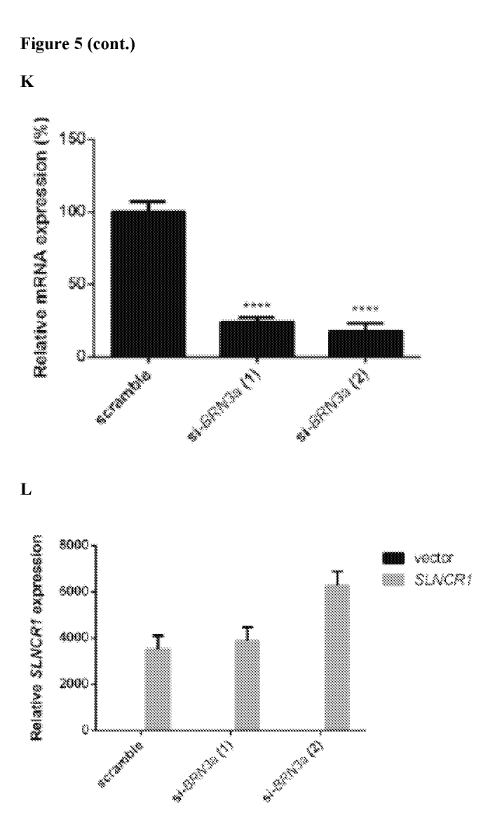

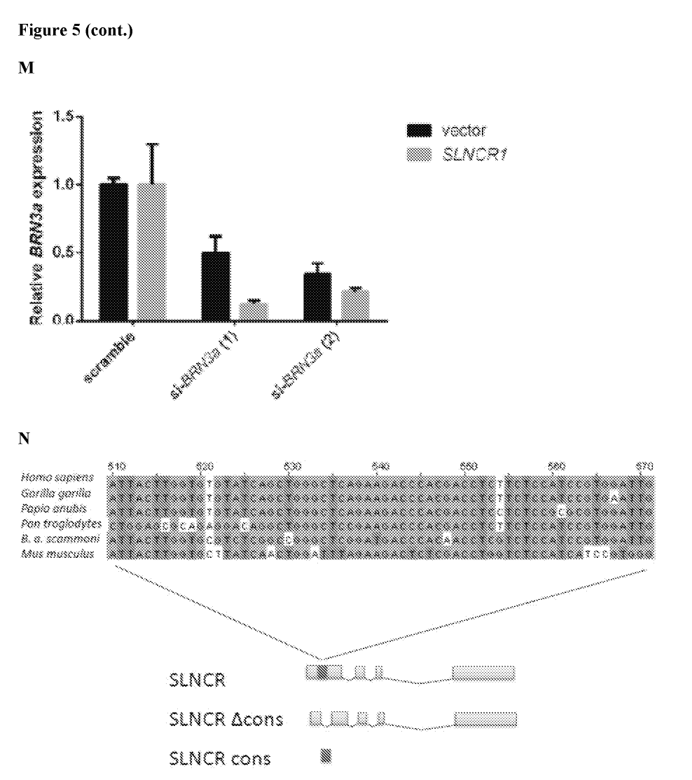

Fractionation of a melanoma short-term culture and subsequent qRT-PCR revealed that SLNCR is slightly enriched in the nucleus (FIG. 4). SLNCR is highly conserved among mammals, indicating a functionally important role for the lncRNA. As described further below, SLNCR functions inter alia to promote the development and/or progression of tumors. Furthermore, a region of ˜300 nucleotides is remarkably well conserved (FIG. 5) and is sufficient for SLNCR function regarding invasion and is believed to regulate additional aspects of the development and/or progression of tumors, as described further below.

### Example 3: siRNA-Mediated Knockdown of SLNCR Decreases Proliferation of Cancer Cells

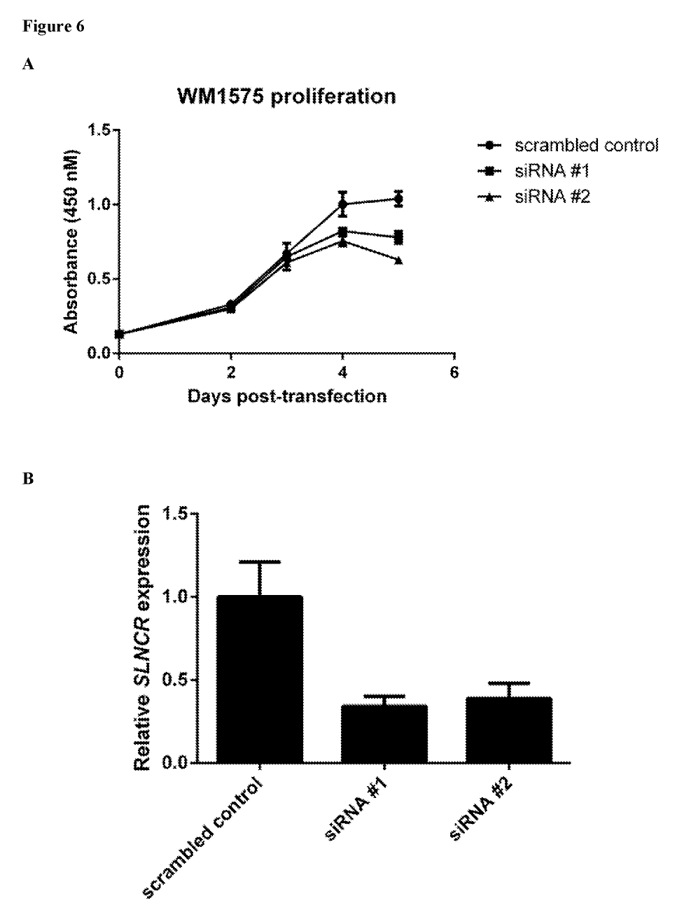

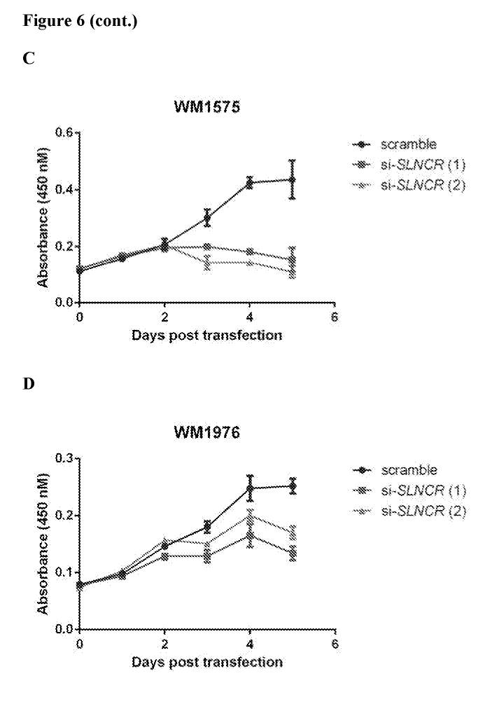

Melanoma short-term cultures have undergone relatively few passages outside of the patient, accurately capturing the genetics of the disease, and have been well characterized (Lin et al. (2008) Cancer Res. 68:664-673). RT-qPCR results indicate that SLNCR is expressed in multiple melanoma short-term cultures tested. Therefore, siRNAs were used to knockdown endogenously expressed SLNCR and phenotypes were screened. The siRNA sequences used in these experiments were (5′ to 3′ direction): siRNA 1: TTAGGTCAAATAGGATCTAAA and siRNA 2: AAAGACGTTTACACCGAGAAA. As shown in FIG. 6, siRNA-mediated knockdown of SLNCR significantly decreased proliferation of WM1575 cells. Importantly, the siRNAs used in this assay do not distinguish between different SLNCR isoforms, and therefore decreases levels of SLNCR, SLNCR2 and SLNCR3.

### Example 4: SLNCR Increases Invasion of Melanoma Cells Through Upregulation of MMP9

RNA-Seq sequencing was used to determine global transcriptional changes upon overexpression of SLNCR. Briefly, the lncRNA was cloned into pcDNA3.1 and was ectopically overexpressed in the stable melanoma cell line A375, where SLNCR is endogenously expressed at very low levels. Expression of SLNCR results in the differential expression of at least 29 genes (Log 2 fold-change>1.5; p value<0.05; FIG. 7).

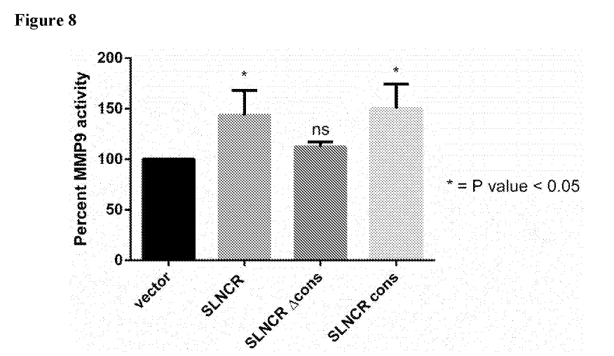

In particular, two genes involved in cell invasion were significantly increase upon expression of SLNCR: MMP9, which encodes gelatinase B and directly degrades the extracellular matrix, and FDCSP, which promotes cancer cell invasion through an uncharacterized mechanism (Wang et al. (2010) Oncol. Rep. 24:933-939). It was determined that overexpression of SLNCR increases both MMP9 mRNA and MMP9 enzymatic activity as measured by gelatin zymography (FIG. 8). Remarkably, the highly conserved ˜300 bp SLNCR sequence is both necessary and sufficient for the observed increase in MMP9 gelatinase activity since deletion of the conserved sequence abrogates activity, whereas overexpression of only this region replicates the results seen with overexpression of full-length SLNCR (FIG. 8).

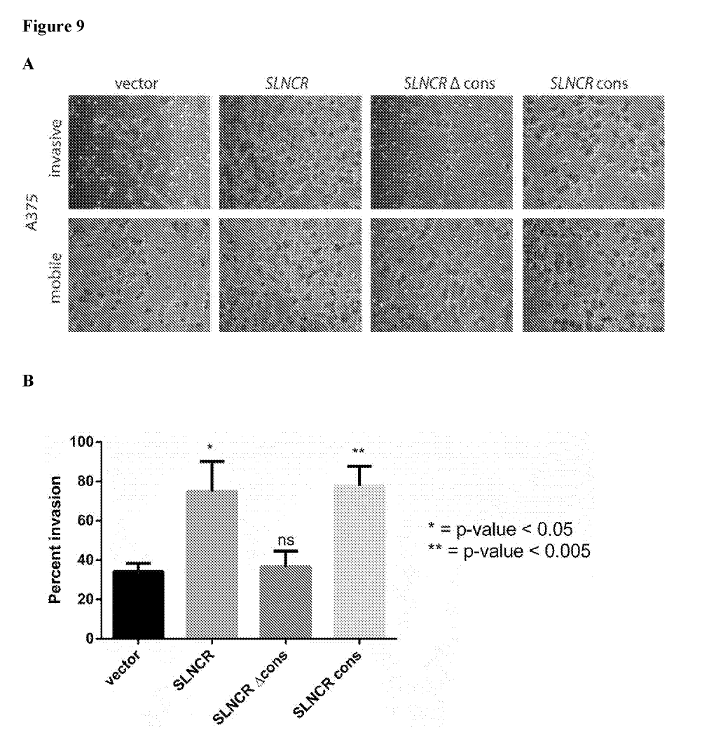

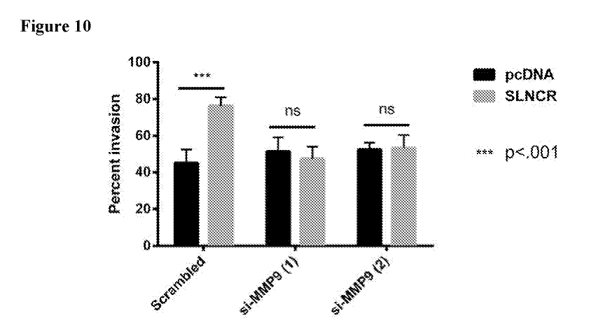

Matrigel invasion assays were then used to investigate if the increase in MMP9 activity correlated with an increase in invasiveness of melanoma cells. As expected, overexpression of SLNCR resulted in a significant increase in invasiveness (FIG. 9), while again the conserved sequence was necessary and sufficient for eliciting this affect. To confirm that the increase in MMP9 activity is responsible for mediating SLNCRs increase in invasion, the invasion assays were repeated using either scrambled siRNAs or 2 siRNAs specific for MMP9. Knockdown of MMP9 attenuates SLNCR mediated invasion in A375 cells (FIG. 10), confirming that SLNCR increases melanoma invasion through upregulation of MMP9.

### Example 5: Knockdown of SLNCR Decreases Invasiveness of Melanoma Cells

In order to confirm that SLNCR regulates invasiveness of melanoma cells, isoform-specific siRNAs that target only the shortest form of the lncRNA were generated. The siRNA sequences span the exon-exon junction that is present only in the shortest isoforms where the alternative exon in the longer isoforms is located. The siRNA sequences used in these experiments were (5′ to 3′ direction): siRNA #1: AAGAGGATGGGAAGGACTGAT and siRNA #2: CTGATGGGAAGGACTGATCCA. As shown in FIG. 11, siRNA mediated knockdown of SLNCR decreases the invasiveness of both WM1976 and WM1575 short-term cultures.

### Example 6: SLNCR is a Nuclear Receptor Coactivator

MetaCore™ network analysis (Thomson Reuters) revealed that all of the transcriptional networks significantly affected by the SLNCR lncRNA as identified through RNA-seq are nuclear receptor (NR) regulated pathways. Moreover, all of the NRs implicated in SLNCR's function are known to bind the coactivator SRC-1, which is known to associate with another lncRNA, SRA-1, that activates SRC-1 (Lanz et al. (1999) Cell 97:17-27).

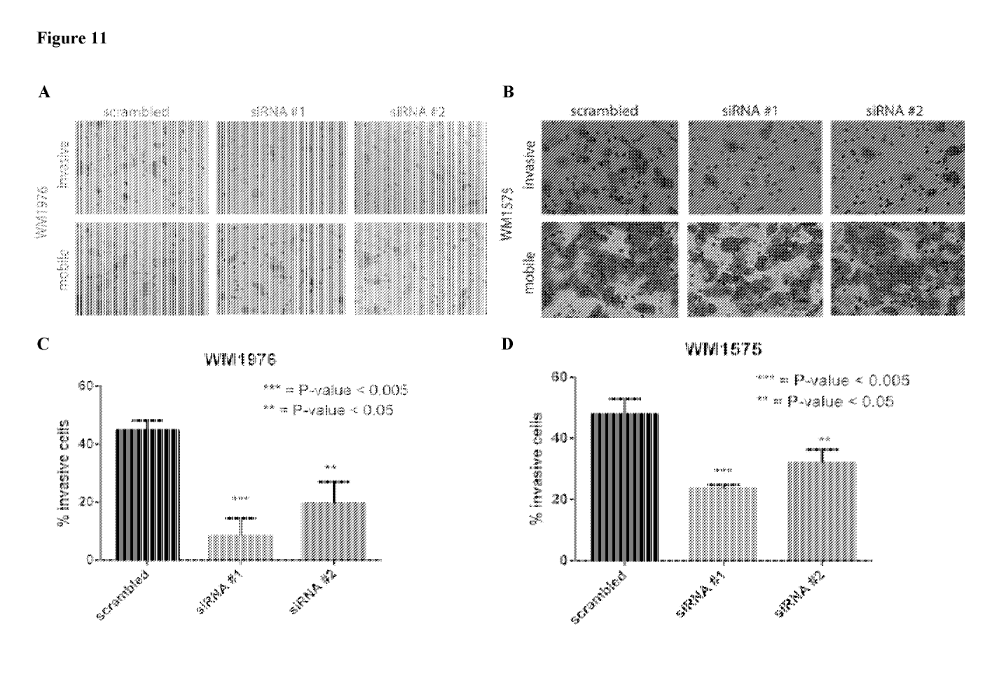

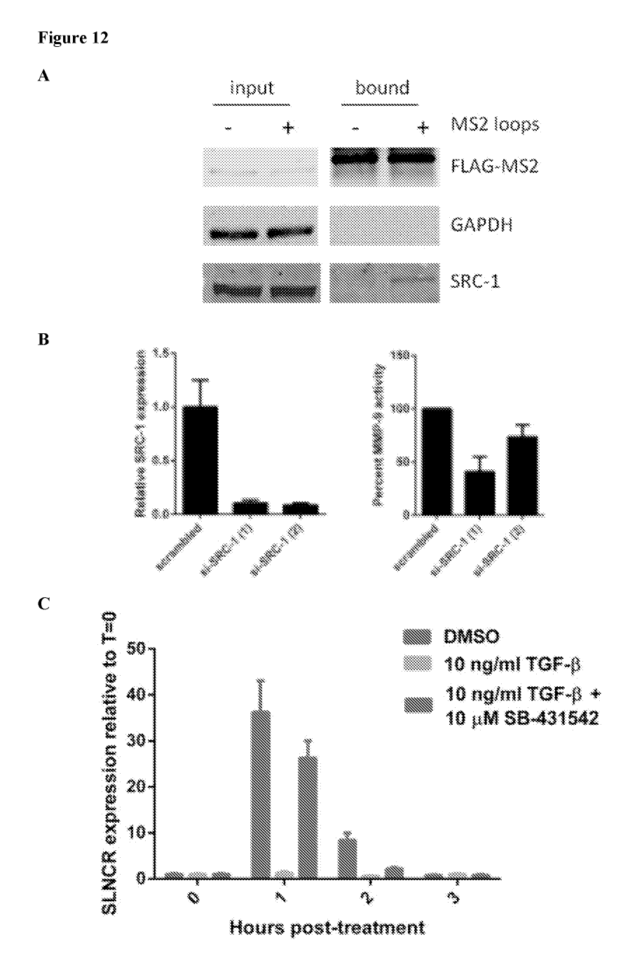

In order to test whether SLNCR also binds SRC-1, an RNA pulldown strategy was used to purify exogenously expressed SLNCR from A375 cells. The bacteriophage coat protein, MS2, interacts with high-affinity to a specific stem-loop structure in the phage genome and has been widely adapted for biochemical purification of mammalian RNAs, including lncRNAs (Gong and Maquat (2015) Meth. Mol. Biol. 1206:81-86). Briefly, multiple copies of the MS2 stem-loop structure are inserted into a gene of interest and purified using a co-expressed epitope-tagged MS2 protein. Importantly, overexpression of SLNCR tagged at the 3′ end with 12 MS2 loops results in gene expression changes comparable to overexpression of the untagged lncRNA, indicating that insertion of epitope tag does not interfere with normal function of the lncRNA. The simian virus nuclear localization signal (SV40-NLS) was successfully cloned upstream of the MS2 ORF in a FLAG tagged hMS2 expressing vector. Nuclear localization was confirmed via fractionation and Western blotting Immunoprecipitation of the flag-tagged MS2 protein routinely shows a ˜50 fold enriched of the MS2 tagged SLNCR transcript compared to an untagged control and specifically co-precipitated SRC-1 (FIG. 12A), demonstrating that SLNCR binds to SRC-1. The determination that SLNCR binds to SRC-1 is important because SRC-1 increases MMP9 activity. FIG. 12B demonstrates that SRC-1 knockdown decreases MMP9 activity. The siRNA sequences used in these experiments were (5′ to 3′ direction): siRNA 1: CAGCGGGAACTGTACAGTCAA and siRNA 2: CTCCTAATATTTCGACATTAA. In addition, FIG. 12C demonstrates that SLNCR expression is inhibited by TGF-β.

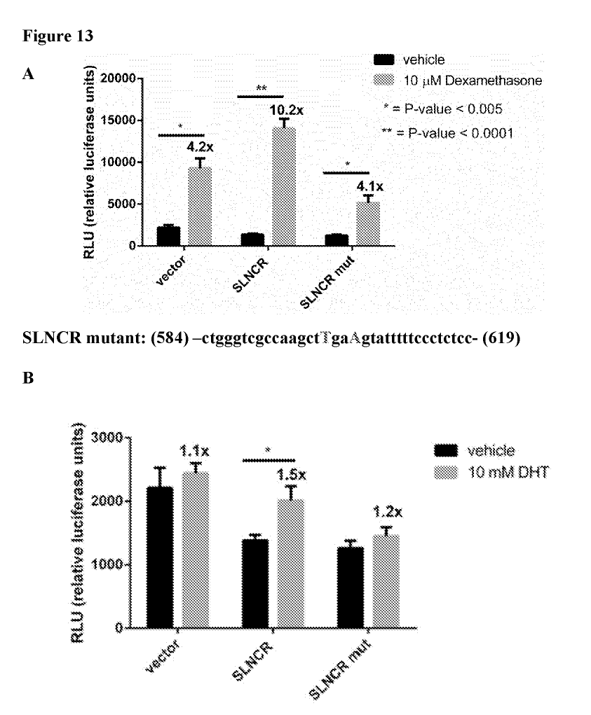

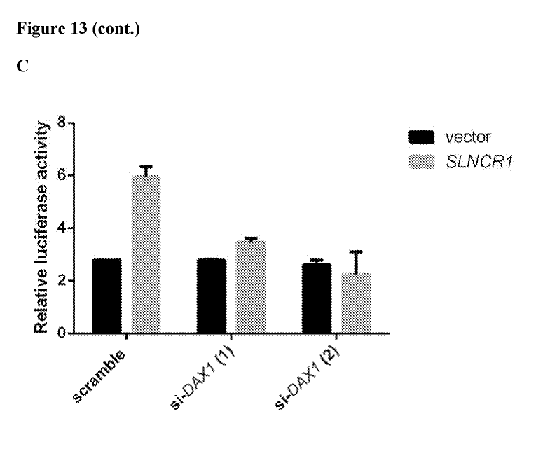

A luciferase reporter was also generated to determine whether SLNCR is a NR coactivator. The pGL4.36 MMTV-luciferase reporter construct (Promega) was co-transfected into A375 cells with either a control or SLNCR-expressing vector. Addition of dexamethasone, a NR ligand (FIG. 13A), or dihydrotestosterone (FIG. 13B), induced expression of luciferase from the MMTV promoter. Overexpression of SLNCR resulted in a 10-fold induction in luciferase activity after addition of dexamethasone compared to uninduced cells, which is a significant increase in induction compared to cells transfected with only a vector control (FIG. 13A). Detailed analyses of the SLNCR sequence revealed significant sequence similarity to SRA1, specifically within stem structures known to be required for NR coactivation function (Lanz et al. (2002) Proc. Natl. Acad. Sci. U.S.A. 99:16081-16086; Novikova et al. (2012) Nucl. Acids Res. 40:5034-5051). Mutation of 2 bases predicted to be in a functionally important stem loop results in complete loss of SLNCR's NR coactivation function (FIG. 13A), despite similar expression levels. Addition of DHT, a ligand with high specificity for the androgen receptor, resulted in similar affects. Collectively, the data indicate that SLNCR regulates nuclear receptor signaling, including, but not limited to, the androgen receptor, likely in cooperation with SRC-1.

### Example 7: Categories of Transcripts Regulated by SLNCR

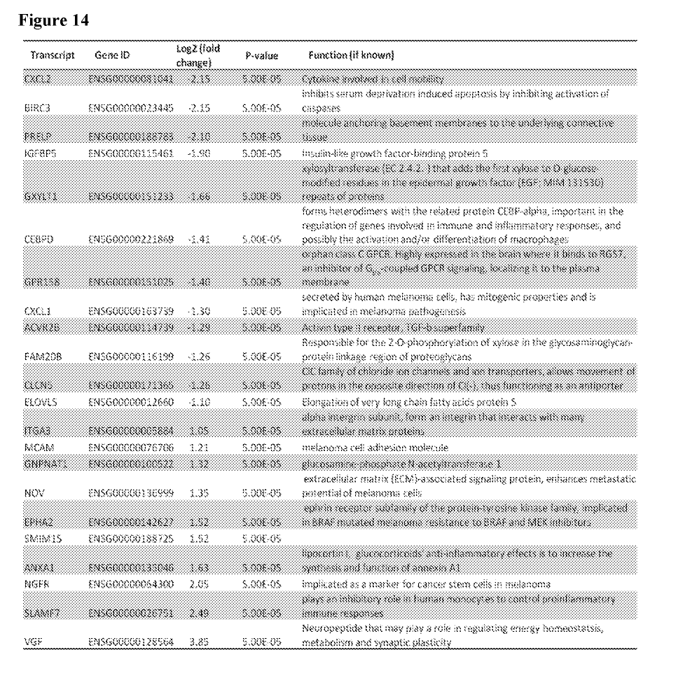

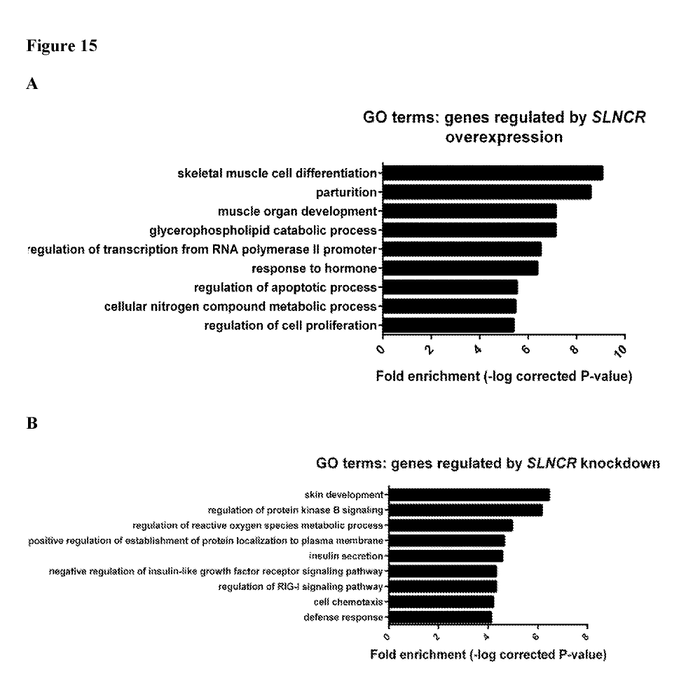

A list of transcripts differentially expressed upon overexpression of SLNCR is provided in FIG. 14. These transcripts can be categorized into genes involved in development and differentiation, regulation of RNA Pol II transcription, metabolic processes, and regulation of apoptosis and cell proliferation (FIG. 15A). These data indicate that SLNCR regulates these processes. Interestingly, SLNCR overexpression also results in the downregulation of multiple tumor suppressors, including EGR-1 and TXNIP.

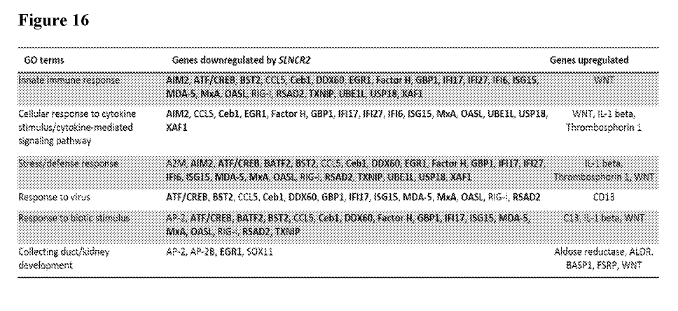

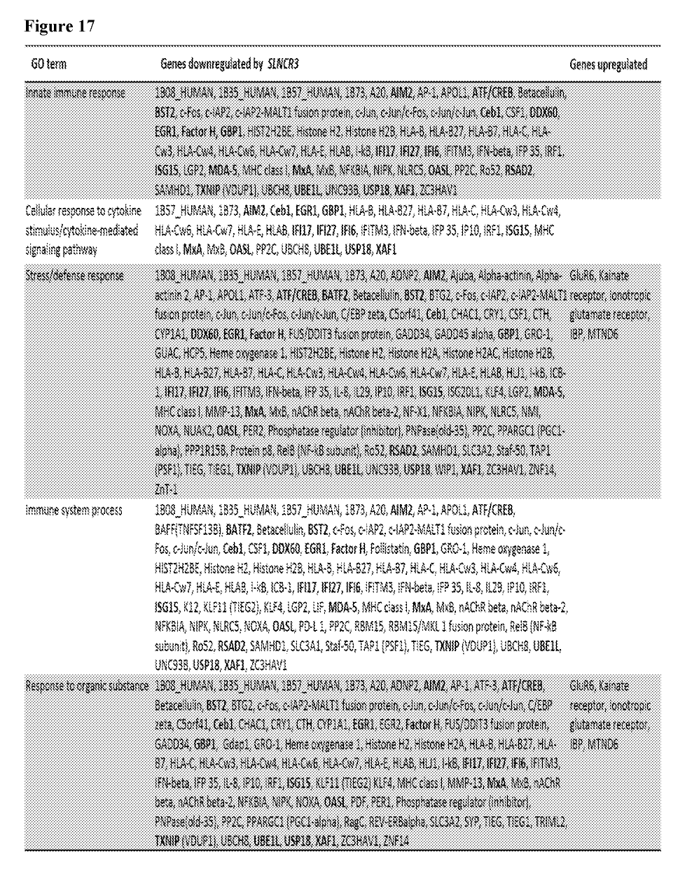

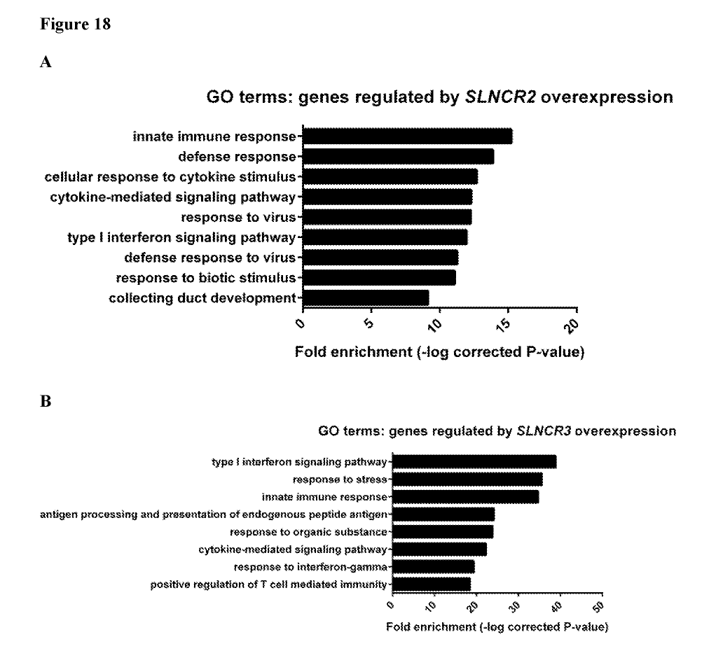

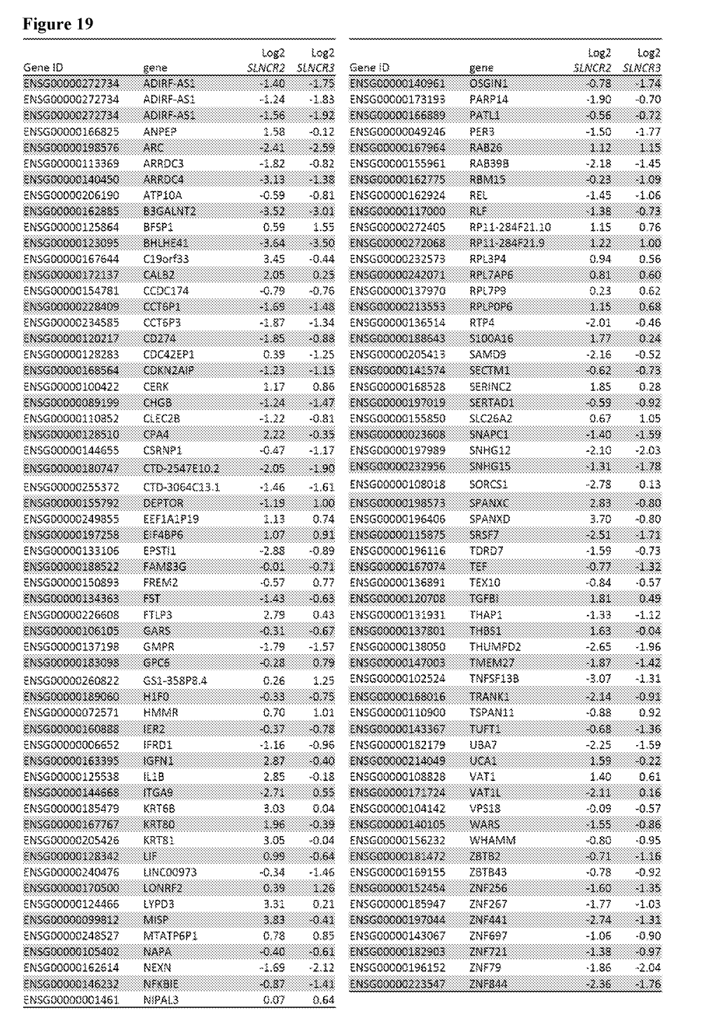

A list of transcripts differentially expressed upon overexpression of SLNCR2 or SLNCR3 is provided in FIGS. 16 and 17, respectively. Overexpression of SLNCR2 or SLNCR3 regulates many genes involved in the immune or stress response (FIGS. 18A and 18B, respectively). These data indicate that SLNCR2 and SLNCR3 are important for mediating or regulating the cell stress response. These isoforms also regulate expression of transcripts that are not directly involved in the immune response (FIG. 19).

### Example 8: Disease Association Analysis Indicates SLNCR Functions in Multiple Other Disease Processes

Transcripts affected upon perturbation of SLNCR expression (overexpression or knockdown) were analyzed for overpresentation in pathways associated with diseases other than melanoma. Disease association analyses (MetaCore™, Thomson Reuters) indicate that transcriptional networks induced upon SLNCR overexpression also contribute to respiratory, cardiac and metabolic diseases, arthritis, and necrosis (FIG. 20A). Transcriptional changes observed upon SLNCR2 or SLNCR3 overexpression are associated with various autoimmune diseases (FIGS. 20B and 20C), indicating that SLNCR2 and/or SLNCR3 plays a role in progression of these diseases.

### Example 9: SLNCR Binds to or Regulates the Action of Multiple Transcription Factors (TFs)

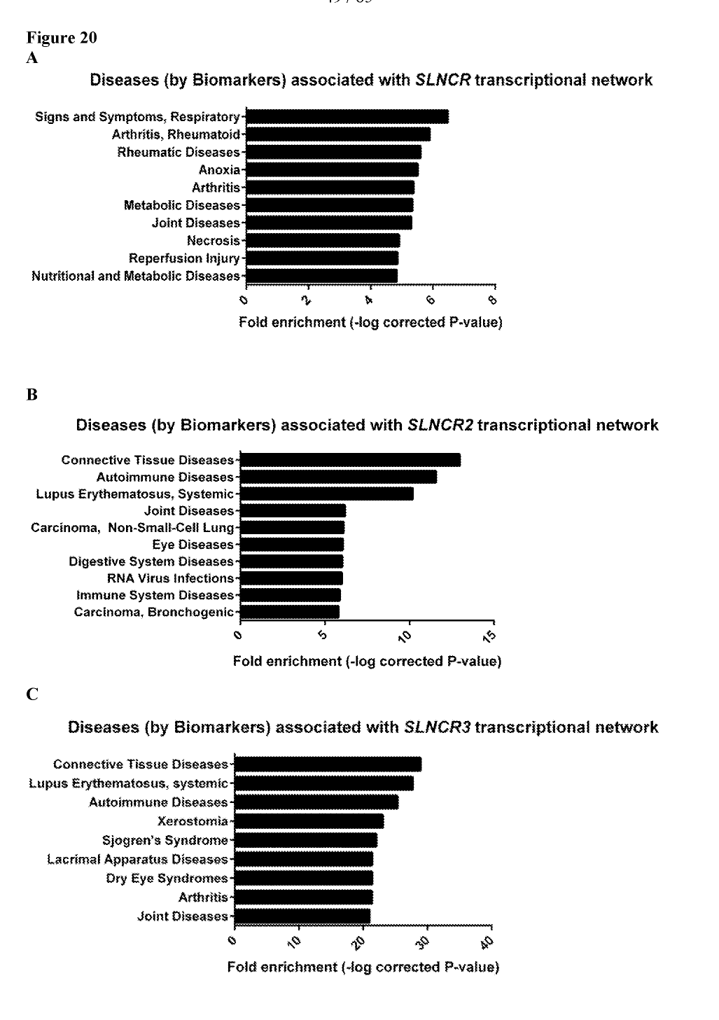

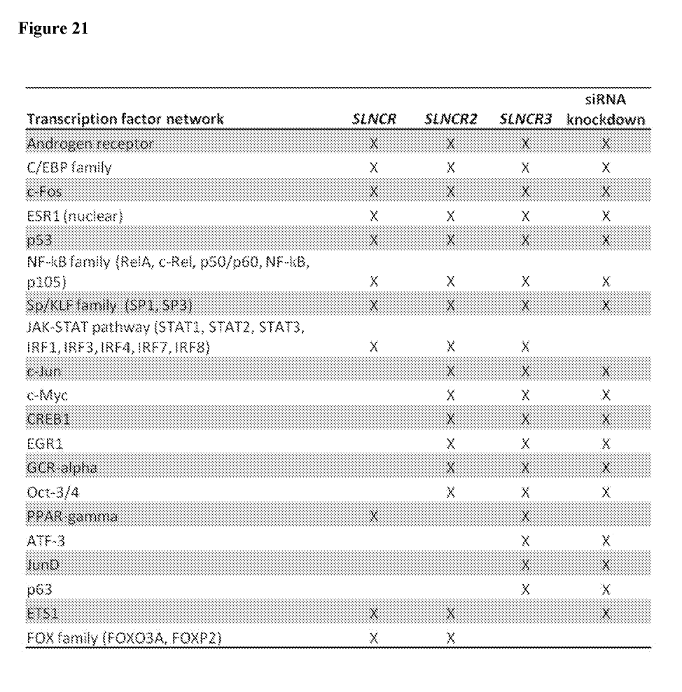

As determined and described above, SLNCR is a nuclear receptor coactivator. In order to determine which TFs SLNCR regulates, transcriptional networks induced upon SLNCR overexpression or knockdown were subjected to network analysis (MetaCore™, Thomson Reuters). As indicated in FIG. 21, SLNCR transcriptional networks map to multiple TFs. The androgen receptor, C/EBP, c-FOS, ESR1, p53, Nf-kB, Sp/KLF and the JAK-STAT pathway were believed to be among the most likely to be regulated by SLNCR.

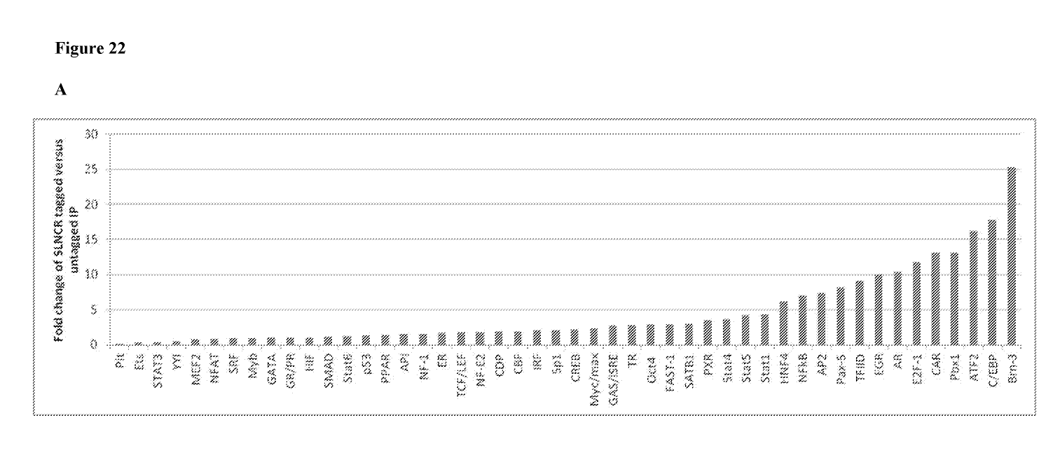

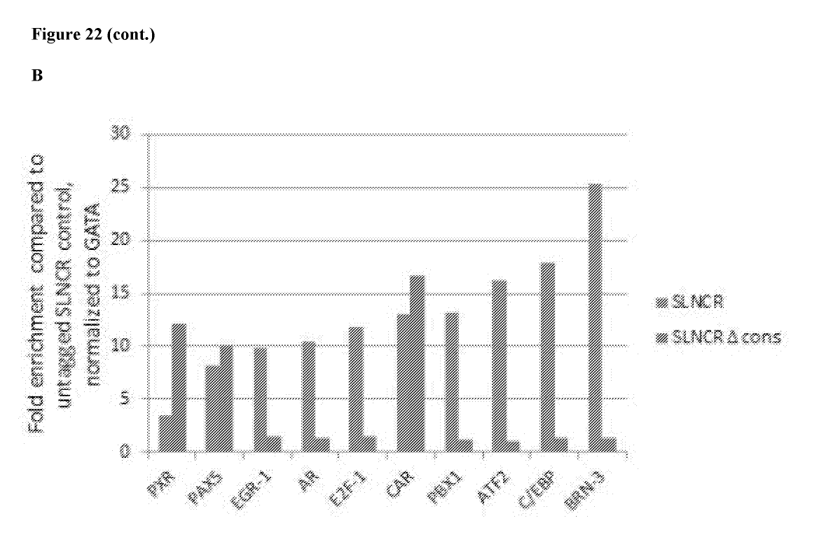

In order to identify which TFs are directly bound by SLNCR, the MS2 RNA pull-down strategy described above was performed with slight modifications. A375 cells transfected with plasmids encoding either tagged or untagged SLNCR were lysed without crosslinking, and FLAG-tagged nuclear MS2 was immunoprecipitated using anti-FLAG antibody (Sigma). Proteins and RNAs co-precipitating with SLNCR were eluted from protein G dynabeads through Flag peptide elution. The eluate was then incubated with biotinylated DNA probe mixture from the Signosis® TF Activation Profiling Plate Array and subjected to downstream analysis, according to manufacturer's instructions. FIG. 22 shows the fold enrichment of each transcription in the MS2-tagged SLNCR IP compared to the untagged control, after normalization to the GATA TF, which is not predicted to bind to SLNCR, mediate its function, and is not implicated in melanoma. As shown in FIG. 22, SLNCR binds either directly or indirectly to multiple TFs, significantly enriching HNF4, NF-kB, AP2, Pax-5, TFIID, EGR-1, AR, E2F-1, CAR, Pbx1, ATF2, C/EBP and Brn3a. Of these 13 TFs, five were directly predicted to play a role in SLNCR function as predicted by network mapping (NF-kB, EGR-1, AR, ATF family, and C/EBP).

The most highly enriched TF upon SLNCR pulldown is Brn3a, or POU4F1, a TF involved in neural crest development. Melanocytes are derived from neural crest cells and it is believed that SLNCR normally functions in neural crest development through interaction with Brn3a. Indeed, aberrant expression of Brn3a has been observed in melanomas, and is implicated in melanoma transformation and tumourigenesis through regulation of cell survival (Hohenauer et al. (2013) EMBO Mol. Med. 5:919-934). Brn3a can interact with and regulate the activity of the p53 tumor suppressor, indicating that these TFs likely have many overlapping predicted targets. Brn3a is known to directly interact with AR, in agreement with the determination that SLNCR associates with both of these TFs (Berwick et al. (2010) J. Biol. Chem. 285:15286-15295). Furthermore, 3 additional TFs enriched with SLNCR are known to genetically interact with Brn3a (E2F-1, HNF4a, and Egr-1) (Odom et al. (2007) Nat. Genet. 39:730-732; Rabinovich et al. (2008) Genome Res. 18:1763-1777; Bolotin et al. (2010) Hepatol. 51:642-653; Smith et al. (1999) Mol. Brain Res. 74:117-125). This is all strong evidence that SLNCR does bind to and regulate Brn3a.

Of the remaining TFs observed to associate with SLNCR, many are implicated in the development of progression of melanoma. AP-2 (or transcription factor activator protein-2) regulates many genes critical to tumor progression, including protease-activated receptor-1 (PAR-1) (Berger et al. (2005) Cancer Res. 65:11185-11192; Tellez et al. (2007) J. Invest. Dermatol. 127:387-393). Pbx-1 (pre-B-cell leukemia transcription factor 1) is highly expressed in melanomas, and regulates melanoma cell growth (Shiraishi et al. (2007) Oncogene 26:339-348). Pax-5 is involved in development of multiple cancers and is known to directly bind to the androgen receptor; it is possible that Pax-5 indirectly binds to SLNCR through its association with AR (Mukhopadhyay et al. (2006) Exp. Cell Res. 312:3782-3795).

The results above indicate that SLNCR interacts with SRC-1. Indeed, 4 TFs identified through the profiling array are known to interact with SRC-1: HNFa, NF-kB, AR and CAR (Eeckhoute et al. (2003) Nucl. Acids Res. 31:6640-6650; Na et al. (1998) J. Biol. Chem. 273:10831-10834; Ueda et al. (2002) J. Biol. Chem. 277:38087-38094; Albers et al. (2005) Mol. Cell. Prot. 4:205-213; Lavery and McEwan (2008) Biochem. 47:3352-3359; Bai et al. (2005) Mol. Cell. Biol. 25:1238-1257). Identification of TFs known to interact with SRC-1 serves as further evidence that the TFs identified through the profiling array are highly predicted to be faithful SLNCR interactors. Collectively, these data indicate that SLNCR interacts with Brn3a and associated TFs.

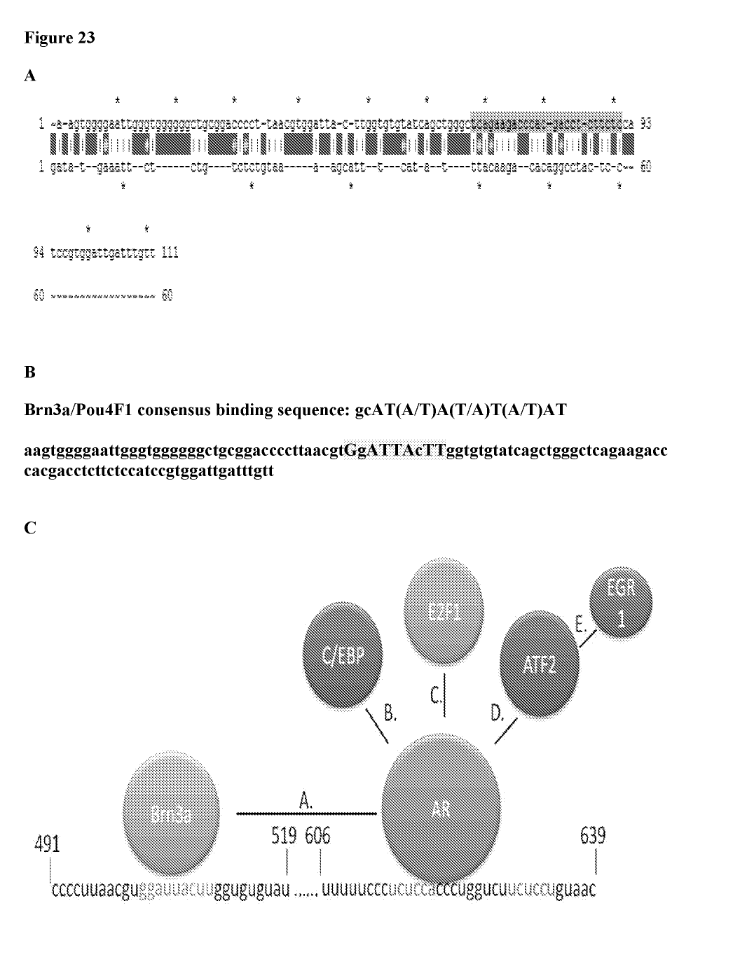

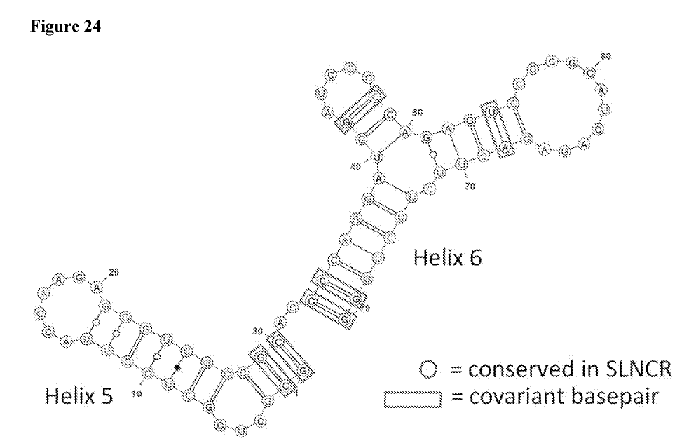

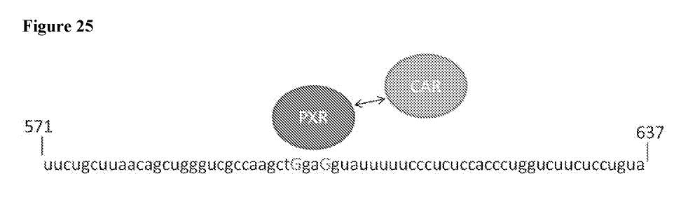

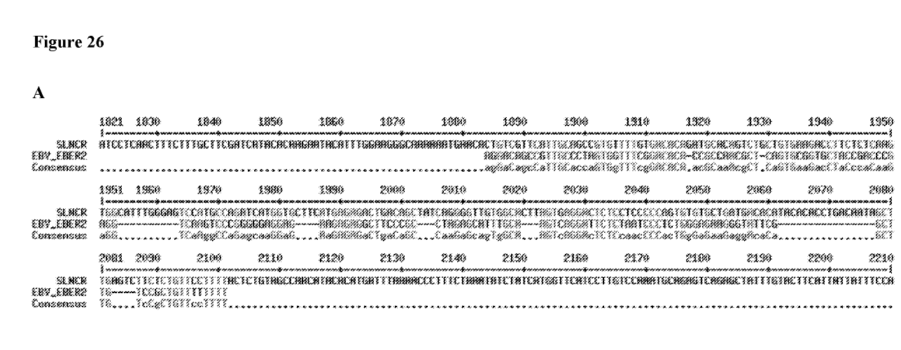

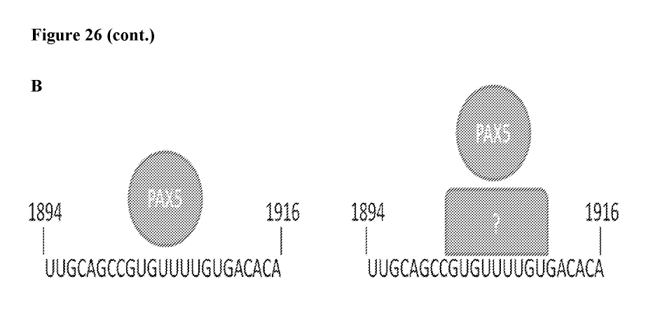

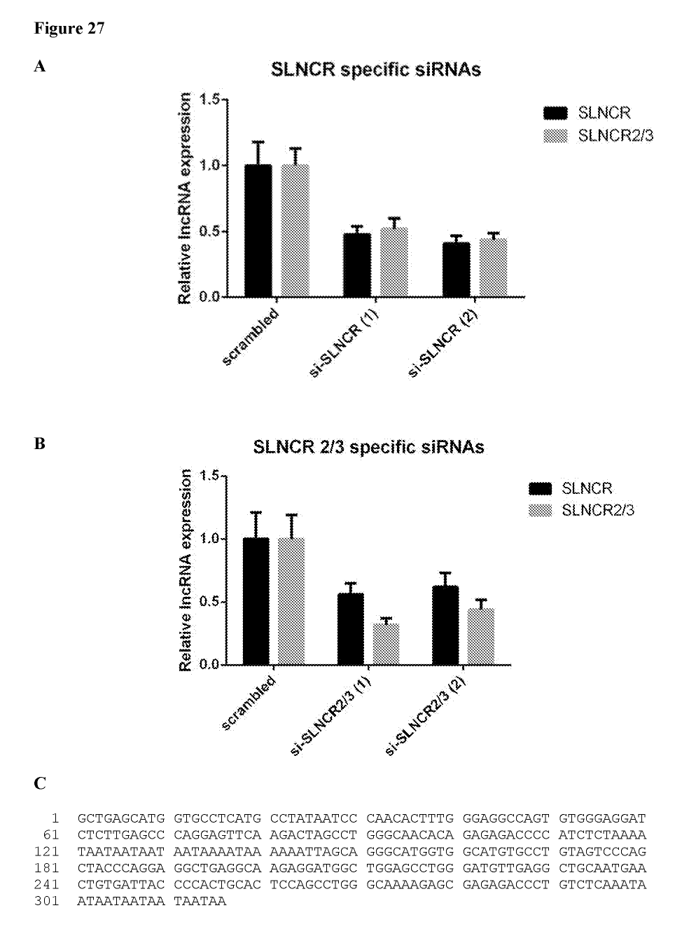

Additional SLNCR structural elements related to SLNCR functionality have been determined. FIG. 23 shows the sequence requirements for AR binding to SLNCR and Brn3a/Pou4F1 to SLNCR. FIG. 24 shows the conserved sequences with respect to the H5 and H6 helices of the SLNCR ortholog, SRA1. FIG. 25 shows sequence requirements for PXR and/or CAR binding to SLNCR. FIG. 26 shows sequence requirements for PAX5 binding to SLNCR. FIG. 27 shows that perturbation of expression (specifically knockdown) of one isoform of SLNCR affects levels of the other isoforms, indicating that the various SLNCR isoforms autoregulate one another (FIGS. 27A-27B). The siRNA sequences used to target the longer SLNCR2 and SLNCR3 isoforms are: siRNA #1: GGGCTGCTTAGTGAAATACAA and siRNA #2: CTCCGTCGAATCTGCAGTGAA. FIG. 27 also shows sequence requirements for SLNCR autoregulation (FIG. 27C).

Based on these structure-function data in combination with experimental data described above, it is believed that SLNCR acts as a scaffold to bring together multiple TFs and associated co-activators or co-repressors. The ability of certain proteins to co-immunoprecipitate other proteins only in the presence of SLNCR indicates that SLNCR mediates the interaction of these proteins. It is believed that the presence of particular co-activators or co-repressors alters the specificity of the associated TFs, resulting in altered transcription of target genes. Assays to determine the particular activity of TFs mediated by SLNCR are well known. For example, the activity of the androgen receptor (AR) can be measured using a prostate-specific antigen (PSA) promoter regulated luciferase reporter plasmid (Shang et al. (2002) Mol. Cell 9:601-610). Stimulation with dihydrotestosterone (DHT), a ligand specific for the androgen receptor, results in transcription from the PSA promoter and a subsequent increase in luciferase which can be easily measured using a luciferase reporter assay system (Promega). SLNCR mediates the translocation of certain transcription factors to the nucleus, which can be assyed tagged TF fusion proteins (e.g., GFP-TF fusions) and microscopy will be used to localize the TFs in the presence or absence of SLNCR (Tilley et al. (1989) Proc. Natl. Acad. Sci. U.S.A. 86:327-331). Similarly, visualization following stimulation of TF-specific ligands, such as DHT, can also be performed. SLNCR recruits TF complexes to specific locations on the chromosome, thereby directly impacting transcription of these downstream targets, which can be analyzed in many ways, such as using active motifs RNA chromatin immunoprecipitation assays (e.g., ChIP-IT® kits, active motifs RIME, etc.).

### Example 10: Materials and Methods for Examples 11-15

Cell Culture

All cells were cultured as adherent cells in DMEM (Dulbecco's modified eagle medium, Invitrogen) without glutamine supplemented with 10% fetal bovine serum (FBS). A375 cells were purchased from ATCC, HEK293T cells were a gift from Ronny Drapkin, ‘CY’ melanomas were a gift from Charles Yoon, and ‘WM’ melanomas were from collections of the Wistar Institute (Philadelphia, Pa.).

For luciferase assays, cells were cultures in phenol-red free DMEM without glutamine (Invitrogen), supplemented with 5% charcoal stripped FBS. Luciferase activity was measured using Promega Dual-Glo® Luciferase Assay system. For fractionation experiments, cells were grown to ˜80% confluency in 10 cm tissue culture treated dishes and fractionated using Thermo Scientific™ NE-PER™ Nuclear and Cytoplasmic Extraction Kit, according to manufacturer's instructions. Nuclear and cytoplasmic fractions were split for protein and RNA analysis. For proliferation assays, cells were transfected with the indicated siRNAs 24 hours post-seeding and proliferation was measured every 24 hours using WST-1 reagent (Roche) according to the manufacturer's instructions.

Invasion Assays

Cells were plated in either BD BioCoat™ matrigel inserts or uncoated control inserts (Corning) in serum-free media and placed into DMEM with 30% FBS (fetal bovine serum). The number of invaded or migrant cells were imaged on 20× magnification in 8 fields of view for 3 independent replicates.

Plasmid Construction

SLNCR1 and a codon-optimized Brn3a were synthesized by Biomatik Corporation and cloned into pCDNA3.1 (−). The simian virus nuclear localization signal (SV40-NLS) was cloned upstream of the MS2 ORF in a FLAG-tagged, hMS2-expressing vector, a gift from Dr. Lynne Maquat, University of Rochester Medical Center. Nuclear localization of tagged MS2 was confirmed via fractionation and western blotting. pEGFP-C1-AR was a gift from Michael Mancini (Addgene plasmid #28235).

Reagents and Antibodies

Lipofectamine® RNAiMax (Life Technologies) was used for all siRNA transfections, and Lipofectamine® 2000 (Life Technologies) was used for all plasmid transfections and siRNA/plasmid cotransfections. Protein G Dynabeads® (Life Technologies) were used for FLAG-MS2 IPs, and Protein A Dynabeads® (Life Technologies) were used for AR and Brn3a IPs. The following antibodies were used: Sigma Monoclonal ANTI-FLAG® M2 antibody; Santa Cruz AR (N-20), Brn3a (14A6), and rabbit IgG control; Cell Signaling GAPDH (14C10); and BD Pharmingen™ mouse IgG control.

RNA Pulldowns

A375 cells were grown to ˜80% confluency in 10 cm dishes, transfected with 10 μg of the plasmid encoding nuclear MS2 and 8 μg of the indicated MS2 stem-loop tagged SLNCR1, and harvested 36-48 hours post-transfection. MS2 RNA pull-downs were completed from non-crosslinked cells a slightly modified protocol from Gong and Maquat (Gong and Maquat (2015) Methods Mol. Biol. 1206:81-86). For samples immediately subjected to western blot analysis, beads were resuspended in 25 μl 2× Laemmilli sample buffer and incubated at 95° C. for 5 minutes. For pulldown extracts subjected to Transcription Factor array analysis, 25 μl of wash buffer containing flag peptide at final concentration of 0.1 mg/ml was added and beads were rotated for 30 minutes at 4° C. Twelve μl of eluate was incubated with biotinylated DNA probe mixture from the Signosis® TF Activation Profiling Plate Array I and subjected to downstream analysis, according to GATA, and represented as a fold enrichment compared to a cells transfected with a plasmid encoding SLNCR1 without the MS2 stem loop tag.

RIP assays were performed from HEK293T cells co-transfected with pEGFP-C1-AR or pCDNA-Brn3a and the indicated SLNCR1 expressing plasmids.

RNA Extraction and cDNA Library Preparation

RNA was isolated using Trizol® (Life Technologies) and Qiagen RNeasy® Mini Kit and treated with DNase. cDNA was generated using SuperScript III (Invitrogen) reverse transcriptase. The indicated transcripts were quantified using Platinum® SYBR® Green qPCR SuperMix-UDG mix on a CFX384 Touch™ Real-Time PCR Detection System.

The T-test statistics, Pearson correlations, hazard ratio and Kaplan-Meier survival analysis were performed using GraphPad Prism version 6.00 for Windows (GraphPad Software, La Jolla Calif. USA). Image quantifications were performed using ImageJ software.

RNA-Sequencing and Bioinformatics

For MSTC RNA-seq, cDNA libraries were prepared from 1 μg of total RNA using the Illumina TruSeq RNA sample preparation kit (v2). Libraries were pooled and sequenced on the Illumina HiSeq 2000 platform. Normalized read counts (FPKM) were generated in Cufflinks v2.1.1 (http://cole-trapnell-lab.github.io/cufflinks/) by mapping onto the hg19 build of the human transcriptome (http://support.illumina.com/sequencing/sequencing_software/igenome.html). For RNA-Seq of SLNCR1 depleted or over-expressing cells, library preparation and analysis was performed by The Center for Cancer Computational Biology, Dana-Farber Cancer Institute, Boston, Mass. RNA was selected using NEBNext® PolyA mRNA Magnetic Isolation Module and libraries were prepared using NEBNext® Ultra™ RNA Library Prep Kit for Illumina®. Libraries were sequenced on Illumina® NextSeq 500 platform with Paired End 75 bp sequencing. Sequencing reads were aligned to the Human Reference Genome (assembly hg19) using RNA-specific STAR aligner (Dobin et al. (2013) Bioinformatics 29:15-21), and quality was assessed using the Broad Institutes's RNA-SeQC tool (DeLuca (2012) Bioinformatics 28:1530-1532). Read-counts were counted using featureCounts tool (Liao et al. (2014) Bioinformatics 30:923-930). All normalizations and differential expression analysis was performed using the DESeq software suite (Anders and Huber (2010) Genome Biol. 11:R106). Heatmaps were generated with Gene-E software (http://www.broadinstitute.org/cancer/software/GENE-E/).

SLNCR homologs were identified through BLAST of the SLNCR sequence against the Reference RNA sequence database (blast.ncbi.nlm.nih.gov). Alignments were performed in Clustal Omega (Goujon et al. (2010) Nucl. Acids Res. 38:W695-W699; Sievers et al. (2011) Mol. Systems Biol. 7:539), and viewed in Jalview (Waterhouse et al. (2009) Bioinformatics 25:1189-1191). BAM files from RNA-seq of melanomas was visualized using the Integrated Genome Viewer (broadinstitute.org/igv/) (Robinson et al. (2011) Nat. Biotechnol. 29:24-26; Thorvaldsdottir et al. (2013) Briefings Bioinformatics 14:178-192).

For TCGA analyses, raw FASTQ files were obtained for 150 randomly selected sub-cutaneous melanoma samples following dbGaP approval using the GeneTorrent software for CGHub (Wilks et al. (2014) Database 2014:bau093). The SLNCR1 consensus transcript was aligned to each FASTQ using Bowtie2 (Langmead and Salzberg (2012) Nature Methods 9:357-359). RPKM values were calculated for cross-sample comparisons. Expression of mRNAs was accessed through the Broad Institute TCGA Genome Data Analysis Center (Harvard B.I.o.M.a. (2015) Broad Institute TCGA Genome Data Analysis Center: Firehose).

Small Interfering RNAs

All siRNAs were purchased from Qiagen. SLNCR1 specific siRNA sequences were custom synthesized and used at a final concentration of 10 nM. MMP-9-targeting siRNAs were used at a final concentration of 7.5 nM, AR-targeting siRNAs at 10 nM, and Brn3a-targeting siRNAs at 5 nM.

Gelatin Zymography

Gelatin zymography was performed as previously described, with slight modifications (Toth and Fridman, 2001). Cells were seeded at a density of 25×10⁴ cells per well in 6-well dishes, and transfected with the indicated plasmids and/or siRNAs 24 hours later. Cells were washed with PBS and transitioned to 900 μl of serum free media 24 hours post-transfection. Supernatant was removed 24 hours later and non-adherent cells were pelleted by centrifugation at 300×g for 5 minutes at 4° C. The remaining supernatant was then concentrated 5-fold using Millipore Amicon Ultra 10 kDa cutoff centrifugal devices. Samples were incubated at room temperature for 10 minutes in SDS sample buffer without a reducing agent, and then electrophoresed on 10% Criterion™ Zymogram Gel (Bio-Rad). After electrophoresis, gels were washed briefly in dH₂O, followed by 2 40 minute washes in 1× renaturation buffer (Bio-Rad); incubated for 18 hours at 37° C. in 1× development buffer (Bio-Rad); and stained with 0.1% Coomassie brilliant blue R250. Ratios of MMP-9 compared to MMP-2 were quantified by ImageJ software densitometric analysis of the 92-kd and 72-kd proteolytic bands, which correspond to MMP-9 and MMP-2, respectively.

Invasion Assays

Twenty-four hours post seeding at 25×10⁴ cells in a 6-well plate, cells were transfected with either 2,500 ng of the indicated plasmid or the indicated siRNAs at 10 nM final concentration. For invasions assays using A375 cells, 2.5×10⁴ cells in serum-free media were plated in either BD BioCoat™ matrigel inserts or uncoated control inserts (Corning), placed into DMEM with 30% FBS (fetal bovine serum), and incubated for 16 hours. For the melanoma short term cultures, 10×10⁴ or 7.5×10⁴ cells, for WM1976 or WM1575, respectively, in serum-free media were seeded in the chambers, placed into DMEM with 30% FBS (fetal bovine serum), and incubated for 22 hours. Cells that did not migrate or invade were removed using a cotton tipped swap, chambers were rinsed twice with PBS, and stained using Fisher HealthCare™ PROTOCOL™ Hema 3™ Fixative and Solutions. Cells were imaged on 20× magnification in 8 fields of view for 3 independent replicates.

RIP Assays

For AR RIP, HEK293T cells were seeded in a 10 cm dish, and 24 hours later were co-transfected with 15 μg pEGFP-C1-AR and 10 μg of SLNCR1- or SLNCR1^{Δcons}- or SLNCR1^{Δ568-637} expressing plasmids. For Brn3a RIP, HEK293T cells were seeded in a 10 cm dish, and co-transfected with 15 μg pCDNA-Brn3a and 10 μg of SLNCR1- or SLNCR1^{Δcons}-expressing plasmids. For UV-crosslinking of Brn3a IPs, cells were washed in PBS, and UV-crosslinked in a UV-Stratalinker at 400 mJ/cm2 in 5 mls of ice cold PBS. For both AR and Brn3a IPs, cells were lysed for 10 minutes in IP lysis buffer (20 mM Tris pH 7.4, 10 mM NaCl, 2 mM EDTA, supplemented with 0.5% Triton-X-100, RNaseOUT (Invitrogen), 1 mM PMSF, and cOmplete Protease Inhibitor Cocktail (Roche), NaCl was added to a final concentration of 400 300 mM and incubated on ice for an additional 10 minutes, and spun at 18,000×g for 10 minutes at 4° C. Lysate was split and immunoprecipitated with 1.5 μg α-AR antibody or IgG negative control, or 1 μg α-Brn3a antibody or IgG negative control, rotating for 18 hours at 4° C. Lysate was incubated with Protein A Dynabeads® (Life Technologies) (25 μl slurry) for 1 hour at 4° C., followed by 4×0.5 ml washes in wash buffer (50 mM Tris pH 7.4, 500 mM NaCl, supplemented with 0.05% Triton-X-100). For non-crosslinked cells, beads were boiled in 2× Laemmli buffer for 5 minutes are 95° C., and bound fractions were split for protein and RNA analysis. For UV-crosslinked cells, beads were resuspended in wash buffer and split for protein and RNA analysis. For RNA, 100 μg of proteinase K was added in proteinase K digestion buffer (300 mM NaCl, 200 mM Tris pH 7.5, 25 mM EDTA, 2% SDS), and incubated at 65° C. for 30 minutes with gentle mixing.

Accession Numbers

RNA sequencing data will be deposited into the Gene Expression Omnibus (GEO).

### Example 11: The lncRNA SLNCR is Robustly-Expressed in Melanomas and is Associated with Melanoma Survival Probability

To identify melanoma-associated lncRNAs, RNA-seq were performed on three melanoma short-term cultures (MSTCs) and fibroblast short-term cultures derived from the tumor microenvironment (unpublished data from Charles Yoon, Brigham and Woman's Hospital, Boston, Mass.). MSTCs have undergone relatively few passages outside of the patient and closely reflect the genetics of melanomas in situ, providing a tractable system to study disease-relevant transcriptional changes. Of the 137 lncRNAs expressed in human melanomas (FPKM>1, Table S1), the third most abundant lncRNA (XLOC_012568; linc00673, Refseq NR_036488.1; average FPKM=55.33) was expressed in MSTCs but not tumor-associated fibroblasts. This lncRNA was also located within a chromosomal region commonly amplified in melanoma, lung and ovarian cancers (www.broadinstitute.com/tumorscape, Table S2). Increased expression of XLOC_012568 was confirmed in the sequenced samples as well as five additional MSTCs by RT-qPCR (Table 1, FIG. 1A). In addition to melanomas, the MiTranscriptome database (mitranscriptome.org) revealed that XLOC_012568 was expressed in cervical, ovarian, and pancreatic cancer, low-grade glioma and glioblastoma multiforme, and that XLOC_012568 expression was increased in lung adenocarcinoma and squamous cell carcinomas compared to corresponding normal tissues (Iyer et al. (2015) Nat. Genet. 47:199-208) (FIG. 1E). Collectively, these data suggested a broader role for this lncRNA in human tumorigenesis.

There are three XLOC_012568 isoforms expressed in melanomas (FIG. 1F). The most prevalent isoform, SLNCR1, is 2257 nucleotides and composed of 4 exons spanning chr17:70399463-70588943 (FIG. 1B). Isoforms 2 and 3 contained an additional short or long exon, respectively, located between exon 3 and 4. Despite the fact that most lncRNAs displayed only modest sequence conservation due to their rapid evolution XLOC_012568 included a highly-conserved region across mammals (FIGS. 1B and 1G and 1H) (Necsulea et al. (2014) Nature 505:635-640). This conserved region was located within a region of high identity (54%) to the steroid receptor RNA Activator-1 (SRA1; FIGS. 1B and 1I). While the SRA1 locus expressed both protein-coding and functional non-coding transcripts, none of the 3 SLNCR isoforms exhibited protein-coding potential (coding potential scores SLNCR1: 0.12, SLNCR2: 0.10, SLNCR3: 0.37) (Chooniedass-Kothari et al. (2004) FEBS Lett. 566:43-47; Kong et al. (2007) Nucl. Acids Res. 35:W345-W349; Lanz et al. (1999) Cell 97:17-27). The conservation of SLNCR1, similarity to a functional non-coding RNA, and abundance suggested a functionally important role for SLNCR1.

To annotate SLNCR expression to clinically-relevant parameters, SLNCR expression was assessed across 150 randomly-selected human melanomas from TCGA. It was important to note that this analysis did not distinguish between SLNCR isoforms. In agreement with results from patient-derived melanomas, SLNCR was expressed in 146 out of 150 randomly selected human melanomas (RPKM>1, Table S1). Tumor depth, as described by Breslow's thickness (T, measured in millimeters), was one of the most important prognostic factors in melanoma treatment. Specifically, while thin tumors (<1 mm thick) were easily treatable by local surgical excision, thicker tumors (>1 mm thick) have a greater possibility of reaching blood vessels and are thus more likely to metastasize, requiring more aggressive treatment. SLNCR expression was significantly higher in tumors at least 1 mm thick, correlating with severity of the melanoma (AJCC staging classification TX/Tis/T0/T1 versus T2/T3/T4; FIG. 1C).

To investigate whether SLNCR expression was related to disease outcome in TCGA melanomas, a Kaplan-Meier survival analysis was performed comparing melanoma patients expressing high (n=72, red line) or low (n=70, blue line) levels of SLNCR1 (FIG. 1D). High expression of SLNCR was associated with shorter overall survival in melanoma patients (p-value=0.0426). The median survival for the low SLNCR group was 14.3 years, while the high SLNCR group had a median survival of only 5.3 years. Additionally, the pooled hazard ratio showed an 84% increase in the risk of death for the high SLNCR group (logrank HR=1.84, 95% confidence interval 1.03 to 3.60). Together, these data suggested a role for SLNCR at a clinically-critical stage of melanomagenesis.

TABLE S1
Expression (FPKM) of SLNCR in patient-derived cells as determined through RNA-seq.

| | | | | | | C10-L22 | C21-L | C01L | |
|---|---|---|---|---|---|---|---|---|---|
| | C02-S1 | C10-S15 | C21A-S | | | cancer | cancer | cancer | C02-L1 |
| | melanoma | melanoma | melanoma | C36 | C36 | associated | associated | associated | cancer |
| | primary | primary | primary | primary | metastatic | fibroblast | fibroblast | fibroblast | associated |
| | culture | culture | culture | tumor | lesion | primary | primary | primary | fibroblast |
| SLNCR | 62.6081 | 61.5303 | 41.8686 | 9.2813 | 0.489589 | 0.340279 | 0.985183 | 2.30418 | 0.154757 |

TABLE S2
SLNCR was located on a chromosomal region commonly
amplified in melanoma, lung and ovarian cancers. Data was taken
from Broad's TCGA Tumorscapes (Berwick et
al. (2010) J. Biol. Chem. 285: 15286-15295).

| Cancer | | | Overall Frequency |
|---|---|---|---|
| Type | Amplified Region | Q-value | of Amplification |
| Melanoma | Chr17: 41471733-78605474 | 0.242 | 49.5% |
| Lung NSC | Chr17: 55353997-78605474 | 0.128 | 39.6% |
| All lung | Chr17: 55353997-78605474 | 0.0949 | 39.9% |
| Ovarian | Chr17: 47316963-78605474 | 0.12 | 30.1% |

### Example 12: SLNCR1 Increased Melanoma Invasion Through Transcriptional Upregulation of MMP9

To gain insights into the role of SLNCR in melanoma formation, global transcriptional profiling was used before and after knockdown of the most abundant isoform, SLNCR1, in the MSTC WM1976. Two custom-designed siRNAs directed against exon 3-4 junction resulted in ˜80-90% knockdown of SLNCR1 (FIGS. 2C and 2D). Due to toxicity and apparent off-target effects of si-SLNCR1 (2) 48 hours post-transfection, only differentially expressed transcripts from duplicate knockdown using si-SLNCR1 (1) were used for RNA-seq analyses (FIGS. 2L and 2M). Knockdown of endogenous SLNCR1 resulted in the differential expression of 121 transcripts (adjusted p-value<0.1, fold change>2, Table S3), indicating that SLNCR1 regulated expression of numerous genes in trans.

TABLE S3
Transcripts that are significantly differentially expressed upon
knockdown of SLNCR1 in WM1976 cells.

| | Log2 (fold | |
|---|---|---|
| Transcript | change) | P-value |
| OPN1SW | −1.8035 | 4.76E−09 |
| ESR1 | −1.72869 | 0.000305 |
| DENND6A | −1.68464 | 5.62E−19 |
| AGPAT9 | −1.61193 | 6.77E−11 |
| FAM134B | −1.53157 | 6.38E−15 |
| PNMA2 | −1.51138 | 3.49E−06 |
| CDK19 | −1.5076 | 1.53E−11 |
| AKT3 | −1.50705 | 5.54E−20 |
| NIM1K | −1.44162 | 8.21E−05 |
| PPP1R2 | −1.44048 | 1.78E−16 |
| RARB | −1.40203 | 3.39E−07 |
| MFAP3L | −1.39078 | 3.59E−14 |
| SOD2 | −1.38602 | 4.31E−17 |
| COL15A1 | −1.3802 | 1.38E−08 |
| RP11- | −1.35583 | 4.61E−05 |
| 215G15.5 | | |
| PDE3B | −1.34432 | 1.18E−15 |
| MAP1A | −1.29707 | 2.87E−08 |
| LINC00511 | −1.28596 | 7.23E−15 |
| AP4E1 | −1.26168 | 2.34E−09 |
| TMOD2 | −1.24586 | 2.10E−06 |
| ARMCX4 | −1.23752 | 0.000128 |
| GOLM1 | −1.23016 | 1.76E−14 |
| YPEL2 | −1.20237 | 1.18E−09 |
| RNF168 | −1.20214 | 7.28E−11 |
| FRMD3 | −1.20038 | 7.13E−09 |
| NDC1 | −1.19726 | 6.64E−12 |
| TNRC6C | −1.171 | 7.74E−11 |
| ANKRD52 | −1.16707 | 4.28E−14 |
| TRMT2B | −1.16686 | 2.87E−10 |
| IL10RB | −1.162 | 3.76E−06 |
| CRADD | −1.15978 | 1.66E−05 |
| GSKIP | −1.15731 | 2.92E−09 |
| PHKB | −1.15139 | 3.78E−10 |
| GTPBP1 | −1.14814 | 3.59E−11 |
| CBFB | −1.14729 | 1.87E−11 |
| GPSM3 | −1.14357 | 5.50E−08 |
| FKBP1A | −1.13838 | 1.10E−11 |
| ANO5 | −1.13469 | 7.51E−08 |
| WWC3 | −1.13161 | 7.58E−12 |
| RIMS3 | −1.12993 | 3.40E−05 |
| PIK3AP1 | −1.12383 | 9.30E−05 |
| GRAMD3 | −1.12208 | 0.000192 |
| ANKS1A | −1.11679 | 5.91E−12 |
| RGS10 | −1.11275 | 1.62E−07 |
| NCAM2 | −1.09688 | 1.96E−06 |
| SGK3 | −1.09361 | 1.40E−05 |
| GALNT7 | −1.07912 | 1.76E−09 |
| FNDC3A | −1.07557 | 2.08E−11 |
| RPS6KA2 | −1.07511 | 3.96E−09 |
| SOX6 | −1.06988 | 4.07E−10 |
| DCUN1D1 | −1.06424 | 2.83E−09 |
| COQ10B | −1.06368 | 8.42E−09 |
| SWT1 | −1.05592 | 1.61E−06 |
| ITGA6 | −1.05054 | 2.18E−11 |
| ITGA2 | −1.0493 | 8.74E−08 |
| FAM46C | −1.04767 | 4.02E−05 |
| ETFDH | −1.04447 | 1.65E−07 |
| TNIK | −1.04345 | 2.68E−09 |
| MGAT4A | −1.04116 | 2.23E−07 |
| IPO8 | −1.03761 | 4.12E−10 |
| GABRA3 | −1.02673 | 1.08E−06 |
| ICAM5 | −1.02525 | 0.000192 |
| SYT11 | −1.01546 | 3.88E−07 |
| CAMK2D | −1.01002 | 1.20E−08 |
| CD200 | −1.00917 | 5.97E−07 |
| TP53INP1 | −1.00808 | 4.39E−09 |
| PIK3C2A | −1.00341 | 5.64E−10 |
| RYK | 0.591759 | 0.000283 |
| H1FX | 0.601909 | 0.000435 |
| NAA50 | 0.610151 | 8.16E−05 |
| GOLT1B | 0.620466 | 0.000238 |
| GXYLT1 | 0.631839 | 8.56E−05 |
| MIS18A | 0.646969 | 0.000657 |
| ELL2 | 0.648194 | 0.000102 |
| RAB15 | 0.650175 | 5.53E−05 |
| ARL5B | 0.656002 | 0.000436 |
| PTPRU | 0.664498 | 8.01E−05 |
| DENND5B | 0.665658 | 3.79E−05 |
| GADD45A | 0.676347 | 7.24E−05 |
| LIMCH1 | 0.699786 | 9.99E−05 |
| TMEM2 | 0.700626 | 3.60E−06 |
| BTG2 | 0.71142 | 7.01E−05 |
| IL1RAP | 0.71508 | 4.36E−06 |
| MAP4K5 | 0.716206 | 2.78E−05 |
| DUSP8 | 0.785871 | 0.000173 |
| WTIP | 0.807765 | 1.75E−05 |
| JUN | 0.812248 | 1.38E−06 |
| TGFBI | 0.819948 | 8.54E−06 |
| KIAA1598 | 0.822298 | 3.49E−07 |
| PROM1 | 0.837309 | 3.75E−05 |
| SMIM15 | 0.872592 | 3.70E−07 |
| CRIM1 | 0.878698 | 3.28E−07 |
| UGT8 | 0.87996 | 7.35E−06 |
| CELSR1 | 0.882447 | 2.40E−08 |
| SGMS2 | 0.897985 | 2.46E−05 |
| NOV | 0.924282 | 4.38E−09 |
| DRAM1 | 0.967168 | 3.76E−07 |
| RP11- | 0.990559 | 0.000158 |
| 429E11.2 | | |
| ZNF367 | 1.019652 | 9.67E−10 |
| DESI2 | 1.02863 | 1.25E−10 |
| OLFML2B | 1.064702 | 1.26E−05 |
| INPP5A | 1.096814 | 1.33E−09 |
| TECPR2 | 1.148571 | 2.34E−11 |
| CREB5 | 1.152999 | 6.78E−06 |
| SERTAD4 | 1.180104 | 1.16E−05 |
| WISP2 | 1.226731 | 0.000713 |
| PLEK2 | 1.275612 | 1.55E−05 |
| CLDN1 | 1.386865 | 3.57E−15 |
| SLC18A1 | 1.570512 | 0.000191 |
| ITK | 1.677054 | 8.85E−05 |
| PRODH | 1.684211 | 1.37E−15 |

Given that SLNCR1 was associated with overall melanoma survival and that tumor invasion was a critical step to melanoma metastasis, it was hypothesized that SLNCR1 contributed to melanoma invasion (FIG. 1D). To test this, invasion in SLNCR-expressing MSTCs was measured using a matrigel invasion assay. Knockdown of SLNCR1 significantly decreased invasion in WM1976 (˜80%) and WM1575 (˜60%) compared to controls, while cell proliferation was not affected (FIGS. 2E, 2F, 2G, 2H, 2L and 2M). This result suggested that endogenous SLNCR1 played a critical role in melanoma invasion.

To independently validate a role for SLNCR1 in melanoma invasion, SLNCR1 was over-expressed in the A375 melanoma cell line, which expressed low levels of this lncRNA. As expected, over-expression of SLNCR1 increased invasion (˜200%; FIGS. 21-2K). Over-expression of a SLNCR1 mutant lacking the highly-conserved sequence (SLNCR1^{Δcons}, nucleotides 462-572 deleted) did not increase invasion of A375 cells, suggesting a requirement for this conserved sequence. To validate this latter observation, the conserved sequence was over-expressed, including ˜100 nucleotides of flanking sequences to ensure proper RNA folding (SLNCR1^{cons}, nucleotides 372-672). Expression of SLNCR1^{cons} increased invasion to the same degree as full-length SLNCR1 (˜200%; FIG. 2I-2K), confirming that the conserved region was necessary and sufficient for SLNCR1-mediated melanoma invasion.

To identify genes that mediate increased melanoma invasion, SLNCR1, SLNCR1^{Δcons}, or SLNCR1^{cons} was over-expressed in A375 melanoma cells and unbiased transcriptional profiling was performed by RNA-seq. Expression of SLNCR1 resulted in the differential expression of 110 genes (adjusted p-value<0.05, fold change>2, Table S4 and FIG. 3J). Because the conserved sequence was necessary and sufficient for SLNCR1-mediated melanoma invasion, transcripts differentially expressed upon over-expression of SLNCR1 and SLNCR1cons was searched, but not SLNCR1Δcons. Two transcripts were identified significantly upregulated (p-value<0.05, fold change>1.5) by SLNCR1's conserved sequence: RARRES2P8, a pseudogene of the retinoic acid receptor responder, and MMP9, a gene that encoded matrix metallopeptidase 9, also known as gelatinase B (FIG. 3C). MMP9 contributed to early melanoma invasion through remodeling of the extracellular matrix (Hofmann et al. (2005) Biochimie 87:307-314; MacDougall et al. (1999) Br. J. Cancer 80:504-512; MacDougall et al. (1995) Cancer Res. 55:4174-4181; van den Oord et al. (1997) Amer. J. Pathol. 151:665-670). Consistent with a role in early tumor dissemination, analysis of the TCGA melanoma cohort revealed that MMP9 expression significantly higher in regional metastases compared to primary tumors (p-value=0.0003, FIG. 3K).

TABLE S4
Significantly differentially expressed transcripts upon SLNCR1
overexpression in A375 cells. Results are from a replicate
RNA-seq experiments shown in FIG. 7 of the patent.

| | Log2 (fold | |
|---|---|---|
| Transcript | change) | P-value |
| RP5- | −5.90821 | 1.50E−19 |
| 983L19.2 | | |
| CACNA1H | −4.67719 | 3.18E−08 |
| NPPB | −4.35964 | 3.47E−06 |
| RP11-161I2.1 | −4.06703 | 2.15E−09 |
| HLF | −3.79364 | 3.46E−12 |
| IL34 | −3.34675 | 2.15E−05 |
| ITGAM | −3.20699 | 1.04E−13 |
| RP11- | −2.89654 | 5.99E−22 |
| 875O11.3 | | |
| C1orf51 | −2.87771 | 1.38E−09 |
| NPFFR2 | −2.82032 | 0.000366 |
| ASNSP1 | −2.78353 | 1.83E−12 |
| NEURL3 | −2.66185 | 5.82E−30 |
| EGR2 | −2.64984 | 8.57E−28 |
| PLA2G4C | −2.58755 | 2.08E−22 |
| TSPAN2 | −2.55111 | 8.70E−23 |
| GFRA1 | −2.54443 | 3.22E−07 |
| PIK3IP1 | −2.52178 | 5.20E−08 |
| BHLHE41 | −2.48136 | 4.67E−56 |
| PDE4C | −2.43944 | 1.99E−07 |
| ALOXE3 | −2.42231 | 1.60E−21 |
| BEST1 | −2.36872 | 4.22E−10 |
| INHBE | −2.34939 | 7.21E−14 |
| PER2 | −2.06258 | 9.82E−38 |
| HIST1H3D | −2.05477 | 6.78E−07 |
| ACTN2 | −2.04205 | 1.95E−16 |
| NHLH2 | −1.97528 | 5.50E−07 |
| GRHL3 | −1.94598 | 3.29E−06 |
| RP11- | −1.78921 | 9.67E−05 |
| 169K16.6 | | |
| PPARGC1A | −1.78713 | 2.73E−09 |
| ADIRF-AS1 | −1.77072 | 9.58E−50 |
| CD99P1 | −1.76531 | 1.51E−10 |
| HIST1H2AK | −1.75443 | 0.00052 |
| C12orf39 | −1.66435 | 1.16E−05 |
| ZNF699 | −1.65725 | 2.36E−08 |
| ACHE | −1.65025 | 7.81E−05 |
| IL32 | −1.64149 | 1.08E−15 |
| WDR66 | −1.64127 | 1.84E−06 |
| CNOT6LP1 | −1.62961 | 0.000378 |
| RP11- | −1.62859 | 1.42E−05 |
| 347C12.2 | | |
| PER1 | −1.61886 | 5.65E−32 |
| HIST2H2BF | −1.60835 | 0.000536 |
| NR1D1 | −1.6079 | 1.01E−45 |
| C8orf46 | −1.55315 | 0.000291 |
| ZNF79 | −1.55075 | 2.07E−23 |
| MMP19 | −1.54702 | 1.49E−20 |
| PER3 | −1.52959 | 6.79E−16 |
| SYP | −1.51797 | 8.65E−06 |
| CHRNB2 | −1.51164 | 6.99E−05 |
| RUNDC3A | −1.50507 | 0.000376 |
| HIST2H3DP1 | −1.49133 | 0.000793 |
| NF1P8 | −1.48329 | 2.94E−07 |
| ZNF441 | −1.47365 | 4.81E−21 |
| WDR63 | −1.46618 | 0.000329 |
| HIST1H2BN | −1.45294 | 0.000833 |
| GS1-124K5.2 | −1.44992 | 0.000842 |
| RASGRF1 | −1.44445 | 2.70E−05 |
| SYDE2 | −1.42172 | 2.14E−05 |
| RP11-345J4.8 | −1.4056 | 0.000853 |
| ANK1 | −1.36853 | 0.00084 |
| CHGB | −1.3674 | 1.48E−06 |
| HDAC9 | −1.34582 | 1.61E−21 |
| HIST1H2AG | −1.33515 | 3.47E−06 |
| SFTA1P | −1.33382 | 6.05E−06 |
| CCT6P3 | −1.32372 | 0.000206 |
| HIST1H4H | −1.30489 | 1.20E−05 |
| REL | −1.29997 | 4.41E−05 |
| FSBP | −1.29439 | 0.000861 |
| TEF | −1.27627 | 1.34E−18 |
| ZNF235 | −1.2688 | 1.45E−06 |
| COL5A3 | −1.24795 | 6.20E−08 |
| HIST1H1D | −1.24272 | 4.55E−05 |
| C7orf63 | −1.23883 | 3.48E−07 |
| KCNT2 | −1.23749 | 1.01E−09 |
| DBP | −1.21614 | 8.82E−20 |
| EGR1 | −1.19778 | 8.91E−29 |
| CTD- | −1.19107 | 8.53E−09 |
| 2140G10.2 | | |
| BSN | −1.18134 | 7.35E−08 |
| ZNF416 | −1.16917 | 9.44E−09 |
| CCDC181 | −1.16822 | 7.92E−10 |
| ZNF221 | −1.1498 | 8.26E−05 |
| ADM2 | −1.14342 | 3.95E−13 |
| TRIML2 | −1.14003 | 4.15E−10 |
| CPEB3 | −1.12993 | 2.39E−05 |
| ZNF433 | −1.12597 | 6.41E−06 |
| PVT1 | −1.12425 | 2.27E−15 |
| BIRC3 | −1.12392 | 5.43E−17 |
| ZNF460 | −1.11703 | 0.000478 |
| THAP9 | −1.0989 | 6.70E−05 |
| ZSCAN12 | −1.09608 | 4.72E−06 |
| ZNF844 | −1.0917 | 1.69E−17 |
| PROX1 | −1.08312 | 0.000992 |
| RRN3P1 | −1.07954 | 0.000356 |
| CREBRF | −1.07771 | 9.52E−17 |
| THUMPD2 | −1.07026 | 8.50E−15 |
| ZNF805 | −1.0671 | 5.47E−06 |
| CD163L1 | −1.06659 | 0.000254 |
| COL17A1 | −1.05634 | 4.92E−07 |
| FAM179B | −1.04278 | 5.39E−13 |
| KIAA1324 | −1.03194 | 0.00026 |
| CTH | −1.0291 | 4.17E−10 |
| ZNF442 | −1.02806 | 0.000886 |
| PLCB4 | −1.02391 | 7.33E−07 |
| HIST2H2BE | −1.02362 | 3.10E−06 |
| KLF15 | −1.02289 | 3.44E−07 |
| NUAK2 | −1.02004 | 1.65E−14 |
| NFKBIE | −1.01324 | 2.70E−15 |
| AKR1C1 | 0.600615 | 0.000304 |
| IL8 | 0.836243 | 1.05E−12 |
| IL6 | 1.00536 | 2.65E−08 |
| RP3- | #NAME? | 0.000529 |
| 399L15.3 | | |

Because SLNCR1^{cons} regulated expression of MMP9 and mediated SLNCR1-induced invasion, it was hypothesized that MMP9 was responsible for SLNCR1-induced invasion. First, RT-qPCR was performed to confirm that expression of SLNCR1^{cons} was necessary and sufficient for increasing MMP9 mRNA (FIG. 3D). Next, gelatin zymography was used to quantify the activity of MMP9 after over-expression of SLNCR1, SLNCR1^{Δcons} and SLNCR1^{cons}. In agreement with changes in MMP9 levels, expression of SLNCR1 or SLNCR1^{cons} resulted in ˜50% increased MMP9 activity (FIG. 3F). Over-expression of lncRNAs, like proteins, may force non-physiological interactions and subsequently cause artefactual downstream affects. To confirm that endogenous SLNCR1 regulated MMP9, MMP9 activity was quantified by gelatin zymography following SLNCR1 knockdown. Consistent with a role in regulation of MMP9 activity, SLNCR1 knockdown decreased MMP9 activity (40-50%) in MSTCs WM1575 and WM1976 (FIGS. 3F and 3G).

If SLNCR1 increasef melanoma invasion by upregulating MMP9, depleting MMP9 should block SLNCR1-mediated increased in melanoma invasion. To test this hypothesis, A375 cells was transfected with empty or SLNCR1-expressing vectors, along with control or MMP9-specific siRNAs (FIG. 3L). As expected, MMP9 knockdown blocked the SLNCR1-mediated increase in MMP9 activity (FIGS. 3M and 3N). Matrigel invasion assays revealed that MMP9 knockdown blocked the SLNCR1-mediated invasion of melanoma cells (FIG. 3H). Together, these data demonstrated that SLNCR1 increased melanoma invasion by upregulating MMP9.

LncRNAs may modulate gene expression either transcriptionally or post-transcriptionally. To test if SLNCR1 transcriptionally upregulated MMP9, a firefly luciferase (FL) reporter was generated under the control of the 2 kb MMP9 promoter (MMPp-FL) and monitored expression in A375 cells (FIG. 3I). When normalized to expression of renilla luciferase from a co-transfected control reporter plasmid, SLNCR1 expression resulted in a significant (˜3.5-fold) increase in FL activity. To further validate the requirement of SLNCR1^{cons}, deletion mutants of SLNCR1 were generated and monitored FL activation. In agreement with previous results showing a requirement of SLNCR1^{cons}, expression of SLNCR1^{Δcons} did not increase FL activity. Interestingly, deletion of 70 bases immediately 3′ to the conserved region (SLNCR1^{Δ568-637}) also failed to increase FL activity, indicating an additional requirement for this sequence in MMP9 regulation. It was important to note that this region was included in the sequence over-expressed in SLNCR1^{cons} (FIG. 2I-2K). Furthermore, because serum-containing media contained exogenous hormones and steroids that affect activity of steroid hormone receptors, the assay was performed in the absence of steroids. SLNCR1 increased FL activity in steroid-deprived cells indicating that SLNCR1-mediated regulation of MMP9 was not dependent on exogenous hormones contained in the media. Collectively, these data confirmed that SLNCR1, specifically nucleotides 462-637, upregulated the MMP9 promoter in ligand-independent manner.

### Example 13: AR and Brn3a Bound to Adjacent Regions within SLNCR1's Conserved Sequence

Previously characterized lncRNAs ‘fine-tune’ gene expression patterns through a range of mechanisms, including direct interaction with TFs (Geisler and Coller (2013) Nat. Rev. Mol. Cell Biol. 14:699-712). The region of SLNCR1 responsible for transcriptionally upregulating MMP9 was similar to the TF binding and regulating lncRNA SRA1 (FIG. 1I), suggesting that SLNCR1 interacted with one or more TFs (Colley and Leedman (2011) Biochimie 93:1966-1972). SLNCR1 levels were sufficient for regulation of TFs, as TCGA patient melanomas exhibited SLNCR expression similar to those observed for SRA1 in other tissues (˜0-60 RPKM, or approximately up to 100 copies per cell) (http://gdac.broadinstitute.org) (Harvard B.I.o.M.a. (2015) Broad Institute TCGA Genome Data Analysis Center: Firehose; Kellis et al. (2014) Proc. Nat. Acad. Sci. U.S.A. 111:6131-6138; Mortazavi et al. (2008) Nat. Methods 5:621-628). Consistent with a nuclear role for this lncRNA, fractionation of the MSTC WM1976 revealed that SLNCR was found in both cytoplasm and nucleus (FIG. 4F). Because SLNCR1 transcriptionally upregulated the MMP9 promoter (FIG. 3I), was present in the nucleus, and was similar to SRA1, it was hypothesized that it bound TFs.

TFs are generally expressed at very low levels, making their identification using standard techniques challenging. A novel approach was designed for identification of RNA-bound TFs that was term RATA (RNA-associated transcription factor array). This technique coupled an RNA pulldown with a high-throughput TF activation array, enabling highly-sensitive and unbiased identification of TFs bound to an RNA of interest (FIG. 4A). The bacteriophage coat protein MS2 interacted with high-affinity to a specific stem-loop structure in the phage genome and had been widely adapted for biochemical purification of mammalian RNAs, including lncRNAs (Gong and Maquat (2015) Methods Mol. Biol. 1206:81-86). Twelve copies of the MS2 binding site was cloned into the 3′ end of SLNCR1, and qPCR of selected SLNCR1 targets was performed to confirm that the epitope tag did not interfere with normal gene-regulatory function (data not shown). SLNCR1 constructs containing MS2 binding sites were co-expressed with a plasmid expressing nuclear FLAG-tagged MS2 protein and immunoprecipitated with anti-FLAG antibodies Immunoprecipitation of the FLAG-tagged MS2 protein routinely showed ˜30-100 fold enrichment of MS2-tagged SLNCR1 or SLNCR1^{Δcons} (FIG. 4G). For subsequent use in the TF activation array, bound RNAs and proteins were eluted from beads using FLAG peptide under non-denaturing conditions (FIG. 4B). The eluate was then subjected to a TF Activation Profiling Plate Array (Signosis), allowing for simultaneous, quantitative analysis of multiple TFs in a single assay. Pulldowns were repeated in triplicate, and TFs showing specific >7-fold enrichment compared to the untagged control in at least 2 experiments were considered potential candidates.

Probes specific for PAX5, a TF involved in early development, were enriched with both SLNCR1 and SLNCR1^{Δcons} immunoprecipitations, suggesting PAX5 binds to SLNCR1 outside of its conserved region (FIG. 4C). Particularly of interest was identifying TFs binding to the critical invasion-regulating sequence (SLNCR1^{cons}). Probes for Brn3a (Pou4F1) routinely showed strong enrichment with SLNCR1, but showed negligible enrichment upon deletion of the conserved sequence, suggesting that Brn3a bound to the conserved region. Although Brn3a had not previously been shown to bind RNA, the TF contained a predicted RNA-binding motif in amino acid position 143-175 (MOTIF Search, http://www.genome.jp/tools/motif/). Modest enrichment of probes specific for the androgen receptor (AR), EGR, E2F-1, ATF2, and AP2 was observed. AR was focused on for several reasons: (1) AP2, ATF2, E2F-1, and EGR directly or indirectly interacted with AR, suggesting that enrichment of these probes was a consequence of these TFs interacting with SLNCR1-bound AR (Altintas et al. (2012) Mol. Endocrinol. 26:1531-1541; Jorgensen and Nilson (2001) Mol. Endocrinol. 15:1496-1504; Verger et al. (2001) J. Biol. Chem. 276:17181-17189; Zhang et al. (2010) Oncogene 29:723-738), (2) transcripts involved in reproduction, and specifically the AR transcriptional network, were significantly enriched among SLNCR1-regulated genes (Table S3: SLNCR1 knockdown, p-value=2.750E-57, z-score=135.3; Table S4: SLNCR1 over-expression, p-value 5.070E-63, z-score=160.15; MetaCore™, Thomson Reuters), (3) AR directly bound other lncRNAs (Yang et al. (2013) Nature 500:598-602; Zhang et al. (2015) Cell Rep. 13:209-221) and (4) AR positively regulated MMP9 in other cancers, including gastric, bladder, and prostate cancers (Ergun et al. (2007) Mol. Sys. Biol. 3:82; Hara et al. (2008) Cancer Res. 68:1128-1135; Wang et al. (2013) Mol. Cancer Ther. 12:1026-1037; Wu et al. (2010) Urology 75:820-827; Zhang et al. (2014) Oncotarget 5:10584-10595). Importantly, melanomas expressed both Brn3a and AR (Allil et al. (2008) Med. Chem. 4:100-105; Hohenauer et al. (2013) EMBO Mol. Med. 5:919-934; Morvillo et al. (2002) Melanoma Res. 12:529-538).

To validate the interactions between SLNCR1^{cons} and either AR or Brn3a, RNA immunoprecipitation (RIP) assays were carried out. Because A375 melanoma cells expressed low levels of endogenous SLNCR1 that would interfere with expression of specific SLNCR1 deletion mutants, RIP was performed from HEK293T (human embryonic kidney) cells. SLNCR1, but not GAPDH or β-ACTIN, was significantly enriched (˜120-fold) in RNAs immunoprecipitating with ectopically expressed AR, confirming that SLNCR1 bound specifically to AR (FIG. 4D). Surprisingly, SLNCR1^{Δcons} was still enriched in AR immunoprecipitates (˜50-fold), though to a lower extent than typically seen with full-length SLNCR1, suggesting that AR bound outside of SLNCR1⁴⁶²⁻⁵⁷². The results also indicated that, in addition to SLNCR1^{cons}, SLNCR1⁵⁶⁸⁻⁶³⁷ was required for MMP9 upregulation (FIG. 3I). RNA secondary structure was often critical to RNA function; thus, deletion of RNA sequences may affect neighboring functional sequences through disruption of secondary structure and subsequent weakening of structure-dependent interactions. It was therefore hypothesized that AR bound to SLNCR1⁵⁶⁸⁻⁶³⁷, and that deletion of the conserved sequence immediately upstream (nucleotides 462-572) disrupted the RNA secondary structure required for AR binding and weakened the lncRNA-protein interaction. Consistent with this hypothesis, the enrichment of SLNCR1 upon deletion of nucleotides 568-637 was not significantly greater than enrichment of background levels of SLNCR in HEK293T cells (˜14-fold, FIG. 4D and data not shown), confirming that AR bound to SLNCR1⁵⁶⁸⁻⁶³⁷. To accurately capture specific Brn3a-RNA interactions, ultraviolet (UV) light was used to crosslink HEK293T cells prior to immunoprecipitation of Brn3a. SLNCR1 was significantly enriched in Brn3a immunoprecipitates (1500-fold) while SLNCR1^{Δcons} ns showed no enrichment (FIG. 4E), confirming that Brn3a bound to SLNCR1's conserved sequence.

### Example 14: Upregulation of MMP9 Required SLNCR1, AR and Brn3a

Because AR and Brn3a bind to SLNCR1's conserved region, it was hypothesized that all 3 components were specifically required for upregulation of MMP9. If true, depletion of either TF should block SLNCR1-mediated upregulation of MMP9 and invasion, even in the presence of the remaining TF and SLNCR1. Toward this end, gelatin zymography was reported using, MMP9p-FL reporter and matrigel invasion assays after over-expressing SLNCR1 and simultaneously knocking down of either TF in the A375 melanoma cell line. Consistent with the hypothesis that AR was required for regulating MMP9 activity, depleting AR prevented MMP9 activation and promoter upregulation after SLNCR1 over-expression (FIGS. 5A-B and 5G-5J). Moreover, SLNCR1 over-expression failed to increase invasion of A375 cells depleted of AR in matrigel invasion assays, confirming that AR was required for SLNCR1-mediated invasion (FIG. 5C).

To test if Brn3a was also required for SLNCR1-mediated invasion, the above assays were repeated using Brn3a-specific siRNAs. Similar to results seen with AR, depletion of Brn3a prevented SLNCR1-mediated upregulation of MMP9 activity as quantified by gelatin zymography, and also prevented activation of the MMP9p-FL reporter construct (FIGS. 5D-E and 5K-5M). Next, invasion assays of A375 melanoma cells expressing vector alone or SLNCR1 were repeated in the presence of scramble or Brn3a-specific siRNAs (FIG. 5F). Interestingly, knockdown of Brn3a resulted in a significant increase in melanoma invasion. However, this invasion occured independently of an increase in MMP9 (FIGS. 5D and 5E), and was not further increased upon over-expression of SLNCR1, indicating that the increase in invasion seen upon Brn3a depletion was independent of SLNCR1. Knockdown of AR or Brn3a completely abrogated upregulation of MMP9 and melanoma invasion, demonstrating a functional requirement for SLNCR1, AR and Brn3a, and suggesting formation of a ternary complex composed of SLNCR1, AR and Brn3a.

### Example 15: The MMP9 Promoter Contained Predicted AR and Brn3a Binding Sites Required for SLNCR1-Mediated Upregulation

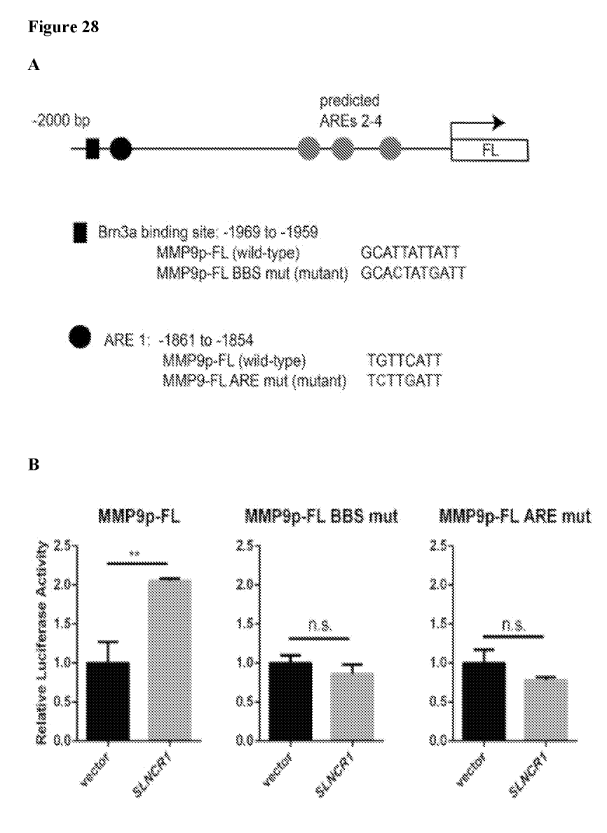

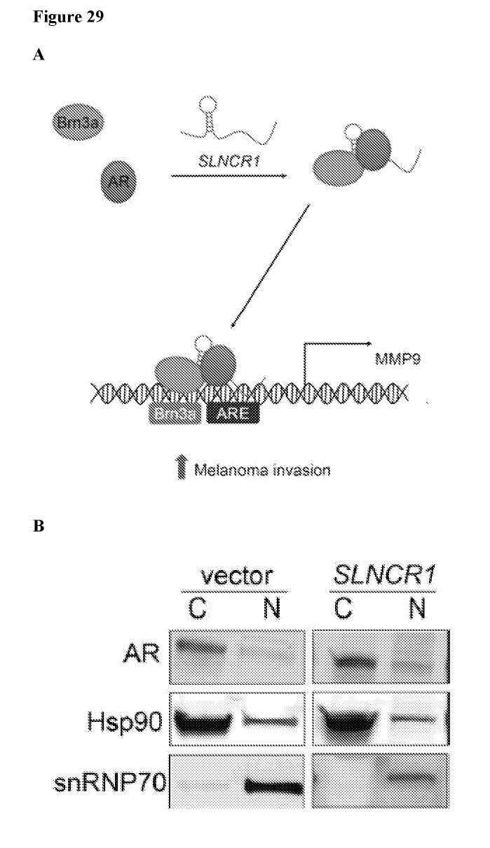

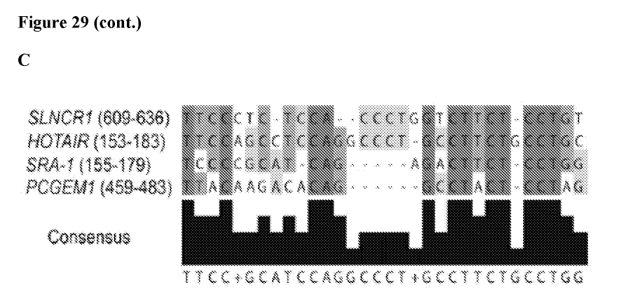

LncRNAs may direct TFs to target regions in the chromosome through direct binding to DNA and formation of an RNA-DNA complex, or by acting as scaffolds to assemble a complex of multiple TFs and regulatory proteins (Geisler and Coller (2013) Nat. Rev. Mol. Cell Biol. 14:699-712; Wang and Chang (2011) Mol. Cell 43:904-914). To distinguish between these possibilities, the MMP9 promoter was examined for sequence similarity to SLNCR1 or for the presence of predicted AR or Brn3a binding sites. No significant similarity was observed between SLNCR1 and the MMP9 promoter, arguing against a direct interaction between SLNCR1 and the DNA. In support of direct TF binding, the MMP9 promoter contained multiple functional AREs (androgen response elements), as well as a near perfect consensus Brn3a binding site (gcAT[A/T]A[T/A]T[A/T]AT) (FIG. 28A) (Gruber et al. (1997) Mol. Cell Biol. 17:2391-2400; Zhang et al. (2014) Oncotarget 5:10584-10595). The Brn3a binding site was located approximately 100 nucleotides upstream of the first ARE, an orientation consistent with cooperative TF binding. To test if these TF binding sites (TFBSs) were required for SLNCR1-mediated transcriptional upregulation of MMP9, MMP9p-FL reporter constructs were generated harboring mutations within the predicted ARE (MMP9-FL ARE mut) or the Brn3a binding site (MMP9-FL BBS mut). While over-expression of SLNCR1 in A375 cells significantly increased luciferase expression from the wild-type MMP9p-FL reporter, mutation of either the predicted Brn3a binding site or the ARE abolished the ability of SLNCR1 to increase luciferase activity (FIG. 28B). These data strongly suggested that binding of both AR and Brn3a to their respective TFBSs in the MMP9 promoter was required for transcriptional activation of MMP9.

### Example 16: ChIP-Seq of the Androgen Receptor from A375

ChIP-seq of the androgen receptor from A375 cells transfected with either vector control or a vector expressing SLNCR1 was performed. The results show that SLNCR1 affects AR occupancy at target genes and other genes relevant for cancer (Table S5 and S6). For example, some hits include CD274 and PD-L1. Table S5 and S6 contain these results (filtered for significant hits, meaning at least one max peak height is 25, and the fold change upon expression of SLNCR1 is at least 1.5 fold).

### Example 17: RNA Sequence Requirements for Androgen Receptor Binding

The RNA electrophoretic mobility gel shift assays (REMSAs) were performed to define the RNA sequence requirements for androgen receptor binding (see FIGS. 30A-D). The single RNA oligos tested were as follows:

(SEQ ID NO: 17)
RNA: UCUCUCUCUCUCUCUCUCUC
(SEQ ID NO: 36)
DNA: TCTCTCTCTCTCTCTCTCTC
(SEQ ID NO: 18)
RNA: UUCUUUCUUUCUUUCUUUCU
(SEQ ID NO: 37)
DNA: TTCTTTCTTTCTTTCTTTCT
(SEQ ID NO: 19)
RNA: CCCUCCCUCCCUCCCUCCCU
(SEQ ID NO: 38)
DNA: CCCTCCCTCCCTCCCTCCCT
(SEQ ID NO: 20)
RNA: CUGGAGGUAUUUUUCCCUCUCCACCCUGGUCUUCUCCUGUA
(SEQ ID NO: 39)
DNA: CTGGAGGTATTTTTCCCTCTCCACCCTGGTCTTCTCCTGTA
(SEQ ID NO: 21)
RNA: CAGGAGGUGACCCUCGUCUUCUCCUG
(SEQ ID NO: 40)
DNA: CAGGAGGTGACCCTCGTCTTCTCCTG
(SEQ ID NO: 22)
RNA: UUCCCUCUCCACCCUGGUCUUCUCCUGU
(SEQ ID NO: 41)
DNA: TTCCCTCTCCACCCTGGTCTTCTCCTGT
(SEQ ID NO: 23)
RNA: UUCCCUCUCCA
(SEQ ID NO: 42)
DNA: TTCCCTCTCCA
(SEQ ID NO: 24)
RNA: CUUCUCCUGU
(SEQ ID NO: 43)
DNA: CTTCTCCTGT
(SEQ ID NO: 25)
RNA: UAUUUUUCCCUCUCCAC
(SEQ ID NO: 44)
DNA: TATTTTTCCCTCTCCAC
(SEQ ID NO: 26)
RNA: UGGAGGUAUUUUUCCCUCUCCA
(SEQ ID NO: 45)
DNA: TGGAGGTATTTTTCCCTCTCCA
(SEQ ID NO: 27)
RNA: CUGGAGGUAUUUUUCCCUCUCCAG
(SEQ ID NO: 46)
DNA: CTGGAGGTATTTTTCCCTCTCCAG
(SEQ ID NO: 28)
RNA: AUCGCUCUCAACCCUGGUCUUCUCCUGU
(SEQ ID NO: 47)
DNA: ATCGCTCTCAACCCTGGTCTTCTCCTGT
(SEQ ID NO: 29)
RNA: UUCCCUCUCCACCCUGGUAGUCUCAGGU
(SEQ ID NO: 48)
DNA: TTCCCTCTCCACCCTGGTAGTCTCAGGT
(SEQ ID NO: 30)
RNA: AUCGCUCUCAACCCUGGUAGUCUCAGGU
(SEQ ID NO: 48)
DNA: ATCGCTCTCAACCCTGGTAGTCTCAGGT
(SEQ ID NO: 31)
RNA: UCCUUUCCUCACCCUGGUUCUCUUCCGU
(SEQ ID NO: 50)
DNA: TCCTTTCCTCACCCTGGTTCTCTTCCGT
(SEQ ID NO: 32)
RNA: UUCCCUCUCCAGCAUGGUCUUCUCCUGU
(SEQ ID NO: 51)
DNA: TTCCCTCTCCAGCATGGTCTTCTCCTGT
(SEQ ID NO: 33)
RNA: UAUUUUUCCCUCUCCACCCU
(SEQ ID NO: 52)
DNA: TATTTTTCCCTCTCCACCCT
(SEQ ID NO: 34)
RNA: UAUUUUUCCCUUCCCACCCU
(SEQ ID NO: 53)
DNA: TATTTTTCCCTTCCCACCCT
(SEQ ID NO: 35)
RNA: UCCCCGCAUCAGAGACUUCUCCUGG
(SEQ ID NO: 54)
DNA: TCCCCGCATCAGAGACTTCTCCTGG

The symbol ‘+’ indicates that a structure is highly predicted, ‘+/−’ indicates that a portion of the RNA sequence is predicted to form a secondary structure, and ‘−’ indicates that no structure is predicted to form. The data indicates that AR requires an unstructured RNA sequence of at least 9 polypyrimidines (Us and Cs) for binding, and specifically requires at least one sequence motif composed of UCUCCA/U, with a slight preference for A in the 6th position. These motifs are highlights in red (preferred UCUCCA) and orange (UCUCCU). ‘AR min’ sequences are sequences derived directly from SLNCR, with any mutated nucleotides denoted in BOLD. The requirement of at least 9 polypyrimidines is highlighted by binding of AR min 5 and AR min 10, and lack of binding of AR min 6 and the SRA-1 sequence. The necessity for an unstructured motif is highlighted by binding of AR min 7 and AR min 15, versus lack of binding of AR min 8 and 9. Finally, the specific requirement of the consensus motif is highlighted with binding of AR min 15, and complete loss of binding with AR min 16, containing only two mutations of nucleotides 616 (U-C) and 617 (C-U). The lack of binding to sequences, SEQ ID NOS 17, 18 and 19, confirm that binding is dictated by exact sequence requirements and not merely a polypyrimidine track. Despite the lack of a perfect consensus sequence, AR likely is able to weakly bind AR min 13 due to the strong polypyrimidine track and the presence of a near consensus sequence (UCUCUU). The SLNCR sequence contains 2 AR binding motifs (red and orange in Panel A). AR likely binds first to the unstructured orange motif, relaxing the SLNCR secondary structure and allowing for subsequent binding to the first red motif. The appearance of a second band in panel D with higher amounts of AR supports the formation of at least 2 unique complexes on the AR min 2 probe. The absolutely minimal required sequence for AR binding is therefore nucleotides 614-619 (UCUCCa) and 629-634 (UCUCCu), located within a longer polypyrimidine track.

### Example 18: SLNCR and AR Regulate Expression of PD-L1 (CD274) in Melanoma

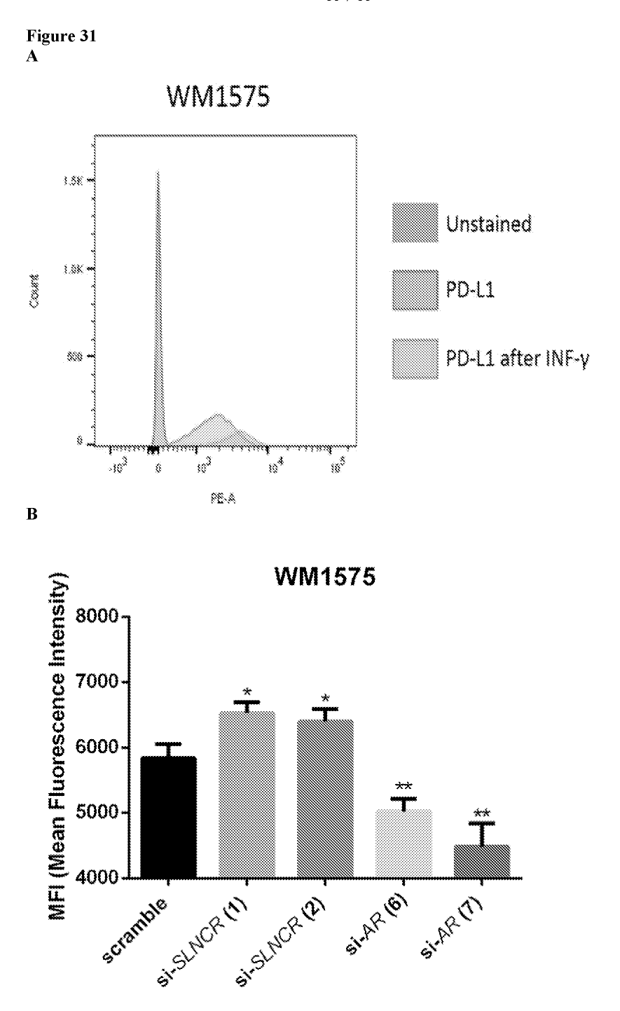

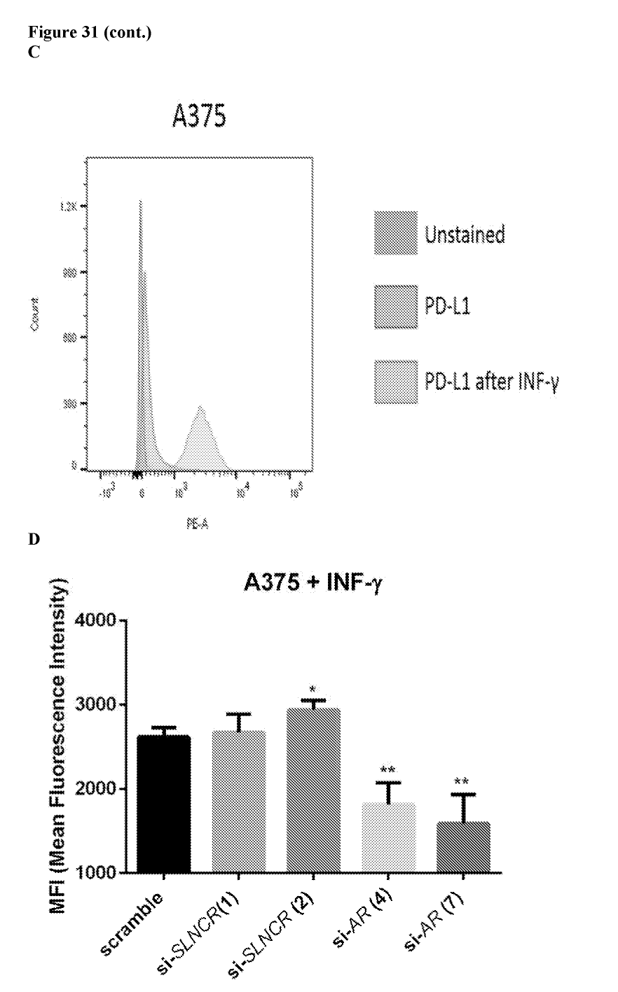

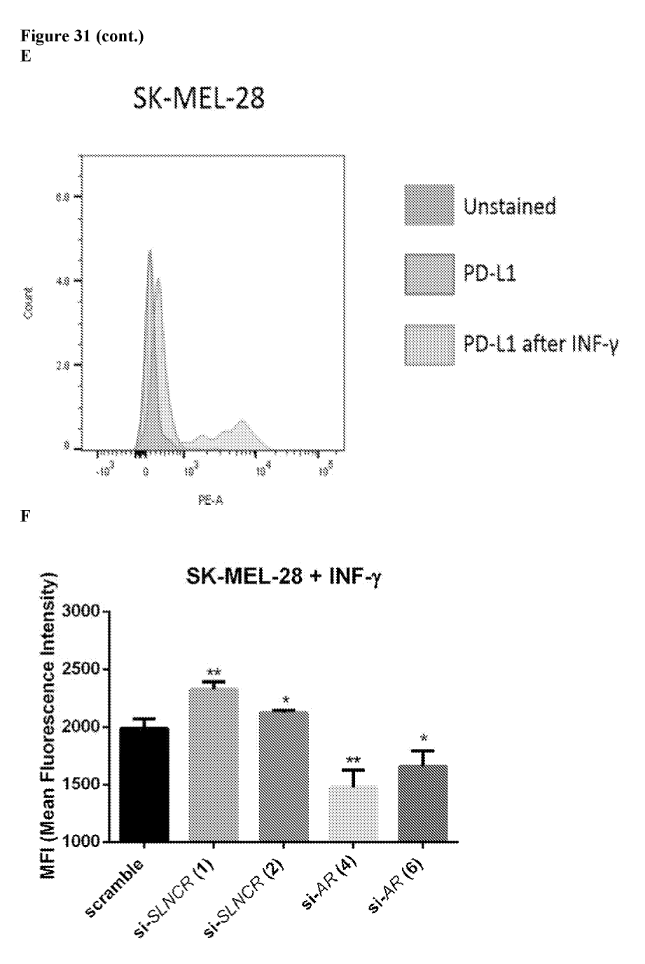

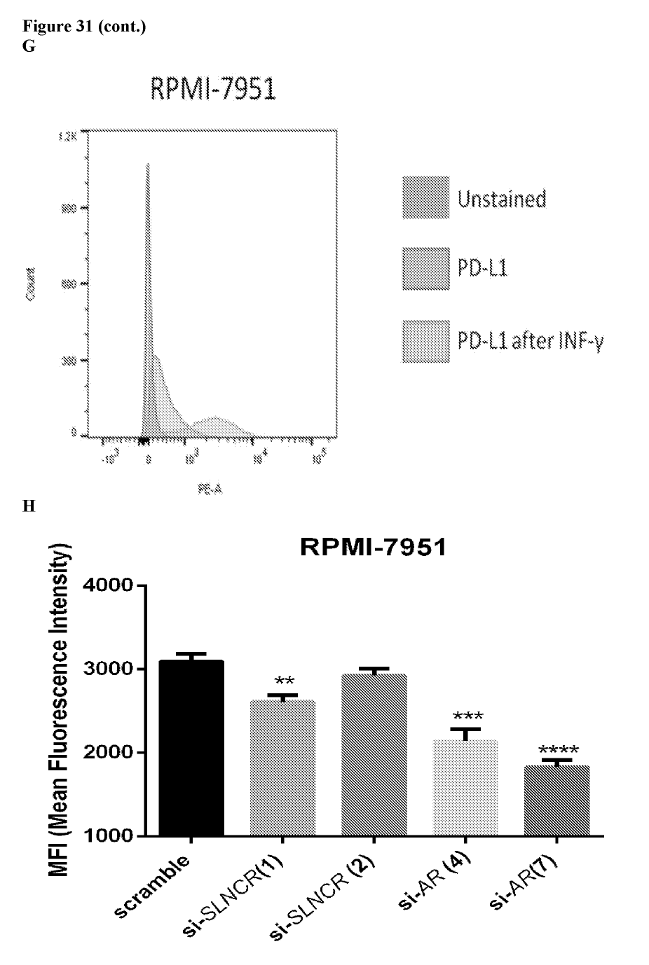

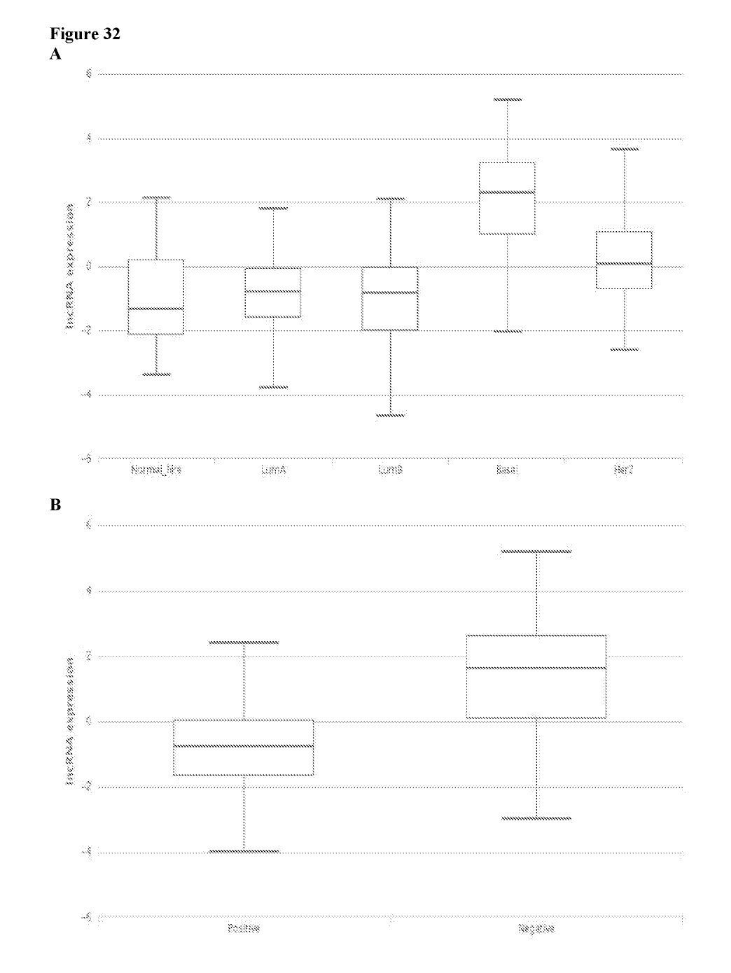

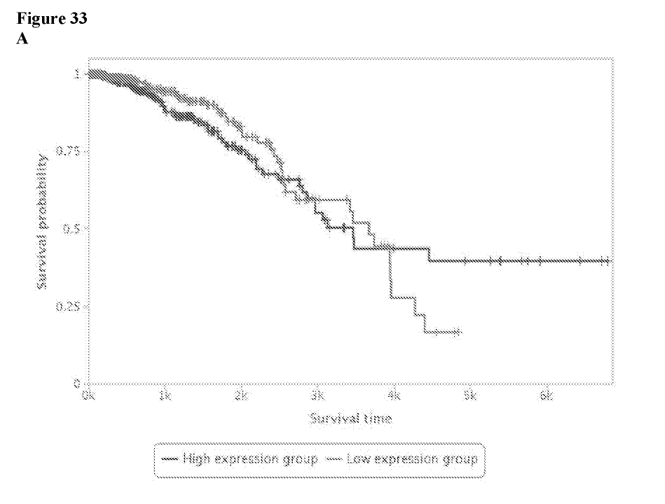

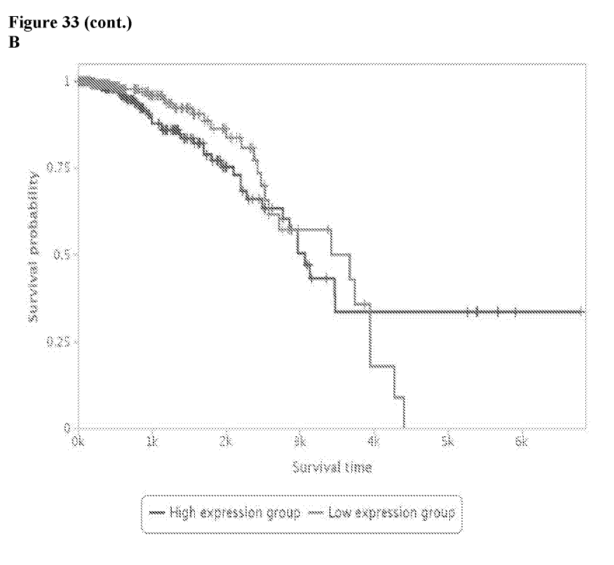

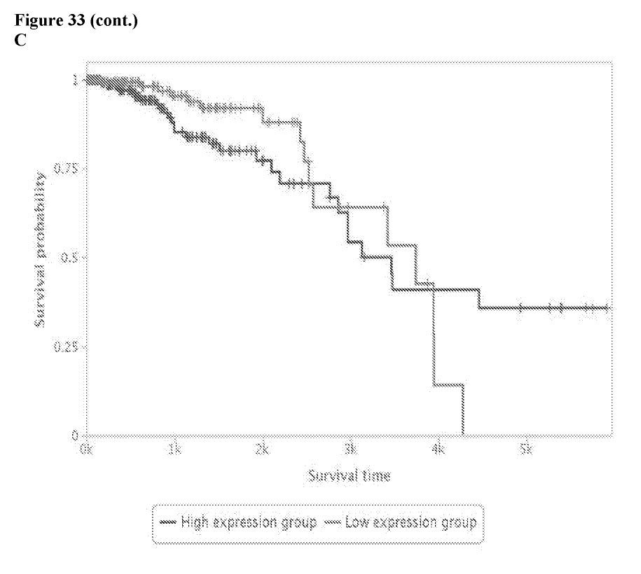

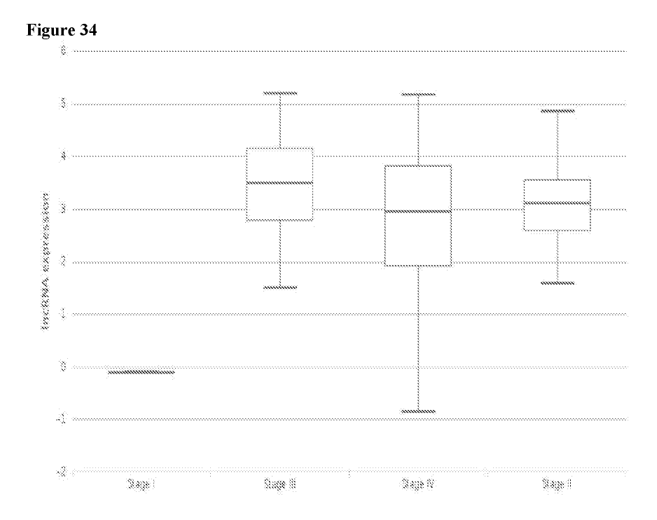

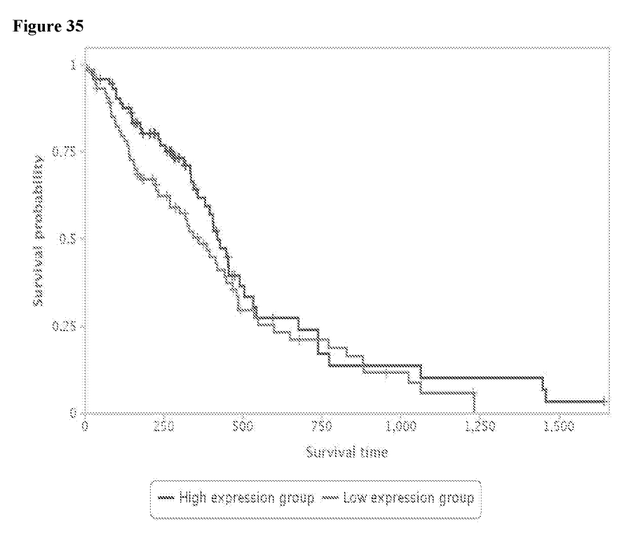

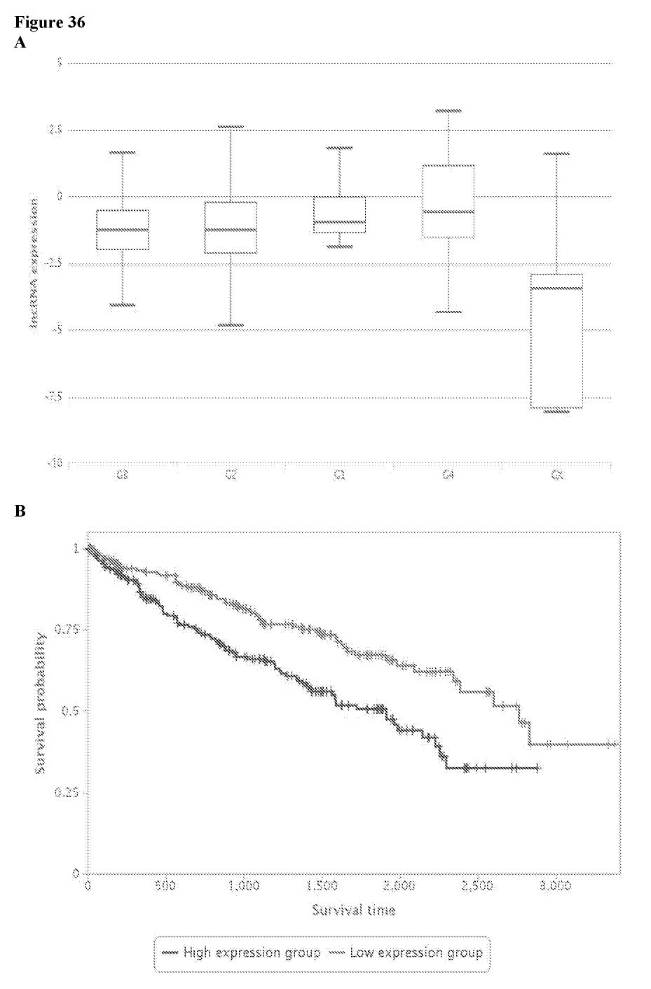

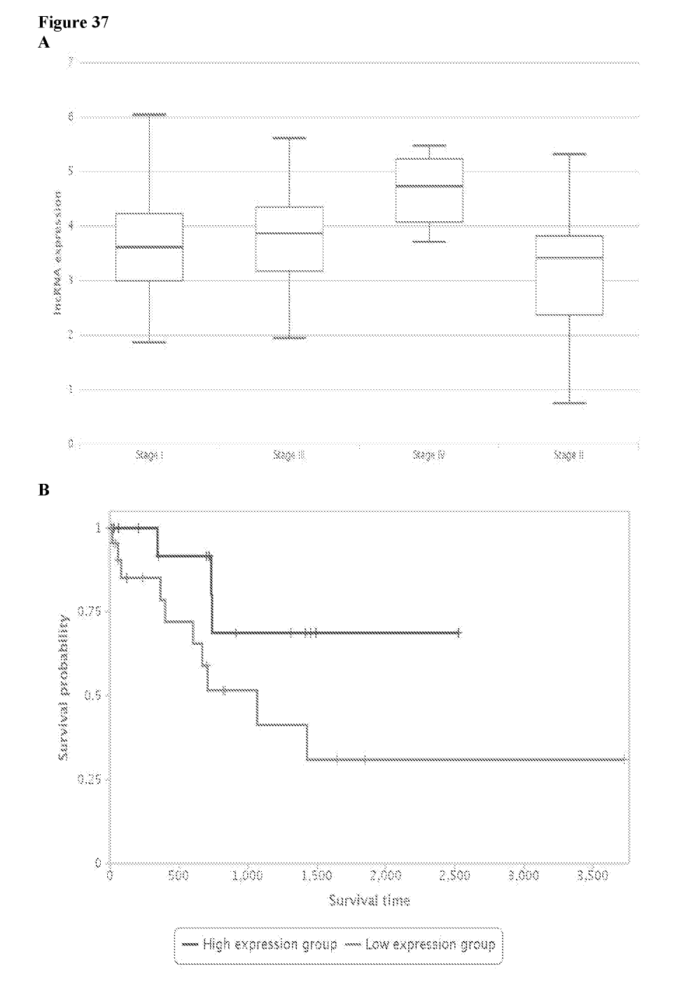

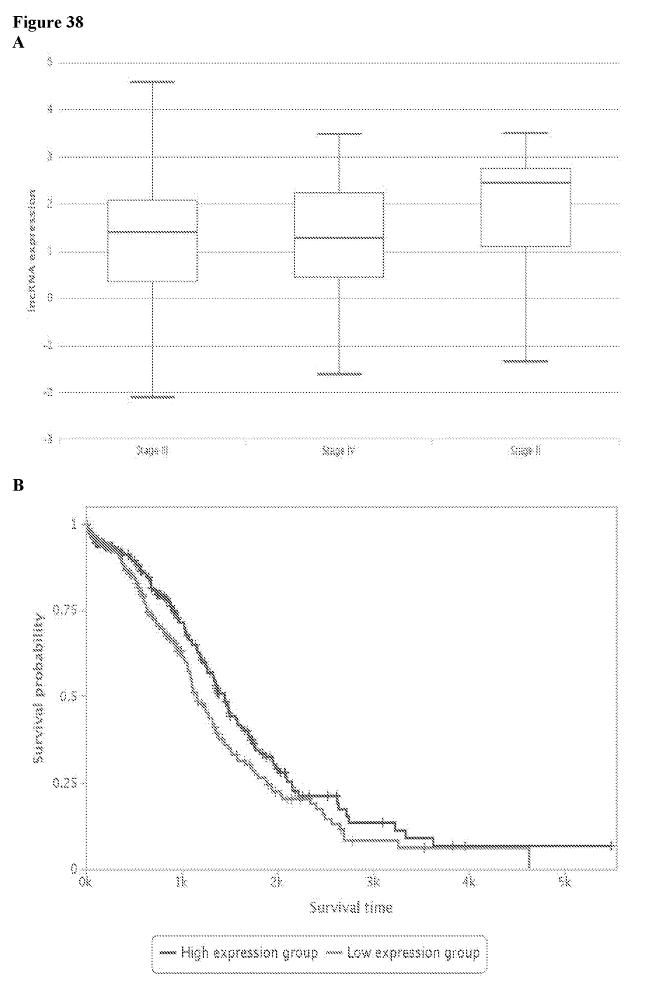

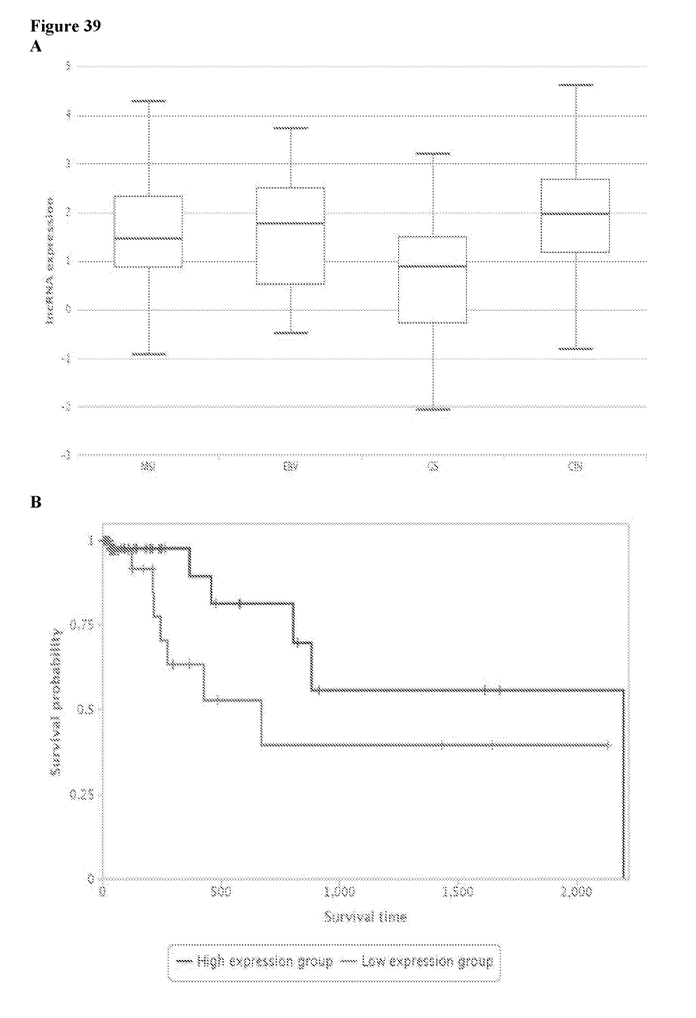

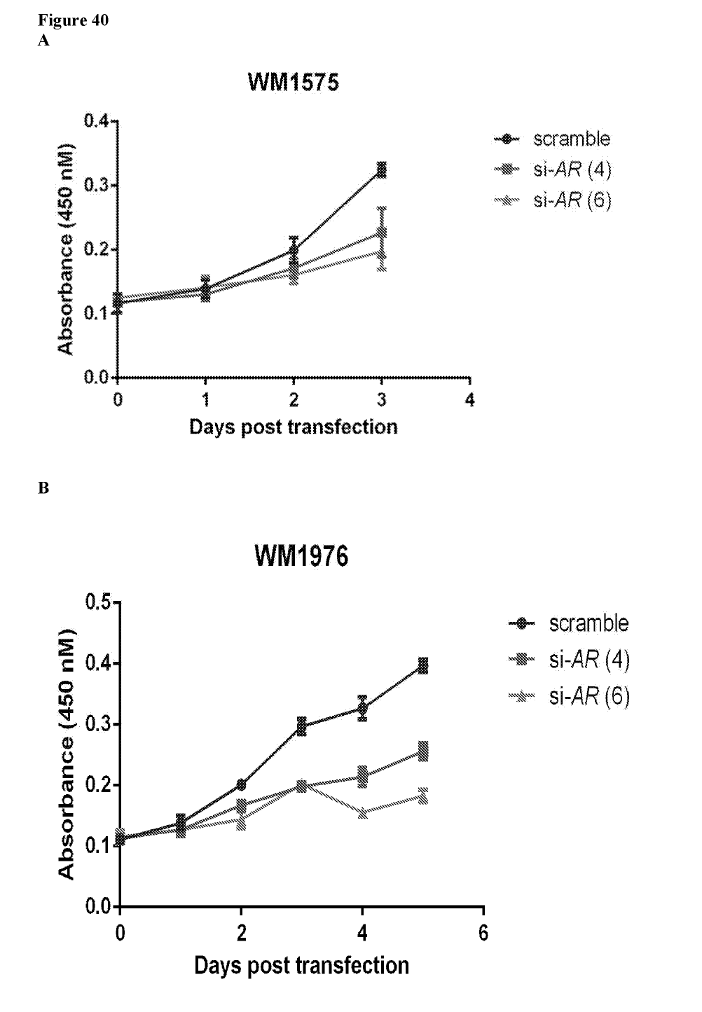

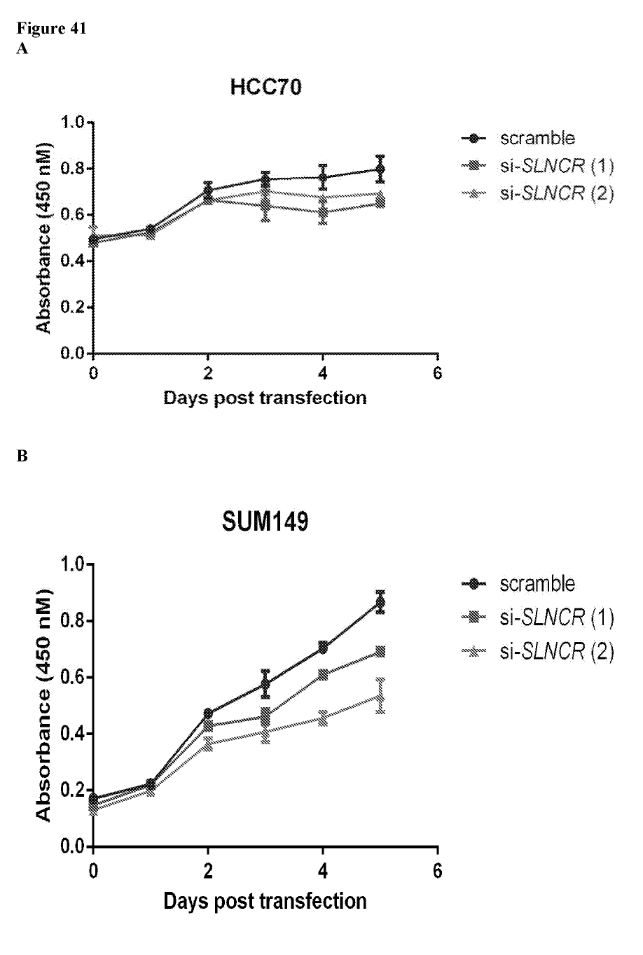

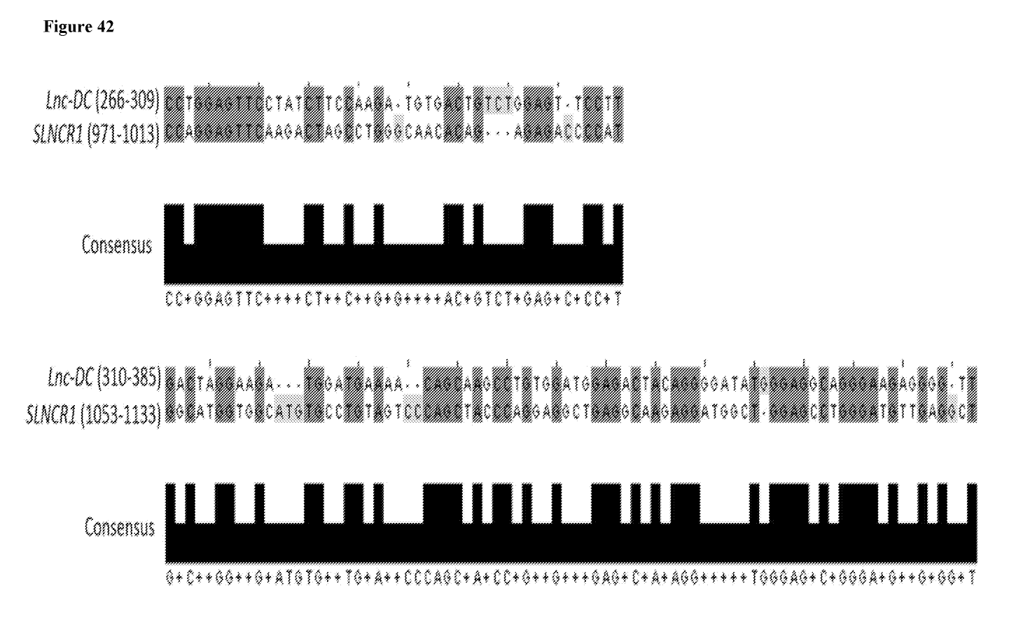

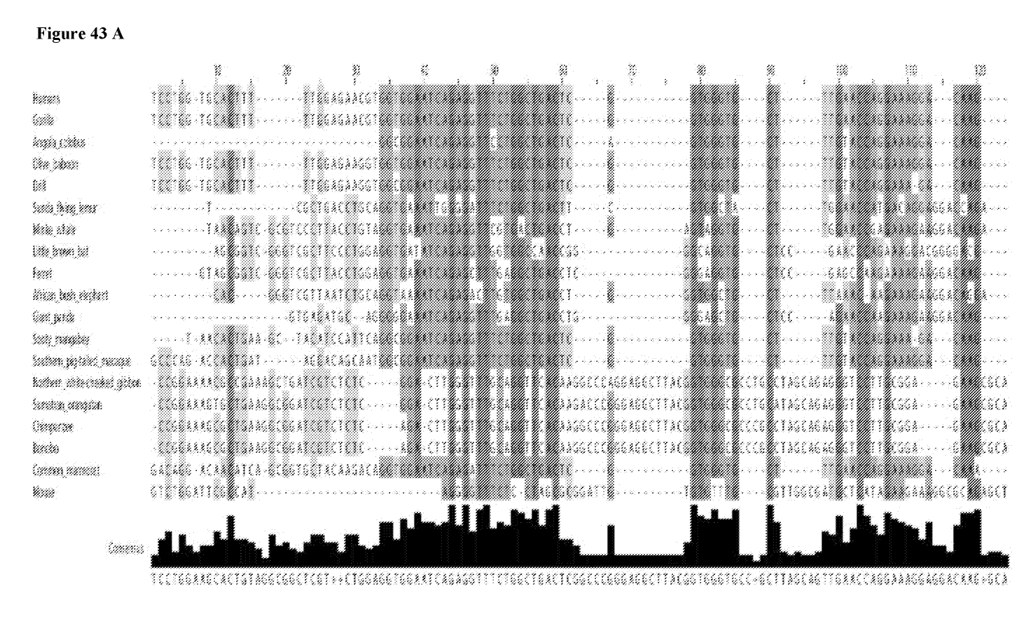

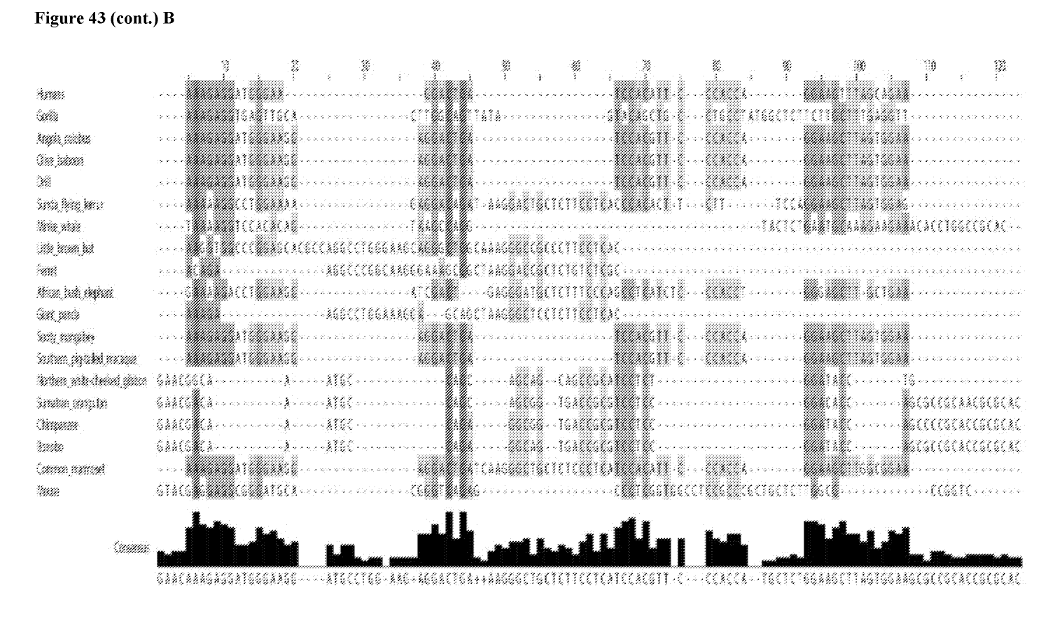

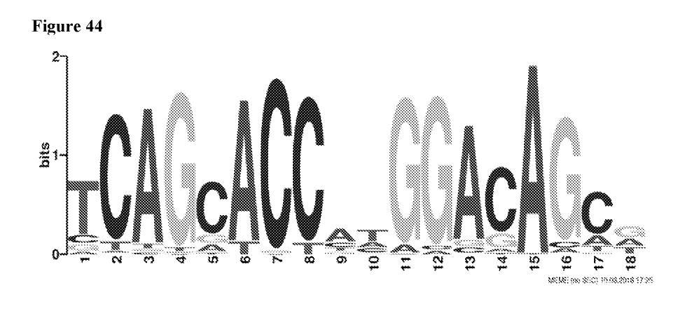

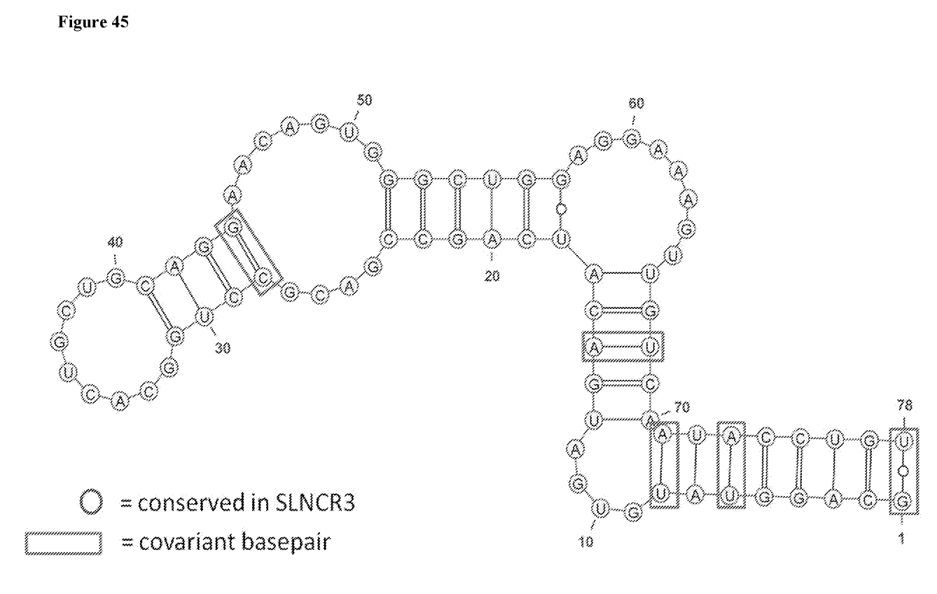

SLNCR and AR regulate expression of PD-L1 (CD274) in melanoma (see FIG. 31, Panels A-D). WM1575, A375, SK-MEL-28 or RPMI-7951 cells were plated at 1×10⁴ cells/well in a 96-well plate, and transfected with the indicated siRNAs (AR siRNAs SI02757265 [4], SI04434178 [6] or SI04434171 [7]; Qiagen) 24 hours later using RNAiMAX. Interferon gamma (if applicable) was added to a final concentration of 100 ng/ml 24 hours post-transfection for WM1575, A375 and SK-MEL-28, or 48 hours post-transfection for RPMI-7951 cells. Cells were incubated for 20 minutes with BD Biosciences PE mouse anti-human CD274 (PD-L1) 48 hours post-transfection (or 72 hours for RPMI-7951 cells) and analyzed on a BD LSRFortessa X-20 analyzer. Histograms represent the number of cells with the given intensity of PE signal for unstained (red), endogenous PD-L1 levels (blue) or INF-γ induced PD-L1 (orange). Bar graphs represent the average mean fluorescence intensity±SD for 3 independent replicates. Significance was calculated using the Student's t-test: * p-value<0.05, ** p-value<0.005, *** p-value<0.0005, and **** p-value<0.00005. The data in FIG. 31 confirm the results from AR ChIP indicating that AR is present on the PD-L1 promoter, and that overexpression of SLNCR1 affects AR occupancy at the promoter.

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. An isolated non-coding nucleic acid molecule selected from the group consisting of:
a) an isolated nucleic acid molecule comprising a sequence having at least 80% identity to the nucleic acid sequence of SEQ ID NO: 1, or a fragment thereof, and does not comprise the sequence of SEQ ID NO: 16;
b) an isolated nucleic acid molecule comprising a sequence having at least 80% identity to the nucleic acid sequence of SEQ ID NO: 1, or a fragment thereof, and comprises at most a sequence having 99% identity to the sequence of SEQ ID NO: 16;
c) an isolated nucleic acid molecule comprising a sequence having at least 80% identity to the nucleic acid sequence of SEQ ID NO: 1 and having at least one of nucleotides G228, A231, T243, C244, T245, C246, C247, A248, T258, C259, T260, C261, C262, and T263, or a fragment thereof, wherein the isolated nucleic acid molecule does not comprise the sequence of SEQ ID NO: 16;
d) an isolated nucleic acid molecule comprising a sequence having at least 80% identity to the nucleic acid sequence of SEQ ID NO: 1 and having at least one of nucleotides G228, A231, T243, C244, T245, C246, C247, A248, T258, C259, T260, C261, C262, and T263, or a fragment thereof, wherein the isolated nucleic acid molecule comprises at most a sequence having 99% identity to the sequence of SEQ ID NO: 16;
e) an isolated nucleic acid molecule comprising a sequence having not more than 61 nucleotide substitutions, deletions, or insertions as compared with the nucleic acid sequence of SEQ ID NO: 1, or fragments thereof, and does not comprise the sequence of SEQ ID NO: 16;
f) an isolated nucleic acid molecule comprising a sequence having not more than 61 nucleotide substitutions, deletions, or insertions as compared with the nucleic acid sequence of SEQ ID NO: 1, or fragments thereof, and comprises at most a sequence having 99% identity to the sequence of SEQ ID NO: 16; and
g) an isolated nucleic acid molecule comprising a sequence having at least 80% identity to the nucleic acid sequence of SEQ ID NO: 22 or 41, or a fragment thereof, and does not comprise the sequence of SEQ ID NO: 16;
optionally wherein (i) the isolated nucleic acid molecule, or fragment thereof, is less than 2,257 nucleotides in length; (ii) the isolated nucleic acid molecule, or fragment thereof, is 301 nucleotides in length or shorter, or is 111 nucleotides in length or shorter; (iii) the isolated nucleic acid molecule, or fragment thereof, is at least 111 nucleotides in length and is less than 2,257 nucleotides in length; (iv) the sequence of the nucleic acid molecule, or fragment thereof, is not derived from a single contiguous locus of a genome; (v) the isolated non-coding nucleic acid molecule, or fragment thereof, is an RNA; and (vi) the isolated non-coding nucleic acid molecule, or fragment thereof, is non-naturally occurring.

2-4. (canceled)

5. The isolated non-coding nucleic acid molecule, or fragment thereof, of claim 1, wherein the isolated nucleic acid molecule, or fragment thereof, comprises a domain selected from the group consisting of an SRA1 H2 helix domain, an SRA1 H3 helix domain, a Brn3a binding domain, an androgen receptor (AR) binding domain, a PXR binding domain, a PAX5 binding domain, an SRA1 H5 helix domain, an SRA1 H6 helix domain, a SLNCR autoregulation domain, a SLNCR cons 2 domain, a SLNCR2 isoform-specific domain, and a SLNCR3 isoform-specific domain;
optionally wherein (i) the isolated nucleic acid molecule, or fragment thereof, comprises a Brn3a binding domain and an androgen receptor (AR) binding domain or (ii) the isolated nucleic acid molecule, or fragment thereof, comprises an SRA H6 helix domain.

6-8. (canceled)

9. The isolated non-coding nucleic acid molecule, or fragment thereof, of claim 1, wherein the isolated non-coding nucleic acid molecule, or fragment thereof, has the ability to:
a) bind to at least one protein transcription factor selected from the group consisting of SRC-1/NCOA-1, PXR/NR1I2, PAX5, EGR-1, AR, E2F-1, CAR/NR1I3, PBX1, ATF2, C/EBP, BRN-3/POU4F1, HNF4, NF-kB, AP2, OCT4/POU5F1, SP1, STAT5, p53, TFIID, SLIRP, STAT3, REST, REST4, and DAX1, optionally wherein the nucleic acid molecule-protein transcription factor complex has the ability to translocate to the nucleus; and/or
b) promote one or more biological activities selected from the group consisting of:
1) the expression or activity of MMP9;
2) downregulation of naturally-occurring SLNCR isoforms;
3) modulation of the expression of one or more genes listed in FIGS. 7, 14, 16, 17, 19, and 31;
4) the expression of PLA2G4C, CT45A6, EGR2, RP11-820L6.1, EGR1, ATF3, VCX3A, SPCS2, FABP5, MAGEA2B, RPL41P1, RPS17, HNRNPA1P10, TXNIP, RPL21P75, EIF3CL, RPL7, CT45A3, GTF2IP1, CDK7, HIST1H1C, CT45A1, BTG2, RPS27, RP11-3P17.3, FDCSP, CITED4, IL34, and PD-L1;
5) cellular proliferation;
6) cell death;
7) cellular migration;
8) genomic replication and/or instability;
9) angiogenesis induction;
10) cellular invasion;
11) cancer metastasis;
12) regulation of immune response and/or immune evasion;
13) modulation of one or more genes listed in Tables S5 and S6 affected by SLNCR overexpression; and
14) binding to one or more of transcription factors selected from the group consisting of SRC-1/NCOA-1, PXR/NR1I2, PAX, EGR-1, AR, E2F-1, CAR/NR1I3, PBX1, ATF2, C/EBP, BRN-3/POU4F1, HNF4, NF-kB, AP2, OCT4/POU5F1, SP1, STAT5, p53, TFIID, SLIRP, STAT3, REST, REST4, and DAX1.

10-19. (canceled)

20. The isolated non-coding nucleic acid molecule, or fragment thereof, of claim 1, further comprising a heterologous nucleic acid sequence, optionally wherein the isolated non-coding nucleic acid molecule, or fragment thereof, is operably linked to a nucleic acid expression promoter.

21. (canceled)

22. A pharmaceutical composition comprising the isolated non-coding nucleic acid molecule, or fragment thereof, of claim 1, and a pharmaceutically acceptable agent selected from the group consisting of excipients, diluents, and carriers, optionally wherein the pharmaceutical composition comprises the isolated non-coding nucleic acid at a purity of at least 75%.

23. (canceled)

24. The pharmaceutical composition of claim 22, wherein the pharmaceutical composition further comprises a nuclear receptor targeting drug,
optionally wherein (i) the nuclear receptor targeting drug is selected from the group consisting of luteinizing hormone-releasing hormone (LHRH) analogs, androgen receptor inhibitors, anti-androgens, hormone blocking drugs, nuclear receptor agonists, nuclear receptor antagonists, selective receptor modulators, selective androgen receptor modulators (SARMs), selective estrogen receptor modulators (SERMs), selective progesterone receptor modulators (SPRMs), selective glucocorticoid receptor agonists (SEGRAs), and selective glucocorticoid receptor modulators (SEGRMs) or (ii) the nuclear receptor target drug is selected from the group consisting of leuprolide (Lupron®, Eligard®), goserelin (Zoladex®), triptorelin (Trelstar®), histrelin (Vantas®), degarelix (Firmagon®), bicalutamide (Casode®), enzalutamide (Xtandi®), flutamide (Eulexin®), nilutamide (Nilandron®), ketoconazole (Nizoral®), abiraterone (Zytiga®), dexamethasone, megestrol acetate (Megace®), medroxyprogesterone acetate (MPA), ethisterone, norethindrone acetate, norethisterone, norethynodrel, ethynodiol diacetate, norethindrone, norgestimate, norgestrel, levonorgestrel, medroxyprogesterone acetate, desogestrel, etonogestrel, drospirenone, norelgestromin, desogestrel, etonogestrel, gestodene, dienogest, drospirenone, elcometrine, nomegestrol acetate, trimegestone, tanaproget, BMS948, mifepristone, 4-hydroxytamoxifen, CINPA1, Cyproterone acetate (Androcur®, Cyprostat®, Siterone®), chlormadinone acetate (Clordion®, Gestafortin®, Lormin®, Non-Ovlon®, Normenon®, Verton®), 17-hydroxyprogesterone (17-OHP), THC, clotrimazole, PK11195 [1-(2-chlorophenyl)-N-methyl-N-(1-methylpropyl)-3-isoquinolinecarboxamide], meclizine, androstanol, CITCO [6-(4-chlorophenyl)imidazo [2,1-b][1,3] thiazole-5-carbaldehyde O-(3,4-dichlorobenzyl) oxime], zearalenone (ZEN), T0901317, 507662, enobosarm, BMS-564,929, LGD-4033, AC-262,356, JNJ-28330835, LGD-2226, LGD-3303, S-40503, S-23, clomifene, femarelle, ormeloxifene, raloxifene, tamoxifen, toremifene, lasofoxifene, ospemifene, afimoxifene, arzoxifene, bazedoxifene, gulvestrant (Faslodex®, ICI-182780), CDB-4124, asoprisnil, proellex, mapracorat (BOL-303242-X, ZK 245186), fosdagrocorat (PF-04171327), ZK 216348, and 55D1E1.

25-26. (canceled)

27. A vector comprising the isolated non-coding nucleic acid molecule, or fragment thereof, of claim 1, or a host cell transfected with said vector.

28-29. (canceled)

30. A method of producing a non-coding nucleic acid molecule comprising culturing the host cell of claim 29 in an appropriate culture medium to, thereby, produce the non-coding nucleic acid molecule,
optionally wherein (i) the host cell is a bacterial cell or a eukaryotic cell or (ii) further comprising isolating the isolated non-coding nucleic acid molecule, or fragment thereof, from the medium or host cell.

31-32. (canceled)

33. A method of treating a subject afflicted with a cancer comprising administering to the subject anti-SLNCR therapy comprising an agent that inhibits the genomic copy number, amount, and/or activity of SLNCR, thereby treating the subject afflicted with the cancer,
optionally wherein
(i) the agent is administered in a pharmaceutically acceptable composition;
(ii) the agent directly binds SLNCR;
(iii) the agent or anti-SLNCR therapy is selected from the group consisting of a small molecule, antisense nucleic acid, interfering RNA, shRNA, siRNA, aptamer, ribozyme, dominant-negative protein, blocking antibody, CRISPR, and combinations thereof, optionally wherein the agent or anti-SLNCR therapy is shRNA or siRNA, antisense oligos (ASO) including RNase-H dependent methods, bicyclic compounds, locked nucleic acids (LNAs), morpholinos, 2′-methyoxyethyl modified nuclei acids, microRNAs, and small molecule inhibitors;
(iv) the at least one biomarker is selected from the group consisting of 1, 2, 3, 4, 5, 6, 7, 8, 9, 10, or more biomarkers;
(v) the at least one biomarker is human SLNCR selected from the group consisting of human SLNCR, human SLNCR2, or human SLNCR3;
(vi) wherein the cancer is selected from the group consisting of melanoma, lung adenocarcinoma, lung squamous cell carcinoma, cervical cancer, ovarian cancer, uterine cancer, pancreatic cancer, colorectal cancer, lower grade glioma, glioblastoma multiforme, breast cancer, endometrial cancer, prostate cancer, testicular cancer, thyroid cancer, osteosarcoma, esophageal cancer, liver cancer and bladder cancer;
(vii) wherein the subject is a mammal;
(viii) wherein the mammal is an animal model of cancer; or
(ix) wherein the mammal is a human.

34-37. (canceled)

38. A method of inhibiting hyperproliferative growth, migration, invasiveness, angiogenesis induction, metastasis, or immune evasion of a cancer cell, or modulating immune responses in a cancer or immune cell, the method comprising contacting the cancer cell or cells with anti-SLNCR therapy comprising an agent that inhibits the genomic copy number, amount, and/or activity of SLNCR, thereby inhibiting hyperproliferative growth, migration, invasiveness, angiogenesis induction, metastasis, or immune evasion of the cancer cell, or modulating immune responses in a cancer or immune cell,
optionally wherein (i) the immune response is upregulated, (ii) the step of contacting occurs in vivo, ex vivo, or in vitro, or (iii) further comprising administering one or more additional anti-cancer agents.

39-43. (canceled)

44. A method of determining whether a subject is afflicted with an invasive or metastatic cancer or at risk for developing an invasive or metastatic cancer comprising:
a) determining the presence, copy number, amount, and/or activity of at least one biomarker listed in Table 1A or 1B in a subject sample;
b) determining the presence, copy number, amount, and/or activity of the at least one biomarker in a control; and
c) comparing the presence, copy number, amount, and/or activity of said at least one biomarker detected in steps a) and b);
wherein the presence or a significant increase in the copy number, amount, and/or activity of the at least one biomarker in the subject sample relative to the control indicates that the subject is afflicted with the invasive or metastatic cancer or at risk for developing the invasive or metastatic cancer,
optionally (i) further comprising recommending, prescribing, or administering an agent that inhibits the copy number, amount, and/or activity of SLNCR if the subject is afflicted with the invasive or metastatic cancer or at risk for developing the invasive or metastatic cancer; (ii) wherein the agent is administered in a pharmaceutically acceptable formulation, the agent directly binds SLNCR, (iii) SLNCR is human SLNCR; (iv) wherein the control sample is determined from a cancerous or non-cancerous sample from either the patient or a member of the same species to which the patient belongs; (v) wherein the cancerous or non-cancerous sample is obtained from the same tissue type as the biological sample; (vi) wherein the control sample comprises cells; or (vii) further comprising determining responsiveness to anti-immune checkpoint inhibitor therapy measured by at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

45-49. (canceled)

50. A method of assessing the efficacy of an agent for treating a cancer in a subject comprising:
a) detecting in a first subject sample and maintained in the presence of the agent the presence, copy number, amount and/or activity of at least one biomarker listed in Table 1A or 1B;
b) detecting the presence, copy number, amount and/or activity of the at least one biomarker listed in Table 1A or 1B in a second subject sample and maintained in the absence of the test compound; and
c) comparing the presence, copy number, amount and/or activity of the at least one biomarker listed in Table 1A or 1B from steps a) and b), wherein the absence or a significantly decreased copy number, amount, and/or activity of the at least one biomarker listed in Table 1A or 1B in the first subject sample relative to the second subject sample, indicates that the agent treats the cancer in the subject.

51. A method of monitoring the progression of a cancer in a subject comprising:
a) detecting in a subject sample at a first point in time the presence, copy number, amount, and/or activity of at least one biomarker listed in Table 1A or 1B;
b) repeating step a) during at least one subsequent point in time after administration of a therapeutic agent; and
c) comparing the presence, copy number, amount, and/or activity detected in steps a) and b), wherein the presence or a significantly increased copy number, amount, and/or activity of the at least one biomarker listed in Table 1 in the first subject sample relative to at least one subsequent subject sample, indicates that the agent treats the cancer in the subject,
optionally, (i) wherein between the first point in time and the subsequent point in time, the subject has undergone treatment, completed treatment, and/or is in remission for the cancer; (ii) wherein between the first point in time and the subsequent point in time, the subject has undergone anti-SLNCR therapy; (iii) wherein the first and/or at least one subsequent sample is selected from the group consisting of ex vivo and in vivo samples; (iv) wherein the first and/or at least one subsequent sample is obtained from an animal model of the cancer, a human model of the cancer, or a primary human cancer; (v) wherein the first and/or at least one subsequent sample is a portion of a single sample or pooled samples obtained from the subject or (vi) further comprising determining the ability of the test agent to bind to the at least one biomarker listed in Table 1A or 1B before or after determining the effect of the test agent on the copy number, level of expression, or level of activity of the at least one biomarker listed in Table 1A or 1B.

52-56. (canceled)

57. A cell-based method for identifying an agent that inhibits a cancer, the method comprising:
a) contacting a cancer cell expressing at least one biomarker listed in Table 1A or 1B with a test agent; and
b) determining the effect of the test agent on the copy number, level of expression, and/or level of activity of the at least one biomarker in Table 1A or 1B to thereby identify an agent that inhibits the cancer, optionally
(i) wherein said cells are isolated from an animal model of a cancer, a human model of the cancer, or a primary human cancer;
(ii) wherein the step of contacting occurs in vivo, ex vivo, or in vitro;
(iii) further comprising determining the ability of the test agent to bind to the at least one biomarker listed in Table 1A or 1B before or after determining the effect of the test agent on the copy number, level of expression, or level of activity of the at least one biomarker listed in Table 1A or 1B;
(iv) wherein the sample comprises cells, cell lines, histological slides, paraffin embedded tissue, fresh frozen tissue, fresh tissue, biopsies, skin, blood, plasma, serum, buccal scrape, saliva, cerebrospinal fluid, urine, stool, mucus, or bone marrow, obtained from the subject;
(v) wherein the presence or copy number is assessed by microarray, quantitative PCR (qPCR), high-throughput sequencing, comparative genomic hybridization (CGH), or fluorescent in situ hybridization (FISH);
(vi) wherein the amount of the at least one biomarker listed in Table 1A or 1B is assessed by detecting the presence in the samples of a polynucleotide molecule encoding the biomarker or a portion of said polynucleotide molecule, optionally wherein the polynucleotide molecule is a mRNA, cDNA, or functional variants or fragments thereof, optionally wherein the step of detecting further comprises amplifying the polynucleotide molecule;
(vii) wherein the amount of the at least one biomarker is assessed by annealing a nucleic acid probe with the sample of the polynucleotide encoding the one or more biomarkers or a portion of said polynucleotide molecule under stringent hybridization conditions;
(viii) wherein the amount of the at least one biomarker is assessed using a reagent which specifically binds with said biomarker, optionally wherein the reagent is selected from the group consisting of a natural protein binding partner, an aptamer, an antibody, an antibody derivative, and an antibody fragment;
(ix) wherein the activity of the at least one biomarker is assessed by determining the magnitude of cellular proliferation, cell death, cellular migration, replication, induction of angiogenesis, cellular invasion/metastasis, immune response, or immune evasion;
(x) wherein the agent or anti-SLNCR therapy is selected from the group consisting of a small molecule, antisense nucleic acid, interfering RNA, shRNA, siRNA, aptamer, ribozyme, dominant-negative protein, blocking antibody, CRISPR, and combinations thereof, optionally wherein the agent or anti-SLNCR therapy is shRNA or siRNA, antisense oligos (ASO) including RNase-H dependent methods, bicyclic compounds, locked nucleic acids (LNAs), morpholinos, 2′-methyoxyethyl modified nuclei acids, microRNAs, and small molecule inhibitors;
(xi) wherein the subject is a mammal, optionally wherein the mammal is an animal model of cancer, or a human;
(xii) wherein the agent or anti-SLNCR therapy further comprises a nuclear receptor targeting drug;
(xiii) wherein the agent or anti-SLNCR therapy further comprises a nuclear receptor targeting drug selected from the group consisting of luteinizing hormone-releasing hormone (LHRH) analogs, androgen receptor inhibitors, anti-androgens, hormone blocking drugs, nuclear receptor agonists, nuclear receptor antagonists, selective receptor modulators, selective androgen receptor modulators (SARMs), selective estrogen receptor modulators (SERMs), selective progesterone receptor modulators (SPRMs), selective glucocorticoid receptor agonists (SEGRAs), and selective glucocorticoid receptor modulators (SEGRMs); or
(xiv) wherein the agent or anti-SLNCR therapy further comprises a nuclear receptor targeting drug selected from the group consisting of leuprolide (Lupron®, Eligard®), goserelin (Zoladex®), triptorelin (Trelstar®), histrelin (Vantas®), degarelix (Firmagon®), bicalutamide (Casode®), enzalutamide (Xtandi®), flutamide (Eulexin®), nilutamide (Nilandron®), ketoconazole (Nizoral®), abiraterone (Zytiga®), dexamethasone, megestrol acetate (Megace®), medroxyprogesterone acetate (MPA), ethisterone, norethindrone acetate, norethisterone, norethynodrel, ethynodiol diacetate, norethindrone, norgestimate, norgestrel, levonorgestrel, medroxyprogesterone acetate, desogestrel, etonogestrel, drospirenone, norelgestromin, desogestrel, etonogestrel, gestodene, dienogest, drospirenone, elcometrine, nomegestrol acetate, trimegestone, tanaproget, BMS948, mifepristone, 4-hydroxytamoxifen, CINPA1, Cyproterone acetate (Androcur®, Cyprostat®, Siterone®), chlormadinone acetate (Clordion®, Gestafortin®, Lormin®, Non-Ovlon®, Normenon®, Verton®), 17-hydroxyprogesterone (17-OHP), THC, clotrimazole, PK11195 [1-(2-chlorophenyl)-N-methyl-N-(1-methylpropyl)-3-isoquinolinecarboxamide], meclizine, androstanol, CITCO [6-(4-chlorophenyl)imidazo [2,1-b][1,3] thiazole-5-carbaldehyde O-(3,4-dichlorobenzyl) oxime], zearalenone (ZEN), T0901317, S07662, enobosarm, BMS-564,929, LGD-4033, AC-262,356, JNJ-28330835, LGD-2226, LGD-3303, S-40503, S-23, clomifene, femarelle, ormeloxifene, raloxifene, tamoxifen, toremifene, lasofoxifene, ospemifene, afimoxifene, arzoxifene, bazedoxifene, gulvestrant (Faslodex®, ICI-182780), CDB-4124, asoprisnil, proellex, mapracorat (BOL-303242-X, ZK 245186), fosdagrocorat (PF-04171327), ZK 216348, and 55D1E1.

58-80. (canceled)


SEQUENCE LISTING (149 sequences)

SEQ ID NO: 1 - DNA, 301 bp, Homo sapiens
gaaggcggccgcctgaggacccccgcccgcgacctccgcgagtctggagcgcagaggacagggtctggctgctctttggccttggatggaaagtggggaattgggtggggggctgcggaccccttaacgtggattacttggtgtgtatcagctgggctcagaagacccacgacctcttctccatccgtggattgatttgttctgcttaacagctgggtcgccaagctggaggtatttttccctctccaccctggtcttctcctgtaacgtgtggccgccttttccagcacggcctcctgcc

SEQ ID NO: 2 - DNA, 2522 bp, Homo sapiens
agggcgcgcaggcggcgcgggtgcgcggtgcggcgctggtatccagaggacgcggtcaccgcctctggcatttgtcgttctgcgcttctccgcaaggaccctctgttaggcaggcgcccaccgtaagcctcccgggccttgtgaacctgcaaacccaagtctgagagacgatccgccttcagcgctttccagcttggcagagaggctttcccggcggggatctttggttggcgctggcgatgcgcggggaagaaaggcgaggagcggcgtccaggctgggtgatgtcccagcacgagtaggcgggatgcgctcgcttggtcctccgggcgcccggtccctgcccgcgtcgcgcgcccacccctggggacgagaaggcggccgcctgaggacccccgcccgcgacctccgcgagtctggagcgcagaggacagggtctggctgctctttggccttggatggaaagtggggaattgggtggggggctgcggaccccttaacgtggattacttggtgtgtatcagctgggctcagaagacccacgacctcttctccatccgtggattgatttgttctgcttaacagctgggtcgccaagctggaggtatttttccctctccaccctggtcttctcctgtaacgtgtggccgccttttccagcacggcctcctgccttcctggtgcactttttggagaacgtggtggaatcagaggtttctggctgactcggtgggtgctttgaaccaggaaaggacaagaaagaggtgccacctcctgagtcctggttcctgcggctccgtcgaatctgcagtgaaggcctttgggaagcgtgggttatgtgggtccaggggtcccatcctcccgtggaggcccttgacctgaggatcactaactcacaccccaccgacttccggcccccaacaagggcagtgttcctcttgctccattctgctctgaagtcccccaaaccgcttcctggggctgcttagtgaaatacaaggctcatctctgaggacctctacagggctggatgggaaggactgatccacattcccaccaggaagtttagcagaacccccgcgtgccacctggaccccttggaaggacctggctcaggctggaccacctcttgagaggcaggagctctggatttgatcaagaattctttgctgagcatggtgcctcatgcctataatcccaacactttgggaggccagtgtgggaggatctcttgagcccaggagttcaagactagcctgggcaacacagagagaccccatctctaaaataataataataataaaataaaaaattagcagggcatggtggcatgtgcctgtagtcccagctacccaggaggctgaggcaagaggatggctggagcctgggatgttgaggctgcaatgaactgtgattaccccactgcactccagcctgggcaaaagagcgagagaccctgtctcaaataataataataataataatcttattttggagaataaagagacctctggatttgaggtgccatttgggtagaaagaaaagacgtttacaccgagaaatagtctgtgttgccctgaaggagcagagggatgcatcgctggaggtgacctacagttgaagaagactcattatgacagaccttgtccttcttccttgtggaaagtgtttcctctgctgctactgctcatgagactcttccccctccctgtcccagggaaccaaagggctttctaccacaccctttcttgccccccgcctcccatgtctgctgtgcctttgtactcagcaattcttgtttgctccattatcttccagccggatacagagtgaatagttaaccacacttaggtcaaataggatctaaatttttgttcctgctccgtgtaaagaggccagtgtttgtgtgttgcaagcagccttggaatagtaactcttctcatttgtttgggatctggccaccaagttccagaatgatacacggatcagtgcagaagttcatcaggctctcggaccttagggctgttggagaaggcttcagcagcagaactgatggtgaaggctcgtgttctccatcctcaactttctttgcttcgatcatacacaagaatacatttggaagggcaaaaaatgaacactgtcgttcattgcagccgtgttttgtgacacagatgcacagtctgctgtgaagaccttctctcaagtggcatttgggagtccatgccagatcatggtgcttcatgagagactgacagctatcaggggttgtggcacttagtgaggactctcctcccccagtgtgtgctgatgacacatacacacctgacaatagcttgagtcttctctgttccttttactctgtagccaacatacacatgatttaaaaccctttctaaatatctatcatggttcatccttgtccaaatgcagagtcagagctatttgtacttcattattatttccaaggcgaatagttggctttctttttgcaaaaataattaaagtttttgtatgttgcagttgc

SEQ ID NO: 3 - DNA, 2625 bp, Homo sapiens
agggcgcgcaggcggcgcgggtgcgcggtgcggcgctggtatccagaggacgcggtcaccgcctctggcatttgtcgttctgcgcttctccgcaaggaccctctgttaggcaggcgcccaccgtaagcctcccgggccttgtgaacctgcaaacccaagtctgagagacgatccgccttcagcgctttccagcttggcagagaggctttcccggcggggatctttggttggcgctggcgatgcgcggggaagaaaggcgaggagcggcgtccaggctgggtgatgtcccagcacgagtaggcgggatgcgctcgcttggtcctccgggcgcccggtccctgcccgcgtcgcgcgcccacccctggggacgagaaggcggccgcctgaggacccccgcccgcgacctccgcgagtctggagcgcagaggacagggtctggctgctctttggccttggatggaaagtggggaattgggtggggggctgcggaccccttaacgtggattacttggtgtgtatcagctgggctcagaagacccacgacctcttctccatccgtggattgatttgttctgcttaacagctgggtcgccaagctggaggtatttttccctctccaccctggtcttctcctgtaacgtgtggccgccttttccagcacggcctcctgccttcctggtgcactttttggagaacgtggtggaatcagaggtttctggctgactcggtgggtgctttgaaccaggaaaggacaagaaagaggaagatcaggttcaagttgccagccagactctgggcttccaggaggagtgggctgtggatggcctggcctcatttgcatgtccctctcctcccggccctgcaggtgccacctcctgagtcctggttcctgcggctccgtcgaatctgcagtgaaggcctttgggaagcgtgggttatgtgggtccaggggtcccatcctcccgtggaggcccttgacctgaggatcactaactcacaccccaccgacttccggcccccaacaagggcagtgttcctcttgctccattctgctctgaagtcccccaaaccgcttcctggggctgcttagtgaaatacaaggctcatctctgaggacctctacagggctggatgggaaggactgatccacattcccaccaggaagtttagcagaacccccgcgtgccacctggaccccttggaaggacctggctcaggctggaccacctcttgagaggcaggagctctggatttgatcaagaattctttgctgagcatggtgcctcatgcctataatcccaacactttgggaggccagtgtgggaggatctcttgagcccaggagttcaagactagcctgggcaacacagagagaccccatctctaaaataataataataataaaataaaaaattagcagggcatggtggcatgtgcctgtagtcccagctacccaggaggctgaggcaagaggatggctggagcctgggatgttgaggctgcaatgaactgtgattaccccactgcactccagcctgggcaaaagagcgagagaccctgtctcaaataataataataataataatcttattttggagaataaagagacctctggatttgaggtgccatttgggtagaaagaaaagacgtttacaccgagaaatagtctgtgttgccctgaaggagcagagggatgcatcgctggaggtgacctacagttgaagaagactcattatgacagaccttgtccttcttccttgtggaaagtgtttcctctgctgctactgctcatgagactcttccccctccctgtcccagggaaccaaagggctttctaccacaccctttcttgccccccgcctcccatgtctgctgtgcctttgtactcagcaattcttgtttgctccattatcttccagccggatacagagtgaatagttaaccacacttaggtcaaataggatctaaatttttgttcctgctccgtgtaaagaggccagtgtttgtgtgttgcaagcagccttggaatagtaactcttctcatttgtttgggatctggccaccaagttccagaatgatacacggatcagtgcagaagttcatcaggctctcggaccttagggctgttggagaaggcttcagcagcagaactgatggtgaaggctcgtgttctccatcctcaactttctttgcttcgatcatacacaagaatacatttggaagggcaaaaaatgaacactgtcgttcattgcagccgtgttttgtgacacagatgcacagtctgctgtgaagaccttctctcaagtggcatttgggagtccatgccagatcatggtgcttcatgagagactgacagctatcaggggttgtggcacttagtgaggactctcctcccccagtgtgtgctgatgacacatacacacctgacaatagcttgagtcttctctgttccttttactctgtagccaacatacacatgatttaaaaccctttctaaatatctatcatggttcatccttgtccaaatgcagagtcagagctatttgtacttcattattatttccaaggcgaatagttggctttctttttgcaaaaataattaaagtttttgtatgttgcagttgc

SEQ ID NO: 4 - DNA, 111 bp, Homo sapiens
aagtggggaattgggtggggggctgcggaccccttaacgtggattacttggtgtgtatcagctgggctcagaagacccacgacctcttctccatccgtggattgatttgtt

SEQ ID NO: 5 - DNA, 13 bp, Homo sapiens
ggacagggtctgg

SEQ ID NO: 6 - DNA, 13 bp, Homo sapiens
gggctgcggaccc

SEQ ID NO: 7 - DNA, 9 bp, Homo sapiens
ggattactt

SEQ ID NO: 8 - DNA, 6 bp, Homo sapiens
tctccw

SEQ ID NO: 9 - DNA, 4 bp, Homo sapiens
ggag

SEQ ID NO: 10 - DNA, 25 bp, Homo sapiens
ttctgcttaacagctgggtcgccaa

SEQ ID NO: 11 - DNA, 42 bp, Homo sapiens
gctggaggtatttttccctctccaccctggtcttctcctgta

SEQ ID NO: 12 - DNA, 316 bp, Homo sapiens
gctgagcatggtgcctcatgcctataatcccaacactttgggaggccagtgtgggaggatctcttgagcccaggagttcaagactagcctgggcaacacagagagaccccatctctaaaataataataataataaaataaaaaattagcagggcatggtggcatgtgcctgtagtcccagctacccaggaggctgaggcaagaggatggctggagcctgggatgttgaggctgcaatgaactgtgattaccccactgcactccagcctgggcaaaagagcgagagaccctgtctcaaataataataataataataa

SEQ ID NO: 13 - DNA, 267 bp, Homo sapiens
tgccacctcctgagtcctggttcctgcggctccgtcgaatctgcagtgaaggcctttgggaagcgtgggttatgtgggtccaggggtcccatcctcccgtggaggcccttgacctgaggatcactaactcacaccccaccgacttccggcccccaacaagggcagtgttcctcttgctccattctgctctgaagtcccccaaaccgcttcctggggctgcttagtgaaatacaaggctcatctctgaggacctctacagggctggat

SEQ ID NO: 14 - DNA, 106 bp, Homo sapiens
aggaagatcaggttcaagttgccagccagactctgggcttccaggaggagtgggctgtggatggcctggcctcatttgcatgtccctctcctcccggccctgcagg

SEQ ID NO: 15 - DNA, 73 bp, Homo sapiens
tggtggaatcagaggtttctggctgactcggtgggtgctttgaaccaggaaaggacaagaaagaggatgggaa

SEQ ID NO: 16 - DNA, 2275 bp, Homo sapiens
agggcgcgcaggcggcgcgggtgcgcggtgcggcgctggtatccagaggacgcggtcaccgcctctggcatttgtcgttctgcgcttctccgcaaggaccctctgttaggcaggcgcccaccgtaagcctcccgggccttgtgaacctgcaaacccaagtctgagagacgatccgccttcagcgctttccagcttggcagagaggctttcccggcggggatctttggttggcgctggcgatgcgcggggaagaaaggcgaggagcggcgtccaggctgggtgatgtcccagcacgagtaggcgggatgcgctcgcttggtcctccgggcgcccggtccctgcccgcgtcgcgcgcccacccctggggacgagaaggcggccgcctgaggacccccgcccgcgacctccgcgagtctggagcgcagaggacagggtctggctgctctttggccttggatggaaagtggggaattgggtggggggctgcggaccccttaacgtggattacttggtgtgtatcagctgggctcagaagacccacgacctcttctccatccgtggattgatttgttctgcttaacagctgggtcgccaagctggaggtatttttccctctccaccctggtcttctcctgtaacgtgtggccgccttttccagcacggcctcctgccttcctggtgcactttttggagaacgtggtggaatcagaggtttctggctgactcggtgggtgctttgaaccaggaaaggacaagaaagaggatgggaaggactgatccacattcccaccaggaagtttagcagaacccccgcgtgccacctggaccccttggaaggacctggctcaggctggaccacctcttgagaggcaggagctctggatttgatcaagaattctttgctgagcatggtgcctcatgcctataatcccaacactttgggaggccagtgtgggaggatctcttgagcccaggagttcaagactagcctgggcaacacagagagaccccatctctaaaataataataataataaaataaaaaattagcagggcatggtggcatgtgcctgtagtcccagctacccaggaggctgaggcaagaggatggctggagcctgggatgttgaggctgcaatgaactgtgattaccccactgcactccagcctgggcaaaagagcgagagaccctgtctcaaataataataataataataatcttattttggagaataaagagacctctggatttgaggtgccatttgggtagaaagaaaagacgtttacaccgagaaatagtctgtgttgccctgaaggagcagagggatgcatcgctggaggtgacctacagttgaagaagactcattatgacagaccttgtccttcttccttgtggaaagtgtttcctctgctgctactgctcatgagactcttccccctccctgtcccagggaaccaaagggctttctaccacaccctttcttgccccccgcctcccatgtctgctgtgcctttgtactcagcaattcttgtttgctccattatcttccagccggatacagagtgaatagttaaccacacttaggtcaaataggatctaaatttttgttcctgctccgtgtaaagaggccagtgtttgtgtgttgcaagcagccttggaatagtaactcttctcatttgtttgggatctggccaccaagttccagaatgatacacggatcagtgcagaagttcatcaggctctcggaccttagggctgttggagaaggcttcagcagcagaactgatggtgaaggctcgtgttctccatcctcaactttctttgcttcgatcatacacaagaatacatttggaagggcaaaaaatgaacactgtcgttcattgcagccgtgttttgtgacacagatgcacagtctgctgtgaagaccttctctcaagtggcatttgggagtccatgccagatcatggtgcttcatgagagactgacagctatcaggggttgtggcacttagtgaggactctcctcccccagtgtgtgctgatgacacatacacacctgacaatagcttgagtcttctctgttccttttactctgtagccaacatacacatgatttaaaaccctttctaaatatctatcatggttcatccttgtccaaatgcagagtcagagctatttgtacttcattattatttccaaggcgaatagttggctttctttttgcaaaaataattaaagtttttgtatgttgcagttgcaaaaaaaaaaaaaaaaaa

SEQ ID NO: 17 - RNA, 20 nt, Artificial Sequence
  Description of Artificial Sequence Synthetic oligonucleotide
ucucucucucucucucucuc

SEQ ID NO: 18 - RNA, 20 nt, Artificial Sequence
  Description of Artificial Sequence Synthetic oligonucleotide
uucuuucuuucuuucuuucu

SEQ ID NO: 19 - RNA, 20 nt, Artificial Sequence
  Description of Artificial Sequence Synthetic oligonucleotide
cccucccucccucccucccu

SEQ ID NO: 20 - RNA, 41 nt, Artificial Sequence
  Description of Artificial Sequence Synthetic oligonucleotide
cuggagguauuuuucccucuccacccuggucuucuccugua

SEQ ID NO: 21 - RNA, 26 nt, Artificial Sequence
  Description of Artificial Sequence Synthetic oligonucleotide
caggaggugacccucgucuucuccug

SEQ ID NO: 22 - RNA, 28 nt, Artificial Sequence
  Description of Artificial Sequence Synthetic oligonucleotide
uucccucuccacccuggucuucuccugu

SEQ ID NO: 23 - RNA, 11 nt, Artificial Sequence
  Description of Artificial Sequence Synthetic oligonucleotide
uucccucucca

SEQ ID NO: 24 - RNA, 10 nt, Artificial Sequence
  Description of Artificial Sequence Synthetic oligonucleotide
cuucuccugu

SEQ ID NO: 25 - RNA, 17 nt, Artificial Sequence
  Description of Artificial Sequence Synthetic oligonucleotide
uauuuuucccucuccac

SEQ ID NO: 26 - RNA, 22 nt, Artificial Sequence
  Description of Artificial Sequence Synthetic oligonucleotide
uggagguauuuuucccucucca

SEQ ID NO: 27 - RNA, 24 nt, Artificial Sequence
  Description of Artificial Sequence Synthetic oligonucleotide
cuggagguauuuuucccucuccag

SEQ ID NO: 28 - RNA, 28 nt, Artificial Sequence
  Description of Artificial Sequence Synthetic oligonucleotide
aucgcucucaacccuggucuucuccugu

SEQ ID NO: 29 - RNA, 28 nt, Artificial Sequence
  Description of Artificial Sequence Synthetic oligonucleotide
uucccucuccacccugguagucucaggu

SEQ ID NO: 30 - RNA, 28 nt, Artificial Sequence
  Description of Artificial Sequence Synthetic oligonucleotide
aucgcucucaacccugguagucucaggu

SEQ ID NO: 31 - RNA, 28 nt, Artificial Sequence
  Description of Artificial Sequence Synthetic oligonucleotide
uccuuuccucacccugguucucuuccgu

SEQ ID NO: 32 - RNA, 28 nt, Artificial Sequence
  Description of Artificial Sequence Synthetic oligonucleotide
uucccucuccagcauggucuucuccugu

SEQ ID NO: 33 - RNA, 20 nt, Artificial Sequence
  Description of Artificial Sequence Synthetic oligonucleotide
uauuuuucccucuccacccu

SEQ ID NO: 34 - RNA, 20 nt, Artificial Sequence
  Description of Artificial Sequence Synthetic oligonucleotide
uauuuuucccuucccacccu

SEQ ID NO: 35 - RNA, 25 nt, Artificial Sequence
  Description of Artificial Sequence Synthetic oligonucleotide
uccccgcaucagagacuucuccugg

SEQ ID NO: 36 - DNA, 20 bp, Artificial Sequence
  Description of Artificial Sequence Synthetic oligonucleotide
tctctctctctctctctctc

SEQ ID NO: 37 - DNA, 20 bp, Artificial Sequence
  Description of Artificial Sequence Synthetic oligonucleotide
ttctttctttctttctttct

SEQ ID NO: 38 - DNA, 20 bp, Artificial Sequence
  Description of Artificial Sequence Synthetic oligonucleotide
ccctccctccctccctccct

SEQ ID NO: 39 - DNA, 41 bp, Artificial Sequence
  Description of Artificial Sequence Synthetic oligonucleotide
ctggaggtatttttccctctccaccctggtcttctcctgta

SEQ ID NO: 40 - DNA, 26 bp, Artificial Sequence
  Description of Artificial Sequence Synthetic oligonucleotide
caggaggtgaccctcgtcttctcctg

SEQ ID NO: 41 - DNA, 28 bp, Artificial Sequence
  Description of Artificial Sequence Synthetic oligonucleotide
ttccctctccaccctggtcttctcctgt

SEQ ID NO: 42 - DNA, 11 bp, Artificial Sequence
  Description of Artificial Sequence Synthetic oligonucleotide
ttccctctcca

SEQ ID NO: 43 - DNA, 10 bp, Artificial Sequence
  Description of Artificial Sequence Synthetic oligonucleotide
cttctcctgt

SEQ ID NO: 44 - DNA, 17 bp, Artificial Sequence
  Description of Artificial Sequence Synthetic oligonucleotide
tatttttccctctccac

SEQ ID NO: 45 - DNA, 22 bp, Artificial Sequence
  Description of Artificial Sequence Synthetic oligonucleotide
tggaggtatttttccctctcca

SEQ ID NO: 46 - DNA, 24 bp, Artificial Sequence
  Description of Artificial Sequence Synthetic oligonucleotide
ctggaggtatttttccctctccag

SEQ ID NO: 47 - DNA, 28 bp, Artificial Sequence
  Description of Artificial Sequence Synthetic oligonucleotide
atcgctctcaaccctggtcttctcctgt

SEQ ID NO: 48 - DNA, 28 bp, Artificial Sequence
  Description of Artificial Sequence Synthetic oligonucleotide
ttccctctccaccctggtagtctcaggt

SEQ ID NO: 49 - DNA, 28 bp, Artificial Sequence
  Description of Artificial Sequence Synthetic oligonucleotide
atcgctctcaaccctggtagtctcaggt

SEQ ID NO: 50 - DNA, 28 bp, Artificial Sequence
  Description of Artificial Sequence Synthetic oligonucleotide
tcctttcctcaccctggttctcttccgt

SEQ ID NO: 51 - DNA, 28 bp, Artificial Sequence
  Description of Artificial Sequence Synthetic oligonucleotide
ttccctctccagcatggtcttctcctgt

SEQ ID NO: 52 - DNA, 20 bp, Artificial Sequence
  Description of Artificial Sequence Synthetic oligonucleotide
tatttttccctctccaccct

SEQ ID NO: 53 - DNA, 20 bp, Artificial Sequence
  Description of Artificial Sequence Synthetic oligonucleotide
tatttttcccttcccaccct

SEQ ID NO: 54 - DNA, 25 bp, Artificial Sequence
  Description of Artificial Sequence Synthetic oligonucleotide
tccccgcatcagagacttctcctgg

SEQ ID NO: 55 - DNA, 21 bp, Artificial Sequence
  Description of Artificial Sequence Synthetic oligonucleotide
aagaggatgggaaggactgat

SEQ ID NO: 56 - DNA, 21 bp, Artificial Sequence
  Description of Artificial Sequence Synthetic oligonucleotide
ctgatgggaaggactgatcca

SEQ ID NO: 57 - DNA, 21 bp, Artificial Sequence
  Description of Artificial Sequence Synthetic oligonucleotide
gggctgcttagtgaaatacaa

SEQ ID NO: 58 - DNA, 21 bp, Artificial Sequence
  Description of Artificial Sequence Synthetic oligonucleotide
ctccgtcgaatctgcagtgaa

SEQ ID NO: 59 - RNA, 62 nt, Homo sapiens
ucagguucaaguugccagccagacucugggcuuccaggaggagugggcuguggauggccugg

SEQ ID NO: 60 - DNA, 21 bp, Artificial Sequence
  Description of Artificial Sequence Synthetic oligonucleotide
ttaggtcaaataggatctaaa

SEQ ID NO: 61 - DNA, 21 bp, Artificial Sequence
  Description of Artificial Sequence Synthetic oligonucleotide
aaagacgtttacaccgagaaa

SEQ ID NO: 62 - DNA, 21 bp, Artificial Sequence
  Description of Artificial Sequence Synthetic oligonucleotide
cagcgggaactgtacagtcaa

SEQ ID NO: 63 - DNA, 21 bp, Artificial Sequence
  Description of Artificial Sequence Synthetic oligonucleotide
ctcctaatatttcgacattaa

SEQ ID NO: 64 - DNA, 11 bp, Unknown
  Description of Unknown consensus Brn3a binding site
gcatwawtwat

SEQ ID NO: 65 - DNA, 148 bp, Homo sapiens
ctttggccttggatggaaagtggggaattgggtggggggctgcggaccccttaacgtggattacttggtgtgtatcagctgggctcagaagacccacgacctcttctccatccgtggattgatttgttctgcttaacagctgggtcgc

SEQ ID NO: 66 - DNA, 148 bp, Gorilla gorilla
ctttggccttggatggaaagtggggaattgggtggggggctgcggaccccttaacgtggattacttggtgtgtatcagctgggctcagaagacccacgacctcttctccatccgtgaattgatttgttctgcttaacagctgggtcgc

SEQ ID NO: 67 - DNA, 148 bp, Colobus angolensis
ctttggccttggatggaaagtggggaattgggtgcggggctgcggatcccttaacgtggattacttggtgtgtatcagctgggctcagaagacccacgacctcctctccgtccgtggattgattcgttctacgtaacagctgggtcgc

SEQ ID NO: 68 - DNA, 148 bp, Papio anubis
ctttggccttggatggaaagtggggaattgggtggggggctgcggacctcttaacgtggattacttggtgtgtatcagctgggctcagaagacccacgacctcctctccgcgcgtggattgattcgttctacttaacagctgggtcgc

SEQ ID NO: 69 - DNA, 148 bp, Mandrillus leucophaeus
ctttggccttggatggaaagtggggaattgggtggggggctgcggaccccttaacgtggattacttggtgtgtatcagctgggctcagaagacccacgacctcctctgcgtccgtggattgattcgttctacttaacagctgggtcgc

SEQ ID NO: 70 - DNA, 122 bp, Galeopterus variegatus
ctttggccttggagagaaacatggggaacggggcggggggcttcggaccccctaacgtggattacttggtgcgtatcagccgggatcagaagagccacgacctcgtctcccttcgtgcgttg

SEQ ID NO: 71 - DNA, 126 bp, Balaenoptera acutorostrata
ctttggccttggatggaaactggggaatggggtaggggcctccggaccccctaacgtggattacttggtgcgtctcggccgggctcggatgacccacaacctcgtctccatccgtggattgatgca

SEQ ID NO: 72 - DNA, 127 bp, Myotis lucifugus
ctttgaccttggatggcaagtgggcagtggggtgggtgctccgggcgcctaatgtggattacttggtgtgtaccagccgggctcggaagactcgcgaccccgtctccggccgtggactgattcattc

SEQ ID NO: 73 - DNA, 114 bp, Mustela putorius furo
gccttggatggaaagtggggaagggctccggacccctaacgtggattacttggtgtgtgtcagccgggctcagaagacccacgacctcgtctccatgcttggatcgattcattc

SEQ ID NO: 74 - DNA, 106 bp, Loxodonta africana
ctttgccgtggatggaaagcagggaatgagtgggggcttccggacgcccctaacgtggattacttggtgcggatcgacggagcttggaagacccacgatttcctcc

SEQ ID NO: 75 - DNA, 111 bp, Ailuropoda melanoleuca
cgctgacctaggagtccgggtagggaattttttcccccaggggaaacccccagaaaggcacaccagggaagagagggaaaagcctgctgcctggccacagtgctggttttg

SEQ ID NO: 76 - DNA, 129 bp, Cercocebus atys
gattttccagctgtccaggggagggggcagggatttccatgagtagctaggtctgagagcctcggttgtaagcctctgcctcccctctttcgcctattccgcactgtaggatttcaaaaggatccctct

SEQ ID NO: 77 - DNA, 104 bp, Macaca nemestrina
gtttgccagcttctgatccaggcaggagcagagtgcatatgcccttctttacctgtctattcccccagcgatggacacctacattgtcccaaactctttggcgc

SEQ ID NO: 78 - DNA, 102 bp, Nomascus leucogenys
tccccaggagtgggcgcgcgacgcgggcggggaccgggcgcctcgcggaccatgcgagcgcatcccgcctcctcgtgctgggacagcacccagcctggacgc

SEQ ID NO: 79 - DNA, 102 bp, Pongo abelii
tccccaggggtgggcgcgcgacgcgggcggggacggggcgccccgcggaccatgcgagcgcatcccgcctactcgcgctgggacagcatccagcctggacgc

SEQ ID NO: 80 - DNA, 102 bp, Pan troglodytes
tccccaggggtgggcgcgcgacgcgggcggggaccgggcgccccgcggaccatgcgagcgcatcccgcctactcgtgctgggacatcacccagcctggacgc

SEQ ID NO: 81 - DNA, 102 bp, Pan paniscus; identical to SEQ ID NO: 80

SEQ ID NO: 82 - DNA, 58 bp, Callithrix jacchus
gagaggccaggggtggtgctttgtgtatttaaacagtgttgactgggtcctgccgcac

SEQ ID NO: 83 - DNA, 72 bp, Mus musculus
tggcgcagaaagtgttcacttcttctgtttcgggctgtacgctgtggttctgcgcctccagggctttgctgc

SEQ ID NO: 84 - DNA, 232 bp, Homo sapiens
tgctctttggccttggatggaaagtggggaattgggtggggggctgcggaccccttaacgtggattacttggtgtgtatcagctgggctcagaagacccacgacctcttctccatccgtggattgatttgttctgcttaacagctgggtcgccaagctggaggtatttttccctctccaccctggtcttctcctgtaacgtgtggccgccttttccagcacggcctcctgcc

SEQ ID NO: 85 - DNA, 226 bp, Homo sapiens
cgcttggcggagctgtacgtgaagccgggcaacaaggaacgcggctggaacgacccgccgcagttctcatacgggctgcagacccaggccggcggacccaggcgctcgctgcttaccaagagggtcgccgcaccccaggatggatcccccagagtccccgcatcagagacttctcctgggcctcccccaatggggcctccacctccttcaagtaaggctcccaggt

SEQ ID NO: 86 - DNA, 62 bp, Homo sapiens
attacttggtgtgtatcagctgggctcagaagacccacgacctcttctccatccgtggattg

SEQ ID NO: 87 - DNA, 62 bp, Gorilla gorilla
attacttggtgtgtatcagctgggctcagaagacccacgacctcttctccatccgtgaattg

SEQ ID NO: 88 - DNA, 62 bp, Papio anubis
attacttggtgtgtatcagctgggctcagaagacccacgacctcctctccgcgcgtggattg

SEQ ID NO: 89 - DNA, 62 bp, Pan troglodytes
ctggagcgcagaggacaggctgggctccgaagacccacgacctcttctccatccgtggattg

SEQ ID NO: 90 - DNA, 62 bp, Balaenoptera acutorostrata scammoni
attacttggtgcgtctcggccgggctcggatgacccacaacctcgtctccatccgtggattg

SEQ ID NO: 91 - DNA, 62 bp, Mus musculus
attacttggtgcttatcaactggatttagaagactctcgacctggtctccatcatccgtggg

SEQ ID NO: 92 - DNA, 111 bp, Homo sapiens; identical to SEQ ID NO: 4

SEQ ID NO: 93 - DNA, 60 bp, Artificial Sequence
  Description of Artificial Sequence Synthetic oligonucleotide
gatatgaaattctctgtctctgtaaaagcatttcatatttacaagacacaggcctactcc

SEQ ID NO: 94 - RNA, 29 nt, Homo sapiens
ccccuuaacguggauuacuugguguguau

SEQ ID NO: 95 - RNA, 34 nt, Homo sapiens
uuuuucccucuccacccuggucuucuccuguaac

SEQ ID NO: 96 - RNA, 79 nt, Homo sapiens
gcgcucgcugcuuaccaagagggucgccgcaccccaggauggaucccccagaguccccgcaucagagacuucuccuggg

SEQ ID NO: 97 - DNA, 67 bp, Artificial Sequence
  Description of Artificial Sequence Synthetic polynucleotide
  Description of Combined DNA/RNA Molecule Synthetic polynucleotide
uucugcuuaacagcugggucgccaagctggagguauuuuucccucuccacccuggucuucuccugua

SEQ ID NO: 98 - DNA, 390 bp, Homo sapiens
atcctcaactttctttgcttcgatcatacacaagaatacatttggaagggcaaaaaatgaacactgtcgttcattgcagccgtgttttgtgacacagatgcacagtctgctgtgaagaccttctctcaagtggcatttgggagtccatgccagatcatggtgcttcatgagagactgacagctatcaggggttgtggcacttagtgaggactctcctcccccagtgtgtgctgatgacacatacacacctgacaatagcttgagtcttctctgttccttttactctgtagccaacatacacatgatttaaaaccctttctaaatatctatcatggttcatccttgtccaaatgcagagtcagagctatttgtacttcattattatttcca

SEQ ID NO: 99 - DNA, 176 bp, Epstein-Barr Virus
aggacagccgttgccctagtggtttcggacacaccgccaacgctcagtgcggtgctaccgacccgaggtcaagtcccgggggaggagaagagaggcttcccgcctagagcatttgcaagtcaggattctctaatccctctgggagaagggtattcggcttgtccgctgtttttttt

SEQ ID NO: 100 - RNA, 23 nt, Homo sapiens
uugcagccguguuuugugacaca

SEQ ID NO: 101 - DNA, 11 bp, Homo sapiens
gcattattatt

SEQ ID NO: 102 - DNA, 11 bp, Artificial Sequence
  Description of Artificial Sequence Synthetic oligonucleotide
gcactatgatt

SEQ ID NO: 103 - DNA, 28 bp, Homo sapiens
ttccctctccaccctggtcttctcctgt

SEQ ID NO: 104 - DNA, 31 bp, Homo sapiens
ttccagcctccaggccctgccttctgcctgc

SEQ ID NO: 105 - DNA, 25 bp, Homo sapiens
tccccgcatcagagacttctcctgg

SEQ ID NO: 106 - DNA, 25 bp, Homo sapiens
ttacaagacacaggcctactcctag

SEQ ID NO: 107 - DNA, 44 bp, Homo sapiens
cctggagttcctatcttccaagatgtgactgtctggagttcctt

SEQ ID NO: 108 - DNA, 43 bp, Homo sapiens
ccaggagttcaagactagcctgggcaacacagagagaccccat

SEQ ID NO: 109 - DNA, 76 bp, Homo sapiens
gactaggaagatggatgaaaacagcaagcctgtggatggagactacaggggatatgggaggcagggaagaggggtt

SEQ ID NO: 110 - DNA, 81 bp, Homo sapiens
ggcatggtggcatgtgcctgtagtcccagctacccaggaggctgaggcaagaggatggctggagcctgggatgttgaggct

SEQ ID NO: 111 - DNA, 83 bp, Homo sapiens
tcctggtgcactttttggagaacgtggtggaatcagaggtttctggctgactcggtgggtgctttgaaccaggaaaggacaag

SEQ ID NO: 112 - DNA, 83 bp, Gorilla gorilla; identical to SEQ ID NO: 111

SEQ ID NO: 113 - DNA, 58 bp, Colobus angolensis
ggcggaatcagaggttcctggctgactcagtgggtgctttgtaccaggaaaggacaag

SEQ ID NO: 114 - DNA, 83 bp, Papio anubis
tcctggtgcactttttggagaaggtggtggaatcagaggtttctggctgactcggtgggtgctttgtaccaggaaaggacaag

SEQ ID NO: 115 - DNA, 82 bp, Mandrillus leucophaeus
tcctggtgcactttttggagaaggtggcggaatcagaggtttctggctgactcggtgggtgctttgtaccaggaaagacaag

SEQ ID NO: 116 - DNA, 75 bp, Galeopterus variegatus
tcgctgacctgcaggtgaaattggggatttctggctgacttcgtggctacttggaaccatgacaggaggaccaga

SEQ ID NO: 117 - DNA, 87 bp, Balaenoptera acutorostrata
taacagtcgcgtcccttacctgtaggtgaaatcagaggttcgtgactgacctgagtaggtgcttggaaccgagaaagaaggacaaga

SEQ ID NO: 118 - DNA, 86 bp, Myotis lucifugus
agcggtcgggtcgcttccctggaggtgatatcagaggttggtggccaaccggggcaggtgctccgaacccagaaaggacggggacg

SEQ ID NO: 119 - DNA, 88 bp, Mustela putorius furo
gtagcggtcgggtcgcttacctggaggtgaaatcagagctttgaggctgacctcgggaggtgctccgagccaagaaaagaaggacaag

SEQ ID NO: 120 - DNA, 81 bp, Loxodonta africana
cacgggtcgttaatctgcaggtaaaatcagagacttgtggctgacctgggtggctgctttaaacaagaaagaaggacagga

SEQ ID NO: 121 - DNA, 74 bp, Ailuropoda melanoleuca
gtgagatgcagggggaaatcagaggtttgaggctgacctggggagctgctccagaaccaagaaagaaggacaag

SEQ ID NO: 122 - DNA, 81 bp, Cercocebus atys
taacactgaagctacatccattcaggcggaatcagaggtttctggctgactcggtgggtgctttgtaccaggaaagacaag

SEQ ID NO: 123 - DNA, 84 bp, Macaca nemestrina
gcccagaccactgataggacagcaatggcggaatcagaggtttctggctgactcggtgggtgctttgtaccaggaaaggacaag

SEQ ID NO: 124 - DNA, 112 bp, Nomascus leucogenys
ccggaaaacgccgaaagctgatcgtctctcggacttgggtttgcaggttcacaaggcccaggaggcttacggtgggcgcctgcctagcagagggtccttgcggagaagcgca

SEQ ID NO: 125 - DNA, 112 bp, Pongo abelii
ccggaaagtgctgaaggcggatcgtctctcggacttgggtttgcaggttcacaagacccgggaggcttacggtgggcgcctgcatagcagagggtccttgcggagaagcgca

SEQ ID NO: 126 - DNA, 112 bp, Pan troglodytes
ccggaaagcgctgaaggcggatcgtctctcagacttgggtttgcaggttcacaaggcccgggaggcttacggtgggcgcccgcctagcagagggtccttgcggagaagcgca

SEQ ID NO: 127 - DNA, 112 bp, Pan paniscus; identical to SEQ ID NO: 126

SEQ ID NO: 128 - DNA, 89 bp, Callithrix jacchus
gacaggacaacatcagcggtgctacaagacaggtggaatcagagatttctggctgactcggtgggtgctttgaaccaggaaaggacaaa

SEQ ID NO: 129 - DNA, 82 bp, Mus musculus
gtctggattcgccataggggtttctcctagcgcggattgtgtgtttgcgttggcgatgctcatagaagaaaggcgcagagct

SEQ ID NO: 130 - DNA, 51 bp, Homo sapiens
aaagaggatgggaaggactgatccacattcccaccaggaagtttagcagaa

SEQ ID NO: 131 - DNA, 68 bp, Gorilla gorilla
aaagaggtgagttgcacttggcagttatagtacagctgcctgcctatggctcttcttgctttgaggtt

SEQ ID NO: 132 - DNA, 54 bp, Colobus angolensis
aaagaggatgggaaggaggactgatccacgttcccaccaggaagcttagtggaa

SEQ ID NO: 133 - DNA, 54 bp, Papio anubis; identical to SEQ ID NO: 132

SEQ ID NO: 134 - DNA, 54 bp, Mandrillus leucophaeus; identical to SEQ ID NO: 132

SEQ ID NO: 135 - DNA, 75 bp, Galeopterus variegatus
aaaaaggcctggaaaagaggacagataaggactgctcttcctcacccacacttctttccaggaagcttagtggag

SEQ ID NO: 136 - DNA, 60 bp, Balaenoptera acutorostrata
taaaaggtccacacagtaagccaggtactctgaatgcaaagaagaaacacctggccgcac

SEQ ID NO: 137 - DNA, 62 bp, Myotis lucifugus
aaggtggcccggagcacgccaggcctgggaagcagggctggcaaagggccgcccttcctcac

SEQ ID NO: 138 - DNA, 47 bp, Mustela putorius furo
acagaaggcccggcaagggaaagcggctaaggaccgctctgtctcgc

SEQ ID NO: 139 - DNA, 71 bp, Loxodonta africana
gaaaagacctggaaggatcgactgagggatgctctttcccagcctcatctcccacctgggagcttgctgaa

SEQ ID NO: 140 - DNA, 44 bp, Ailuropoda melanoleuca
aaagaaggcctggaaaggagcagctaagggctcctcttcctcac

SEQ ID NO: 141 - DNA, 54 bp, Cercocebus atys; identical to SEQ ID NO: 132

SEQ ID NO: 142 - DNA, 54 bp, Macaca nemestrina; identical to SEQ ID NO: 132

SEQ ID NO: 143 - DNA, 45 bp, Nomascus leucogenys
gaacggcaaatgccagcagcagcagccgcatcctctggatacctg

SEQ ID NO: 144 - DNA, 60 bp, Pongo abelii
gaacgacaaatgccagcagcggtgaccgcgtcctccggacaccagcgccgcaacgcgcac

SEQ ID NO: 145 - DNA, 60 bp, Pan troglodytes
gaacgacaaatgccagaggcggtgaccgcgtcctccggataccagccccgcaccgcgcac

SEQ ID NO: 146 - DNA, 60 bp, Pan paniscus
gaacgacaaatgccagaggcagtgaccgcgtcctccggataccagcgccgcaccgcgcac

SEQ ID NO: 147 - DNA, 74 bp, Callithrix jacchus
aaagaggatgggaaggaggactgatcaagggctgctctccctcatccacattcccaccaggaagcttggcggaa

SEQ ID NO: 148 - DNA, 68 bp, Mus musculus
gtacgaggaggcgggatgcacgggtcagagccctcggtggcctccgcccgctgctcttggcgccggtc

SEQ ID NO: 149 - RNA, 78 nt, Homo sapiens
gcagguaugugaugacaucagccgacgccuggcacugcugcaggaacagugggcuggaggaaaguugucaauaccugu